SEQUENCE LISTING (50 sequences)

SEQ ID NO: 1 - DNA, 1335 bp, Arabidopsis thaliana
  source 1..1335: mol_type = other DNA
atgggccaccaaaacgccgccgtttcagagaatcaaaaccatgatgacggcgctgcgtcgtcgccgggattcaagctcgtcggattttccaagttcgtaagaaagaatccaaagtctgataaattcaaggttaagcgcttccatcacatcgagttctggtgcggcgacgcaaccaacgtcgctcgtcgcttctcctggggtctggggatgagattctccgccaaatccgatctttccaccggaaacatggttcacgcctcttacctactcacctccggtgacctccgattccttttcactgctccttactctccgtctctctccgccggagagattaaaccgacaaccacagcttctatcccaagtttcgatcacggctcttgtcgttccttcttctcttcacatggtctcggtgttagagccgttgcgattgaagtagaagacgcagagtcagctttctccatcagtgtagctaatggcgctattccttcgtcgcctcctatcgtcctcaatgaagcagttacgatcgctgaggttaaactatacggcgatgttgttctccgatatgttagttacaaagcagaagataccgaaaaatccgaattcttgccagggttcgagcgtgtagaggatgcgtcgtcgttcccattggattatggtatccggcggcttgaccacgccgtgggaaacgttcctgagcttggtccggctttaacttatgtagcggggttcactggttttcaccaattcgcagagttcacagcagacgacgttggaaccgccgagagcggtttaaattcagcggtcctggctagcaatgatgaaatggttcttctaccgattaacgagccagtgcacggaacaaagaggaagagtcagattcagacgtatttggaacataacgaaggcgcagggctacaacatctggctctgatgagtgaagacatattcaggaccctgagagagatgaggaagaggagcagtattggaggattcgacttcatgccttctcctccgcctacttactaccagaatctcaagaaacgggtcggcgacgtgctcagcgatgatcagatcaaggagtgtgaggaattagggattcttgtagacagagatgatcaagggacgttgcttcaaatcttcacaaaaccactaggtgacaggccgacgatatttatagagataatccagagagtaggatgcatgatgaaagatgaggaagggaaggcttaccagagtggaggatgtggtggttttggcaaaggcaatttctctgagctcttcaagtccattgaagaatacgaaaagactcttgaagccaaacagttagtggga

SEQ ID NO: 2 - protein, 445 aa, Arabidopsis thaliana
MGHQNAAVSENQNHDDGAASSPGFKLVGFSKFVRKNPKSDKFKVKRFHHIEFWCGDATNVARRFSWGLGMRFSAKSDLSTGNMVHASYLLTSGDLRFLFTAPYSPSLSAGEIKPTTTASIPSFDHGSCRSFFSSHGLGVRAVAIEVEDAESAFSISVANGAIPSSPPIVLNEAVTIAEVKLYGDVVLRYVSYKAEDTEKSEFLPGFERVEDASSFPLDYGIRRLDHAVGNVPELGPALTYVAGFTGFHQFAEFTADDVGTAESGLNSAVLASNDEMVLLPINEPVHGTKRKSQIQTYLEHNEGAGLQHLALMSEDIFRTLREMRKRSSIGGFDFMPSPPPTYYQNLKKRVGDVLSDDQIKECEELGILVDRDDQGTLLQIFTKPLGDRPTIFIEIIQRVGCMMKDEEGKAYQSGGCGGFGKGNFSELFKSIEEYEKTLEAKQLVG

SEQ ID NO: 3 - DNA, 1341 bp, Oryza sativa
  source 1..1341: mol_type = other DNA
atgcctcccactcccacccccaccgccaccaccggcgccgtctcggccgctgcggcggcgggggagaacgcggggttccgcctcgtcgggcaccgccgcttcgtccgcgccaacccgcggagcgaccggttccaggcgctcgcgttccaccacgtcgagctctggtgcgccgacgccgcgtccgccgcgggccggttcgccttcgccctgggcgcgccgctcgccgccaggtccgacctctccacggggaactccgcgcacgcctccctcctcctccgctccgcctccgtcgcgttcctcttcaccgccccctacggcggcgaccacggcgtcggcgcggacgcggccaccaccgcctccatcccttccttctccccaggcgccgcgcggaggttcgccgcggaccacggcctcgcggtgcacgccgtggcgctgcgcgtcgccgacgcggccgacgccttccgcgccagcgtcgcggccggtgcgcgcccggcgttccagcccgccgacctcggcggtggcttcggcctcgcggaggtggagctctacggcgacgtcgtgctccgcttcgtcagccacccggacggcgccgacgcgcccttcctcccgggtttcgagggcgtcagcaacccgggcgccgtggactacggcctccgccggttcgaccacgtcgtcggcaacgtgccggagctcgctccggtagccgcgtacatctccgggttcaccgggttccacgagttcgccgagttcaccgccgaggacgtgggcaccgccgagagcggcctcaactcggtggtgctcgccaacaacgcggagaccgtgctgctgccgctcaacgagccggtgcacggcaccaagcggcggagccagatacagacgtacctggaccaccacggcggcccgggggtgcagcacatcgcgctggccagcgacgacgtgctcgggacgctgagggagatgcgggcgcgctccgccatgggcggcttcgagttcttggcgccgccgccgcccaactactacgacggcgtgcggcggcgcgccggggacgtgctctcggaggagcagatcaacgagtgccaggagctcggggtgctcgtggacagggatgaccagggggtgttgctccagatcttcaccaagccagtaggagacaggccaacctttttcttggagatgatacaaaggattgggtgcatggagaaggatgagagtgggcaggagtaccagaagggcggctgcggcgggtttgggaagggcaacttctcggagctgttcaagtccattgaggagtatgagaaatcccttgaagccaagcaagcccctacagttcaaggatcctag

SEQ ID NO: 4 - protein, 446 aa, Oryza sativa
MPPTPTPTATTGAVSAAAAAGENAGFRLVGHRRFVRANPRSDRFQALAFHHVELWCADAASAAGRFAFALGAPLAARSDLSTGNSAHASLLLRSASVAFLFTAPYGGDHGVGADAATTASIPSFSPGAARRFAADHGLAVHAVALRVADAADAFRASVAAGARPAFQPADLGGGFGLAEVELYGDVVLRFVSHPDGADAPFLPGFEGVSNPGAVDYGLRRFDHVVGNVPELAPVAAYISGFTGFHEFAEFTAEDVGTAESGLNSVVLANNAETVLLPLNEPVHGTKRRSQIQTYLDHHGGPGVQHIALASDDVLGTLREMRARSAMGGFEFLAPPPPNYYDGVRRRAGDVLSEEQINECQELGVLVDRDDQGVLLQIFTKPVGDRPTFFLEMIQRIGCMEKDESGQEYQKGGCGGFGKGNFSELFKSIEEYEKSLEAKQAPTVQGS

SEQ ID NO: 5 - DNA, 1281 bp, Zea mays
  source 1..1281: mol_type = other DNA
atgcccccgacccccacagccgccgcagccggcgccgccgtggcggcggcatcagcagcggagcaggcggcgttccgcctcgtgggccaccgcaacttcgtccgcttcaacccgcgctccgaccgcttccacacgctcgcgttccaccacgtggagctctggtgcgccgacgcggcctccgccgcgggccgcttctccttcggcctgggcgcgccgctcgccgcacgctccgacctctccacgggcaactccgcgcacgcgtccctgctgctccgctccggctccctctccttcctcttcacggcgccctacgcgcacggcgccgacgctgccaccgccgcgctgccctccttctccgccgccgccgcgcggcgcttcgcagccgaccacggcctcgcggtgcgcgccgtcgcgctccgcgtcgccgacgccgaggacgccttccgcggcttccgcctcgccgaggtcgagctctacggcgacgtcgtgctccggtacgtgagctacccggacggcgccgcgggcgagcccttcctgccggggttcgagggcgtggccagccccggggcggccgactacgggctgagcaggttcgaccacatcgtcggcaacgtgccggagctggcgcccgccgccgcctacttcgccggcttcacggggttccacgagttcgccgagttcacgacggaggacgtgggcaccgcggagagcggcctcaactccatggtgctcgccaacaactcggagaacgtgctgctcccgctcaacgagccggtgcacggcaccaagcgccgcagccagatacaaacgttcctggaccaccacggcggccccggcgtgcagcacatggcgctggccagcgacgacgtgctcaggacgctgagggagatgcaggcgcgctcggccatgggcggcttcgagttcatggcgcctcccacatccgactactatgacggcgtgaggcggcgcgccggggacgtgctcacggaagcacagattaaggagtgccaggagctaggggtgctggtggacagggatgaccagggcgtgctgctccaaatcttcaccaagccagtgggggacaggccaacgctgttcttggaaatcatccaaaggatcgggtgcatggagaaggatgagaaggggcaagaataccaaaagggtggctgcggcgggttcggcaagggaaacttctcgcagctgttcaagtccatcgaggattatgagaagtcccttgaagccaagcaagctgctgcagcagctgcagctcagggatcctag

SEQ ID NO: 6 - protein, 426 aa, Zea mays
MPPTPTAAAAGAAVAAASAAEQAAFRLVGHRNFVRFNPRSDRFHTLAFHHVELWCADAASAAGRFSFGLGAPLAARSDLSTGNSAHASLLLRSGSLSFLFTAPYAHGADAATAALPSFSAAAARRFAADHGLAVRAVALRVADAEDAFRGFRLAEVELYGDVVLRYVSYPDGAAGEPFLPGFEGVASPGAADYGLSRFDHIVGNVPELAPAAAYFAGFTGFHEFAEFTTEDVGTAESGLNSMVLANNSENVLLPLNEPVHGTKRRSQIQTFLDHHGGPGVQHMALASDDVLRTLREMQARSAMGGFEFMAPPTSDYYDGVRRRAGDVLTEAQIKECQELGVLVDRDDQGVLLQIFTKPVGDRPTLFLEIIQRIGCMEKDEKGQEYQKGGCGGFGKGNFSQLFKSIEDYEKSLEAKQAAAAAAAQGS

SEQ ID NO: 7 - DNA, 1323 bp, Avena sativa
  source 1..1323: mol_type = other DNA
atgccgcccacccccgccaccgccaccggcgccgccgcggccgccgtgactccagagcacgcggcccggagctttccccgagtggtccgcgtcaacccgcgcagcgaccgcttccccgtgctctccttccaccacgtcgagctctggtgcgccgacgccgcctcagcggccggacgcttctccttcgcgctcggcgcgccgctcgccgcccggtccgacctctccacggggaactccgcgcacgcctccctcctgctccgctcgggcgccctcgccttcctcttcacggcgccctacgcgccgccgccgcaggaggccgccacggccgcagccaccgcctccatcccctccttctccgccgacgccgcgcggacgttcgccgccgcccacggcctcgcggtgcgctccgtcggggtccgcgtcgctgacgccgccgaggccttccgcgtcagcgtagccggcggcgctcgcccggccttcgccccagccgacctcggccatggcttcggcctcgccgaggtcgagctctacggcgacgtcgtgctacgcttcgtcagctacccggacgagacagacctgccattcctgccagggttcgagcgcgtgagcagccccggcgccgtggactacggcctcacgcggttcgaccacgtcgtgggcaacgtcccggagatggccccggtcatagactacatgaaaggcttcttggggttccacgagttcgccgagttcaccgccgaggacgtgggcacgaccgagagcgggctcaactcggtggtgctcgccaacaactccgaggccgtgctgctgccgctcaacgagcccgtgcacggcacaaagcgacggagccagatacagacgtacctggagtatcacggcgggcccggcgtgcagcacatcgcgctcgccagcaacgacgtgctcaggacgctcagggagatgcgggcgcgcacgcccatgggcggcttcgagttcatggcgccaccgcaggcgaaatactatgaaggcgtgcggcgcatcgcaggtgacgtgctctcggaagagcagatcaaggaatgccaggagctgggggtgctagtcgacagggatgatcaaggggtgttgctccaaatcttcaccaagccagtaggggacaggccaacgtttttcctggagatgatccaaagaatcgggtgcatggagaaggacgaggtcgggcaagagtaccagaagggtggctgcggcgggtttggcaagggcaatttctccgagctgttcaagtccattgaggactatgagaaatcccttgaggtcaagcaatctgttgtagctcagaaatcctag

SEQ ID NO: 8 - protein, 440 aa, Avena sativa
MPPTPATATGAAAAAVTPEHAARSFPRVVRVNPRSDRFPVLSFHHVELWCADAASAAGRFSFALGAPLAARSDLSTGNSAHASLLLRSGALAFLFTAPYAPPPQEAATAAATASIPSFSADAARTFAAAHGLAVRSVGVRVADAAEAFRVSVAGGARPAFAPADLGHGFGLAEVELYGDVVLRFVSYPDETDLPFLPGFERVSSPGAVDYGLTRFDHVVGNVPEMAPVIDYMKGFLGFHEFAEFTAEDVGTTESGLNSVVLANNSEAVLLPLNEPVHGTKRRSQIQTYLEYHGGPGVQHIALASNDVLRTLREMRARTPMGGFEFMAPPQAKYYEGVRRIAGDVLSEEQIKECQELGVLVDRDDQGVLLQIFTKPVGDRPTFFLEMIQRIGCMEKDEVGQEYQKGGCGGFGKGNFSELFKSIEDYEKSLEVKQSVVAQKS

SEQ ID NO: 9 - DNA, 1077 bp, Pseudomonas fluorescens
  source 1..1077: mol_type = other DNA
atggcagatctatacgaaaacccaatgggcctgatgggctttgaattcatcgaattcgcgtcgccgacgccgggtaccctggagccgatcttcgagatcatgggcttcaccaaagtcgcgacccaccgttccaagaacgtgcacctgtaccgccagggcgagatcaacctgatcctcaacaacgagcccaacagcatcgcctcctactttgcggccgaacacggcccgtcggtgtgcggcatggcgttccgcgtgaaggactcgcaaaaggcctacaaccgcgccctggaactcggcgcccagccgatccatattgacaccgggccgatggaattgaacctgccggcgatcaagggcatcggcggcgcgccgttgtacctgatcgaccgtttcggcgaaggcagctcgatctacgacatcgacttcgtgtacctcgaaggtgtggagcgcaatccggtcggtgcaggtctcaaagtcatcgaccacctgacccacaacgtctatcgcggccgcatggtctactgggccaacttctacgagaaattgttcaacttccgtgaagcgcgttacttcgatatcaagggcgagtacaccggcctgacttccaaggccatgagtgcgccggacggcatgatccgcatcccgctgaacgaagagtcgtccaagggcgcggggcagatcgaagagttcctgatgcagttcaacggcgaaggcatccagcacgtggcgttcctcaccgacgacctggtcaagacctgggacgcgttgaagaaaatcggcatgcgcttcatgaccgcgccgccagacacttattacgaaatgctcgaaggccgcctgcctgaccacggcgagccggtggatcaactgcaggcacgcggtatcctgctggacggatcttccgtggaaggcgacaaacgcctgctgctgcagatcttctcggaaaccctgatgggcccggtgttcttcgaattcatccagcgcaagggcgacgatgggtttggcgagggcaacttcaaggcgctgttcgagtccatcgaacgtgaccaggtgcgtcgtggtgtattgaccgccgattaa

SEQ ID NO: 10 - protein, 358 aa, Pseudomonas fluorescens
MADLYENPMGLMGFEFIEFASPTPGTLEPIFEIMGFTKVATHRSKNVHLYRQGEINLILNNEPNSIASYFAAEHGPSVCGMAFRVKDSQKAYNRALELGAQPIHIDTGPMELNLPAIKGIGGAPLYLIDRFGEGSSIYDIDFVYLEGVERNPVGAGLKVIDHLTHNVYRGRMVYWANFYEKLFNFREARYFDIKGEYTGLTSKAMSAPDGMIRIPLNEESSKGAGQIEEFLMQFNGEGIQHVAFLTDDLVKTWDALKKIGMRFMTAPPDTYYEMLEGRLPDHGEPVDQLQARGILLDGSSVEGDKRLLLQIFSETLMGPVFFEFIQRKGDDGFGEGNFKALFESIERDQVRRGVLTAD

SEQ ID NO: 11-12 - skipped (2 entries)

SEQ ID NO: 13 - DNA, 1053 bp, Synechococcus sp.
  source 1..1053: mol_type = other DNA
atgaacccgtccattcgaattgtccaagggatccaccacctgcacttctacctttgggatctgccccgttggcgggaacacttttgtcgggtttggggcttccgggtggcaagcgacgccggcaacaccctggagctggagcagggatccctgcgcttgcgcctgtctcagccggcacgggcgggggacgaggtggaccgccatttgcagcggcatgggccgggggtggtggatgtggccttggcggtgggagagcaggagctaccggccttggcggagctgttgcggggccgaggcgcccaactggcgtggatcccggcagcagcggcgctctgcctccacaccccctacgggatccggcattctctgatccctggccccttggatgccgcccctgccgaagcgggcctgttttcccactgggatcacgtggtgttgaacgtggagcagggatccctgcaggcggcagccgactggtatgggcgggtgctgggctggcggcggctgtaccgctacagcatcggcaccgccacctccggcctggaaagcgtggtggtgggggatccggaagcggggatccaatgggccatcaacgagcccacctgtgccgcttcccagattcaggagtttttgcatgcccatggcggcccgggcattcagcacgcggcgctgcacagctcagacattgttgccagcctgcgccggttgcggcaggggggagtggactttttgcaagtggcgccgcagtactacaccagcctggaaagggagctggggttggcgctccgttctgcccttgggcaggccatctcctggcaagacctggtggagcagcagatccttctggatgctaccctgcccgcttctgatggccaggatcgcccccttctgctgcagacctttacccagcccctctttggtcggcccacctttttctttgaagtcattcaacggctaggcggggccacgggctttggcgaggccaattttcaggctttgttcgaggccctggaacggcaacagcgacagcgacaccaggcgctgaccccttag

SEQ ID NO: 14 - protein, 350 aa, Synechococcus sp.
MNPSIRIVQGIHHLHFYLWDLPRWREHFCRVWGFRVASDAGNTLELEQGSLRLRLSQPARAGDEVDRHLQRHGPGVVDVALAVGEQELPALAELLRGRGAQLAWIPAAAALCLHTPYGIRHSLIPGPLDAAPAEAGLFSHWDHVVLNVEQGSLQAAADWYGRVLGWRRLYRYSIGTATSGLESVVVGDPEAGIQWAINEPTCAASQIQEFLHAHGGPGIQHAALHSSDIVASLRRLRQGGVDFLQVAPQYYTSLERELGLALRSALGQAISWQDLVEQQILLDATLPASDGQDRPLLLQTFTQPLFGRPTFFFEVIQRLGGATGFGEANFQALFEALERQQRQRHQALTP

SEQ ID NO: 15 - DNA, 1149 bp, Blepharisma japonicum
  source 1..1149: mol_type = other DNA
atgacttattacgacaagcaagaaacgcgtccagatcttggcgaattctatggtttccatcacgttcgtttttacgtctccaactcagagcaagccgcttcgttctacacatctcgctttgggttttctccggttgcctatgaaggattggaaacaggaaaccaaaaattctgtaccaatgtcgtccgaagcaaccatgtagtcatcgcttttacctcagctctcactcctgaagacaatgaagtgaaccgtcacgttggcaagcatagtgatggagttcaagacattgcctttagtgtaagtgacgcaagagggatgtatgagaaagcgatagctaaaggctgtaaaagcttccgtgagccacaggttttacaagatcaatttggatctgttataatagcgtctctccagacttatggagacactgttcacacattagtccaaaatgtcgactatacaggaccctttttgcctggcttcagagcaatcacaaaagatgatccattaaactctgcctttcctcaggtaaattatgacattattgatcatgttgtaggaaatcagcctggtggcgatatgactcctacagtagaatggtatgagaaatatctagaatttcatcgatattggtctgctgatgagtctgtaatccataccgattattcagcattaaggtctgttgtggttgctgattgggatgaagtgatcaaaatgcctattaatgagcctgctgatggacttagaaaaagtcaaatccaagaatatgtcgaatattatggtggagcaggcgtacaacatattgccttaaaagtcaatgatattatttcagtaataagcaccttaagggctagaggtgtggaattcttagaagttcctcctaaatattatgatagcttaagaaaaagacttgcgcattctgcggtacaaattgaagaagacttaaaaagaattgaagaccttcatattttggttgactttgacgaccgtgggtatttacttcagattttcacaaaaccagtagaagacagacctactctgttttatgaaattattcaaagacataataacaatggattcggaattggaaattttaaagccctatttgaatcattggaacaagagcaagaaagaagaggtaatttgatctaa

SEQ ID NO: 16 - protein, 382 aa, Blepharisma japonicum
MTYYDKQETRPDLGEFYGFHHVRFYVSNSEQAASFYTSRFGFSPVAYEGLETGNQKFCTNVVRSNHVVIAFTSALTPEDNEVNRHVGKHSDGVQDIAFSVSDARGMYEKAIAKGCKSFREPQVLQDQFGSVIIASLQTYGDTVHTLVQNVDYTGPFLPGFRAITKDDPLNSAFPQVNYDIIDHVVGNQPGGDMTPTVEWYEKYLEFHRYWSADESVIHTDYSALRSVVVADWDEVIKMPINEPADGLRKSQIQEYVEYYGGAGVQHIALKVNDIISVISTLRARGVEFLEVPPKYYDSLRKRLAHSAVQIEEDLKRIEDLHILVDFDDRGYLLQIFTKPVEDRPTLFYEIIQRHNNNGFGIGNFKALFESLEQEQERRGNLI

SEQ ID NO: 17 - DNA, 1206 bp, Rhodococcus sp.
  source 1..1206: mol_type = other DNA
atgacgatcgagcagactctcaccgacaaggaacgcctggcaggtctcgacctcggccagctcgagcagttggtcgggctcgtcgagtacgacggcacccgcgacccgttcccggtcagcggctgggatgccgtcgtctgggtggtcggcaacgccacccagaccgcccactacttccagtccgcgttcgggatgaccctcgtcgcctactccggacccaccaccggcaaccgcgaccaccacagcttcgtcctcgaatccggggccgtccgcttcgtcatcaaaggcgccgtgaacccggacagccccctgatcgaccaccaccgcacccacggcgacggcgtcgtcgacatcgccctcgccgtccccgacgtcgacaagtgcatcgcccacgcccgcgcccagggcgccaccgtcctcgacgaaccccacgacgtgaccgacgaccacggcaccgtccgcctcgccgcgatcgccacctacggcgacacccgccacaccctcgtcgaccgcagccactacaccggcccctacctgcccggctacaccgcccgcacctccggccacaccaaacgggacggggcacccaagcgcctgttccaggccctcgaccacgtcgtcggcaacgtcgaactcggcaagatggaccactgggtcgacttctacaaccgggtcatgggctttacgaacatggccgagttcgtcggcgaggacatcgccaccgactactccgcgctgatgagcaaggtcgtctccaacggcaaccaccgggtcaagttccccctcaacgaacccgccctcgccaagaaacgctcgcagatcgacgaatacctcgacttctaccgcggccccggcgcccagcacctggccctggccaccaatgacatcctcaccgccgtcgaccagctgaccgccgagggcgtcgagttcctggccacccccgactcctactacgaggaccccgaactgcgggcccggatcggcaacgtccgcgcccccatcgccgaactgcagaaacgcggcatcctcgtcgaccgcgacgaagacggctacctgctgcagatcttcaccaaacccctcgtcgaccggcccaccgtgttcttcgaactcatcgaacgccacggctccctcggcttcggcatcggcaacttcaaagccctcttcgaggccatcgaacgcgaacaagccgcccgcggaaacttctga

SEQ ID NO: 18 - protein, 401 aa, Rhodococcus sp.
MTIEQTLTDKERLAGLDLGQLEQLVGLVEYDGTRDPFPVSGWDAVVWVVGNATQTAHYFQSAFGMTLVAYSGPTTGNRDHHSFVLESGAVRFVIKGAVNPDSPLIDHHRTHGDGVVDIALAVPDVDKCIAHARAQGATVLDEPHDVTDDHGTVRLAAIATYGDTRHTLVDRSHYTGPYLPGYTARTSGHTKRDGAPKRLFQALDHVVGNVELGKMDHWVDFYNRVMGFTNMAEFVGEDIATDYSALMSKVVSNGNHRVKFPLNEPALAKKRSQIDEYLDFYRGPGAQHLALATNDILTAVDQLTAEGVEFLATPDSYYEDPELRARIGNVRAPIAELQKRGILVDRDEDGYLLQIFTKPLVDRPTVFFELIERHGSLGFGIGNFKALFEAIEREQAARGNF

SEQ ID NO: 19 - DNA, 1107 bp, Picrophilus torridus
  source 1..1107: mol_type = other DNA
atgtatggcaaaaatttaatctcagaactaagggaaaaggagatctttaaacgattacatcacgtggaattttacgttagcagtgccaaaacatggtcatatttcatgaacaggggtcttggatttaaaacagtggcatatgccggtccagaaaccgggataagggacaagatatcctatgttatgtcccagggcactgcaaggatatcttttacatcatcaatgaatgatgatagctatatatcgaatcatgttaaaaaacacggggatggcgtaaaggatatagcacttgaggtcgatgatctggacgaggcaaaaagcctgatagaaaagtatggaacaaaggtttcaaaaataaatgaaataaaggatggaaatggaaagataagaactgcagagataaaaacgtacggtgaaaccgttcatacattaatagaaaccggggattacaatggcgtattcatgcccggttatgaggaatctgaaataaattcaaaaaacactgggataaaaaagatcgatcatatagttggaaatgtctatgagggcgagatggatagctgggttaatttttacatagaaaaacttggctttgagcatttaataacctttgatgataaagatataagaactgattacagcgcattaagatcaaaggttgtaaaatacaatgacgatatcgtatttccaataaatgagcctgcaaagggcttaagaaaatcacagatagaggaatatcttgactattacaggtctgagggcgttcagcacatagcactgttaactgatgatataataaaaactgtatccatgatggaggaaaacggcatagaatttttaaaaacaccaggatcatactatgaatccctatcatcaaggataggctcaatagacgaggatttaaatgaaatagagaaacataacatacttgtggatcgtgatgagaacggatacctattacagatcttcacaaagcctgttactgacaggccaacgttcttctttgaggtcatacagagaaagggtgcaaggtcattcggcaacggtaactttaaggcactttttgaggcgatagaaagggagcaggcaaagagaggaaacctatga

SEQ ID NO: 20 - protein, 368 aa, Picrophilus torridus
MYGKNLISELREKEIFKRLHHVEFYVSSAKTWSYFMNRGLGFKTVAYAGPETGIRDKISYVMSQGTARISFTSSMNDDSYISNHVKKHGDGVKDIALEVDDLDEAKSLIEKYGTKVSKINEIKDGNGKIRTAEIKTYGETVHTLIETGDYNGVFMPGYEESEINSKNTGIKKIDHIVGNVYEGEMDSWVNFYIEKLGFEHLITFDDKDIRTDYSALRSKVVKYNDDIVFPINEPAKGLRKSQIEEYLDYYRSEGVQHIALLTDDIIKTVSMMEENGIEFLKTPGSYYESLSSRIGSIDEDLNEIEKHNILVDRDENGYLLQIFTKPVTDRPTFFFEVIQRKGARSFGNGNFKALFEAIEREQAKRGNL

SEQ ID NO: 21 - DNA, 1164 bp, Kordia algicida
  source 1..1164: mol_type = other DNA
atggcagcagaaataaaaaacttaaaagatttacaaaatacagaatacggactcaaaaaattatttgacgaagcagaagactttcttccacttttaggaacagactacgtagaattatacgtcgggaacgccaaacaatcggcacatttctacaaaacggcttttggttttcaatcagaagcttacgcaggattggaaacaggattaaccgacagagtttcatacgtattaaaacaagataaaattcgcttggtcttaacaacaccattaggaaaaggtggcgaaatcaatgagcatatcgatttacacggcgatggcgtaaaagtagtagcactttgggtagaagatgctacaaaagcctttgaagaaacgaccaaaagaggcgcaaaaccgtacatggaaccaacaaaagaagaagatgaaaacggatatgtaattcgctcaggaatctatacgtacggagaaacggttcatgtttttgtagaacgtaaaaactataacggagtctttttaccaggatatcaaagatgggaatctcactacaatccggagccagttggcttaaaattcatcgatcacatggtaggaaatgtaggttggggagaaatgaaagaatggtgtgaattctacgcgaaagtaatgggatttgcgcaaattatctcctttacagatgatgatatttctaccgattttactgcgttgatgagtaaagtaatgagtaatggaaatggtagaatcaaatttccaatcaatgaacccgcagaaggaaaaaagaaatcgcaaattgaagaatatctagacttttacaatggttcaggagtacaacatattgcggttgctacagacaatattattgatacggtttcgcaaatgcgcgaacgtggagtagaattcttatacgttccagatacatattatgatgacttgttagaacgtgttggcgacatcgatgaagatgtagaagaactcaaaaaacacggaatcttaattgatcgtgatgaagaaggatacttattgcagttatttaccaaaaccattgtagacagaccaacaatgttctttgaagtcattcagcgtaaaggcgcacaatcatttggagtaggaaactttaaagctttatttgaagcgatagaaagagaacaagctgctcgcggaacattgtaa

SEQ ID NO: 22 - protein, 387 aa, Kordia algicida
MAAEIKNLKDLQNTEYGLKKLFDEAEDFLPLLGTDYVELYVGNAKQSAHFYKTAFGFQSEAYAGLETGLTDRVSYVLKQDKIRLVLTTPLGKGGEINEHIDLHGDGVKVVALWVEDATKAFEETTKRGAKPYMEPTKEEDENGYVIRSGIYTYGETVHVFVERKNYNGVFLPGYQRWESHYNPEPVGLKFIDHMVGNVGWGEMKEWCEFYAKVMGFAQIISFTDDDISTDFTALMSKVMSNGNGRIKFPINEPAEGKKKSQIEEYLDFYNGSGVQHIAVATDNIIDTVSQMRERGVEFLYVPDTYYDDLLERVGDIDEDVEELKKHGILIDRDEEGYLLQLFTKTIVDRPTMFFEVIQRKGAQSFGVGNFKALFEAIEREQAARGTL

SEQ ID NO: 23 - DNA, 1323 bp, Sorghum bicolor
  source 1..1323: mol_type = other DNA
atgcccccgacccccaccacagccgccgcaaccggcgccgccgtggcggcggcatcagcggagcaggcggcgttccgcctcgtgggccaccgcaacttcgtccgcgtgaacccgcgctccgaccgcttccacacgctcgcgttccaccacgtggagctctggtgcgccgacgcggcctccgccgcgggccgcttctccttcgggctcggcgcgccgctcgccgcgcggtccgacctctccacggggaacaccgcgcacgcgtccctgctgctccgctcgggcgccctcgcgttcctcttcacggcgccctacgcgcacggcgccgacgccgccacggcctcgctgccctccttctccgccgccgaggcgcggcgcttcgcggccgaccacggcctcgcggtgcgcgccgtcgcgctccgcgtggccgacgcggaggacgccttccgcgccagcgtcgcggccggcgcgcgcccggcgttcgagcccgtcgagctcggcctcggcttccgcctcgccgaagtcgagctctacggcgacgtcgtgctccggtacgtgagctacccggacgacgcggacgcgtccttcctgccggggttcgtgggcgtgaccagccccggcgcggcggactacgggctgaggaggttcgaccacatcgtcggcaacgtgccggagctggcgccggcggccgcctacttcgctggcttcacggggttccacgagttcgccgagttcacggcggaggacgtgggcaccacggagagcgggctcaactcgatggtgctcgccaacaacgcggagaacgtgctgctcccactcaacgagccggtgcacggcaccaagcgccgcagccagatacagacgtacttggaccaccacggcggccccggcgtgcagcacatggcgctggccagcgacgacgtgctcaggacgctgagggagatgcaagcgcgctcggccatgggcggcttcgagttcatggcgcctccggcgcccgaatactatgacggcgtgaggcggcgcgccggggacgtgctcacggaggcacagattaaggagtgtcaggaactaggggtgctggtggacagagatgaccagggcgtgctgctccagatcttcaccaagccagtgggggacaggccaacgttgttcttggagatcattcaaaggatcgggtgcatggagaaggatgagaaggggcaagaataccagaagggtggctgtggcgggtttggcaagggaaacttctcccagctgttcaaatccattgaggattatgagaagtcccttgaagctaagcaagctgcagcagctcagggatcctag

SEQ ID NO: 24 - protein, 440 aa, Sorghum bicolor
MPPTPTTAAATGAAVAAASAEQAAFRLVGHRNFVRVNPRSDRFHTLAFHHVELWCADAASAAGRFSFGLGAPLAARSDLSTGNTAHASLLLRSGALAFLFTAPYAHGADAATASLPSFSAAEARRFAADHGLAVRAVALRVADAEDAFRASVAAGARPAFEPVELGLGFRLAEVELYGDVVLRYVSYPDDADASFLPGFVGVTSPGAADYGLRRFDHIVGNVPELAPAAAYFAGFTGFHEFAEFTAEDVGTTESGLNSMVLANNAENVLLPLNEPVHGTKRRSQIQTYLDHHGGPGVQHMALASDDVLRTLREMQARSAMGGFEFMAPPAPEYYDGVRRRAGDVLTEAQIKECQELGVLVDRDDQGVLLQIFTKPVGDRPTLFLEIIQRIGCMEKDEKGQEYQKGGCGGFGKGNFSQLFKSIEDYEKSLEAKQAAAAQGS

SEQ ID NO: 25 - DNA, 1311 bp, Triticum aestivum
  source 1..1311: mol_type = other DNA
atgccgcccacccccaccacccccgcagccaccggcgccggcgctgccgccgcggtgacgccggagcacgcgcggccgcgccgaatggtccgcttcaacccgcgcagcgaccgcttccacacgctctccttccaccacgtcgagttctggtgcgcggacgccgcctccgccgccggccgcttcgccttcgcgctcggcgcgccgctcgccgccaggtccgacctctccacggggaactccgtgcacgcctcccagctgctccgctcgggcaacctcgccttcctcttcaccgcgccctacgcgaacggctgcgacgccgccaccgcctccctgccctccttctccgccgacgccgcgcgccggttctccgcggaccacgggctcgcagtgcgctccatagcactgcgcgtcgcagacgccgcagaggccttccgcgccagcgtcgacggaggcgcgcgcccggccttcagccccgtggacctcggccgcggcttcggcttcgcggaggtcgagctctacggcgacgtcgtgctccgcttcgtcagtcacccggatgacacggacgtgcccttcttgccggggttcgagggcgtgagcaacccggatgccgtggactacggcctgacgcggttcgaccacgtcgtcggcaacgtcccggagcttgcccccgccgccgcatacgtcgccgggttcgcggggttccacgagttcgccgagttcacgacggaggacgtgggcacggccgagagcgggctcaactcgatggtgctcgccaacaactcggagggcgtgctgctgccgctcaacgagccggtgcacggcaccaagcgccggagccagatacagacgttcctggaacaccacggcggctcgggcgtgcagcacatcgcggtggccagcagcgacgtgctcaggacgctcagggagatgcgtgcgcgctccgccatgggcggcttcgacttcctgccaccccgctgccgaaagtactacgaaggcgtgcggcgcatcgccggggatgtgctctcggaggcgcagatcaaggaatgccaggagctgggggtgctcgtcgacagggacgaccaaggggtgttgctacaaatcttcacaaagccagtgggggacaggccaacgctgttcctggagatgatccaaaggatcgggtgcatggagaaggacgagagaggggaagagtaccagaagggtggctgcggcgggttcggcaaaggcaacttctccgagctgttcaagtccattgaagattacgagaagtcccttgaagccaagcaatctgctgcagttcagggatcatag

SEQ ID NO: 26 - protein, 436 aa, Triticum aestivum
MPPTPTTPAATGAGAAAAVTPEHARPRRMVRFNPRSDRFHTLSFHHVEFWCADAASAAGRFAFALGAPLAARSDLSTGNSVHASQLLRSGNLAFLFTAPYANGCDAATASLPSFSADAARRFSADHGLAVRSIALRVADAAEAFRASVDGGARPAFSPVDLGRGFGFAEVELYGDVVLRFVSHPDDTDVPFLPGFEGVSNPDAVDYGLTRFDHVVGNVPELAPAAAYVAGFAGFHEFAEFTTEDVGTAESGLNSMVLANNSEGVLLPLNEPVHGTKRRSQIQTFLEHHGGSGVQHIAVASSDVLRTLREMRARSAMGGFDFLPPRCRKYYEGVRRIAGDVLSEAQIKECQELGVLVDRDDQGVLLQIFTKPVGDRPTLFLEMIQRIGCMEKDERGEEYQKGGCGGFGKGNFSELFKSIEDYEKSLEAKQSAAVQGS

SEQ ID NO: 27 - DNA, 1305 bp, Hordeum vulgare
  source 1..1305: mol_type = other DNA
atgccgcccacccccaccacccccgcggctaccggcgccgccgccgcggtgacgccggagcacgcgcgaccgcaccgaatggtccgcttcaacccgcgcagcgaccgcttccacacgctctccttccaccacgtcgagttctggtgcgcggacgccgcctccgccgccggccgcttcgcgttcgcgctcggcgcgccgctcgccgccaggtccgacctctccacggggaactccgcgcacgcctcccagctgctccgctcgggctccctcgccttcctcttcaccgcgccctacgccaacggctgcgacgccgccaccgcctccctgccctccttctccgccgacgccgcgcgccggttctccgccgaccacgggatcgcggtgcgctccgtagcgctgcgcgtcgcagacgccgccgaggccttccgcgccagtcgtcgacggggcgcgcgcccggccttcgcccccgtggacctcggccgcggcttcgcgttcgcggaggtcgagctctacggcgacgtcgtgctccgcttcgtcagccacccggacggcacggacgtgcccttcttgccggggttcgagggcgtaaccaacccggacgccgtggactacggcctgacgcggttcgaccacgtcgtcggcaacgtcccggagcttgcccccgccgcagcctacatcgccgggttcacggggttccacgagttcgccgagttcacggcggaggacgtgggcacgaccgagagcgggctcaactcggtggtgctcgccaacaactcggagggcgtgctgctgccgctcaacgagccggtgcacggcaccaagcgccggagccagatacagacgttcctggaacaccacggcggcccgggcgtgcagcacatcgcggtggccagcagtgacgtgctcaggacgctcaggaagatgcgtgcgcgctccgccatgggcggcttcgacttcctgccacccccgctgccgaagtactacgaaggcgtgcgacgccttgccggggatgtcctctcggaggcgcagatcaaggaatgccaggagctgggtgtgctcgtcgatagggacgaccaaggggtgttgctccaaatcttcaccaagccagtaggggacaggccgaccttgttcctggagatgatccagaggatcgggtgcatggagaaggacgagagaggggaagagtaccagaagggtggctgcggcgggttcggcaaaggcaacttctccgagctgttcaagtccattgaagattacgagaagtcccttgaagccaagcaatctgctgcagttcagggatcatag

SEQ ID NO: 28 - protein, 434 aa, Hordeum vulgare
MPPTPTTPAATGAAAAVTPEHARPHRMVRFNPRSDRFHTLSFHHVEFWCADAASAAGRFAFALGAPLAARSDLSTGNSAHASQLLRSGSLAFLFTAPYANGCDAATASLPSFSADAARRFSADHGIAVRSVALRVADAAEAFRASRRRGARPAFAPVDLGRGFAFAEVELYGDVVLRFVSHPDGTDVPFLPGFEGVTNPDAVDYGLTRFDHVVGNVPELAPAAAYIAGFTGFHEFAEFTAEDVGTTESGLNSVVLANNSEGVLLPLNEPVHGTKRRSQIQTFLEHHGGPGVQHIAVASSDVLRTLRKMRARSAMGGFDFLPPPLPKYYEGVRRLAGDVLSEAQIKECQELGVLVDRDDQGVLLQIFTKPVGDRPTLFLEMIQRIGCMEKDERGEEYQKGGCGGFGKGNFSELFKSIEDYEKSLEAKQSAAVQGS

SEQ ID NO: 29 - DNA, 1209 bp, Rhodococcus sp.
  source 1..1209: mol_type = other DNA
atgactaccgccgacattcgcctgacgccccgcgaggtggccgcacatctggagaccgacgagctccggcagttggtcgggctcgtcgaacacgacgacgcgtcggatccgtttcccgtggtcgcgatggatgccgtggtgttcgtgtgcggcaacgcgacgcagagcacgcagtacttcgtctccacgtggggcatgaccctcgtcgcctacgccgggccggagaccggtcagcgctcgcacaagtccttcgtcctcgagtcggggtcggcacggttcgtgctgcacggcgccgtcgatccgaagagcccgctcgcggaccatcaccgggcgcacggcgacggcgtggtggacctggcgatggaagttctcgacgtcgaccgctgcatcgcgcatgcacgctcgcagggggccaccattctcgaggagccgcgcgacgtcacggatcagttcggcaccgtgcggctcgcggcgatcgccacgtacggcagcacccggcacaccatcgtcgaccgaagccgatacgacggcccctacctccccggattcgtcgcgcgctccagcggtttcgcggcgcgaccgggtaaacccccgcgattgttccaggcgctcgaccacgccgtcggcaacgtcgagatgggccggatggatcactgggtccggttctacaaccgcgtcatgggcttcacgaacatggccgaattcgtcggcgacgacatcgccacggagtactcggcgctgatgtcgaaggtcgtggcgaacggcaatcaccgggtgaagttcccgctcaacgaacccgcggtgggaaagaagaagtcgcagatcgacgaatatctcgagttctacggtgagccgggctgccagcatctggccctcgcgaccggagacatcctcgcgacggtggacgcgttgcgggccgagggtgtcgaattcctgaacacacccgacgcgtactacgaggacccacagctgcgcgcccggatcggcagggtgcgggtgccggtggaggaactgcagaagcgcggaatcctcgtcgaccgcgacgaggacggatacctcctgcagatcttcaccaaaccgctcggcgaccggccgaccgtgttcttcgaggtgatcgaacggcacggttcgctcgggttcggggcgggtaacttccaggccctgttcgaatccatcgagcgtgagcaggcggcgcgcggcaatctgtga

SEQ ID NO: 30 - protein, 402 aa, Rhodococcus sp.
MTTADIRLTPREVAAHLETDELRQLVGLVEHDDASDPFPVVAMDAVVFVCGNATQSTQYFVSTWGMTLVAYAGPETGQRSHKSFVLESGSARFVLHGAVDPKSPLADHHRAHGDGVVDLAMEVLDVDRCIAHARSQGATILEEPRDVTDQFGTVRLAAIATYGSTRHTIVDRSRYDGPYLPGFVARSSGFAARPGKPPRLFQALDHAVGNVEMGRMDHWVRFYNRVMGFTNMAEFVGDDIATEYSALMSKVVANGNHRVKFPLNEPAVGKKKSQIDEYLEFYGEPGCQHLALATGDILATVDALRAEGVEFLNTPDAYYEDPQLRARIGRVRVPVEELQKRGILVDRDEDGYLLQIFTKPLGDRPTVFFEVIERHGSLGFGAGNFQALFESIEREQAARGNL

SEQ ID NO: 31 - DNA, 1353 bp, synthetic construct
  misc_feature 1..1353: note = Nucleic acid sequence encoding Arabidopsis HPPD plus containingat the 5' end a nucleic acid encoding an Ala and 6 His aminoacids
  misc_feature 4..6: note = sequence coding for an Ala
  misc_feature 7..24: note = sequence coding for an His Tag containing 6 His
  source 1..1353: mol_type = other DNA
atggctcatcaccatcaccatcaccaaaacgccgccgtttcagagaatcaaaaccatgatgacggcgctgcgtcgtcgccgggattcaagctcgtcggattttccaagttcgtaagaaagaatccaaagtctgataaattcaaggttaagcgcttccatcacatcgagttctggtgcggcgacgcaaccaacgtcgctcgtcgcttctcctggggtctggggatgagattctccgccaaatccgatctttccaccggaaacatggttcacgcctcttacctactcacctccggtgacctccgattccttttcactgctccttactctccgtctctctccgccggagagattaaaccgacaaccacagcttctatcccaagtttcgatcacggctcttgtcgttccttcttctcgtcacatggtctcggtgttagagccgttgcgattgaagtagaagacgcagagtcagctttctccatcagtgtagctaatggcgctattccttcgtcgcctcctatcgtcctcaatgaagcagttacgatcgctgaggttaaactatacggcgatgttgttctccgatatgttagttacaaagcagaagataccgaaaaatccgaattcttgccagggttcgagcgtgtagaggatgcgtcgtcgttcccattggattatggtatccggcggcttgaccacgccgtgggaaacgttcctgagcttggtccggctttaacttatgtagcggggttcactggttttcaccaattcgcagagttcacagcagacgacgttggaaccgccgagagcggtttaaattcagcggtcctggctagcaatgatgaaatggttcttctaccgattaacgagccagtgcacggaacaaagaggaagagtcagattcagacgtatttggaacataacgaaggcgcagggctacaacatctggctctgatgagtgaagacatattcaggaccctgagagagatgaggaagaggagcagtattggaggattcgacttcatgccttctcctccgcctacttactaccagaatctcaagaaacgggtcggcgacgtgctcagcgatgatcagatcaaggagtgtgaggaattagggattcttgtagacagagatgatcaagggacgttgcttcaaatcttcacaaaaccactaggtgacaggccgacgatatttatagagataatccagagagtaggatgcatgatgaaagatgaggaagggaaggcttaccagagtggaggatgtggtggttttggcaaaggcaatttctctgagctcttcaagtccattgaagaatacgaaaagactcttgaagccaaacagttagtgggatga

SEQ ID NO: 32 - protein, 450 aa, synthetic construct
  REGION 1..450: note = encoded by SEQ ID No. 31
  SITE 2: note = MISC_FEATURE - Ala
  REGION 3..8: note = MISC_FEATURE - His Tag made of 6 His
MAHHHHHHQNAAVSENQNHDDGAASSPGFKLVGFSKFVRKNPKSDKFKVKRFHHIEFWCGDATNVARRFSWGLGMRFSAKSDLSTGNMVHASYLLTSGDLRFLFTAPYSPSLSAGEIKPTTTASIPSFDHGSCRSFFSSHGLGVRAVAIEVEDAESAFSISVANGAIPSSPPIVLNEAVTIAEVKLYGDVVLRYVSYKAEDTEKSEFLPGFERVEDASSFPLDYGIRRLDHAVGNVPELGPALTYVAGFTGFHQFAEFTADDVGTAESGLNSAVLASNDEMVLLPINEPVHGTKRKSQIQTYLEHNEGAGLQHLALMSEDIFRTLREMRKRSSIGGFDFMPSPPPTYYQNLKKRVGDVLSDDQIKECEELGILVDRDDQGTLLQIFTKPLGDRPTIFIEIIQRVGCMMKDEEGKAYQSGGCGGFGKGNFSELFKSIEEYEKTLEAKQLVG

SEQ ID NO: 33 - DNA, 1353 bp, synthetic construct
  misc_feature 1..1353: note = nucleotide sequence encoding mutant HPPD polypeptide
  misc_feature 4..6: note = sequence coding for Ala
  misc_feature 7..24: note = sequence coding an His Tag containing 6 His
  source 1..1353: mol_type = other DNA
atggctcatcaccatcaccatcaccaaaacgccgccgtttcagagaatcaaaaccatgatgacggcgctgcgtcgtcgccgggattcaagctcgtcggattttccaagttcgtaagaaagaatccaaagtctgataaattcaaggttaagcgcttccatcacatcgagttctggtgcggcgacgcaaccaacgtcgctcgtcgcttctcctggggtctggggatgagattctccgccaaatccgatctttccaccggaaacatggttcacgcctcttacctactcacctccggtgacctccgattccttttcactgctccttactctccgtctctctccgccggagagattaaaccgacaaccacagcttctatcccaagtttcgatcacggctcttgtcgttccttcttctcgtcacatggtctcggtgttagagccgttgcgattgaagtagaagacgcagagtcagctttctccatcagtgtagctaatggcgctattccttcgtcgcctcctatcgtcctcaatgaagcagttacgatcgctgaggttaaactatacggcgatgttgttctccgatatgttagttacaaagcagaagataccgaaaaatccgaattcttgccagggttcgagcgtgtagaggatgcgtcgtcgttcccattggattatggtatccggcggcttgaccacgccgtgggaaacgttcctgagcttggtccggctttaacttatgtagcggggttcactggttttcaccaattcgcagagttcacagcagacgacgttggaaccgccgagagcggtttaaatgcggcggtcctggctagcaatgatgaaatggttcttctaccgattaacgagccagtgcacggaacaaagaggaagagtcagattcagacgtatttggaacataacgaaggcgcagggctacaacatctggctctgatgagtgaagacatattcaggaccctgagagagatgaggaagaggagcagtattggaggattcgacttcatgccttctcctccgcctacttactaccagaatctcaagaaacgggtcggcgacgtgctcagcgatgatcagatcaaggagtgtgaggaattagggattcttgtagacagagatgatcaagggacgttgcttcaaatcttcacaaaaccactaggtgacaggccgacgatatttatagagataatccagagagtaggatgcatgatgaaagatgaggaagggaaggcttaccagagtggaggatgtggtggttttggcaaaggcaatttctctgagctcttcaagtccattgaagaatacgaaaagactcttgaagccaaacagttagtgggatga

SEQ ID NO: 34 - protein, 450 aa, synthetic construct
  REGION 1..450: note = encoded by SEQ ID No. 33
  SITE 2: note = MISC_FEATURE - Ala
  REGION 3..8: note = MISC_FEATURE - His Tag made of 6 His
MAHHHHHHQNAAVSENQNHDDGAASSPGFKLVGFSKFVRKNPKSDKFKVKRFHHIEFWCGDATNVARRFSWGLGMRFSAKSDLSTGNMVHASYLLTSGDLRFLFTAPYSPSLSAGEIKPTTTASIPSFDHGSCRSFFSSHGLGVRAVAIEVEDAESAFSISVANGAIPSSPPIVLNEAVTIAEVKLYGDVVLRYVSYKAEDTEKSEFLPGFERVEDASSFPLDYGIRRLDHAVGNVPELGPALTYVAGFTGFHQFAEFTADDVGTAESGLNAAVLASNDEMVLLPINEPVHGTKRKSQIQTYLEHNEGAGLQHLALMSEDIFRTLREMRKRSSIGGFDFMPSPPPTYYQNLKKRVGDVLSDDQIKECEELGILVDRDDQGTLLQIFTKPLGDRPTIFIEIIQRVGCMMKDEEGKAYQSGGCGGFGKGNFSELFKSIEEYEKTLEAKQLVG

SEQ ID NO: 35 - DNA, 1353 bp, synthetic construct
  misc_feature 1..1353: note = nucleotide sequence encoding mutant HPPD polypeptide
  misc_feature 4..6: note = sequence coding for Ala
  misc_feature 7..24: note = sequence coding for an His Tag containing 6 His
  source 1..1353: mol_type = other DNA
atggctcatcaccatcaccatcaccaaaacgccgccgtttcagagaatcaaaaccatgatgacggcgctgcgtcgtcgccgggattcaagctcgtcggattttccaagttcgtaagaaagaatccaaagtctgataaattcaaggttaagcgcttccatcacatcgagttctggtgcggcgacgcaaccaacgtcgctcgtcgcttctcctggggtctggggatgagattctccgccaaatccgatctttccaccggaaacatggttcacgcctcttacctactcacctccggtgacctccgattccttttcactgctccttactctccgtctctctccgccggagagattaaaccgacaaccacagcttctatcccaagtttcgatcacggctcttgtcgttccttcttctcgtcacatggtctcggtgttagagccgttgcgattgaagtagaagacgcagagtcagctttctccatcagtgtagctaatggcgctattccttcgtcgcctcctatcgtcctcaatgaagcagttacgatcgctgaggttaaactatacggcgatgttgttctccgatatgttagttacaaagcagaagataccgaaaaatccgaattcttgccagggttcgagcgtgtagaggatgcgtcgtcgttcccattggattatggtatccggcggcttgaccacgccgtgggaaacgttcctgagcttggtccggctttaacttatgtagcggggttcactggttttcaccaattcgcagagttcacagcagacgacgttggaaccgccgagagcggtttaaattcagcggtcctggctagcaatgatgaaatggttcttctaccgattgccgagccagtgcacggaacaaagaggaagagtcagattcagacgtatttggaacataacgaaggcgcagggctacaacatctggctctgatgagtgaagacatattcaggaccctgagagagatgaggaagaggagcagtattggaggattcgacttcatgccttctcctccgcctacttactaccagaatctcaagaaacgggtcggcgacgtgctcagcgatgatcagatcaaggagtgtgaggaattagggattcttgtagacagagatgatcaagggacgttgcttcaaatcttcacaaaaccactaggtgacaggccgacgatatttatagagataatccagagagtaggatgcatgatgaaagatgaggaagggaaggcttaccagagtggaggatgtggtggttttggcaaaggcaatttctctgagctcttcaagtccattgaagaatacgaaaagactcttgaagccaaacagttagtgggatga

SEQ ID NO: 36 - protein, 450 aa, synthetic construct
  REGION 1..450: note = encoded by SEQ ID No. 35
  SITE 2: note = MISC_FEATURE - Ala
  REGION 3..8: note = MISC_FEATURE - His Tag made of 6 His
MAHHHHHHQNAAVSENQNHDDGAASSPGFKLVGFSKFVRKNPKSDKFKVKRFHHIEFWCGDATNVARRFSWGLGMRFSAKSDLSTGNMVHASYLLTSGDLRFLFTAPYSPSLSAGEIKPTTTASIPSFDHGSCRSFFSSHGLGVRAVAIEVEDAESAFSISVANGAIPSSPPIVLNEAVTIAEVKLYGDVVLRYVSYKAEDTEKSEFLPGFERVEDASSFPLDYGIRRLDHAVGNVPELGPALTYVAGFTGFHQFAEFTADDVGTAESGLNSAVLASNDEMVLLPIAEPVHGTKRKSQIQTYLEHNEGAGLQHLALMSEDIFRTLREMRKRSSIGGFDFMPSPPPTYYQNLKKRVGDVLSDDQIKECEELGILVDRDDQGTLLQIFTKPLGDRPTIFIEIIQRVGCMMKDEEGKAYQSGGCGGFGKGNFSELFKSIEEYEKTLEAKQLVG

SEQ ID NO: 37 - DNA, 1353 bp, synthetic construct
  misc_feature 1..1353: note = nucleotide sequence encoding mutant HPPD polypeptide
  misc_feature 4..6: note = sequence coding for Ala
  misc_feature 7..24: note = sequence coding for an His Tag containing 6 His
  source 1..1353: mol_type = other DNA
atggctcatcaccatcaccatcaccaaaacgccgccgtttcagagaatcaaaaccatgatgacggcgctgcgtcgtcgccgggattcaagctcgtcggattttccaagttcgtaagaaagaatccaaagtctgataaattcaaggttaagcgcttccatcacatcgagttctggtgcggcgacgcaaccaacgtcgctcgtcgcttctcctggggtctggggatgagattctccgccaaatccgatctttccaccggaaacatggttcacgcctcttacctactcacctccggtgacctccgattccttttcactgctccttactctccgtctctctccgccggagagattaaaccgacaaccacagcttctatcccaagtttcgatcacggctcttgtcgttccttcttctcgtcacatggtctcggtgttagagccgttgcgattgaagtagaagacgcagagtcagctttctccatcagtgtagctaatggcgctattccttcgtcgcctcctatcgtcctcaatgaagcagttacgatcgctgaggttaaactatacggcgatgttgttctccgatatgttagttacaaagcagaagataccgaaaaatccgaattcttgccagggttcgagcgtgtagaggatgcgtcgtcgttcccattggattatggtatccggcggcttgaccacgccgtgggaaacgttcctgagcttggtccggctttaacttatgtagcggggttcactggttttcaccaattcgcatctttcacagcagacgacgttggaaccgccgagagcggtttaaattcagcggtcctggctagcaatgatgaaatggttcttctaccgattaacgagccagtgcacggaacaaagaggaagagtcagattcagacgtatttggaacataacgaaggcgcagggctacaacatctggctctgatgagtgaagacatattcaggaccctgagagagatgaggaagaggagcagtattggaggattcgacttcatgccttctcctccgcctacttactaccagaatctcaagaaacgggtcggcgacgtgctcagcgatgatcagatcaaggagtgtgaggaattagggattcttgtagacagagatgatcaagggacgttgcttcaaatcttcacaaaaccactaggtgacaggccgacgatatttatagagataatccagagagtaggatgcatgatgaaagatgaggaagggaaggcttaccagagtggaggatgtggtggttttggcaaaggcaatttctctgagctcttcaagtccattgaagaatacgaaaagactcttgaagccaaacagttagtgggatga

SEQ ID NO: 38 - protein, 450 aa, synthetic construct
  REGION 1..450: note = encoded by SEQ ID No. 37
  SITE 2: note = MISC_FEATURE - Ala
  REGION 3..8: note = MISC_FEATURE - His Tag made of 6 His
MAHHHHHHQNAAVSENQNHDDGAASSPGFKLVGFSKFVRKNPKSDKFKVKRFHHIEFWCGDATNVARRFSWGLGMRFSAKSDLSTGNMVHASYLLTSGDLRFLFTAPYSPSLSAGEIKPTTTASIPSFDHGSCRSFFSSHGLGVRAVAIEVEDAESAFSISVANGAIPSSPPIVLNEAVTIAEVKLYGDVVLRYVSYKAEDTEKSEFLPGFERVEDASSFPLDYGIRRLDHAVGNVPELGPALTYVAGFTGFHQFASFTADDVGTAESGLNSAVLASNDEMVLLPINEPVHGTKRKSQIQTYLEHNEGAGLQHLALMSEDIFRTLREMRKRSSIGGFDFMPSPPPTYYQNLKKRVGDVLSDDQIKECEELGILVDRDDQGTLLQIFTKPLGDRPTIFIEIIQRVGCMMKDEEGKAYQSGGCGGFGKGNFSELFKSIEEYEKTLEAKQLVG

SEQ ID NO: 39 - DNA, 1353 bp, synthetic construct
  misc_feature 1..1353: note = nucleotide sequence encoding mutant HPPD polypeptide
  misc_feature 4..6: note = sequence coding for Ala
  misc_feature 7..24: note = sequence coding for an His Tag containing 6 His
  source 1..1353: mol_type = other DNA
atggctcatcaccatcaccatcaccaaaacgccgccgtttcagagaatcaaaaccatgatgacggcgctgcgtcgtcgccgggattcaagctcgtcggattttccaagttcgtaagaaagaatccaaagtctgataaattcaaggttaagcgcttccatcacatcgagttctggtgcggcgacgcaaccaacgtcgctcgtcgcttctcctggggtctggggatgagattctccgccaaatccgatctttccaccggaaacatggttcacgcctcttacctactcacctccggtgacctccgattccttttcactgctccttactctccgtctctctccgccggagagattaaaccgacaaccacagcttctatcccaagtttcgatcacggctcttgtcgttccttcttctcgtcacatggtctcggtgttagagccgttgcgattgaagtagaagacgcagagtcagctttctccatcagtgtagctaatggcgctattccttcgtcgcctcctatcgtcctcaatgaagcagttacgatcgctgaggttaaactatacggcgatgttgttctccgatatgttagttacaaagcagaagataccgaaaaatccgaattcttgccagggttcgagcgtgtagaggatgcgtcgtcgttcccattggattatggtatccggcggcttgaccacgccgtgggaaacgttcctgagcttggtccggctttaacttatgtagcggggttcactggttttcaccaattcgcagagttcacagcagacgacgttggaaccgccgagagcggtttaaattcagcggcgctggctagcaatgatgaaatggttcttctaccgattaacgagccagtgcacggaacaaagaggaagagtcagattcagacgtatttggaacataacgaaggcgcagggctacaacatctggctctgatgagtgaagacatattcaggaccctgagagagatgaggaagaggagcagtattggaggattcgacttcatgccttctcctccgcctacttactaccagaatctcaagaaacgggtcggcgacgtgctcagcgatgatcagatcaaggagtgtgaggaattagggattcttgtagacagagatgatcaagggacgttgcttcaaatcttcacaaaaccactaggtgacaggccgacgatatttatagagataatccagagagtaggatgcatgatgaaagatgaggaagggaaggcttaccagagtggaggatgtggtggttttggcaaaggcaatttctctgagctcttcaagtccattgaagaatacgaaaagactcttgaagccaaacagttagtgggatga

SEQ ID NO: 40 - protein, 450 aa, synthetic construct
  REGION 1..450: note = encoded by SEQ ID No. 39
  SITE 2: note = MISC_FEATURE - Ala
  REGION 3..8: note = MISC_FEATURE - His Tag made of 6 His
MAHHHHHHQNAAVSENQNHDDGAASSPGFKLVGFSKFVRKNPKSDKFKVKRFHHIEFWCGDATNVARRFSWGLGMRFSAKSDLSTGNMVHASYLLTSGDLRFLFTAPYSPSLSAGEIKPTTTASIPSFDHGSCRSFFSSHGLGVRAVAIEVEDAESAFSISVANGAIPSSPPIVLNEAVTIAEVKLYGDVVLRYVSYKAEDTEKSEFLPGFERVEDASSFPLDYGIRRLDHAVGNVPELGPALTYVAGFTGFHQFAEFTADDVGTAESGLNSAALASNDEMVLLPINEPVHGTKRKSQIQTYLEHNEGAGLQHLALMSEDIFRTLREMRKRSSIGGFDFMPSPPPTYYQNLKKRVGDVLSDDQIKECEELGILVDRDDQGTLLQIFTKPLGDRPTIFIEIIQRVGCMMKDEEGKAYQSGGCGGFGKGNFSELFKSIEEYEKTLEAKQLVG

SEQ ID NO: 41 - DNA, 1353 bp, synthetic construct
  misc_feature 1..1353: note = nucleotide sequebc encoding mutant HPPD polypeptide
  misc_feature 4..6: note = sequence coding for Ala
  misc_feature 7..24: note = sequence coding for an His Tag containing 6 His
  source 1..1353: mol_type = other DNA
atggctcatcaccatcaccatcaccaaaacgccgccgtttcagagaatcaaaaccatgatgacggcgctgcgtcgtcgccgggattcaagctcgtcggattttccaagttcgtaagaaagaatccaaagtctgataaattcaaggttaagcgcttccatcacatcgagttctggtgcggcgacgcaaccaacgtcgctcgtcgcttctcctggggtctggggatgagattctccgccaaatccgatctttccaccggaaacatggttcacgcctcttacctactcacctccggtgacctccgattccttttcactgctccttactctccgtctctctccgccggagagattaaaccgacaaccacagcttctatcccaagtttcgatcacggctcttgtcgttccttcttctcgtcacatggtctcggtgttagagccgttgcgattgaagtagaagacgcagagtcagctttctccatcagtgtagctaatggcgctattccttcgtcgcctcctatcgtcctcaatgaagcagttacgatcgctgaggttaaactatacggcgatgttgttctccgatatgttagttacaaagcagaagataccgaaaaatccgaattcttgccagggttcgagcgtgtagaggatgcgtcgtcgttcccattggattatggtatccggcggcttgaccacgccgtgggaaacgttcctgagcttggtccggctttaacttatgtagcggggttcactggttttcaccaattcgcagagttcacagcagacgacgttggaaccgccgagagcggtttaaattcagcggtcctggctagcaatgatgaaatggttcttctagcgattaacgagccagtgcacggaacaaagaggaagagtcagattcagacgtatttggaacataacgaaggcgcagggctacaacatctggctctgatgagtgaagacatattcaggaccctgagagagatgaggaagaggagcagtattggaggattcgacttcatgccttctcctccgcctacttactaccagaatctcaagaaacgggtcggcgacgtgctcagcgatgatcagatcaaggagtgtgaggaattagggattcttgtagacagagatgatcaagggacgttgcttcaaatcttcacaaaaccactaggtgacaggccgacgatatttatagagataatccagagagtaggatgcatgatgaaagatgaggaagggaaggcttaccagagtggaggatgtggtggttttggcaaaggcaatttctctgagctcttcaagtccattgaagaatacgaaaagactcttgaagccaaacagttagtgggatga

SEQ ID NO: 42 - protein, 450 aa, synthetic construct
  REGION 1..450: note = encoded by SEQ ID No. 41
  SITE 2: note = MISC_FEATURE - Ala
  REGION 3..8: note = MISC_FEATURE - His Tag made of 6 His
MAHHHHHHQNAAVSENQNHDDGAASSPGFKLVGFSKFVRKNPKSDKFKVKRFHHIEFWCGDATNVARRFSWGLGMRFSAKSDLSTGNMVHASYLLTSGDLRFLFTAPYSPSLSAGEIKPTTTASIPSFDHGSCRSFFSSHGLGVRAVAIEVEDAESAFSISVANGAIPSSPPIVLNEAVTIAEVKLYGDVVLRYVSYKAEDTEKSEFLPGFERVEDASSFPLDYGIRRLDHAVGNVPELGPALTYVAGFTGFHQFAEFTADDVGTAESGLNSAVLASNDEMVLLAINEPVHGTKRKSQIQTYLEHNEGAGLQHLALMSEDIFRTLREMRKRSSIGGFDFMPSPPPTYYQNLKKRVGDVLSDDQIKECEELGILVDRDDQGTLLQIFTKPLGDRPTIFIEIIQRVGCMMKDEEGKAYQSGGCGGFGKGNFSELFKSIEEYEKTLEAKQLVG

SEQ ID NO: 43 - DNA, 1353 bp, synthetic construct
  misc_feature 1..1353: note = nucleotide sequence encoding mutant HPPD polypeptide
  misc_feature 4..6: note = sequence coding for Ala
  misc_feature 7..24: note = sequence coding for an His Tag containing 6 His
  source 1..1353: mol_type = other DNA
atggctcatcaccatcaccatcaccaaaacgccgccgtttcagagaatcaaaaccatgatgacggcgctgcgtcgtcgccgggattcaagctcgtcggattttccaagttcgtaagaaagaatccaaagtctgataaattcaaggttaagcgcttccatcacatcgagttctggtgcggcgacgcaaccaacgtcgctcgtcgcttctcctggggtctggggatgagattctccgccaaatccgatctttccaccggaaacatggttcacgcctcttacctactcacctccggtgacctccgattccttttcactgctccttactctccgtctctctccgccggagagattaaaccgacaaccacagcttctatcccaagtttcgatcacggctcttgtcgttccttcttctcgtcacatggtctcggtgttagagccgttgcgattgaagtagaagacgcagagtcagctttctccatcagtgtagctaatggcgctattccttcgtcgcctcctatcgtcctcaatgaagcagttacgatcgctgaggttaaactatacggcgatgttgttctccgatatgttagttacaaagcagaagataccgaaaaatccgaattcttgccagggttcgagcgtgtagaggatgcgtcgtcgttcccattggattatggtatccggcggcttgaccacgccgtgggaaacgttcctgagcttggtccggctttaacttatgtagcggggttcactggttttcaccaattcgcagagttcacagcagacgacgttggaaccgccgagagcggtttaaattcagcggtcctggctagcaatgatgaaatggttcttctaccgattaacgagccagtgcacggaacaaagaggaagagtcagattcagacgtatttggaacataacgaaggcgcagggctacaacatctggctctgatgagtgaagacatattcaggaccctgagagagatgaggaagaggagcagtattggaggattcgacttcatgccttctcctccgcctacttactaccagaatctcaagaaacgggtcggcgacgtgctcagcgatgatcagatcaaggagtgtgaggaattagggattatggtagacagagatgatcaagggacgttgcttcaaatcttcacaaaaccactaggtgacaggccgacgatatttatagagataatccagagagtaggatgcatgatgaaagatgaggaagggaaggcttaccagagtggaggatgtggtggttttggcaaaggcaatttctctgagctcttcaagtccattgaagaatacgaaaagactcttgaagccaaacagttagtgggatga

SEQ ID NO: 44 - protein, 450 aa, synthetic construct
  REGION 1..450: note = encoded by SEQ ID No. 43
  SITE 2: note = MISC_FEATURE - Ala
  REGION 3..8: note = MISC_FEATURE - His Tag made of 6 His
MAHHHHHHQNAAVSENQNHDDGAASSPGFKLVGFSKFVRKNPKSDKFKVKRFHHIEFWCGDATNVARRFSWGLGMRFSAKSDLSTGNMVHASYLLTSGDLRFLFTAPYSPSLSAGEIKPTTTASIPSFDHGSCRSFFSSHGLGVRAVAIEVEDAESAFSISVANGAIPSSPPIVLNEAVTIAEVKLYGDVVLRYVSYKAEDTEKSEFLPGFERVEDASSFPLDYGIRRLDHAVGNVPELGPALTYVAGFTGFHQFAEFTADDVGTAESGLNSAVLASNDEMVLLPINEPVHGTKRKSQIQTYLEHNEGAGLQHLALMSEDIFRTLREMRKRSSIGGFDFMPSPPPTYYQNLKKRVGDVLSDDQIKECEELGIMVDRDDQGTLLQIFTKPLGDRPTIFIEIIQRVGCMMKDEEGKAYQSGGCGGFGKGNFSELFKSIEEYEKTLEAKQLVG

SEQ ID NO: 45 - DNA, 1353 bp, synthetic construct
  misc_feature 1..1353: note = nucleotide sequence encoding mutant HPPD polypeptide
  misc_feature 4..6: note = sequence coding for Ala
  misc_feature 7..24: note = sequence coding for an His Tag containing 6 His
  source 1..1353: mol_type = other DNA
atggctcatcaccatcaccatcaccaaaacgccgccgtttcagagaatcaaaaccatgatgacggcgctgcgtcgtcgccgggattcaagctcgtcggattttccaagttcgtaagaaagaatccaaagtctgataaattcaaggttaagcgcttccatcacatcgagttctggtgcggcgacgcaaccaacgtcgctcgtcgcttctcctggggtctggggatgagattctccgccaaatccgatctttccaccggaaacatggttcacgcctcttacctactcacctccggtgacctccgattccttttcactgctccttactctccgtctctctccgccggagagattaaaccgacaaccacagcttctatcccaagtttcgatcacggctcttgtcgttccttcttctcgtcacatggtctcggtgttagagccgttgcgattgaagtagaagacgcagagtcagctttctccatcagtgtagctaatggcgctattccttcgtcgcctcctatcgtcctcaatgaagcagttacgatcgctgaggttaaactatacggcgatgttgttctccgatatgttagttacaaagcagaagataccgaaaaatccgaattcttgccagggttcgagcgtgtagaggatgcgtcgtcgttcccattggattatggtatccggcggcttgaccacgccgtgggaaacgttcctgagcttggtccggctttaacttatgtagcggggttcactggttttcaccaattcgcagagttcacagcagacgacgttggaaccgccgagagcggtttaaattcagcggtcctggctagcaatgatgaaatggttcttctaccgattaacgagccagtgcacggaacaaagaggaagagtcagattcagacgtatttggaacataacgaaggcgcagggctacaacatctggctctgatgagtgaagacatattcaggaccctgagagagatgaggaagaggagcagtattggaggattcgacttcatgccttctcctccgcctacttactaccagaatctcaagaaacgggtcggcgacgtgctcagcgatgatcagatcaaggagtgtgaggaattagggattcttgtagacagagatgatcaagggacgttgcttcaaatcttcacaaaaccactaggtgacaggccgacgatatttatagagataatccagagagtaggatgcatgatgaaagatgaggaagggaaggcttaccagagtggaggatgtggtggttttggcattggcaatttctctgagctcttcaagtccattgaagaatacgaaaagactcttgaagccaaacagttagtgggatga

SEQ ID NO: 46 - protein, 450 aa, synthetic construct
  REGION 1..450: note = encoded by SEQ ID No. 45
  SITE 2: note = MISC_FEATURE - Ala
  REGION 3..8: note = MISC_FEATURE - His Tag made of 6 His
MAHHHHHHQNAAVSENQNHDDGAASSPGFKLVGFSKFVRKNPKSDKFKVKRFHHIEFWCGDATNVARRFSWGLGMRFSAKSDLSTGNMVHASYLLTSGDLRFLFTAPYSPSLSAGEIKPTTTASIPSFDHGSCRSFFSSHGLGVRAVAIEVEDAESAFSISVANGAIPSSPPIVLNEAVTIAEVKLYGDVVLRYVSYKAEDTEKSEFLPGFERVEDASSFPLDYGIRRLDHAVGNVPELGPALTYVAGFTGFHQFAEFTADDVGTAESGLNSAVLASNDEMVLLPINEPVHGTKRKSQIQTYLEHNEGAGLQHLALMSEDIFRTLREMRKRSSIGGFDFMPSPPPTYYQNLKKRVGDVLSDDQIKECEELGILVDRDDQGTLLQIFTKPLGDRPTIFIEIIQRVGCMMKDEEGKAYQSGGCGGFGIGNFSELFKSIEEYEKTLEAKQLVG

SEQ ID NO: 47 - DNA, 1353 bp, synthetic construct
  misc_feature 1..1353: note = nucleotide sequence encoding mutant HPPD polypeptide
  misc_feature 4..6: note = sequence coding for Ala
  misc_feature 7..24: note = sequence coding for an His Tag containing 6 His
  source 1..1353: mol_type = other DNA
atggctcatcaccatcaccatcaccaaaacgccgccgtttcagagaatcaaaaccatgatgacggcgctgcgtcgtcgccgggattcaagctcgtcggattttccaagttcgtaagaaagaatccaaagtctgataaattcaaggttaagcgcttccatcacatcgagttctggtgcggcgacgcaaccaacgtcgctcgtcgcttctcctggggtctggggatgagattctccgccaaatccgatctttccaccggaaacatggttcacgcctcttacctactcacctccggtgacctccgattccttttcactgctccttactctccgtctctctccgccggagagattaaaccgacaaccacagcttctatcccaagtttcgatcacggctcttgtcgttccttcttctcgtcacatggtctcggtgttagagccgttgcgattgaagtagaagacgcagagtcagctttctccatcagtgtagctaatggcgctattccttcgtcgcctcctatcgtcctcaatgaagcagttacgatcgctgaggttaaactatacggcgatgttgttctccgatatgttagttacaaagcagaagataccgaaaaatccgaattcttgccagggttcgagcgtgtagaggatgcgtcgtcgttcccattggattatggtatccggcggcttgaccacgccgtgggaaacgttcctgagcttggtccggctttaacttatgtagcggggttcactggttttcaccaattcgcagagttcacagcagacgacgttggaaccgccgagagcggtttaaattcagcggtcctggctagcaatgatgaaatggttcttctaccgattaacgagccagtgcacggaacaaagaggaagagtcagattcagacgtatttggaacataacgaaggcgcagggctacaacatctggctctgatgagtgaagacatattcaggaccctgagagagatgaggaagaggagcagtattggaggattcgacttcatgccttctcctccgcctacttactaccagaatctcaagaaacgggtcggcgacgtgctcagcgatgatcagatcaaggagtgtgaggaattagggattcttgtagacagagatgatcaagggacgttgcttcaaatcttcacaaaaccactaggtgacaggccgacgatatttatagagataatccagagagtaggatgcatgatgaaagatgaggaagggaaggcttaccagagtggaggatgtggtggttttggccagggcaatttctctgagctcttcaagtccattgaagaatacgaaaagactcttgaagccaaacagttagtgggatga

SEQ ID NO: 48 - protein, 450 aa, synthetic construct
  REGION 1..450: note = encoded by SEQ ID No. 47
  SITE 2: note = MISC_FEATURE - Ala
  REGION 3..8: note = MISC_FEATURE - His Tag made of 6 His
MAHHHHHHQNAAVSENQNHDDGAASSPGFKLVGFSKFVRKNPKSDKFKVKRFHHIEFWCGDATNVARRFSWGLGMRFSAKSDLSTGNMVHASYLLTSGDLRFLFTAPYSPSLSAGEIKPTTTASIPSFDHGSCRSFFSSHGLGVRAVAIEVEDAESAFSISVANGAIPSSPPIVLNEAVTIAEVKLYGDVVLRYVSYKAEDTEKSEFLPGFERVEDASSFPLDYGIRRLDHAVGNVPELGPALTYVAGFTGFHQFAEFTADDVGTAESGLNSAVLASNDEMVLLPINEPVHGTKRKSQIQTYLEHNEGAGLQHLALMSEDIFRTLREMRKRSSIGGFDFMPSPPPTYYQNLKKRVGDVLSDDQIKECEELGILVDRDDQGTLLQIFTKPLGDRPTIFIEIIQRVGCMMKDEEGKAYQSGGCGGFGQGNFSELFKSIEEYEKTLEAKQLVG

SEQ ID NO: 49 - DNA, 1353 bp, synthetic construct
  misc_feature 1..1353: note = nucleotide sequence encoding mutant HPPD polypeptide
  misc_feature 4..6: note = sequence coding for Ala
  misc_feature 7..24: note = sequence coding for an His Tag containing 6 His
  source 1..1353: mol_type = other DNA
atggctcatcaccatcaccatcaccaaaacgccgccgtttcagagaatcaaaaccatgatgacggcgctgcgtcgtcgccgggattcaagctcgtcggattttccaagttcgtaagaaagaatccaaagtctgataaattcaaggttaagcgcttccatcacatcgagttctggtgcggcgacgcaaccaacgtcgctcgtcgcttctcctggggtctggggatgagattctccgccaaatccgatctttccaccggaaacatggttcacgcctcttacctactcacctccggtgacctccgattccttttcactgctccttactctccgtctctctccgccggagagattaaaccgacaaccacagcttctatcccaagtttcgatcacggctcttgtcgttccttcttctcgtcacatggtctcggtgttagagccgttgcgattgaagtagaagacgcagagtcagctttctccatcagtgtagctaatggcgctattccttcgtcgcctcctatcgtcctcaatgaagcagttacgatcgctgaggttaaactatacggcgatgttgttctccgatatgttagttacaaagcagaagataccgaaaaatccgaattcttgccagggttcgagcgtgtagaggatgcgtcgtcgttcccattggattatggtatccggcggcttgaccacgccgtgggaaacgttcctgagcttggtccggctttaacttatgtagcggggttcactggttttcaccaattcgcagagttcacagcagacgacgttggaaccgccgagagcggtttaaattcagcggtcctggctagcaatgatgaaatggttcttctaccgattaacgagccagtgcacggaacaaagaggaagagtcagattcagacgtatttggaacataacgaaggcgcagggctacaacatctggctctgatgagtgaagacatattcaggaccctgagagagatgaggaagaggagcagtattggaggattcgacttcatgccttctcctccgcctacttactaccagaatctcaagaaacgggtcggcgacgtgctcagcgatgatcagatcaaggagtgtgaggaattagggattcttgtagacagagatgatcaagggacgttgcttcaaatcttcacaaaaccactaggtgacaggccgacgatatttatagagataatccagagagtaggatgcatgatgaaagatgaggaagggaaggcttaccagagtggaggatgtggtggttttggcgtgggcaatttctctgagctcttcaagtccattgaagaatacgaaaagactcttgaagccaaacagttagtgggatga

SEQ ID NO: 50 - protein, 450 aa, synthetic construct
  REGION 1..450: note = encoded by SEQ ID No. 49
  SITE 2: note = MISC_FEATURE - Ala
  REGION 3..8: note = MISC_FEATURE - His Tag made of 6 His
MAHHHHHHQNAAVSENQNHDDGAASSPGFKLVGFSKFVRKNPKSDKFKVKRFHHIEFWCGDATNVARRFSWGLGMRFSAKSDLSTGNMVHASYLLTSGDLRFLFTAPYSPSLSAGEIKPTTTASIPSFDHGSCRSFFSSHGLGVRAVAIEVEDAESAFSISVANGAIPSSPPIVLNEAVTIAEVKLYGDVVLRYVSYKAEDTEKSEFLPGFERVEDASSFPLDYGIRRLDHAVGNVPELGPALTYVAGFTGFHQFAEFTADDVGTAESGLNSAVLASNDEMVLLPINEPVHGTKRKSQIQTYLEHNEGAGLQHLALMSEDIFRTLREMRKRSSIGGFDFMPSPPPTYYQNLKKRVGDVLSDDQIKECEELGILVDRDDQGTLLQIFTKPLGDRPTIFIEIIQRVGCMMKDEEGKAYQSGGCGGFGVGNFSELFKSIEEYEKTLEAKQLVG

(19) United States
(12) Patent Application Publication
(10) Pub. No.: US 2025/0223607 A1
(43) Pub. Date: Jul. 10, 2025

(54) HPPD Variants and Methods of Use

(71) Applicant: BASF Agricultural Solutions Seed US LLC, Research Triangle Park, NC (US)

(72) Inventors: Gudrun Lange, Research Triangle Park, NC (US); Fabien Poree, Research Triangle Park, NC (US); Bernd Laber, Research Triangle Park, NC (US); Joerg Freigang, Research Triangle Park, NC (US); Arno Schultz, Research Triangle Park, NC (US)

(73) Assignee: BASF Agricultural Solutions Seed US LLC, Research Triangle Park, NC (US)

(21) Appl. No.: 18/954,204

(22) Filed: Jan. 27, 2025

Related U.S. Application Data
(62) Division of application No. 13/789,533, filed on Mar. 7, 2013, now abandoned.

(51) Int. Cl. C12N 15/82; C12N 9/02

(52) U.S. Cl. CPC C12N 15/8274; C12N 9/0008; C12Y 102/03013

(57) ABSTRACT

The present invention relates to an isolated nucleic acid comprising a nucleotide sequence encoding a mutated HPPD protein, wherein said mutated HPPD protein has HPPD activity, wherein in said mutated HPPD protein at least one amino acid has been replaced so that the resulting amino acid sequence comprises at least one amino acid selected from certain amino acids at specific positions important for conferring an increased HPPD inhibitor tolerance. The present invention also relates to proteins encoded by the nucleic acid of the invention, to chimeric genes, plant cells comprising the nucleic acid of the invention operably linked to a plant-expressible promoter and optionally a transcription termination and polyadenylation region, plants essentially consisting of the plant cells of the invention and methods of obtaining transgenic plants.

### CROSS REFERENCE TO RELATED APPLICATIONS

The present application is a divisional of Ser. No. 13/789,533, filed on Mar. 7, 2013, which is hereby incorporated by reference in its entirety. This application is also related to EP2453012 A1, the content of which is incorporated herein by reference in its entirety.

### SEQUENCE LISTING

The Sequence Listing associated with this application is filed in electronic format via EFS-Web and hereby incorporated by reference into the specification in its entirety. The name of the text file containing the Sequence Listing is 181227US03_Sequence_Listing. The size of the text file is 105 KB, and the file was created on Nov. 20, 2024.

### BACKGROUND

### Field of the Invention

The present invention relates to an isolated nucleic acid comprising a nucleotide sequence encoding a mutated HPPD protein, wherein said mutated HPPD protein has HPPD activity, wherein in said mutated HPPD protein at least one amino acid has been replaced so that the resulting amino acid sequence comprises at least one amino acid selected from certain amino acids at specific positions important for conferring an increased HPPD inhibitor tolerance. The present invention also relates to proteins encoded by the nucleic acid of the invention, to chimeric genes, plant cells comprising the nucleic acid of the invention operably linked to a plant-expressible promoter and optionally a transcription termination and polyadenylation region, plants essentially consisting of the plant cells of the invention and methods of obtaining transgenic plants.

### Description of Related Art

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

HPPD (hydroxyphenylpyruvate dioxygenase) proteins are enzymes which catalyse the reaction in which para-hydroxyphenylpyruvate (abbreviated herein as HPP), a tyrosine degradation product, is transformed into homogentisate (abbreviated herein as HG), the precursor in plants of tocopherol and plastoquinone (Crouch N. P. et al. (1997) Tetrahedron, 53, 20, 6993-7010, Fritze et al., (2004), Plant Physiology 134:1388-1400). Tocopherol acts as a membrane-associated antioxidant. Plastoquinone, firstly acts as an electron carrier between PSII and the cytochrome b6/f complex and secondly, is a redox cofactor for phytoene desaturase, which is involved in the biosynthesis of carotenoids.

Up to now, more than 700 nucleic acid sequences from various organisms present in NCBI database were annotated as coding for a putative protein having an HPPD domain. But for most of these sequences, it has not been proven that the protein would have an HPPD enzymatic activity either in an in vitro assay or an in in planta approach, nor that such HPPD protein can confer herbicide tolerance to HPPD inhibitor herbicides when expressed in a plant. Several HPPD proteins and their primary sequences have been described in the state of the art, in particular the HPPDs of bacteria such as Pseudomonas (Ruetschi et al., Eur. J. Biochem., 205, 459-466, 1992, WO 96/38567), of plants such as Arabidopsis (WO 96/38567, Genebank AF047834), carrot (WO 96/38567, Genebank 87257), Avena sativa (WO 02/046387), wheat (WO 02/046387), Brachiaria platyphylla (WO 02/046387), Cenchrus echinatus (WO 02/046387), Lolium rigidum (WO 02/046387), Festuca arundinacea (WO 02/046387), Setaria faberi (WO 02/046387), Eleusine indica (WO 02/046387), Sorghum (WO 02/046387), Coccicoides (Genebank COITRP), of Coptis japonica (WO 06/132270), Chlamydomonas reinhardtii (ES 2275365), or of mammals such as mouse or pig.

Most plants synthesize tyrosine via arrogenate (Abou-Zeid et al. (1995), Applied Env Microb 41: 1298-1302; Bonner et al., (1995), Plant Cells Physiol. 36, 1013-1022; Byng et al., (1981), Phytochemistry 6: 1289-1292; Connely and Conn (1986), Z. Naturforsch 41c: 69-78; Gaines et al., (1982), Plants 156: 233-240). In these plants, the HPP is derived only from the degradation of tyrosine. On the other hand, in organisms such as the yeast Sacharomyces cerevisiae or the bacterium Escherichia coli, HPP is a tyrosine precursor, and it is synthesized by the action of an enzyme, prephenate dehydrogenase (hereinafter referred to as PDH), which converts prephenate to HPP (Lingens et al., (1967) European J. Biochem 1: 363-374; Sampathkumar and Morrisson (1982), Bioch Biophys Acta 701: 204-211). In these organisms, the production of HPP is therefore directly connected to the aromatic amino acid biosynthetic pathway (shikimate pathway), and not to the tyrosine degradation pathway.

Inhibition of HPPD leads to uncoupling of photosynthesis, deficiency in accessory light-harvesting pigments and, most importantly, to destruction of chlorophyll by UV-radiation and reactive oxygen species (bleaching) due to the lack of photo protection normally provided by carotenoids (Norris et al. (1995), Plant Cell 7: 2139-2149). Bleaching of photosynthetically active tissues leads to growth inhibition and plant death.

At present, most commercially available HPPD inhibitor herbicides belong to one of these four chemical families:
1) the triketones, e.g. sulcotrione [i.e. 2-[2-chloro-4-(methylsulfonyl)benzoyl]-1,3-cyclohexanedione], mesotrione [i.e. 2-[4-(methylsulfonyl)-2-nitrobenzoyl]-1,3-cyclohexanedione]; tembotrione [i.e. 2-[2-chloro-4-(methylsulfonyl)-3-[(2,2,2,-tri-fluoroethoxy)methyl]benzoyl]-1,3-cyclo-hexanedione]; tefuryltrione [i.e. 2-[2-chloro-4-(methylsulfonyl)-3-[[(tetrahydro-2-furanyl)methoxy]methyl]benzoyl]-1,3 cyclohexanedione]]; bicyclopyrone [i.e. 4-hydroxy-3-[[2-[(2-methoxyethoxy)methyl]-6-(trifluoromethyl)-3-pyridinyl]carbonyl]bicyclo[3.2.1]oct-3-en-2-one]; Benzobicyclon [i.e. 3-(2-chloro-4-mesylbenzoyl)-2-phenylthiobicyclo[3.2.1]oct-2-en-4-one]
2) The diketonitriles, e.g. 2-cyano-3-cyclopropyl-1-(2-methylsulphonyl-4-trifluoromethylphenyl)-propane-1,3-dione and 2-cyano-1-[4-(methylsulphonyl)-2-trifluoromethylphenyl]-3-(1-methylcyclopropyl)propane-1,3-dione;
3) the isoxazoles, e.g. isoxaflutole [i.e. (5-cyclopropyl-4-isoxazolyl)[2-(methylsulfonyl)-4-(trifluoromethyl)phenyl]methanone]. In plants, the isoxaflutole is rapidly metabolized in DKN, a diketonitrile compound which exhibits the HPPD inhibitor property; and
4) the pyrazolinates, e.g. topramezone [i.e. [3-(4,5-dihydro-3-isoxazolyl)-2-methyl-4-(methylsulfonyl) phenyl](5-hydroxy-1-methyl-iH-pyrazol-4-yl)methanone], and pyrasulfotole [(5-hydroxy-1,3-dimethylpyrazol-4-yl(2-mesyl-4-trifluaromethylphenyl)methanone]; pyrazofen [2-[4-(2,4-dichlorobenzoyl)-1,3-dimethylpyrazol-5-yloxy]acetophenone].

These HPPD-inhibiting herbicides can be used against grass and/or broad leaf weeds in crop plants that display metabolic tolerance, such as maize (Zea mays) in which they are rapidly degraded (Schulz et al., (1993). FEBS letters, 318, 162-166; Mitchell et al., (2001) Pest Management Science, Vol 57, 120-128; Garcia et al., (2000) Biochem., 39, 7501-7507; Pallett et al., (2001) Pest Management Science, Vol 57, 133-142). In order to extend the scope of these HPPD-inhibiting herbicides, several efforts have been developed in order to confer to plants, particularly plants without or with an underperforming metabolic tolerance, a tolerance level acceptable under agronomic field conditions.

Besides the attempt of by-passing HPPD-mediated production of homogentisate (U.S. Pat. No. 6,812,010), overexpressing the sensitive enzyme so as to produce quantities of the target enzyme in the plant which are sufficient in relation to the herbicide has been performed (WO96/38567). Overexpression of HPPD resulted in better pre-emergence tolerance to the diketonitrile derivative (DKN) of isoxaflutole (IFT), but tolerance was not sufficient for tolerance to post-emergence treatment (Matringe et al., (2005), Pest Management Science 61: 269-276).

In WO 04/024928, the inventors have sought to increase the prenylquinone biosynthesis (e.g., synthesis of plastoquinones, tocopherols) in the cells of plants by increasing the flux of the HPP precursor into the cells of these plants. This has been done by connecting the synthesis of said precursor to the “shikimate” pathway by overexpression of a PDH enzyme. They have also noted that the transformation of plants with a gene encoding a PDH enzyme makes it possible to increase the tolerance of said plants to HPPD inhibitors.

Another strategy was to mutate the HPPD in order to obtain a target enzyme which, while retaining its properties of catalysing the transformation of HPP into homogentisate, is less sensitive to HPPD inhibitors than is the native HPPD before mutation.

This strategy has been successfully applied for the production of plants tolerant to 2-cyano-3-cyclopropyl-1-(2-methylsulphonyl-4-trifluoromethylphenyl)-propane-1,3-dione and to 2-cyano-1-[4-(methylsulphonyl)-2-trifluoromethylphenyl]-3-(1-methylcyclopropyl)propane-1,3-dione (EP496630), two HPPD-inhibiting herbicides belonging to the diketonitriles family (WO 99/24585). Pro215Leu, Gly336Glu, Gly336Ile, and more particularly Gly336Trp (positions of the mutated amino acid are indicated with reference to the Pseudomonas HPPD) were identified as mutations which are responsible for an increased tolerance to pre-emergence treatment with these diketonitrile herbicides without causing an alteration of the activity of the enzyme.

More recently, introduction of a Pseudomonas HPPD gene into the plastid genome of tobacco and soybean has shown to be more effective than nuclear transformation, conferring even tolerance to post-emergence application of isoxaflutole (Dufourmantel et al., 2007, Plant Biotechnol J. 5(1):118-33).

In the patent application WO 2009/144079, a nucleic acid sequence encoding a mutated hydroxyphenylpyruvate dioxygenase (HPPD) at position 336 of the Pseudomonas fluorescens HPPD protein and its use for obtaining plants which are tolerant to HPPD inhibitor herbicides is disclosed.

In WO 2002/046387, several domains of HPPD proteins originated from plants have been identified that may be relevant to confer tolerance to various HPPD inhibitor herbicides but no in planta nor biochemical data have been shown to confirm the impact of the as described domain functions.

In WO 2008/150473, the combination of two distinct tolerance mechanisms—a modified Avena sativa gene coding for a mutant HPPD enzyme and a CYP450 Maize monooxygenase (nsfl gene)—was exemplified in order to obtain an improved tolerance to HPPD inhibitor herbicides, but no data have been disclosed demonstrating the synergistic effects based on the combination of both proteins.

US 2010/0197503 suggests a number of mutations at different positions within or close to the active site of the HPPD taken from Avena sativa and examined some of them for their inhibition by certain HPPD inhibitors such as sulcotrione in vitro and in planta.

Despite these successes obtained for the development of plants showing tolerance to several HPPD inhibitors herbicides described above, it is still necessary to develop and/or improve the tolerance of plants to newer or to several different HPPD inhibitors, particularly HPPD inhibitors belonging to the classes of the triketones (e.g. sulcotrione, mesotrione, tembotrione, benzobicyclon and bicyclopyrone) and the pyrazolinates (e.g., topramezone and pyrasulfotole).

### SUMMARY

Accordingly, the present invention relates to an isolated nucleic acid comprising a nucleotide sequence encoding a mutated HPPD protein, wherein said mutated HPPD protein has HPPD activity,
wherein in said mutated HPPD protein at least one amino acid has been replaced so that the resulting amino acid sequence comprises at least one amino acid selected from:
a) Ala, Asp, Glu, Phe, Gly, Lys, Gln, Arg, Ser, Thr, Val, Tyr, Ile, Leu or Met at a position in an HPPD protein, said position corresponding to position 250 of the amino acid sequence of SEQ ID No. 2;
b) Ala, Asp, Glu, Ile, Lys, Leu, Asn, Pro, Arg, Ser Gly, His or Tyr, at a position in an HPPD protein, said position corresponding to position 251 of the amino acid sequence of SEQ ID No. 2;
c) Glu, Phe, Gly, His, Ile, Leu, Met, Asn, Gln, Arg, Ser, Val, Thr or Tyr at a position in an HPPD protein, said position corresponding to position 252 of the amino acid sequence of SEQ ID No.;
d) Ala, Phe, His, Gln, Val, Trp, Ile, Leu or Met at a position in an HPPD protein, said position corresponding to position 253 of the amino acid sequence of SEQ ID No. 2;
e) Leu, Val, or Met at a position in an HPPD protein, said position corresponding to position 265 of the amino acid sequence of SEQ ID No. 2;
f) Leu, Gln, Arg, Val, Tyr, Ala, Ile, Lys or Met at a position in an HPPD protein, said position corresponding to position 268 of the amino acid sequence of SEQ ID No. 2;
g) Ala, Thr or Val at a position in an HPPD protein, said position corresponding to position 269 of the amino acid sequence of SEQ ID No. 2;
h) Ala, Ile, Asn, Pro, Thr or Val at a position in an HPPD protein, said position corresponding to position 280 of the amino acid sequence of SEQ ID No. 2;
i) Leu, Gln, Val, Ala, Phe, Gly, Met, Arg or Ser at a position in an HPPD protein, said position corresponding to position 293 of the amino acid sequence of SEQ ID No. 2;
j) Ile, Met, Ala, Pro, Ser, Thr or Val at a position in an HPPD protein, said position corresponding to position 294 of the amino acid sequence of SEQ ID No. 2;
k) Gln, His or Asn at a position in an HPPD protein, said position corresponding to position 307 of the amino acid sequence of SEQ ID No. 2;
l) Ile, Met, Asn or Leu at a position in an HPPD protein, said position corresponding to position 335 of the amino acid sequence of SEQ ID No. 2;
m) Leu or Met at a position in an HPPD protein, said position corresponding to position 368 of the amino acid sequence of SEQ ID No. 2;
n) Phe or Tyr at a position in an HPPD protein, said position corresponding to position 381 of the amino acid sequence of SEQ ID No. 2;
o) Phe or Ser at a position in an HPPD protein, said position corresponding to position 392 of the amino acid sequence of SEQ ID No. 2;
p) Phe or Tyr at a position in an HPPD protein, said position corresponding to position 419 of the amino acid sequence of SEQ ID No. 2;
q) Asp, Lys, Asn, Ala, Cys, Glu, Gly, His, Ile, Leu, Met, Gln, Ser, Thr or Val at a position in an HPPD protein, said position corresponding to position 421 of the amino acid sequence of SEQ ID No. 2;
r) Ala, Gly, Met, Pro or Thr at a position in an HPPD protein, said position corresponding to position 422 of the amino acid sequence of SEQ ID No. 2;
s) Ala, Phe, Ile or Val at a position in an HPPD protein, said position corresponding to position 424 of the amino acid sequence of SEQ ID No. 2;
t) Ile, Pro, Arg, Ser Ala, Gly, Lys, Asn or Gln at a position in an HPPD protein, said position corresponding to position 425 of the amino acid sequence of SEQ ID No. 2;
u) Glu, Phe, Thr, Val Ala, Gln or Ser at a position in an HPPD protein, said position corresponding to position 426 of the amino acid sequence of SEQ ID No. 2;
v) Ile, Met, Gln, Val Phe or Leu at a position in an HPPD protein, said position corresponding to position 431 of the amino acid sequence of SEQ ID No. 2; and
w) at least one amino acid deletion or replacement at any one of positions 228, 248, 270, 271, 379 and/or 427.

### BRIEF DESCRIPTION OF THE DRAWINGS

FIG. 1A illustrates the superposition of the x-ray structure of HPPD from Arabidopsis thaliana (dark grey) and (a) Pseudomonas fluorescens (light grey). The structures are shown as ribbon plot.

FIG. 1B illustrates the superposition of the x-ray structure of HPPD from Arabidopsis thaliana (dark grey) and (a) Pseudomonas fluorescens (light grey), (b) Streptomyces avermitilis, (c) Homo sapiens, (d) Rattus norvegicus. The structures are shown as ribbon plot.

FIG. 1C illustrates the superposition of the x-ray structure of HPPD from Arabidopsis thaliana (dark grey) and (c) Homo sapiens. The structures are shown as ribbon plot.

FIG. 1D illustrates the superposition of the x-ray structure of HPPD from Arabidopsis thaliana (dark grey) and Rattus norvegicus. The structures are shown as ribbon plot.

FIG. 2A illustrates the binding amino acids forming the binding site of Arabidopsis thaliana, FIG. 2B illustrates the binding amino acids forming the binding site of Pseudomonas fluorescens; FIG. 2C illustrates the binding amino acids forming the binding site of Streptomyces avermitilis; and FIG. 2D illustrates the binding amino acids forming the binding site of Homo sapiens, FIG. 2E illustrates the binding amino acids forming the binding site of Rattus norvegicus (represented by bold lines).

FIG. 3 illustrates brown test color in Escherichia coli

### DETAILED DESCRIPTION OF A PREFERRED EMBODIMENT

Unless indicated otherwise, the specific definitions or specific features of certain embodiments can be introduced into any other embodiment of the present invention.

According to the present invention, a “nucleic acid” is understood as being a nucleotide sequence which can be of the DNA or RNA type, preferably of the DNA type, and in particular double-stranded, whether it be of natural or synthetic origin, in particular a DNA sequence in which the codons which encode the HPPD according to the invention have been optimized in accordance with the host organism in which it is to be expressed (e.g., by replacing codons with those codons more preferred or most preferred in codon usage tables of such host organism or the group to which such host organism belongs, compared to the original or source organism).

An “isolated nucleic acid/DNA/protein”, as used in the present application, refers to a nucleic acid/DNA/protein which is not naturally-occurring (such as an artificial or synthetic DNA with a different nucleotide sequence than the naturally-occurring DNA, or a modified protein) or which is no longer in the natural environment wherein it was originally present, e.g., a DNA coding sequence associated with a heterologous regulatory element (such as a bacterial coding sequence operably-linked to a plant-expressible promoter) in a chimeric gene, a DNA transferred into another host cell, such as a transgenic plant cell.

The terminology relating to nucleic acid or protein “comprising” a certain nucleotide sequence or amino acid sequence, as used throughout the text, refers to a nucleic acid or protein including or containing at least the described sequence, so that other nucleotide or amino acid sequences can be included at the 5′ (or N-terminal) and/or 3′ (or C-terminal) end, e.g. (the nucleotide sequence of) a selectable marker protein, (the nucleotide sequence of) a transit peptide, and/or a 5′ leader sequence or a 3′ trailer sequence. Similarly, use of the term “comprise”, “comprising” or “comprises” throughout the text and the claims of this application should be understood to imply the inclusion of a stated integer or step or group of integers or steps but not the exclusion of any other integer or step or group of integers or steps. The term “comprising” also includes the term “consisting of”.

In accordance with the present invention, the term “mutated HPPD protein” which is interchangeably used with the term “mutant HPPD protein” denotes an HPPD protein having an amino acid sequence which does not occur in nature. As opposed to the term “isolated” referred to above, the term “mutated” cannot refer to the environment of the (amino acid or protein) sequence in question, such as it being isolated from its natural environment or being coupled to a heterologous (amino acid or protein) sequence, but only refers to the amino acid sequence defining said mutated HPPD protein which cannot be found anywhere in nature but arose from a unmutated or wild-type starting amino acid sequence. In other words, in arriving at the nucleic acid of the present invention encoding a mutated HPPD protein, a starting amino acid sequence of a naturally existing protein has to be taken and to be modified by man by replacing at least one amino acid as defined in the present application.

The sequence which encodes an original unmutated HPPD which will be mutated according to the invention can be of any origin. In particular, it can be of bacterial, plant or animal origin. Advantageous examples which may be cited are bacteria of the Pseudomonas sp. type, for example Pseudomonas fluorescens, or otherwise cyanobacteria of the Synechocystis genus. The sequence can also be of plant origin, in particular derived from dicotyledonous plants, umbelliferous plants, or otherwise monocotyledonous plants. Advantageous examples which may be cited are plants such as tobacco, Arabidopsis, Daucus carotta, Zea mays (corn), wheat, barley, Avena sativa, wheat, Brachiaria platyphylla, Cenchrus echinatus, Lolium rigidum, Festuca arundinacea, Setaria faberi, Eleusine indica, and Sorghum. The coding sequences, and the way of isolating and cloning them, are described in the previously cited references. In a particular embodiment of the invention, the HPPD is from a bacterial origin, particularly from Pseudomonas sp., more particularly from Pseudomonas fluorescens, Rhodococcus sp., Blepharisma japonicum, Synechococcus sp., Picrophilus torridus, Kordia algicida or from a plant origin, particularly from Arabidopsis thaliana or Avena sativa. The HPPD to make the mutation (s) in for the purpose of the invention, can be any naturally-occurring HPPD, or any active fragment thereof or any variant thereof wherein some amino acids (1 to 10 amino acids) have been replaced, added or deleted for cloning purposes, to make a transit peptide fusion, and the like, which retains HPPD activity, i.e. the property of catalysing the conversion of para-hydroxyphenylpyruvate to homogentisate.

The mutated HPPD protein according to the present invention has HPPD activity, i. e., as described above, catalyses the reaction in which para-hydroxyphenylpyruvate is transformed into homogentisate. Preferentially, the catalytic activity of the isolated mutated HPPD of the present invention, when tested in vitro, does not differ from that of the unmutated reference HPPD protein by more than 70%, preferably more than 50%, more preferably more than 30%, even more preferably more than 20% when assayed under identical conditions and in the absence of the HPPD inhibitor herbicides described above. The catalytic activity of an HPPD enzyme may be defined by various methods well-known in the art. WO 2009/144079 describes various suitable screening methods.

Initial screens may be performed with the nucleic acid encoding the mutated HPPD protein of the invention being expressed in bacteria.

### Colorimetric Screening Test for Active HPPD Enzymes:

A YT-broth-type culture medium with 1% agarose, 5 mM L-Tyrosine and 42 mM Succinate, which contains the selection agent for the vector pSE420 (Invitrogen, Karlsruhe, Germany) is poured into deep well plates. E. coli culture in the exponentional growth phase which contains the vector pSE420-HPPDx (HPPDx means any gene coding for a putative HPPD enzyme/protein) is applied to each well. After 16 hours at 37° C., the wells which do not contain the culture medium, those which have been seeded with an E. coli culture containing the empty vector pSE420 are transparent, or those which have been seeded with an E. coli culture containing a vector pSE420-HPPDx containing a gene coding for an inactive HPPD are transparent, while the weels seeded with an E. coli culture containing the vector pSE420-HPPDx coding for an active HPPD are brown. It has been previously demonstrated that this test reflects the HPPD activity, whatever the origin of this activity is, and allows the identification of HPPD activities (U.S. Pat. No. 6,768,044), i.e. at a qualitative level.

Further and more elaborate screens may be carried out in plant cells or plants expressing the mutated HPPD protein of the invention.

The same screenings may also be used when examining of whether a mutated HPPD protein is capable of modulating, such as decreasing or increasing, the tolerance of a plant to at least one HPPD herbicide inhibitor which will be referred to further below, with the difference that at least one of such an HPPD inhibitor is added. Examples of HPPD inhibitors to be used in those screenings include tembotrione, mesotrione, pyrasulfotole, bicyclopyrone, topramezone and sulcotrione. A screening method which is simple to implement is to determine the dose of HPPD inhibitor which fully inhibits the original unmutated HPPD, and which is lethal for the cells which express this unmutated HPPD, and to subject the mutated cells to this predetermined dose, and thereafter to isolate the mutated cells which have withstood this lethal dose, and then to isolate and to clone the gene which encodes the mutated HPPD.

Alternatively, at the quantitative level data like pI50 (pI50-value means the log value of the concentration of inhibitor necessary to inhibit 50% of the enzyme activity in molar concentration) can be obtained by employing the isolated and purified HPPD polypeptide, i.e. the mutated vs. the unmutated HPPD polypeptide and in presence or absence of the any respective HPPD inhibitor herbicide.

The terms “tolerance”, “tolerant” or “less sensitive” denotes the lack of susceptibility of a plant expressing the mutated HPPD protein of the present invention to substances, particularly herbicides, which inhibit HPPD proteins, optionally in comparison with the plant's own HPPD protein or with any known HPPD protein. More specifically, said terms mean the relative levels of inherent tolerance of the HPPD screened according to a visible indicator phenotype of the strain or plant transformed with a nucleic acid comprising the gene coding for the respective HPPD protein in the presence of different concentrations of the various HPPD inhibitors. Dose responses and relative shifts in dose responses associated with these indicator phenotypes (formation of brown colour, growth inhibition, bleaching, herbicidal effect etc) are conveniently expressed in terms, for example, of GR50 (concentration for 50% reduction of growth) or MIC (minimum inhibitory concentration) values where increases in values correspond to increases in inherent tolerance of the expressed HPPD, in the normal manner based upon plant damage, meristematic bleaching symptoms etc. at a range of different concentrations of herbicides. These data can be expressed in terms of, for example, GR50 values derived from dose/response curves having “dose” plotted on the x-axis and “percentage kill”, “herbicidal effect”, “numbers of emerging green plants” etc. plotted on the y-axis where increased GR50 values correspond to increased levels of inherent tolerance of the expressed HPPD. Herbicides can suitably be applied pre-emergence or post emergence.

Likewise, tolerance level of the nucleic acid or gene encoding an HPPD protein according to the invention, or the mutated HPPD protein of the invention is screened via transgenesis, regeneration, breeding and spray testing of a test plant such as tobacco, or a crop plant such as soybean or cotton. In line with the results obtained by such screening, such plants are at least 2-4 times more tolerant to HPPD inhibitors like tembotrione, mesotrione, diketonitrile, and/or bicyclopyrone, pyrasulfotole, than plants that do not contain any exogenous gene encoding an HPPD protein, or than plants that contain a gene comprising an Arabidopsis thaliana HPPD-encoding DNA, under control of the same promoter as the nucleic acid encoding the mutated HPPD protein of the invention. Accordingly, the term “capable of increasing the tolerance of a plant to at least one herbicide acting on HPPD” denotes a tolerance increased in a plant by at least the factor of 2, alternatively at least the factor of 3 or 4 or even 5 or 6 as compared to a plant only expressing it's endogenous HPPD or a plant expressing an Arabidopsis thaliana HPPD. In this regard, the term “herbicide acting on HPPD” is not limited to substances which are known and/or used as herbicides but to any substances which inhibits the catalytic activity of HPPD proteins.

In an alternative embodiment of the nucleic acid encoding a mutated HPPD polypeptide comprising at least one of the mutations as defined above, the HPPD protein comprises
a His at a position in an HPPD protein, said position corresponding to position 226 of the amino acid sequence of SEQ ID No. 2;
a Ser at a position in an HPPD protein, said position corresponding to position 267 of the amino acid sequence of SEQ ID No. 2;
an Asn at a position in an HPPD protein, said position corresponding to position 282 of the amino acid sequence of SEQ ID No. 2;
a His at a position in an HPPD protein, said position corresponding to position 308 of the amino acid sequence of SEQ ID No. 2;
a Tyr at a position in an HPPD protein, said position corresponding to position 342 of the amino acid sequence of SEQ ID No. 2;
a Glu at a position in an HPPD protein, said position corresponding to position 394 of the amino acid sequence of SEQ ID No. 2;
a Gly at a position in an HPPD protein, said position corresponding to position 420 of the amino acid sequence of SEQ ID No. 2; and
an Asn at a position in an HPPD protein, said position corresponding to position 423 of the amino acid sequence of SEQ ID No. 2

In the mutated HPPD protein encoded by the nucleic acid of the invention at least one amino acid has been deleted or replaced as defined above.

The replacement or deletion can be effected in the nucleic acid sequence which encodes the original unmutated, i. e. naturally occurring HPPD as defined above by any means which is appropriate for replacing, in the said sequence, the codon which encodes the amino acid to be replaced with the codon which corresponds to the amino acid which is to replace it, or by deleting a codon, with the said codons being widely described in the literature and well known to the skilled person. Several molecular biological methods can be used to achieve this replacement or deletion. A preferred method for preparing a mutated nucleic acid sequence according to the invention and the corresponding protein comprises carrying out site-directed mutagenesis on codons encoding one or more amino acids which are selected in advance. The methods for obtaining these site-directed mutations are well known to the skilled person and widely described in the literature (in particular: Directed Mutagenesis: A Practical Approach, 1991, Edited by M. J. McPHERSON, IRL PRESS), or are methods for which it is possible to employ commercial kits (for example the U. S. E. mutagenesis kit from PHARMACIA). After the site-directed mutagenesis, it is useful to select the cells which contain a mutated HPPD which is less sensitive to an HPPD inhibitor by using an appropriate screening aid. Appropriate screening methods to achieve this have been described above.

In accordance with the present invention, the term “said position corresponding to position X”, X being any number to be found in the respective context in the present application, does not only include the respective position in the SEQ ID No. referred to afterwards but also includes any sequence encoding an HPPD protein, where, after alignment with the reference SEQ ID No., the respective position might have a different number but corresponds to that indicated for the reference SEQ ID No. Whereas HPPD sequences may be very diverse and may only show a low sequence identity of about 30%, HPPD proteins are characterized by a common three dimensional consensus structure which is achieved despite a low sequence identity. Due to specific positions being conserved within HPPD proteins, alignment of HPPD proteins can be effected by applying various alignment tools in a senseful manner.

Methods of aligning nucleic acid or amino acid sequences and, accordingly, determining the sequence identity of two or more sequences, are well-known in the art. They include performing mathematical algorithms such as the algorithm of Myers and Miller (1988) CABIOS 4:11-17 or the local alignment algorithm of Smith and Waterman (1981) Adv. Appl. Math. 2:482-489; the global alignment algorithm of Needleman and Wunsch (1970) J. Mol. Biol. 48:443-453; the algorithm of Karlin and Altschul (1990) Proc. Natl. Acad. Sci USA 872264 and that of Brutlag et al. (Comp. App. Biosci. 6:237-245 (1990)).

Such algorithms can be implemented in computer programs including but not limited to CLUSTALX, ALIGN, GAP, BESTFIT, BLAST, FASTDB and FASTA.

For example, when using BESTFIT (Wisconsin Sequence Analysis Package, Version 8 for Unix, Genetics Computer Group, University Research Park, 575 Science Drive, Madison, WI 53711) or any other sequence alignment program to determine whether a particular sequence is, for instance, 95% identical to a reference sequence, the parameters are set, of course, such that the percentage of identity is calculated over the full length of the reference nucleotide sequence and that gaps in homology of up to 5% of the total number of nucleotides in the reference sequence are allowed.

The identity between a first sequence and a second sequence, also referred to as a global sequence alignment, is determined using the FASTDB computer program based on the algorithm of Brutlag and colleagues (Comp. App. Biosci. 6:237-245 (1990)). In a sequence alignment the query and subject sequences are both DNA sequences. The result of said global sequence alignment is in percent identity. Preferred parameters used in a FASTDB alignment of DNA sequences to calculate percent identity are: Matrix=Unitary, k-tuple=4, Mismatch Penalty=1, Joining Penalty=30, Randomization Group Length=0, Cutoff Score=1, Gap Penalty=5, Gap Size Penalty 0.05, Window Size=500 or the length of the subject nucleotide sequence, whichever is shorter.

The present invention is based on the results of a combination of a comparison of the amino acid sequences of HPPD proteins from various organisms and the analysis of the substrate binding and inhibitor binding site of selected HPPD proteins using X-ray crystallography. Using this combined approach, it was possible to determine key positions in HPPD proteins, where an amino acid can be replaced with one of a defined set of other amino acids in order to modulate HPPD catalytic activity and the affinity to at least one HPPD herbicide inhibitor to a plant expressing the mutated HPPD protein.

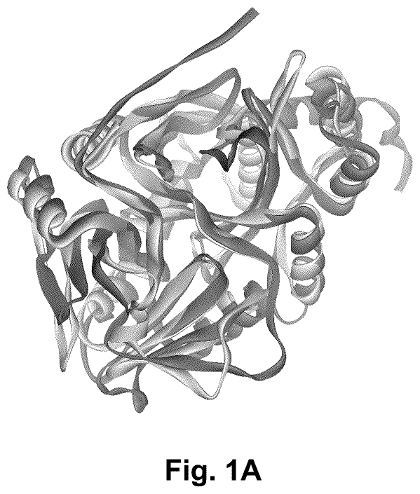

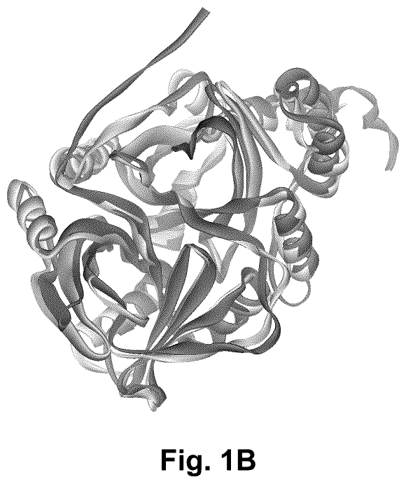

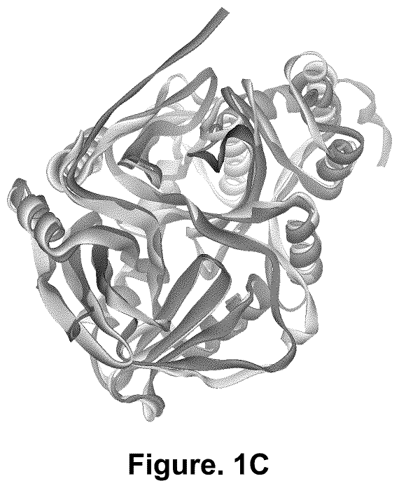

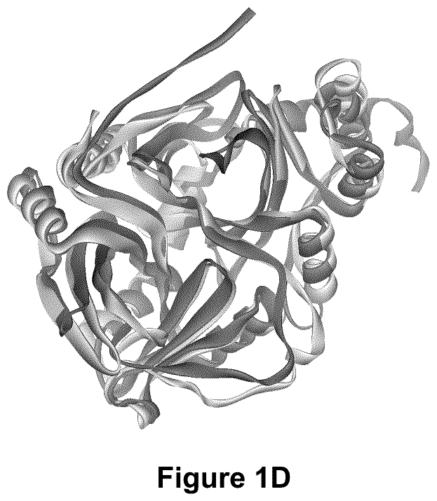

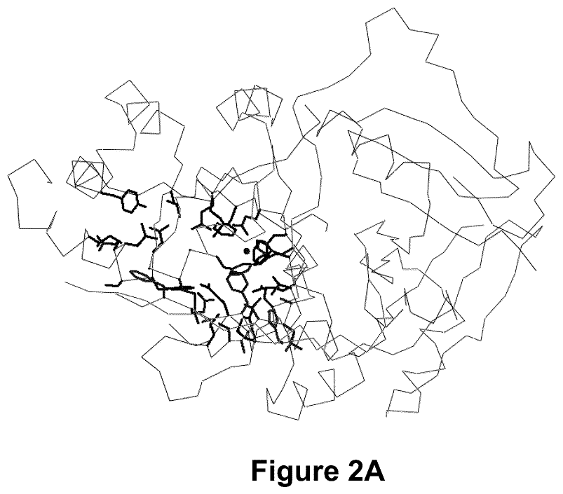

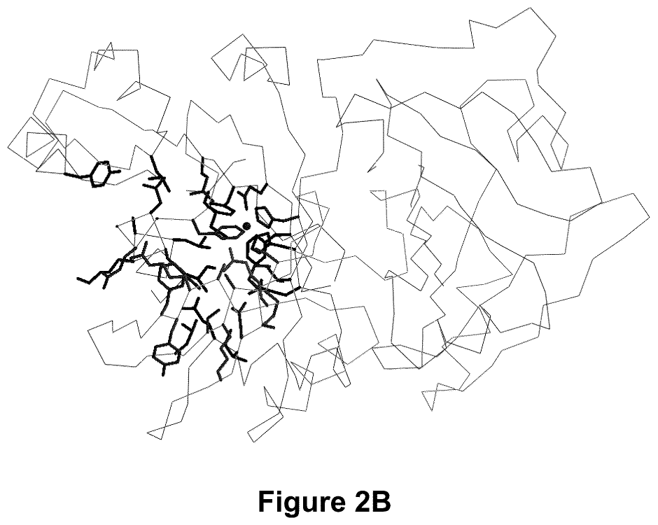

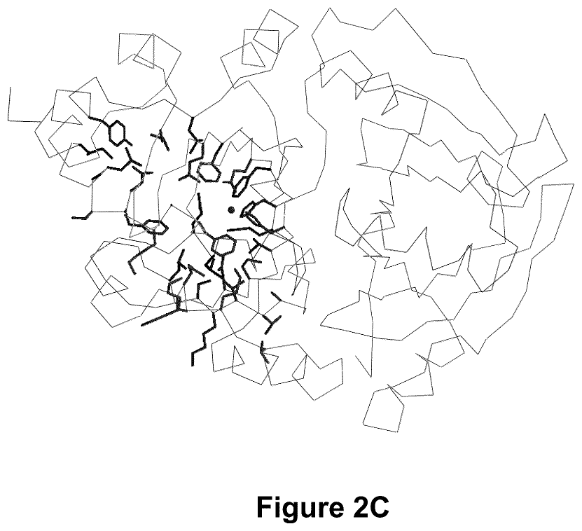

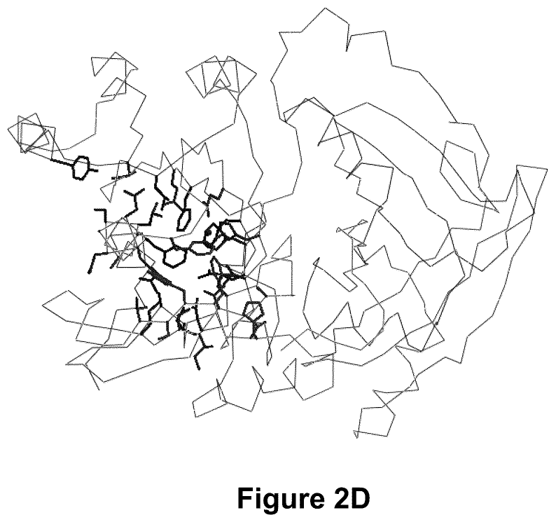

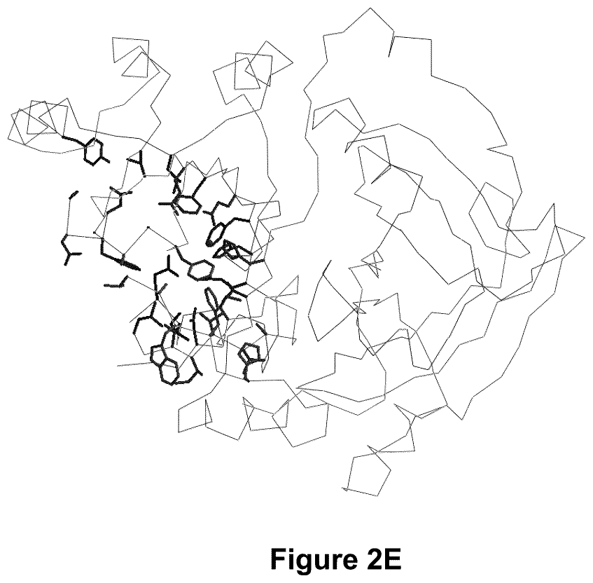

Superposition of the 3D structure of HPPD from Arabidopsis thaliana (1TFZ) (Yang et al., 2004, Biochemistry 43, 10414-10423) with the 3D structures of HPPD from other species such as Pseudomonas fluorescens (1CJX) (Serre et al., 1999, Structure Fold Des. 7, 977-988), Streptomyces avermitilis (1T47) (Brownlee et al., 2004, Biochemistry 43, 6370-6377), Homo sapiens (3ISQ) (PDB ID: 3isq Pilka et al., Structural Genomics Consortium (SGC). Crystal structure of human 4-Hydroxyphenylpyruvate dioxygenase), Rattus norvegicus (l SQI) (Yang et al., 2004, Biochemistry 43, 10414-10423) shows that they have the same folding and corresponding amino acids are at equivalent position in the 3D structure of the protein. Since the species with known 3D structures are very diverse in their amino acid sequence, it can be assumed that all HPPD sequences have the same basic folding even though the overall sequence identity is low. The sequence and the 3D structure of Arabidopsis thaliana has been used as reference structure in the present invention. FIG. 1 shows the superposition of the structure of A. thaliana HPPD with the structure of (a) Pseudomonas fluorescens, (b) Streptomyces avermitilis, (c) Homo sapiens and (d) Rattus norvegicus. In order to define the binding site of the substrate and/or inhibitors, amino acids were selected which play a role in catalysis or inhibitor binding. This includes amino acids in the active site and amino acids from the C-terminal helix. The 3D arrangement is demonstrated in FIG. 2 which displays the amino acids defined as binding site in case of (a) Arabidopsis thaliana, (b) Pseudomonas fluorescens, (c) Streptomyces avermitilis, (d) Homo sapiens and (e) Rattus norvegicus. The amino acid numbering of the Pseudomonas Fluorescens structure (1cjx) was changed into the numbering according to SEQ ID No. 10. The 36 amino acids defining the binding site including their position are listed in Table 1 for (a) Arabidopsis thaliana, (b) Pseudomonas fluorescens, (c) Streptomyces avermitilis, (d) Homo sapiens and (e) Rattus norvegicus.

TABLE 1
Amino acids forming the binding site in A. thaliana,
P. fluorescens, S. avermitilis, H. sapiens, R. norvegicus

| Arabidopsis | | Pseudomonas | | Streptomyces | | Homo | | Rattus | |
|---|---|---|---|---|---|---|---|---|---|
| thaliana | | fluorescens | | avermitilis | | sapiens | | norvegicus | |
| | Amino | | Amino | | Amino | | Amino | | Amino |
| Position | Acid | Position | Acid | Position | Acid | Position | Acid | Position | Acid |
| 226 | H | 162 | H | 187 | H | 183 | H | 183 | H |
| 228 | V | 164 | T | 189 | V | 185 | V | 185 | V |
| 248 | H | 186 | R | 211 | T | 207 | H | 207 | H |
| 250 | F | 188 | A | 213 | M | 209 | F | 209 | F |
| 251 | A | 189 | R | 214 | K | 210 | W | 210 | W |
| 252 | E | 190 | Y | 215 | E | 211 | S | 211 | S |
| 253 | F | 191 | F | 216 | F | 212 | V | 212 | V |
| 265 | L | 200 | L | 228 | L | 224 | L | 224 | L |
| 267 | S | 202 | S | 230 | S | 226 | S | 226 | S |
| 268 | A | 203 | K | 231 | K | 227 | I | 227 | I |
| 269 | V | 204 | A | 232 | V | 228 | V | 228 | V |
| 270 | L | 205 | M | 233 | V | 229 | V | 229 | V |
| 271 | A | 206 | S | 234 | A | 230 | A | 230 | A |
| 280 | P | 215 | P | 243 | P | 239 | P | 239 | P |
| 282 | N | 217 | N | 245 | N | 241 | N | 241 | N |
| 293 | Q | 226 | Q | 255 | Q | 251 | Q | 251 | Q |
| 294 | I | 227 | I | 256 | I | 252 | I | 252 | I |
| 307 | Q | 240 | Q | 269 | Q | 265 | Q | 265 | Q |
| 308 | H | 241 | H | 270 | H | 266 | H | 266 | H |
| 335 | M | 264 | M | 293 | L | 289 | L | 289 | L |
| 342 | Y | 271 | Y | 299 | Y | 295 | Y | 295 | Y |
| 368 | L | 295 | L | 323 | L | 323 | L | 323 | L |
| 379 | Q | 310 | Q | 334 | Q | 334 | Q | 334 | Q |
| 381 | F | 312 | F | 336 | F | 336 | F | 336 | F |
| 392 | F | 321 | F | 347 | F | 347 | F | 347 | F |
| 394 | E | 323 | E | 349 | E | 349 | E | 349 | E |
| 419 | F | 333 | F | 359 | F | 359 | F | 359 | F |
| 420 | G | 334 | G | 360 | G | 360 | G | 360 | G |
| 421 | K | 335 | E | 361 | K | 361 | A | 361 | A |
| 422 | G | 336 | G | 362 | G | 362 | G | 362 | G |
| 423 | N | 337 | N | 363 | N | 363 | N | 363 | N |
| 424 | F | 338 | F | 364 | F | 364 | F | 364 | F |
| 425 | S | 339 | K | 365 | K | 365 | N | 365 | N |
| 426 | E | 340 | A | 366 | A | 366 | S | 366 | S |
| 427 | L | 341 | L | 367 | L | 367 | L | 367 | L |
| 431 | I | 345 | I | 371 | I | 371 | F | 371 | F |

An exemplary alignment of HPPD proteins is given in Table 2a for the HPPD proteins with known 3D structures. Table 2a gives the numbering of the amino acids of the Arabidopsis sequence and also the amino acids which are common within these HPPD sequences, with these amino acids being designated by an asterisk. On the basis of such an alignment and from the definition of the Arabidopsis amino acid by its position and its nature, it is easy to identify the position of the corresponding amino acid in another HPPD sequence. FIG. 2 shows that this can be done with the alignment of sequences of different plant, mammalian and bacterial origin, demonstrating that this method of alignment, which is well known to a skilled person, can be generalized to any other sequence. An alignment of different HPPD sequences is also described in Patent Application WO 97/49816.

TABLE 2a
Alignment of HPPD sequences with known crystal structures i.e. A.
thaliana, P. fluorescens, S. avermitilis, H. sapiens, R. norvegicus

| | 1TFZ | Pos. | 1CJX | Pos. | 1T47 | Pos. | 3ISQ | Pos. | 1SQI | Common |
|---|---|---|---|---|---|---|---|---|---|---|
| Pos. | A. thaliana | P. fluorescens | | S. avermitilis | | H. Sapiens | | R. norvegicus | | amino acid |
| 1 | M | | — | | — | | — | | | |
| 2 | G | | — | | — | | — | | | |
| 3 | H | | — | | — | | — | | | |
| 4 | Q | | — | | — | | — | | | |
| 5 | N | | — | | — | | — | | | |
| 6 | A | | — | | — | | — | | | |
| 7 | A | | — | | — | | — | | | |
| 8 | V | | — | | — | | — | | | |
| 9 | S | | — | | — | | — | | | |
| 10 | E | | — | | — | | — | | | |
| 11 | N | | — | | — | | — | | | |
| 12 | Q | | — | | — | | — | | | |
| 13 | N | | — | | — | | — | | | |
| 14 | H | | — | 1 | M | | — | | | |
| 15 | D | | — | 2 | T | | — | | | |
| 16 | D | | — | 3 | Q | | — | | | |
| 17 | G | | — | 4 | T | | — | | | |
| 18 | A | | — | 5 | T | | — | | | |
| 19 | A | | — | 6 | H | 8 | G | 8 | G | |
| 20 | S | | — | 7 | H | 9 | A | 9 | P | |
| 21 | S | | — | 8 | T | 10 | K | 10 | K | |
| 22 | P | | — | 9 | P | 11 | P | 11 | P | |
| 23 | G | | — | | — | | — | | | |
| 24 | F | | — | | — | | — | | | |
| 25 | K | | — | | — | | — | | | |
| 26 | L | | — | | — | | — | | | |
| 27 | V | | — | | — | | — | | | |
| 28 | G | | — | | — | | — | | | |
| 29 | F | | — | | — | | — | | | |
| 30 | S | | — | | — | | — | | | |
| 31 | K | | — | | — | | — | | | |
| 32 | F | | — | | — | | — | | | |
| 33 | V | | — | | — | | — | | | |
| 34 | R | | — | 10 | D | 12 | E | 12 | E | |
| 35 | K | 2 | A | 11 | T | 13 | R | 13 | R | |
| 36 | N | 3 | D | 12 | A | 14 | G | 14 | G | |
| 37 | P | 4 | L | 13 | R | 15 | R | 15 | R | |
| 38 | K | 5 | Y | 14 | Q | 16 | F | 16 | F | |
| 39 | S | 6 | E | 15 | A | 17 | L | 17 | L | |
| 40 | D | 7 | N | 16 | D | 18 | H | 18 | H | |
| 41 | K | 8 | P | 17 | P | | — | | | |
| 42 | F | 9 | M | 18 | F | | — | | | |
| 43 | K | 10 | G | 19 | P | | — | | | |
| 44 | V | 11 | L | 20 | V | | — | | | |
| 45 | K | 12 | M | 21 | K | | — | | | |
| 46 | R | 13 | G | 22 | G | | — | | | |
| 47 | F | 14 | F | 23 | M | 19 | F | 19 | F | |
| 48 | H | 15 | E | 24 | D | 20 | H | 20 | H | |
| 49 | H | 16 | F | 25 | A | 21 | S | 21 | S | |
| 50 | I | 17 | I | 26 | V | 22 | V | 22 | V | |
| 51 | E | 18 | E | 27 | V | 23 | T | 23 | T | |
| 52 | F | 19 | F | 28 | F | 24 | F | 24 | F | x |
| 53 | W | 20 | A | 29 | A | 25 | W | 25 | W | |
| 54 | C | 21 | S | 30 | V | 26 | V | 26 | V | |
| 55 | G | 22 | P | 31 | G | 27 | G | 27 | G | |
| 56 | D | 23 | T | 32 | N | 28 | N | 28 | N | |
| 57 | A | 24 | P | 33 | A | 29 | A | 29 | A | |
| 58 | T | 25 | G | 34 | K | 30 | K | 30 | K | |
| 59 | N | 26 | T | 35 | Q | 31 | Q | 31 | Q | |
| 60 | V | 27 | L | 36 | A | 32 | A | 32 | A | |
| 61 | A | 28 | E | 37 | A | 33 | A | 33 | A | |
| 62 | R | 29 | P | 38 | H | 34 | S | 34 | S | |
| 63 | R | 30 | I | 39 | Y | 35 | F | 35 | F | |
| 64 | F | 31 | F | 40 | Y | 36 | Y | 36 | Y | |
| 65 | S | 32 | E | 41 | S | 37 | C | 37 | C | |
| 66 | W | 33 | I | 42 | T | 38 | S | 38 | N | |
| 67 | G | 34 | M | 43 | A | 39 | K | 39 | K | |
| 68 | L | 35 | G | 44 | F | 40 | M | 40 | M | |
| 69 | G | 36 | F | 45 | G | 41 | G | 41 | G | |
| 70 | M | 37 | T | 46 | M | 42 | F | 42 | F | |
| 71 | R | 38 | K | 47 | Q | 43 | E | 43 | E | |
| 72 | F | 39 | V | 48 | L | 44 | P | 44 | P | |
| 73 | S | 40 | A | 49 | V | 45 | L | 45 | L | |
| 74 | A | 41 | T | 50 | A | 46 | A | 46 | A | |
| 75 | K | 42 | H | 51 | Y | 47 | Y | 47 | Y | |
| 76 | S | 43 | R | 52 | S | 48 | R | 48 | K | |
| 77 | D | 44 | S | 53 | G | 49 | G | 49 | G | |
| 78 | L | 45 | K | 54 | P | 50 | L | 50 | L | |
| 79 | S | 46 | N | 55 | E | 51 | E | 51 | E | |
| 80 | T | | — | 56 | N | 52 | T | 52 | T | |
| 81 | G | | — | 57 | G | 53 | G | 53 | G | |
| 82 | N | | — | 58 | S | 54 | S | 54 | S | |
| 83 | M | | — | 59 | R | 55 | R | 55 | R | |
| 84 | V | | — | 60 | E | 56 | E | 56 | E | |
| 85 | H | | — | 61 | T | 57 | V | 57 | V | |
| 86 | A | | — | 62 | A | 58 | V | 58 | V | |
| 87 | S | 47 | V | 63 | S | 59 | S | 59 | S | |
| 88 | Y | 48 | H | 64 | Y | 60 | H | 60 | H | |
| 89 | L | 49 | L | 65 | V | 61 | V | 61 | V | |
| 90 | L | 50 | Y | 66 | L | 62 | I | 62 | I | |
| 91 | T | 51 | R | 67 | T | 63 | K | 63 | K | |
| 92 | S | 52 | Q | 68 | N | 64 | Q | 64 | Q | |
| 93 | G | 53 | G | 69 | G | 65 | G | 65 | G | X |
| 94 | D | 54 | E | 70 | S | 66 | K | 66 | K | |
| 95 | L | 55 | I | 71 | A | 67 | I | 67 | I | |
| 96 | R | 56 | N | 72 | R | 68 | V | 68 | V | |
| 97 | F | 57 | L | 73 | F | 69 | F | 69 | F | |
| 98 | L | 58 | I | 74 | V | 70 | V | 70 | V | |
| 99 | F | 59 | L | 75 | L | 71 | L | 71 | L | |
| 100 | T | 60 | N | 76 | T | 72 | S | 72 | C | |
| 101 | A | 61 | N | 77 | S | 73 | S | 73 | S | |
| 102 | P | 62 | E | 78 | V | 74 | A | 74 | A | |
| 103 | Y | 63 | P | 79 | I | 75 | L | 75 | L | |
| 104 | S | 64 | N | 80 | K | 76 | N | 76 | N | |
| 105 | P | 65 | S | 81 | P | | — | | | |
| 106 | S | 66 | I | 82 | A | | — | | | |
| 107 | L | 67 | A | 83 | T | | — | | | |
| 108 | S | 68 | S | 84 | P | 77 | P | 77 | P | |
| 109 | A | | — | 85 | W | 78 | W | 78 | W | |
| 110 | G | | — | 86 | G | 79 | N | 79 | N | |
| 111 | E | | — | 87 | H | 80 | K | 80 | K | |
| 112 | I | | — | 88 | F | 81 | E | 81 | E | |
| 113 | K | | — | 89 | L | 82 | M | 82 | M | |
| 114 | P | | — | 90 | A | 83 | G | 83 | G | |
| 115 | T | | — | | — | | — | | | |
| 116 | T | | — | | — | | — | | | |
| 117 | T | | — | | — | | — | | | |
| 118 | A | | — | | — | | — | | | |
| 119 | S | | — | | — | | — | | | |
| 120 | I | | — | | — | | — | | | |
| 121 | P | | — | | — | | — | | | |
| 122 | S | | — | | — | | — | | | |
| 123 | F | | — | | — | | — | | | |
| 124 | D | | — | | — | | — | | | |
| 125 | H | | — | | — | | — | | | |
| 126 | G | | — | | — | | — | | | |
| 127 | S | | — | | — | | — | | | |
| 128 | C | | — | | — | | — | | | |
| 129 | R | | — | | — | | — | | | |
| 130 | S | 69 | Y | 91 | D | 84 | D | 84 | D | |
| 131 | F | 70 | F | 92 | H | 85 | H | 85 | H | |
| 132 | F | 71 | A | 93 | V | 86 | L | 86 | L | |
| 133 | S | 72 | A | 94 | A | 87 | V | 87 | V | |
| 134 | S | 73 | E | 95 | E | 88 | K | 88 | K | |
| 135 | H | 74 | H | 96 | H | 89 | H | 89 | H | X |
| 136 | G | 75 | G | 97 | G | 90 | G | 90 | G | X |
| 137 | L | 76 | P | 98 | D | 91 | D | 91 | D | |
| 138 | G | 77 | S | 99 | G | 92 | G | 92 | G | |
| 139 | V | 78 | V | 100 | V | 93 | V | 93 | V | X |
| 140 | R | 79 | C | 101 | V | 94 | K | 94 | K | |
| 141 | A | 80 | G | 102 | D | 95 | D | 95 | D | |
| 142 | V | 81 | M | 103 | L | 96 | I | 96 | I | |
| 143 | A | 82 | A | 104 | A | 97 | A | 97 | A | X |
| 144 | I | 83 | F | 105 | I | 98 | F | 98 | F | |
| 145 | E | 84 | R | 106 | E | 99 | E | 99 | E | |
| 146 | V | 85 | V | 107 | V | 100 | V | 100 | V | X |
| 147 | E | 86 | K | 108 | P | 101 | E | 101 | E | |
| 148 | D | 87 | D | 109 | D | 102 | D | 102 | D | X |
| 149 | A | 88 | S | 110 | A | 103 | C | 103 | C | |
| 150 | E | 89 | Q | 111 | R | 104 | D | 104 | E | |
| 151 | S | 90 | K | 112 | A | 105 | Y | 105 | H | |
| 152 | A | 91 | A | 113 | A | 106 | I | 106 | I | |
| 153 | F | 92 | Y | 114 | H | 107 | V | 107 | V | |
| 154 | S | 93 | N | 115 | A | 108 | Q | 108 | Q | |
| 155 | I | 94 | R | 116 | Y | 109 | K | 109 | K | |
| 156 | S | 95 | A | 117 | A | 110 | A | 110 | A | |
| 157 | V | 96 | L | 118 | I | 111 | R | 111 | R | |
| 158 | A | 97 | E | 119 | E | 112 | E | 112 | E | |
| 159 | N | 98 | L | 120 | H | 113 | R | 113 | R | |
| 160 | G | 99 | G | 121 | G | 114 | G | 114 | G | X |
| 161 | A | 100 | A | 122 | A | 115 | A | 115 | A | |
| 162 | I | 101 | Q | 123 | R | 116 | K | 116 | K | |
| 163 | P | | — | 124 | S | 117 | I | 117 | I | |
| 164 | S | | — | 125 | V | 118 | M | 118 | V | |
| 165 | S | | — | 126 | A | 119 | R | 119 | R | |
| 166 | P | | — | 127 | E | 120 | E | 120 | E | |
| 167 | P | 102 | P | 128 | P | 121 | P | 121 | P | X |
| 168 | I | 103 | I | 129 | Y | 122 | W | 122 | W | |
| 169 | V | 104 | H | 130 | E | 123 | V | 123 | V | |
| 170 | L | 105 | I | 131 | L | 124 | E | 124 | E | |
| 171 | N | 106 | D | 132 | K | 125 | Q | 125 | E | |
| 172 | E | 107 | T | 133 | D | 126 | D | 126 | D | |
| 173 | A | 108 | G | 134 | E | 127 | K | 127 | K | |
| 174 | V | 109 | P | 135 | H | 128 | F | 128 | F | |
| 175 | T | 110 | M | 136 | G | 129 | G | 129 | G | |
| 176 | I | 111 | E | 137 | T | 130 | K | 130 | K | |
| 177 | A | 112 | L | 138 | V | 131 | V | 131 | V | |
| 178 | E | 113 | N | 139 | V | 132 | K | 132 | K | |
| 179 | V | 114 | L | 140 | L | 133 | F | 133 | F | |
| 180 | K | 115 | P | 141 | A | 134 | A | 134 | A | |
| 181 | L | 116 | A | 142 | A | 135 | V | 135 | V | |
| 182 | Y | 117 | I | 143 | I | 136 | L | 136 | L | |
| 183 | G | 118 | K | 144 | A | 137 | Q | 137 | Q | |
| 184 | D | 119 | G | 145 | T | 138 | T | 138 | T | |
| 185 | V | 120 | I | 146 | Y | 139 | Y | 139 | Y | |
| 186 | V | 121 | G | 147 | G | 140 | G | 140 | G | |
| 187 | L | 122 | G | 148 | K | 141 | D | 141 | D | |
| 188 | R | 123 | A | 149 | T | 142 | T | 142 | T | |
| 189 | Y | 124 | P | 150 | R | 143 | T | 143 | T | |
| 190 | V | 125 | L | 151 | H | 144 | H | 144 | H | |
| 191 | S | 126 | Y | 152 | T | 145 | T | 145 | T | |
| 192 | Y | 127 | L | 153 | L | 146 | L | 146 | L | |
| 193 | K | 128 | I | 154 | V | 147 | V | 147 | V | |
| 194 | A | 129 | D | 155 | D | 148 | E | 148 | E | |
| 195 | E | 130 | R | 156 | R | 149 | K | 149 | K | |
| 196 | D | 131 | F | 157 | T | 150 | M | 150 | I | |
| 197 | T | 132 | G | 158 | G | 151 | N | 151 | N | |
| 198 | E | 133 | E | 159 | Y | 152 | Y | 152 | Y | |
| | — | 134 | G | | — | | — | | | |
| 199 | K | 135 | S | 160 | D | 153 | I | 153 | T | |
| 200 | S | 136 | S | 161 | G | 154 | G | 154 | G | |
| 201 | E | 137 | I | 162 | P | 155 | Q | 155 | R | |
| 202 | F | 138 | Y | 163 | Y | 156 | F | 156 | F | |
| 203 | L | 139 | D | 164 | L | 157 | L | 157 | L | |
| 204 | P | 140 | I | 165 | P | 158 | P | 158 | P | |
| 205 | G | 141 | D | 166 | G | 159 | G | 159 | G | |
| 206 | F | 142 | F | 167 | Y | 160 | Y | 160 | F | |
| 207 | E | 143 | V | 168 | V | 161 | E | 161 | E | |
| 208 | R | 144 | Y | 169 | A | 162 | A | 162 | A | |
| 209 | V | 145 | L | 170 | A | 163 | P | 163 | P | |
| 210 | E | 146 | E | 171 | A | 164 | A | 164 | T | |
| 211 | D | 147 | G | | — | 165 | F | 165 | Y | |
| | — | | — | | — | 166 | M | 166 | K | |
| | — | | — | | — | 167 | D | 167 | D | |
| | — | | — | 172 | P | 168 | P | 168 | T | |
| 212 | A | 148 | V | 173 | I | 169 | L | 169 | L | |
| 213 | S | 149 | E | 174 | V | 170 | L | 170 | L | |
| 214 | S | 150 | R | 175 | E | 171 | P | 171 | P | |
| 215 | F | 151 | N | 176 | P | 172 | K | 172 | K | |
| 216 | P | 152 | P | 177 | P | 173 | L | 173 | L | |
| 217 | L | 153 | V | 178 | A | 174 | P | 174 | P | |
| 218 | D | 154 | G | 179 | H | 175 | K | 175 | S | |
| 219 | Y | 155 | A | 180 | R | 176 | C | 176 | C | |
| 220 | G | 156 | G | 181 | T | 177 | S | 177 | N | |
| 221 | I | 157 | L | 182 | F | 178 | L | 178 | L | |
| 222 | R | 158 | K | 183 | Q | 179 | E | 179 | E | |
| 223 | R | 159 | V | 184 | A | 180 | M | 180 | I | |
| 224 | L | 160 | I | 185 | I | 181 | I | 181 | I | |
| 225 | D | 161 | D | 186 | D | 182 | D | 182 | D | x |
| 226 | H | 162 | H | 187 | H | 183 | H | 183 | H | x |
| 227 | A | 163 | L | 188 | C | 184 | I | 184 | I | |
| 228 | V | 164 | T | 189 | V | 185 | V | 185 | V | |
| 229 | G | 165 | H | 190 | G | 186 | G | 186 | G | |
| 230 | N | 166 | N | 191 | N | 187 | N | 187 | N | x |
| 231 | V | 167 | V | 192 | V | 188 | Q | 188 | Q | |
| 232 | P | 168 | Y | 193 | E | 189 | P | 189 | P | |
| | — | 169 | R | 194 | L | 190 | D | 190 | D | |
| | — | 170 | G | 195 | G | 191 | Q | 191 | Q | |
| 233 | E | 171 | R | 196 | R | 192 | E | 192 | E | |
| 234 | L | 172 | M | 197 | M | 193 | M | 193 | M | |
| 235 | G | 173 | V | 198 | N | 194 | V | 194 | E | |
| 236 | P | 174 | Y | 199 | E | 195 | S | 195 | S | |
| 237 | A | 175 | W | 200 | W | 196 | A | 196 | A | |
| 238 | L | 176 | A | 201 | V | 197 | S | 197 | S | |
| 239 | T | 177 | N | 202 | G | 198 | E | 198 | E | |
| 240 | Y | 178 | F | 203 | F | 199 | W | 199 | W | |
| 241 | V | 179 | Y | 204 | Y | 200 | Y | 200 | Y | |
| 242 | A | 180 | E | 205 | N | 201 | L | 201 | L | |
| 243 | G | 181 | K | 206 | K | 202 | K | 202 | K | |
| 244 | F | 182 | L | 207 | V | 203 | N | 203 | N | |
| 245 | T | 183 | F | 208 | M | 204 | L | 204 | L | |
| 246 | G | 184 | N | 209 | G | 205 | Q | 205 | Q | |
| 247 | F | 185 | F | 210 | F | 206 | F | 206 | F | x |
| 248 | H | 186 | R | 211 | T | 207 | H | 207 | H | |
| 249 | Q | 187 | E | 212 | N | 208 | R | 208 | R | |
| 250 | F | 188 | A | 213 | M | 209 | F | 209 | F | |
| 251 | A | 189 | R | 214 | K | 210 | W | 210 | W | |
| 252 | E | 190 | Y | 215 | E | 211 | S | 211 | S | |
| 253 | F | 191 | F | 216 | F | 212 | V | 212 | V | |
| 254 | T | 192 | D | 217 | V | 213 | D | 213 | D | |
| 255 | A | 193 | I | 218 | G | 214 | D | 214 | D | |
| 256 | D | 194 | K | 219 | D | 215 | T | 215 | T | |
| 257 | D | 195 | G | 220 | D | 216 | Q | 216 | Q | |
| 258 | V | 196 | E | 221 | I | 217 | V | 217 | V | |
| 259 | G | | — | 222 | A | 218 | H | 218 | H | |
| 260 | T | | — | 223 | T | 219 | T | 219 | T | |
| 261 | A | | — | 224 | E | 220 | E | 220 | E | |
| 262 | E | 197 | Y | 225 | Y | 221 | Y | 221 | Y | |
| 263 | S | 198 | T | 226 | S | 222 | S | 222 | S | |
| 264 | G | 199 | G | 227 | A | 223 | S | 223 | S | |
| 265 | L | 200 | L | 228 | L | 224 | L | 224 | L | x |
| 266 | N | 201 | T | 229 | M | 225 | R | 225 | R | |
| 267 | S | 202 | S | 230 | S | 226 | S | 226 | S | x |
| 268 | A | 203 | K | 231 | K | 227 | I | 227 | I | |
| 269 | V | 204 | A | 232 | V | 228 | V | 228 | V | |
| 270 | L | 205 | M | 233 | V | 229 | V | 229 | V | |
| 271 | A | 206 | S | 234 | A | 230 | A | 230 | A | x |
| 272 | S | 207 | A | 235 | D | 231 | N | 231 | N | |
| 273 | N | 208 | P | 236 | G | 232 | Y | 232 | Y | |
| 274 | D | 209 | D | 237 | T | 233 | E | 233 | E | |
| 275 | E | 210 | G | 238 | L | 234 | E | 234 | E | |
| 276 | M | 211 | M | 239 | K | 235 | S | 235 | S | |
| 277 | V | 212 | I | 240 | V | 236 | I | 236 | I | |
| 278 | L | 213 | R | 241 | K | 237 | K | 237 | K | |
| 279 | L | 214 | I | 242 | F | 238 | M | 238 | M | |
| 280 | P | 215 | P | 243 | P | 239 | P | 239 | P | x |
| 281 | I | 216 | L | 244 | I | 240 | I | 240 | I | |
| 282 | N | 217 | N | 245 | N | 241 | N | 241 | N | x |
| 283 | E | 218 | E | 246 | E | 242 | E | 242 | E | x |
| 284 | P | 219 | E | 247 | P | 243 | P | 243 | P | |
| 285 | V | 220 | S | 248 | A | 244 | A | 244 | A | |
| 286 | H | | — | 249 | L | 245 | P | 245 | P | |
| 287 | G | | — | 250 | A | 246 | G | 246 | G | |
| 288 | T | 221 | S | 251 | K | 247 | K | 247 | R | |
| 289 | K | 222 | K | 252 | K | 248 | K | 248 | K | x |
| 290 | R | 223 | G | | — | | — | | | |
| 291 | K | 224 | A | 253 | K | 249 | K | 249 | K | |
| 292 | S | 225 | G | 254 | S | 250 | S | 250 | S | |
| 293 | Q | 226 | Q | 255 | Q | 251 | Q | 251 | Q | x |
| 294 | I | 227 | I | 256 | I | 252 | I | 252 | I | x |
| 295 | Q | 228 | E | 257 | D | 253 | Q | 253 | Q | |
| 296 | T | 229 | E | 258 | E | 254 | E | 254 | E | |
| 297 | Y | 230 | F | 259 | Y | 255 | Y | 255 | Y | |
| 298 | L | 231 | L | 260 | L | 256 | V | 256 | V | |
| 299 | E | 232 | M | 261 | E | 257 | D | 257 | D | |
| 300 | H | 233 | Q | 262 | F | 258 | Y | 258 | Y | |
| 301 | N | 234 | F | 263 | Y | 259 | N | 259 | N | |
| 302 | E | 235 | N | 264 | G | 260 | G | 260 | G | |
| 303 | G | 236 | G | 265 | G | 261 | G | 261 | G | x |
| 304 | A | 237 | E | 266 | A | 262 | A | 262 | A | |
| 305 | G | 238 | G | 267 | G | 263 | G | 263 | G | x |
| 306 | L | 239 | I | 268 | V | 264 | V | 264 | V | |
| 307 | Q | 240 | Q | 269 | Q | 265 | Q | 265 | Q | x |
| 308 | H | 241 | H | 270 | H | 266 | H | 266 | H | x |
| 309 | L | 242 | V | 271 | I | 267 | I | 267 | I | |
| 310 | A | 243 | A | 272 | A | 268 | A | 268 | A | x |
| 311 | L | 244 | F | 273 | L | 269 | L | 269 | L | |
| 312 | M | 245 | L | 274 | N | 270 | K | 270 | R | |
| 313 | S | 246 | T | 275 | T | 271 | T | 271 | T | |
| 314 | E | 247 | D | 276 | G | 272 | E | 272 | E | |
| 315 | D | 248 | D | 277 | D | 273 | D | 273 | D | x |
| 316 | I | 249 | L | 278 | I | 274 | I | 274 | I | |
| 317 | F | 250 | V | 279 | V | 275 | I | 275 | I | |
| 318 | R | 251 | K | 280 | E | 276 | T | 276 | T | |
| 319 | T | 252 | T | 281 | T | 277 | A | 277 | T | |
| 320 | L | 253 | W | 282 | V | 278 | I | 278 | I | |
| 321 | R | 254 | D | 283 | R | 279 | R | 279 | R | |
| 322 | E | 255 | A | 284 | T | 280 | H | 280 | H | |
| 323 | M | 256 | L | 285 | M | 281 | L | 281 | L | |
| 324 | R | 257 | K | 286 | R | 282 | R | 282 | R | |
| 325 | K | 258 | K | 287 | A | 283 | E | 283 | E | |
| 326 | R | 259 | I | 288 | A | 284 | R | 284 | R | |
| 327 | S | | — | | — | | — | | | |
| 328 | S | | — | | — | | — | | | |
| 329 | I | | — | | — | | — | | | |
| 330 | G | | — | | — | | — | | | |
| 331 | G | 260 | G | 289 | G | 285 | G | 285 | G | x |
| 332 | F | 261 | M | 290 | V | 286 | L | 286 | M | |
| 333 | D | 262 | R | 291 | Q | 287 | E | 287 | E | |
| 334 | F | 263 | F | 292 | F | 288 | F | 288 | F | x |
| 335 | M | 264 | M | 293 | L | 289 | L | 289 | L | |
| 336 | P | 265 | T | 294 | D | 290 | S | 290 | A | |
| 337 | S | 266 | A | 295 | T | 291 | V | 291 | V | |
| 338 | P | 267 | P | 296 | P | 292 | P | 292 | P | x |
| 339 | P | 268 | P | | — | | — | | | |
| 340 | P | 269 | D | 297 | D | 293 | S | 293 | S | |
| 341 | T | 270 | T | 298 | S | 294 | T | 294 | S | |
| 342 | Y | 271 | Y | 299 | Y | 295 | Y | 295 | Y | x |
| 343 | Y | 272 | Y | 300 | Y | 296 | Y | 296 | Y | x |
| 344 | Q | 273 | E | 301 | D | 297 | K | 297 | R | |
| 345 | N | 274 | M | 302 | T | 298 | Q | 298 | L | |
| 346 | L | 275 | L | 303 | L | 299 | L | 299 | L | x |
| 347 | K | 276 | E | 304 | G | 300 | R | 300 | R | |
| 348 | K | 277 | G | 305 | E | 301 | E | 301 | E | |
| 349 | R | 278 | R | 306 | W | 302 | K | 302 | N | |
| 350 | V | 279 | L | 307 | V | 303 | L | 303 | L | |
| 351 | G | 280 | P | 308 | G | 304 | K | 304 | K | |
| 352 | D | 281 | D | 309 | D | 305 | T | 305 | T | |
| 353 | V | 282 | H | 310 | T | 306 | A | 306 | S | |
| 354 | L | 283 | G | 311 | R | 307 | K | 307 | K | |
| 355 | S | 284 | E | 312 | V | 308 | I | 308 | I | |
| 356 | D | 285 | P | 313 | P | 309 | K | 309 | Q | |
| 357 | D | 286 | V | 314 | V | 310 | V | 310 | V | |
| 358 | Q | 287 | D | | — | 311 | K | 311 | K | |
| | — | 288 | Q | | — | 312 | E | 312 | E | |
| | — | 289 | L | | — | 313 | N | 313 | N | |
| 359 | I | 290 | Q | | — | 314 | I | 314 | M | |
| 360 | K | 291 | A | 315 | D | 315 | D | 315 | D | |
| 361 | E | 292 | R | 316 | T | 316 | A | 316 | V | |
| 362 | C | 293 | G | 317 | L | 317 | L | 317 | L | |
| 363 | E | 294 | I | 318 | R | 318 | E | 318 | E | |
| 364 | E | 295 | L | 319 | E | 319 | E | 319 | E | |
| 365 | L | 296 | L | 320 | L | 320 | L | 320 | L | x |
| 366 | G | 297 | D | 321 | K | 321 | K | 321 | K | |
| 367 | I | 298 | G | 322 | I | 322 | I | 322 | I | |
| 368 | L | 299 | S | 323 | L | 323 | L | 323 | L | |
| 369 | V | 300 | S | 324 | A | 324 | V | 324 | V | |
| 370 | D | 301 | V | 325 | D | 325 | D | 325 | D | |
| 371 | R | 302 | E | 326 | R | 326 | Y | 326 | Y | |
| 372 | D | 303 | G | 327 | D | 327 | D | 327 | D | |
| 373 | D | 304 | D | 328 | E | 328 | E | 328 | E | |
| 374 | Q | 305 | K | 329 | D | 329 | K | 329 | K | |
| 375 | G | 306 | R | 330 | G | 330 | G | 330 | G | |
| 376 | T | 307 | L | 331 | Y | 331 | Y | 331 | Y | |
| 377 | L | 308 | L | 332 | L | 332 | L | 332 | L | x |
| 378 | L | 309 | L | 333 | L | 333 | L | 333 | L | x |
| 379 | Q | 310 | Q | 334 | Q | 334 | Q | 334 | Q | x |
| 380 | I | 311 | I | 335 | I | 335 | I | 335 | I | x |
| 381 | F | 312 | F | 336 | F | 336 | F | 336 | F | x |
| 382 | T | 313 | S | 337 | T | 337 | T | 337 | T | |
| 383 | K | 314 | E | 338 | K | 338 | K | 338 | K | |
| 384 | P | 315 | T | 339 | P | 339 | P | 339 | P | |
| 385 | L | 316 | L | 340 | V | 340 | V | 340 | M | |
| 386 | G | 317 | M | 341 | Q | 341 | Q | 341 | Q | |
| 387 | D | 318 | G | 342 | D | 342 | D | 342 | D | |
| 388 | R | | — | 343 | R | 343 | R | 343 | R | |
| 389 | P | | — | 344 | P | 344 | P | 344 | P | |
| 390 | T | 319 | P | 345 | T | 345 | T | 345 | T | |
| 391 | I | 320 | V | 346 | V | 346 | L | 346 | L | |
| 392 | F | 321 | F | 347 | F | 347 | F | 347 | F | x |
| 393 | I | 322 | F | 348 | F | 348 | L | 348 | L | |
| 394 | E | 323 | E | 349 | E | 349 | E | 349 | E | x |
| 395 | I | 324 | F | 350 | I | 350 | V | 350 | V | |
| 396 | I | 325 | I | 351 | I | 351 | I | 351 | I | x |
| 397 | Q | 326 | Q | 352 | E | 352 | Q | 352 | Q | |
| 398 | R | 327 | R | 353 | R | 353 | R | 353 | R | x |
| 399 | V | 328 | K | 354 | H | 354 | H | 354 | H | |
| 400 | G | | — | | — | | — | | | |
| 401 | C | | — | | — | | — | | | |
| 402 | M | | — | | — | | — | | | |
| 403 | M | | — | | — | | — | | | |
| 404 | K | | — | | — | | — | | | |
| 405 | D | | — | | — | | — | | | |
| 406 | E | | — | | — | | — | | | |
| 407 | E | | — | | — | | — | | | |
| 408 | G | | — | | — | | — | | | |
| 409 | K | | — | | — | | — | | | |
| 410 | A | | — | | — | | — | | | |
| 411 | Y | | — | | — | | — | | | |
| 412 | Q | | — | | — | | — | | | |
| 413 | S | | — | | — | | — | | | |
| 414 | G | | — | | — | | — | | | |
| 415 | G | 329 | G | 355 | G | 355 | N | 355 | N | |
| 416 | C | 330 | D | 356 | S | 356 | H | 356 | H | |
| 417 | G | 331 | D | 357 | M | 357 | Q | 357 | Q | |
| 418 | G | 332 | G | 358 | G | 358 | G | 358 | G | x |
| 419 | F | 333 | F | 359 | F | 359 | F | 359 | F | x |
| 420 | G | 334 | G | 360 | G | 360 | G | 360 | G | x |
| 421 | K | 335 | E | 361 | K | 361 | A | 361 | A | |
| 422 | G | 336 | G | 362 | G | 362 | G | 362 | G | x |
| 423 | N | 337 | N | 363 | N | 363 | N | 363 | N | x |
| 424 | F | 338 | F | 364 | F | 364 | F | 364 | F | x |
| 425 | S | 339 | K | 365 | K | 365 | N | 365 | N | |
| 426 | E | 340 | A | 366 | A | 366 | S | 366 | S | |
| 427 | L | 341 | L | 367 | L | 367 | L | 367 | L | x |
| 428 | F | 342 | F | 368 | F | 368 | F | 368 | F | x |
| 429 | K | 343 | E | 369 | E | 369 | K | 369 | K | |
| 430 | S | 344 | S | 370 | A | 370 | A | 370 | A | |
| 431 | I | 345 | I | 371 | I | 371 | F | 371 | F | |
| 432 | E | 346 | E | 372 | E | 372 | E | 372 | E | x |
| 433 | E | 347 | R | 373 | R | 373 | E | 373 | E | |
| 434 | Y | 348 | D | 374 | E | 374 | E | 374 | E | |
| 435 | E | 349 | Q | 375 | Q | 375 | Q | 375 | Q | |
| 436 | K | 350 | V | 376 | E | 376 | N | 376 | A | |
| 437 | T | 351 | R | 377 | K | 377 | L | 377 | L | |
| 438 | L | 352 | R | 378 | R | 378 | R | 378 | R | |
| 439 | E | 353 | G | 379 | G | 379 | G | 379 | G | |
| 440 | A | 354 | V | 380 | N | 380 | N | 380 | N | |
| 441 | K | 355 | L | 381 | L | 381 | L | 381 | L | |
| 442 | Q | 356 | A | | | 382 | T | 382 | T | |
| 443 | L | 357 | T | | | 383 | N | 383 | D | |
| 444 | V | 358 | D | | | 384 | M | 384 | L | |
| 445 | G | | | | | 385 | E | 385 | E | |
| | | | | | | 386 | T | 386 | T | |
| | | | | | | 387 | N | 387 | N | |
| | | | | | | 388 | G | 388 | G | |
| | | | | | | 389 | V | 389 | V | |
| | | | | | | 390 | V | 390 | R | |
| | | | | | | 391 | P | 391 | S | |
| | | | | | | 392 | G | 392 | G | |
| | | | | | | 393 | M | 393 | M | |
| | | | | | | 394 | A | 394 | | |
| | | | | | | 395 | E | 395 | | |
| | | | | | | 396 | N | 396 | | |
| | | | | | | 397 | L | 397 | | |
| | | | | | | 398 | Y | 398 | | |
| | | | | | | 399 | F | 399 | | |
| | | | | | | 400 | Q | 400 | | |

A sequence analysis of more than 700 HPPD sequences from public data bases including sequences of HPPD proteins and predicted HPPD proteins such as from plants, mammals, fungi and bacteria was performed using ClustalX. The alignment was corrected using the information of the available 3D structures. Identical amino acid sequences with different identifiers were included only once and some sequences with obvious sequence errors were excluded. The alignment also includes incomplete sequences. Table 2b shows the sequence alignment for a representative set of HPPD proteins and includes sequences from plants, bacteria, mammals.

TABLE 2b
Alignment of a representative set of HPPD sequences
CLUSTAL X (1.81) multiple sequence alignment

| | |
|---|---|
| RATTUS_NORVEGICUS | ---MTT-----------------------------------YSNKGPKPE |
| HOMO-SAPIENS | ---------------------------------------------GAKPE |
| XENOPUS-LAEVIS | ---MTS-----------------------------------YTDKGPKPD |
| BLEPHARISMA-JAPONICUS | ---MTY-----------------------------------YDKQETRPD |
| MYCOSPHAERELLA-GRAMINICOLA | ---MAPGALLVTSQNG------------RTSPLYDSDGYVPAPAALVVGG |
| ASPERGILLUS-FUMIGATUS | ---MAPSAISTS----------------------------PPPTDRVSSS |
| MAGNAPORTHE-GRISEA | ---MSPSAITESPRN----------------SVVDHTSGLQVDSLAVQGP |
| CANDIDA-ALBICANS | -------------------------------------------------- |
| PICHIA-STIPITIS | ----LLKELPFLPTSS------------DPITEPDIDELLSDGHVNSKYP |
| RHODOCOCCUS-SP. | ---MTIEQTLTD------------KERLAGLDLGQLEQLVGLVEYDGTRD |
| RHODOCOCCUS-ERYTHROPOLIS | ---MTVEQTLTD------------DEQLAGLDLEQLRQLVGLVEYDSDHD |
| JANIBACTER-SP.-HICC2649 | MTDTTITNPTTDTASQPTPLDLTPQEREANLNLEQLKQLVGLVEYDESKD |
| STREPTOMYCES-AVERMITILIS | ---MTQTTHHTP-------------------------------DTARQAD |
| KORDIA-ALGICIDA | ---MAAEIKNLKD--------------------LQNTEYGLKKLFDEAED |
| LEEUWENHOEKIELLA-BLANDENSIS-ME | ---MSKELK--------------------------SVDYGLEKIFDGAQD |
| GEMMATIMONAS-AURANTIACA | ---MATLTT---------------------------------PEIGTEQD |
| PICROPHILUS-TORRIDUS | --------------------------------------MYGKNLISELRE |
| ARABIDOPSIS-THALIANA | --------MGHQNAAVSENQNHDDGAAS--SPGFKLVGFSKFVRKNPKSD |
| BRASSICA-RAPA-SUBSP.-PEKINENSI | --------MGHENAAVSENQHHDDAATTSASPGFKLVGFSKFVRKNPKSD |
| COPTIS-JAPONICA-VAR.-DISSECTA | --------------MVPST-----------ASNLKLVGHTNFVHNNPKSD |
| HEVEA-BRASILIENSIS | --------MGKENDSVPSS-----------APGFKLLGFSNFVRTNPRSD |
| MEDICAGO-TRUNCATULA | --------MAIETETQTQT-----------QTGFKLVGFKNFVRANPKSD |
| GLYCINE-MAX | -------MCNEIQAQAQAQ----------AQPGFKLVGFKNFVRTNPKSD |
| SOLANUM-LYCOPERSICUM | ----MG--MGKETLS--TTDTTG--------ATFKLVGFNNFIRANPRSD |
| SOLANUM-TUBEROSUM | ----MG--MGKETLSTITTDETG--------ATFKLG-FNNFIRANPRSD |
| NICOTIANA-BENTHAMIANA | ----MGKLETVTTTSATAADDSSEL-----TTNFKLVGFKNFIRTNPRSD |
| DAUCUS-CARDIA | ----MGKKQSEAEILSSNSSNTSP-------ATFKLVGFNNFVRANPKSD |
| SOLENOSTEMON-SCUTELLARIOIDES | ----MG------QESTAAAAVVP--------AEFKLVGHKNFVRSNPMSD |
| HORDEUM-VULGARE | --------MPPTPTTPAAT--GAAAAVTPEHARP-----HRMVRFNPRSD |
| TRITICUM-AESTIVUM | --------MPPTPTTPAATGAGAAAAVTPEHARP-----RRMVRFNPRSD |
| AVENA-SATIVA | --------MPPTPATATGA---AAAAVTPEHAARS---FPRVVRVNPRSD |
| ORYZA-SATIVA | --------MPPTPTPTATTGAVSAAAAAGENAGFRLVGHRRFVRANPRSD |
| SORGHUM-BICOLOR | --------MPPTPTTAAAT-GAAVAAASAEQAAFRLVGHRNFVRVNPRSD |
| ZEA-MAYS | --------MPPTPTAAAAG-AAVAAASAAEQAAFRLVGHRNFVRFNPRSD |
| ABO95005_OLUCIMARINUS | --------MATVPS------------------KRKLVGCANFVRSNPLSD |
| OTAURI | --------MTTSAS------------------GRKLVGHANFVRCNPLSD |
| MICROMONAS-PUSILLA-CCMP1545 | --------MASSEANAAPA-----------AKRHKLVGCKNFVRNNPKSD |
| SYNECHOCOCCUS-SP. | --------------------------------------------MNPSIR |
| VIBRIO-SP.-MED222 | --------------------------------------------MVDTYN |
| MARINOMONAS-SP.-MED121 | --------------------------------------MNTALKIDYSIN |
| PSEUDOMONAS-FLUORESCENS | --------------------------------------------ADLYEN |
| SULFITOBACTER-SP.-NAS-14.1 | ---------------------------------MGPFPHDAEKSKITDEN |
| OCEANICOLA-BATSENSIS-HTCC2597 | ---------------------------------MGPFPHDAPKSEITDEN |
| BDELLOVIBRIO-BACTERIOVORUS | ------------------------------------------MAQVTEKN |
| RATTUS_NORVEGICUS | RGRFLHFHSVTFWVGNAK--QAASFYCNKMGFEPLAYKGLEIGSREVVSH |
| HOMO-SAPIENS | RGRFLHFHSVTFWVGNAK--QAASFYCSKMGFEPLAYRGLETGSREVVSH |
| XENOPUS-LAEVIS | VGRYLAFDHITFYVGNAK--QAAAYYATRFGFNPIAYRGLETGHRDVCTH |
| BLEPHARISMA-JAPONICUS | LGEFYGFHHVRFYVSNSE--QAASFYISRFGFSPVAYEGLETGNQKFCTN |
| MYCOSPHAERELLA-GRAMINICOLA | EVNYRGYHHAEWWVGNAK--QVAQFYITRMGFEPVAHKGLETGSRFFASH |
| ASPERGILLUS-FUMIGATUS | LASYKGYDHVHWYVGNAK--QAASYYITRMGFKRIAYRGLETGCRSVCSH |
| MAGNAPORTHE-GRISEA | FPSFHGYDHVTWWVGNAK--QAASYYNTLFGMKIIAYRGLETGSRYFASY |
| CANDIDA-ALBICANS | ---------------------MSKYLQLAMGFKEVAYKGLETGSKLIGAH |
| PICHIA-STIPITIS | TDGFIKFFSLKICSSNAK--QMSKYLQLAMDFKEIAYKGLENDSRLVGAH |
| RHODOCOCCUS-SP. | PFPVSGWDAVVWVVGNAT--QTAHYFQSAFGMTLVAYSGPTTGNRDHHSF |
| RHODOCOCCUS-ERYTHROPOLIS | PFPVSGWDGLEWIVGNAT--QTSHFFQSAFGMELVAYSGPSTGNRDHHAF |
| JANIBACTER-SP.-HICC2649 | PFPVTGWDAIVFVVGNAT--QAAAYYQGTWGMELVGYSGPENGNRDHKAF |
| STREPTOMYCES-AVERMITILIS | PFPVKGMDAVVFAVGNAK--QAAHYYSTAFGMQLVAYSGPENGSRETASY |
| KORDIA-ALGICIDA | FLPLLGTDYVELYVGNAK--QSAHFYKTAFGFQSEAYAGLETGLTDRVSY |
| LEEUWENHOEKIELLA-BLANDENSIS-ME | FLPLLGTDYVEFYVGNAK--QAAHFYKTAFGFQSEAYSGLETGKKDRVSY |
| GEMMATIMONAS-AURANTIACA | AFPINGTDYVEFYVGNAK--QASHYYRAAFGYSLVAYRGPETGVRDRASY |
| PICROPHILUS-TORRIDUS | KEIFKRLHHVEFYVSSAK--TWSYFMNRGLGFKIVAYAGPETGIRDKISY |
| ARABIDOPSIS-THALIANA | KFKVKRFHHIEFWCGDAT--NVARRFSWGLGMRFSAKSDLSTGNMVHASY |
| BRASSICA-RAPA-SUBSP.-PEKINENSI | KFKVKRFHHIEFWCGDAT--NVARRFSWGLGMRFSAKSDLSTGNMVHASY |
| COPTIS-JAPONICA-VAR.-DISSECTA | KFHVKKFHHIEFWSTDAT--NTARRFSWGLGMPMVAKSDLSTGNMVHASY |
| HEVEA-BRASILIENSIS | LFKVKRFHHVEFWCIDAT--NTACRFSWGLGMPFVAKSDLSTGNVTHASY |
| MEDICAGO-TRUNCATULA | RFNVKRFHHVEFWCIDAT--NTARRFSHGLGMPIVAKSDLSTGNLTHASY |
| GLYCINE-MAX | RFQVNRFHHIEFWCIDAT--NASRRFSWGLGMPIVAKSDLSTGNQIHASY |
| SOLANUM-LYCOPERSICUM | FFSVKRFHHIEFWCGDAT--NTSRRFSWSLGMPITAKSDLSTGNSVHASY |
| SOLANUM-TUBEROSUM | FFSVKRFHHIEFWCGDAT--NTSRRFSWSLGMPITAKSDLSTGNSVHASY |
| NICOTIANA-BENTHAMIANA | FFSVKRFHHIEFWCGDAT--NTSRRFSWSLGMPIAAKSDLSTGNSVHASY |
| DAUCUS-CARDIA | HFAVKRFHHIEFWCGDAT--NTSRRFSWGLGMPLVAKSDLSTGNSVHASY |
| SOLENOSTEMON-SCUTELLARIOIDES | HFPVHRFHHVEFWCGDAT--NTSRRFSWGLGMPLVAKSDLSTGNSAHASY |
| HORDEUM-VULGARE | RFHTLSFHHVEFWCADAA--SAAGRFAFALGAPLAARSDLSTGNSAHASQ |
| TRITICUM-AESTIVUM | RFHTLSFHHVEFWCADAA--SAAGRFAFALGAPLAARSDLSTGNSVHASQ |
| AVENA-SATIVA | RFPVLSFHHVELWCADAA--SAAGRFSFALGAPLAARSDLSTGNSAHASL |
| ORYZA-SATIVA | RFQALAFHHVELWCADAA--SAAGRFAFALGAPLAARSDLSTGNSAHASL |
| SORGHUM-BICOLOR | RFHTLAFHHVELWCADAA--SAAGRFSFGLGAPLAARSDLSTGNTAHASL |
| ZEA-MAYS | RFHTLAFHHVELWCADAA--SAAGRFSFGLGAPLAARSDLSTGNSAHASL |
| ABO95005_OLUCIMARINUS | AFECEKFDHIEFWCGDAT--NAAARFGVGLGMGLRCKSDATTGNGTYASY |
| OTAURI | AFECVGFDHVEFWCGDAT--NAASRFGVGLGMSLRAKSDASTGNGIYASY |
| MICROMONAS-PUSILLA-CCMP1545 | LFTMRKFHHVEFWCRDAT--TTAARFAVGLGMQLVAKSDLTTGNARYASY |
| SYNECHOCOCCUS-SP. | --IVQGIHHLHFYLWDLP--RWREHFCRVWGFRVASDAGN--------TL |
| VIBRIO-SP.-MED222 | PLGTDGFEFVEYTAVDHKGIEQLKALLVSLGFAEIAKHRSKE------AW |
| MARINOMONAS-SP.-MED121 | PLGTDGFEFVEYTAADEKGIADLKALFVSLGFTEVAKHRSKE------VW |
| PSEUDOMONAS-FLUORESCENS | PMGLMGFEFIEFASPTPG---TLEPIFEIMGFTKVATHRSKN------VH |
| SULFITOBACTER-SP.-NAS-14.1 | PAGTDGFEFVEFASADPQ---ELRDLFTRMGYAHVANHKTQK------IE |
| OCEANICOLA-BATSENSIS-HTCC2597 | PAGTDGFEFVEFAHPDPQ---ELRDLFSKMGYELVGRHKSKD------VE |
| BDELLOVIBRIO-BACTERIOVORUS | PVGLNGVDFIEYSGPDAH---FFEQVFKRYAFKEVGQVHGKN------IK |
| RATTUS_NORVEGICUS | VIKQGK--IVFVLCSALNPWN----------------------------- |
| HOMO-SAPIENS | VIKQGK--IVFVLSSALNPWN----------------------------- |
| XENOPUS-LAEVIS | VVRQNN--ATFVFQSPLNPGN----------------------------- |
| BLEPHARISMA-JAPONICUS | VVRSNH--VVIAFTSALTPED----------------------------- |
| MYCOSPHAERELLA-GRAMINICOLA | VVQNNG--VRFVFTSPVRSSA----------------------------- |
| ASPERGILLUS-FUMIGATUS | VVRNGD--ITFILTSPLRS------------------------------- |
| MAGNAPORTHE-GRISEA | LVGKED--VRFVFTSPIRSHVH---------------------------- |
| CANDIDA-ALBICANS | VMQNGS--ITLEIINTLETIDDDNVLKFPFFQNDLNKFRNINHEYFLENF |
| PICHIA-STIPITIS | VIRNGD--VTLEIVNTLETVEDDNVLKFPYFEKDLKQFPQLNESKYLRDF |
| RHODOCOCCUS-SP. | VLESGA--VRFVIKGAVNPDSP---------------------------- |
| RHODOCOCCUS-ERYTHROPOLIS | VLKSGA--VRFVVKGAVDPASP---------------------------- |
| JANIBACTER-SP.-HICC2649 | VLKSGS--IRFVLKGAVSPDSP---------------------------- |
| STREPTOMYCES-AVERMITILIS | VLTNGS--ARFVLTSVIKPATPW--------------------------- |
| KORDIA-ALGICIDA | VLKQDK--IRLVLTTPLGKGGE---------------------------- |
| LEEUWENHOEKIELLA-BLANDENSIS-ME | VLRQDK--IRLVLTSPLGSASP---------------------------- |
| GEMMATIMONAS-AURANTIACA | LMQQGK--IRLVLTTSITADTP---------------------------- |
| PICROPHILUS-TORRIDUS | VMSQGT--ARISFTSSMNDDSY---------------------------- |
| ARABIDOPSIS-THALIANA | LLTSGD--LRFLFTAPYSP---SLSAGEI--------------------- |
| BRASSICA-RAPA-SUBSP.-PEKINENSI | LLTSGD--LRFLFTAPYSP---SLSAGEN--------------------- |
| COPTIS-JAPONICA-VAR.-DISSECTA | LLRSGE--LNFLFTAPYSP---SIAGNT---------------------- |
| HEVEA-BRASILIENSIS | LLRSGD--LSFLFTAPYSP---TIASMENF-------------------- |
| MEDICAGO-TRUNCATULA | LLRSGD--LNFLFSAAYSP---SISLSS---------------------- |
| GLYCINE-MAX | LLRSGD--LSFLFSAPYSP---SLSAGSS--------------------- |
| SOLANUM-LYCOPERSICUM | LLRSVSGELQFVFTAPYSP---SISVPS---------------------- |
| SOLANUM-TUBEROSUM | LLRSVSGELQFVFTAPYSP---SISVPS---------------------- |
| NICOTIANA-BENTHAMIANA | LLRPVSGSLQFLFTAPYSP---SISTPS---------------------- |
| DAUCUS-CARDIA | LVRSAN--LSFVFTAPYSP---STTTSSG--------------------- |
| SOLENOSTEMON-SCUTELLARIOIDES | LLRSGE--LSFVFTAPYSP---SLAEPS---------------------- |
| HORDEUM-VULGARE | LLRSGS--LAFLFTAPYAN-----GCDAA--------------------- |
| TRITICUM-AESTIVUM | LLRSGN--LAFLFTAPYAN-----GCDAA--------------------- |
| AVENA-SATIVA | LLRSGA--LAFLFTAPYAPPPQEAATAAA--------------------- |
| ORYZA-SATIVA | LLRSAS--VAFLFTAPYGGDHGVGADAAT--------------------- |
| SORGHUM-BICOLOR | LLRSGA--LAFLFTAPYAH-----GADAA--------------------- |
| ZEA-MAYS | LLRSGS--LSFLFTAPYAH-----GADAA--------------------- |
| ABO95005_OLUCIMARINUS | AMKSND--LTFVFTAPYG-----VESGGSR-------------------- |
| OTAURI | AMKSHD--LTFVFTAPYGDDERAVGCGGSS-------------------- |
| MICROMONAS-PUSILLA-CCMP1545 | VLQSND--LRFVFSAPYDVPEGEENDDARS-------------------- |
| SYNECHOCOCCUS-SP. | ELEQGS--LRLRLSQPARAG------------------------------ |
| VIBRIO-SP.-MED222 | LYRQGD--INFIVNEQPHS------------------------------- |
| MARINOMONAS-SP.-MED121 | LYRQND--INFIVNSEPNS------------------------------- |
| PSEUDOMONAS-FLUORESCENS | LYRQGE--INLILNNEPNS------------------------------- |
| SULFITOBACTER-SP.-NAS-14.1 | LWQQGD--ITYVLNADPDS------------------------------- |
| OCEANICOLA-BATSENSIS-HTCC2597 | LWQQGD--ITYIINAEPGT------------------------------- |
| BDELLOVIBRIO-BACTERIOVORUS | LFRQGD--INFILNCEPHT------------------------------- |
| RATTUS_NORVEGICUS | -------------------------------------------------- |
| HOMO-SAPIENS | -------------------------------------------------- |
| XENOPUS-LAEVIS | -------------------------------------------------- |
| BLEPHARISMA-JAPONICUS | -------------------------------------------------- |
| MYCOSPHAERELLA-GRAMINICOLA | -------------------------------------------------- |
| ASPERGILLUS-FUMIGATUS | -------------------------------------------------- |
| MAGNAPORTHE-GRISEA | -------------------------------------------------- |
| CANDIDA-ALBICANS | KLITDDLIFDFVNSRIESLSFKSDYLKFGKQFYNNIIRSDDYQESMAKVS |
| PICHIA-STIPITIS | KITTNDLVFDFVNSRIESFSVSPNAHYFRRKLYNKIVSSRAFRNNMFDYN |
| RHODOCOCCUS-SP. | -------------------------------------------------- |
| RHODOCOCCUS-ERYTHROPOLIS | -------------------------------------------------- |
| JANIBACTER-SP.-HICC2649 | -------------------------------------------------- |
| STREPTOMYCES-AVERMITILIS | -------------------------------------------------- |
| KORDIA-ALGICIDA | -------------------------------------------------- |
| LEEUWENHOEKIELLA-BLANDENSIS-ME | -------------------------------------------------- |
| GEMMATIMONAS-AURANTIACA | -------------------------------------------------- |
| PICROPHILUS-TORRIDUS | -------------------------------------------------- |
| ARABIDOPSIS-THALIANA | -------------------------------------------------- |
| BRASSICA-RAPA-SUBSP.-PEKINENSI | -------------------------------------------------- |
| COPTIS-JAPONICA-VAR.-DISSECTA | -------------------------------------------------- |
| HEVEA-BRASILIENSIS | -------------------------------------------------- |
| MEDICAGO-TRUNCATULA | -------------------------------------------------- |
| GLYCINE-MAX | -------------------------------------------------- |
| SOLANUM-LYCOPERSICUM | -------------------------------------------------- |
| SOLANUM-TUBEROSUM | -------------------------------------------------- |
| NICOTIANA-BENTHAMIANA | -------------------------------------------------- |
| DAUCUS-CARDIA | -------------------------------------------------- |
| SOLENOSTEMON-SCUTELLARIOIDES | -------------------------------------------------- |
| HORDEUM-VULGARE | -------------------------------------------------- |
| TRITICUM-AESTIVUM | -------------------------------------------------- |
| AVENA-SATIVA | -------------------------------------------------- |
| ORYZA-SATIVA | -------------------------------------------------- |
| SORGHUM-BICOLOR | -------------------------------------------------- |
| ZEA-MAYS | -------------------------------------------------- |
| ABO95005_OLUCIMARINUS | -------------------------------------------------- |
| OTAURI | -------------------------------------------------- |
| MICROMONAS-PUSILLA-CCMP1545 | -------------------------------------------------- |
| SYNECHOCOCCUS-SP. | -------------------------------------------------- |
| VIBRIO-SP.-MED222 | -------------------------------------------------- |
| MARINOMONAS-SP.-MED121 | -------------------------------------------------- |
| PSEUDOMONAS-FLUORESCENS | -------------------------------------------------- |
| SULFITOBACTER-SP.-NAS-14.1 | -------------------------------------------------- |
| OCEANICOLA-BATSENSIS-HTCC2597 | -------------------------------------------------- |
| BDELLOVIBRIO-BACTERIOVORUS | -------------------------------------------------- |
| RATTUS_NORVEGICUS | --------------------KEMGDHLVKHGDGVKDIAFEVEDCEHIVQK |
| HOMO-SAPIENS | --------------------KEMGDHLVKHGDGVKDIAFEVEDCDYIVQK |
| XENOPUS-LAEVIS | --------------------HPISDHVAMHGDGVKDVAFSVEDCRGIYKR |
| BLEPHARISMA-JAPONICUS | --------------------NEVNRHVGKHSDGVQDIAFSVSDARGMYEK |
| MYCOSPHAERELLA-GRAMINICOLA | -----RQTLKAAPLADQARLDEMYDHLDKHGDGVKDVAFEVDDVLAVYEN |
| ASPERGILLUS-FUMIGATUS | -----LDQVDRFPPEEQELLKEIHAHLEKHGDGVKDVAFEVDSVDSVFYA |
| MAGNAPORTHE-GRISEA | -----LPEDEPISDEDRALLKEMHAHLEKHGDAVKDVCFEVDNVQGVYER |
| CANDIDA-ALBICANS | NFIVQTINNSEEIYNDMMECTLIQKFLKLHAEGVMDIAFNVNDVDTIFNR |
| PICHIA-STIPITIS | NLILNVINNSEVIYNDIMECTLIQKFLKTHGEGVMDISFLVEDVITIFDK |
| RHODOCOCCUS-SP. | ----------------------LIDHHRTHGDGVVDIALAVPDVDKCIAH |
| RHODOCOCCUS-ERYTHROPOLIS | ----------------------LIEHHSRHGDGIRDIALSVPDVDKCIAH |
| JANIBACTER-SP.-HICC2649 | ----------------------LIAHHTKHGDGVVDISLEVPDVDKCIAQ |
| STREPTOMYCES-AVERMITILIS | -------------------GHFLADHVAEHGDGVVDLAIEVPDARAAHAY |
| KORDIA-ALGICIDA | ----------------------INEHIDLHGDGVKVVALWVEDATKAFEE |
| LEEUWENHOEKIELLA-BLANDENSIS-ME | ----------------------INEHIVKHGDGVKVVALWVEDARSAFEE |
| GEMMATIMONAS-AURANTIACA | ----------------------IAEHVHRHGDGVRDYALWVDDARLAYET |
| PICROPHILUS-TORRIDUS | ----------------------ISNHVKKHGDGVKDIALEVDDLDEAKSL |
| ARABIDOPSIS-THALIANA | -------KPTTTASIPSFDHGSCRSFFSSHGLGVRAVAIEVEDAESAFSI |
| BRASSICA-RAPA-SUBSP.-PEKINENSI | -------PPTTTASIPSFDHVTYRSFFSSHGLGVRAVAVEVEDAEAAFSI |
| COPTIS-JAPONICA-VAR.-DISSECTA | --------LTHTASIPTYSHNLARLFASTHGLAVRAIAIEVQDAELAYNI |
| HEVEA-BRASILIENSIS | -------SHTATASIPTFSHEACRNFSAKHGLGVRAIAIEVEDAEIAYNT |
| MEDICAGO-TRUNCATULA | --------PSSTAAIPTFSASTCFSFSASHGLAVRAVAVEVEDAEVAFTT |
| GLYCINE-MAX | --------AASSASIPSFDAATCLAFAAKHGFGVRAIALEVADAEAAFSA |
| SOLANUM-LYCOPERSICUM | -----------TAGIPSFSTPTYRDFTAKHGLGVRAVALEVENAYLAFSA |
| SOLANUM-TUBEROSUM | -----------TAGIPSFSTSTHRDFTAKHGLGVRAVALEVENAYLAFSA |
| NICOTIANA-BENTHAMIANA | -----------SAAIPSFSTSTHRSFAATHGLGVRAVALEVENAYTAFSA |
| DAUCUS-CARDIA | -----------SAAIPSFSASGFHSFAAKHGLAVRAIALEVADVAAAFEA |
| SOLENOSTEMON-SCUTELLARIOIDES | -----------SASIPTFSFSDHRAFTSSHGLAVRAVAIQVDSASSAYSA |
| HORDEUM-VULGARE | -----------TASLPSFSADAARRFSADHGIAVRSVALRVADAAEAFRA |
| TRITICUM-AESTIVUM | -----------TASLPSFSADAARRFSADHGLAVRSIALRVADAAEAFRA |
| AVENA-SATIVA | -----------TASIPSFSADAARTFAAAHGLAVRSVGVRVADAAEAFRV |
| ORYZA-SATIVA | -----------TASIPSFSPGAARRFAADHGLAVHAVALRVADAADAFRA |
| SORGHUM-BICOLOR | -----------TASLPSFSAAEARRFAADHGLAVRAVALRVADAEDAFRA |
| ZEA-MAYS | -----------TAALPSFSAAAARRFAADHGLAVRAVALRVADAEDAFR- |
| ABO95005_OLUCIMARINUS | -------GEAPHPG---HEGRAMMRFFEKHGLAARAVGVRVKDARAAYEE |
| OTAURI | -------VNVPHPG---NERGAMMRFFERHGLAARAVGLRVGDARAAYEE |
| MICROMONAS-PUSILLA-CCMP1545 | -------SMFEKSGVLSHDPSFMRTFCERHGLAVRAVCLLVDDAAVAFYT |
| SYNECHOCOCCUS-SP. | --------------------DEVDRHLQRHGPGVVDVALAVGEQELPALA |
| VIBRIO-SP.-MED222 | ---------------------QAEAFAKVHGPSVCGMAFRVNEATAAMEQ |
| MARINOMONAS-SP.-MED121 | ---------------------QSEAFARIHGPSVCGMAFRVKDASLAMQH |
| PSEUDOMONAS-FLUORESCENS | ---------------------IASYFAAEHGPSVCGMAFRVKDSQKAYNR |
| SULFITOBACTER-SP.-NAS-14.1 | ---------------------FAAGFVAEHGPCAPSMGWRVVDAQKALDH |
| OCEANICOLA-BATSENSIS-HTCC2597 | ---------------------HAATFIEEHGPCAPSMGWRVVDAQHAFDH |
| BDELLOVIBRIO-BACTERIOVORUS | ---------------------FATDFAKLHGPCVNATGFRVIDADQAFKT |
| RATTUS_NORVEGICUS | ARERGAKIVREPWVEED------KFGKVKFAVLQTYG-DTTHTLVEK--- |
| HOMO-SAPIENS | ARERGAKIMREPWVEQD------KFGKVKFAVLQTYG DTTHTLVEK |
| XENOPUS-LAEVIS | AIERGAKSVREPWEESD------EFGTVVMATIQTYG-DTTHTFVER--- |
| BLEPHARISMA-JAPONICUS | AIAKGCKSFREPQVLQD------QFGSVIIASLQTYG-DTVHTLVQN--- |
| MYCOSPHAERELLA-GRAMINICOLA | AVANGAESVSSPHTDSC------DEGDVISAAIKTYG-DTTHTFIQR--- |
| ASPERGILLUS-FUMIGATUS | ATNNGAKIVSQPRTLED------DNGQVRVATIQTYG-ETTHTLVER--- |
| MAGNAPORTHE-GRISEA | AVQQGAVSIAPPKTLSDK-----EHGSVTMAVIQTYG-DTTHTLLSR--- |
| CANDIDA-ALBICANS | AIKAGSGIIRLPKIISD------ENGVVKLATISIPNSDIQHTLIEN--- |
| PICHIA-STIPITIS | AVAAGAGIIRLPKIISD------CNGSVRLGTISIPKTDIQHTLIEN--- |
| RHODOCOCCUS-SP. | ARAQGATVLDEPHDVTD------DHGTVRLAATATYG-DTRHTLVDR--- |
| RHODOCOCCUS-ERYTHROPOLIS | AITQGATVLSEPHDITD------EHGTVRLASIATYG-ETRHTLVDR--- |
| JANIBACTER-SP.-HICC2649 | AKAAGARVVQEAETVSD------EFGSVRIGAIATYG-ETRHTLVQRTVD |
| STREPTOMYCES-AVERMITILIS | AIEHGARSVAEPYELKD------EHGTVVLAATATYG-KTRHTLVDR--- |
| KORDIA-ALGICIDA | TTKRGAKPYMEPTKEED------ENGYVIRSGIYTYG-ETVHVFVER--- |
| LEEUWENHOEKIELLA-BLANDENSIS-ME | TTKRGAKPFMEPEVEKD------EHGEVVRSGIYTYG-ETVHMFVER--- |
| GEMMATIMONAS-AURANTIACA | AIARGAIPIQEPQVYSD------EHGEVVIAAIGTYG-DTIHSLVER--- |
| PICROPHILUS-TORRIDUS | IEKYGTKVS-KINEIKD------GNGKIRTAEIKTYG ETVHTLIET--- |
| ARABIDOPSIS-THALIANA | SVANGAIPSSPPIVLNE---------AVTIAEVKLYG-DVVLRYVSYKAE |
| BRASSICA-RAPA-SUBSP.-PEKINENSI | SVSNGAVPSSPPIVLND---------AVTIAEVKLYG-DVVLRYVSYKVA |
| COPTIS-JAPONICA-VAR.-DISSECTA | SVANGAKPSSSPIKLDE---------GVVLSEIQLYG-DVVLRYLSFKNT |
| HEVEA-BRASILIENSIS | SVARGALPMGGPITLDN---------RAVVAEVHLYG-DVVLRYISYKNS |
| MEDICAGO-TRUNCATULA | SVNLGAIPSSPPVILEN---------NVKLAEVHLYG-DVVLRYVSYNDL |
| GLYCINE-MAX | SVAKGAEPASPPVLVDD---------RTGFAEVRLYG-DVVLRYVSYKDA |
| SOLANUM-LYCOPERSICUM | SVARGAKPRFEPVTIDE---------HVAVAEVHLYG-DVVLRFVSLVKD |
| SOLANUM-TUBEROSUM | SVSRGAKPRFEPVTIDE---------HVAVAEVHLYG-DVVLRFVSFVKD |
| NICOTIANA-BENTHAMIANA | SVSRGAKPMFEPVTIDG---------QVAMAEVHLYG-DVVLRFMS-LKD |
| DAUCUS-CARDIA | SVARGARPASAPVELDD---------QAWLAEVELYG-DVVLRFVSFGRE |
| SOLENOSTEMON-SCUTELLARIOIDES | AVSRGAKPVSPPVVLADC--------ETAIAEVHLYG-DTVLRFVSCGSG |
| HORDEUM-VULGARE | SRRRGARPAFAPVDLGR---------GFAFAEVELYG-DVVLRFVSHPDG |
| TRITICUM-AESTIVUM | SVDGGARPAFSPVDLGR---------GFGFAEVELYG-DVVLRFVSHPDD |
| AVENA-SATIVA | SVAGGARPAFAPADLGH---------GFGLAEVELYG-DVVLRFVSYPDE |
| ORYZA-SATIVA | SVAAGARPAFQPADLGG---------GFGLAEVELYG-DVVLRFVSHPDG |
| SORGHUM-BICOLOR | SVAAGARPAFEPVELGL---------GFRLAEVELYG-DVVLRYVSYPDD |
| ZEA-MAYS | --------------------------GFRLAEVELYG-DVVLRYVSYPDG |
| ABO95005_OLUCIMARINUS | AVKRGARGVLAPTVLTHTVDDGCAKGGQVIAEIELYG-DVVLRFVNATDG |
| OTAURI | AMKRGARGVLEPTEMRHEKHDGCVMGTQIISEVELYG-DVVLRFVSRADG |
| MICROMONAS-PUSILLA-CCMP1545 | SGQHGGR-----SPAFSSACDGFAR----VSEVELYG-DVVLRYYSFHAG |
| SYNECHOCOCCUS-SP. | ELLRGRG-----------------------AQLAWIP-AAAALCLHTPYG |
| VIBRIO-SP.-MED222 | AFKGGGEEYK-TEIGP---------MELSIPAIYGIG-ESLLYFVDRYGK |
| MARINOMONAS-SP.-MED121 | ALANGAKEFS-GNLGA---------MELKLPAVYGIG-ESTLYFIDRYGD |
| PSEUDOMONAS-FLUORESCENS | ALELGAQPIH-IDTGP---------MELNLPAIKGIG-GAPLYLIDRFGE |
| SULFITOBACTER-SP.-NAS-14.1 | AVSKGAEEYT-GAG-----------KVLDVPAIKGIG-GSLIYFVDQYYD |
| OCEANICOLA-BATSENSIS-HTCC2597 | AVKNGATPYE-GDG-----------KVMDVPAILGIG-GSLIYFIEDYYE |
| BDELLOVIBRIO-BACTERIOVORUS | AVARGARPYEGNEHQK---------GATPFPAIYGIG-DSLIYFMDQKNQ |
| RATTUS_NORVEGICUS | -INYTG-----RFLPGFEAPTYKDT-----LLPKLPSCNLEIIDHIVGNQ |
| HOMO-SAPIENS | -MNYIG-----QFLPGYEAPAFMDP-----LLPKLPKCSLEMIDHIVGNQ |
| XENOPUS-LAEVIS | -TNYKAP--AHVFLPNYRA-CEVDP-----INNVLPTVKLLNVDHVVGNQ |
| BLEPHARISMA-JAPONICUS | -VDYTG-----PFLPGFRAITKDDP-----LNSAFPQVNYDIIDHVVGNQ |
| MYCOSPHAERELLA-GRAMINICOLA | -TTYTG-----PFLPGYRSCTTVDS-----ANKFLPPVNLEAIDHCVGNQ |
| ASPERGILLUS-FUMIGATUS | -GSYHG-----AFLPGYRMETGVEDP----ISQLLPGVHLNRIDHCVGNQ |
| MAGNAPORTHE-GRISEA | -DNFRG-----TFLPGFRDVNRQPAA----YSALAP-VPLQRIDHCVGNQ |
| CANDIDA-ALBICANS | -INYTG-----PFLPGFSQPIYPLADYYQVQLNMMPPVNLTVLDHCVENY |
| PICHIA-STIPITIS | -IDYTG-----PFLPNYSESVTQYNSKYYDQMQNIPTVSFQCIDHCVENY |
| RHODOCOCCUS-SP. | -SHYTG-----PYLPGYTARTSGHT-----KRDGAPKRLFQALDHVVGNV |
| RHODOCOCCUS-ERYTHROPOLIS | -SRYTG-----PYLPGYVERTSSYR-----KRDGAPKRIFQALDHVVGNV |
| JANIBACTER-SP.-HICC2649 | GQTYSG-----PYLPGYVARSSSFV-----KRDGAPKRLFQALDHIVGNV |
| STREPTOMYCES-AVERMITILIS | -TGYDG-----PYLPGYVAAAP--------IVEPPAHRTFQAIDHCVGNV |
| KORDIA-ALGICIDA | -KNYNG-----VFLPGYQRWES---------HYNPEPVGLKFIDHMVGNV |
| LEEUWENHOEKIELLA-BLANDENSIS-ME | -KNYNG-----QFLPGYRKWES---------DYNPEPTGLKYIDHMVGNV |
| GEMMATIMONAS-AURANTIACA | -RNYNG-----VFLPGFKAVTP---------HYQPSDVGLKYIDHCVGNV |
| PICROPHILUS-TORRIDUS | -GDYNG-----VFMPGYEESEI-----------NSKNTGIKKIDHIVGNV |
| ARABIDOPSIS-THALIANA | DTE------KSEFLPGFERVEDASSF-------P-LDYGIRRLDHAVGNV |
| BRASSICA-RAPA-SUBSP.-PEKINENSI | TV----------FLPRFETVDDTSSF-------P-LDYGIRRLDHAVGNV |
| COPTIS-JAPONICA-VAR.-DISSECTA | N----QSCP---FLPGFEEVGEVSSS-------RGLDFGIRRLDHAVGNV |
| HEVEA-BRASILIENSIS | NPNLNDSSPDSWFLPKFESVDEASSF-------P-LDYGIRRLDHAVGNV |
| MEDICAGO-TRUNCATULA | NP---NQNPNLFFLPGFERVSDESSN-------SSLDFGIRRLDHAVGNV |
| GLYCINE-MAX | APQAPHADPSRWFLPGFEAAASSSSF-------PELDYGIRRLDHAVGNV |
| SOLANUM-LYCOPERSICUM | ADTL-------IFLPGFEAMDETSSF-------KELDYGIHRLDHAVGNV |
| SOLANUM-TUBEROSUM | EDSL-------IFLPGFEAMDETSSF-------KELDYGIRRLDHAVGNV |
| NICOTIANA-BENTHAMIANA | ADSL-------VFLPGFNAMDETASY-------KELDYGIRRLDHAVGNV |
| DAUCUS-CARDIA | EG---------LFLPGFEAVEGTASF-------PDLDYGIRRLDHAVGNV |
| SOLENOSTEMON-SCUTELLARIOIDES | ADG--------WFLPGFEVVGDGVSC-------QELDYGIRRLDHAVGNV |
| HORDEUM-VULGARE | TDVP--------FLPGFEGVTNP----------DAVDYGLTRFDHVVGNV |
| TRITICUM-AESTIVUM | TDVP--------FLPGFEGVSNP----------DAVDYGLTRFDHVVGNV |
| AVENA-SATIVA | TDLP--------FLPGFERVSSP----------GAVDYGLTRFDHVVGNV |
| ORYZA-SATIVA | ADAP--------FLPGFEGVSNP----------GAVDYGLRRFDHVVGNV |
| SORGHUM-BICOLOR | ADAS--------FLPGFVGVTSP----------GAADYGLRRFDHIVGNV |
| ZEA-MAYS | AAGEP-------FLPGFEGVASP----------GAADYGLSRFDHIVGNV |
| ABO95005_OLUCIMARINUS | --------FDGDFLCNYSATRDAP----------DVSYGLQRLDHAVGNV |
| OTAURI | --------FDGDFLCNYEATRDVP----------SVSYGLRRLDHAVGNV |
| MICROMONAS-PUSILLA-CCMP1545 | EKNA----KPKTFLPGYEDVPLEPPHT------TPLTYGLQRLDHAVGNV |
| SYNECHOCOCCUS-SP. | IRHS--------LIPGPLDAAPAEAG------------LFSHWDHVVLNV |
| VIBRIO-SP.-MED222 | -QSIYD----VDFRFYDDAEQRMAEA----------NVGLYEIDHLTHNV |
| MARINOMONAS-SP.-MED121 | -KSIYD----VDFNFYENYQEKMLSH----------QAGLYEVDHLTHNV |
| PSEUDOMONAS-FLUORESCENS | GSSIYD----IDFVYLEGVERNPVG------------AGLKVIDHLTHNV |
| SULFITOBACTER-SP.-NAS-14.1 | -TSPYN----EEYDWIAQS--KPAG------------VGFYYLDHLTHNV |
| OCEANICOLA-BATSENSIS-HTCC2597 | -TSPYN----AEFDWLAQS--KPRG------------VGFYYLDHLTHNV |
| BDELLOVIBRIO-BACTERIOVORUS | -DKLYN----EIFQVKPED-KAPVG------------VGFTVVDHFTNNV |
| RATTUS_NORVEGICUS | PDQEMESASEWYLKNLQFHRFWSVDDTQVHTEYSSLRSIVVANYEESIKM |
| HOMO-SAPIENS | PDQEMVSASEWYLKNLQFHRFWSVDDTQVHTEYSSLRSIVVANYEESIKM |
| XENOPUS-LAEVIS | PDDMMVPVAEWYEKMLMFHRFWSVDDTQMHTDYSALRSIVVTDYDEVIKM |
| BLEPHARISMA-JAPONICUS | PGGDMTPTVEWYEKYLEFHRYWSADESVIHTDYSALRSVVVADWDEVIKM |
| MYCOSPHAERELLA-GRAMINICOLA | DWDEMSDACDFYERCLGFHRFWSVDDKDICTEFSALKSIVMSSPNQVVKM |
| ASPERGILLUS-FUMIGATUS | DWDEMDKVCEYYEKALGFHRFWSVDDKQICTEYSALKSIVMASPNEVVKM |
| MAGNAPORTHE-GRISEA | DWDDMRAACDFYERCLSFHRFWSVDDNQISTDFSALNSIVMASPNNVVKM |
| CANDIDA-ALBICANS | SWNQMMEQAKLYADMFGFHKYWSVDEDDISTGFTALRSIVMSSSNGQIKM |
| PICHIA-STIPITIS | SWNQMMAQAKLYASLFGFHKYWSADDHDIATDNTALRSIVMASGNGKIKM |
| RHODOCOCCUS-SP. | ELGKMDHWVDFYNRVMGFTNMAEFVGEDIATDYSALMSKVVSNGNHRVKF |
| RHODOCOCCUS-ERYTHROPOLIS | ELGKMDQWVDFYNRVMGFTNMAEFVGGDIATDYSALMSKVVSSGNHRVKF |
| JANIBACTER-SP.-HICC2649 | ELGKMDEWVSFYNRVMGFVNMAEFVGDDIATDYSALMSKVVANGNHRVKF |
| STREPTOMYCES-AVERMITILIS | ELGRMNEWVGFYNKVMGFTNMKEFVGDDIATEYSALMSKVVADGTLKVKF |
| KORDIA-ALGICIDA | GWGEMKEWCEFYAKVMGFAQIISFTDDDISTDFTALMSKVMSNGNGRIKF |
| LEEUWENHOEKIELLA-BLANDENSIS-ME | GWGEMNTWVKWYEDVMGFVNFLTFDDKQITTEYSALMSKVMSNGNGRIKF |
| GEMMATIMONAS-AURANTIACA | ELGKMNQWVGYYADVLGFRNLITFDDTDINTEYSSLMSKVMANGNDRIKF |
| PICROPHILUS-TORRIDUS | YEGEMDSWVNFYIEKLGFEHLITFDDKDIRTDYSALRSKVVKY-NDDIVF |
| ARABIDOPSIS-THALIANA | P--ELGPALTYVAGFTGFHQFAEFTADDVGTAESGLNSAVLASNDEMVLL |
| BRASSICA-RAPA-SUBSP.-PEKINENSI | P--ELGPALTYLSRLTGFHQFAEFTADDVGTAESGLNSAVLANNDETVLL |
| COPTIS-JAPONICA-VAR.-DISSECTA | P--NLAEAIGYLKEFTGFHEFAEFTAEDVGTTESGLNSIVLASNDEMVLL |
| HEVEA-BRASILIENSIS | P--ELAPAVSYVKEFTGFHEFAEFTAEDVGTSESGLNSLVLANNEDTVLL |
| MEDICAGO-TRUNCATULA | P--ELSSAVKYVKQFTGFHEFAEFTAEDVGTSESGLNSVVLANNEETVLL |
| GLYCINE-MAX | P--ELAPAVRYLKGFSGFHEFAEFTAEDVGTSESGLNSVVLANNSETVLL |
| SOLANUM-LYCOPERSICUM | P--ELGPVVDYIKAFTGFHEFAEFTAEDVGTAESGLNSVVLANNDETVLL |
| SOLANUM-TUBEROSUM | P--ELGPVVDYIKEFTGFHEFAEFTAEDVGTAESGLNSVVLANNDETVLL |
| NICOTIANA-BENTHAMIANA | P--ELGPAVDYIKRFTGFHEFAEFTSEDVGTAESGLNSMVVANNDETVLL |
| DAUCUS-CARDIA | T--ELGPVVEYIKGFTGFHEFAEFTAEDVGTLESGLNSVVLANNEEMVLL |
| SOLENOSTEMON-SCUTELLARIOIDES | P--KLEPVVDYLKKFTGFHEFAEFTAEDVGTAESGLNSVVLANNNENVLF |
| HORDEUM-VULGARE | P--ELAPAAAYIAGFTGFHEFAEFTAEDVGTTESGLNSVVLANNSEGVLL |
| TRITICUM-AESTIVUM | P--ELAPAAAYVAGFAGFHEFAEFTTEDVGTAESGLNSMVLANNSEGVLL |
| AVENA-SATIVA | P--EMAPVIDYMKGFLGFHEFAEFTAEDVGTTESGLNSVVLANNSEAVLL |
| ORYZA-SATIVA | P--ELAPVAAYISGFTGFHEFAEFTAEDVGTAESGLNSVVLANNAETVLL |
| SORGHUM-BICOLOR | P--ELAPAAAYFAGFTGFHEFAEFTAEDVGTTESGLNSMVLANNAENVLL |
| ZEA-MAYS | P--ELAPAAAYFAGFTGFHEFAEFTTEDVGTAESGLNSMVLANNSENVLL |
| ABO95005_OLUCIMARINUS | H--DLIETVDYITKVTGFHEFAEFTAEDIGTIDSGLNSMVLANNNEYVLL |
| OTAURI | H--NLLETVDYIMKITGFHEFAEFTAEDIGTIDSGLNSMVLANNNEYVLL |
| MICROMONAS-PUSILLA-CCMP1545 | P--NLLETVDYITAMTGMHEFAEFTAEDVGTVDSGLNSMVLANDDEMILL |
| SYNECHOCOCCUS-SP. | EQGSLQAAADWYGRVLGWRRLYRYS---IGTATSGLESVVVGDPEAGIQW |
| VIBRIO-SP.-MED222 | KQGNMDVWSGFYERLGNFREIRYFDIEGKLTG---LVSRAMTSPCGKIRI |
| MARINOMONAS-SP.-MED121 | MRGNMDHWAGFYENIGNFREIRYFDIEGKLTG---LVSRAMTSPCGKIRI |
| PSEUDOMONAS-FLUORESCENS | YRGRMVYWANFYEKLFNFREARYFDIKGEYTG---LTSKAMSAPDGMIRI |
| SULFITOBACTER-SP.-NAS-14.1 | FKGNMDVWFKFYGDLFNFREIRFFDIEGKFTG---LTSRALTSPCGRIRI |
| OCEANICOLA-BATSENSIS-HTCC2597 | FKGNMDTWFRFYGDLFNFREIRFFDIQGKYTG---LFSRALTSPCGRIRI |
| BDELLOVIBRIO-BACTERIOVORUS | PKGEMDKWQHFYEDIFGFYEAKYFDIRGSKTG---LLSRAMRSPCGKFSV |
| RATTUS_NORVEGICUS | PINEPAPGRK-KSQIQEYVDYNGGAGVQHIALRTEDIITTIRHLRER--- |
| HOMO-SAPIENS | PINEPAPGKK-KSQIQEYVDYNGGAGVQHIALKTEDIITAIRHLRER--- |
| XENOPUS-LAEVIS | PINEPAPGKK-KSQIQEFVEYYGGAGVQHIALRTDDILRDVSAMRAR--- |
| BLEPHARISMA-JAPONICUS | PINEPADGLR-KSQIQEYVEYYGGAGVQHIALKVNDIISVISTLRAR--- |
| MYCOSPHAERELLA-GRAMINICOLA | PINEPAHGKK-KSQIEEYVDFYNGPGVQHIALRTPNIIEAVSNLRSR--- |
| ASPERGILLUS-FUMIGATUS | PINEPAKGKK-QSQIEEYVDFYNGAGVQHIALLTDDIIRDITNLKAR--- |
| MAGNAPORTHE-GRISEA | PINEPAKGKK-RSQIEEYVTFNSGAGVQHIALLTSDIITTVEAMRSR--- |
| CANDIDA-ALBICANS | PINEPVKSIM-KGQIEEFNDFNGGPGIQHIAFRTNNIIETVMALMQR--- |
| PICHIA-STIPITIS | PINEPVKSKM-RGQIEEFHDFNGGPGVQHIALRTNDIIDTVCALLAR--- |
| RHODOCOCCUS-SP. | PLNEPALAKK-RSQIDEYLDFYRGPGAQHLALATNDILTAVDQLTAE--- |
| RHODOCOCCUS-ERYTHROPOLIS | PLNEPAIAKK-RSQIDEYLEFYQGPGAQHLALATNDILGAVDALVDE--- |
| JANIBACTER-SP.-HICC2649 | PLNEPAIAKK-RSQIDEYLDFYQGPGAQHLAVATNDILRSVDELRKE--- |
| STREPTOMYCES-AVERMITILIS | PINEPALAKK-KSQIDEYLEFYGGAGVQHIALNTGDIVETVRTMRAA--- |
| KORDIA-ALGICIDA | PINEPAEGKK-KSQIEEYLDFYNGSGVQHIAVATDNIIDTVSQMRER--- |
| LEEUWENHOEKIELLA-BLANDENSIS-ME | PINEPAEGIK-KSQIEEYLDFYEGPGVQHLAVATDDIVKTVAALKAR--- |
| GEMMATIMONAS-AURANTIACA | PINEPASGKK-KSQIEEYLDFYGGPGAQHLALATDDILATVTALRDR--- |
| PICROPHILUS-TORRIDUS | PINEPAKGLR-KSQIEEYLDYYRSEGVQHIALLTDDIIKTVSMMEEN--- |
| ARABIDOPSIS-THALIANA | PINEPVHGTKRKSQIQTYLEHNEGAGLQHLALMSEDIFRTLREMRKRSS- |
| BRASSICA-RAPA-SUBSP.-PEKINENSI | PVNEPVHGTKRKSQIQTYLEHNEGAGVQHLALMSEDIFRTLREMRKRSG- |
| COPTIS-JAPONICA-VAR.-DISSECTA | PMNEPVYGTKRKSQIQTYLEHNEGAGVQHLALVSEDIFTTLREMRRRSG- |
| HEVEA-BRASILIENSIS | PLNEPVFGTKRKSQIQTYLEHNEGAGLQHLALVSEDIFKTLREMRRRSG- |
| MEDICAGO-TRUNCATULA | PMNEPVYGTKRKSQIETYLEHNEGAGLQHLALMSADIFRTLREMRKRSG- |
| GLYCINE-MAX | PLNEPVYGTKRKSQIETYLEHNEGAGVQHLALVTHDIFTTLREMRKRSF- |
| SOLANUM-LYCOPERSICUM | PLNEPVYGTKRKSQIQTYLEHNEGAGVQHLALVTEDIFRTLREMWKRSG- |
| SOLANUM-TUBEROSUM | PMNEPVYGTKRKSQIQTYLEHNEGAGVQHLALVTEDIFRTLREMRKRSG- |
| NICOTIANA-BENTHAMIANA | PLNEPVYGTKRKSQIQTYLEHNEGAGVQHLALVTEDIFKTLKEMRKRSG- |
| DAUCUS-CARDIA | PLNEPVYGTKRKSQIQTYLEHNEGAGVQHLALVSEDIFRTLREMRKRSC- |
| SOLENOSTEMON-SCUTELLARIOIDES | PLNEPVYGTKRKSQIQTYLDHNEGAGVQHLALITEDIFRTLREMRKRSE- |
| HORDEUM-VULGARE | PLNEPVHGTKRRSQIQTFLEHHGGPGVQHIAVASSDVLRTLRKMRARSA- |
| TRITICUM-AESTIVUM | PLNEPVHGTKRRSQIQTFLEHHGGSGVQHIAVASSDVLRTLREMRARSA- |
| AVENA-SATIVA | PLNEPVHGTKRRSQIQTYLEYHGGPGVQHIALASNDVLRTLREMRARTP- |
| ORYZA-SATIVA | PLNEPVHGTKRRSQIQTYLDHHGGPGVQHIALASDDVLGTLREMRARSA- |
| SORGHUM-BICOLOR | PLNEPVHGTKRRSQIQTYLDHHGGPGVQHMALASDDVLRTLREMQARSA- |
| ZEA-MAYS | PLNEPVHGTKRRSQIQTFLDHHGGPGVQHMALASDDVLRTLREMQARSA- |
| ABO95005_OLUCIMARINUS | PVNEPTFGTKRKSQIQTYLEQNNGPGLQHLALKTDDIFATVREMRKYSHL |
| OTAURI | PVNEPTFGTKRKSQIQTYLEQNNGPGLQHLALKTDDIFTTVREMRKYSHM |
| MICROMONAS-PUSILLA-CCMP1545 | PVNEPTFGTKRKSQIQTYLECNNGAGLQHLALKSDDVFATVREMRKHGGG |
| SYNECHOCOCCUS-SP. | AINEPTCAAS---QIQEFLHAHGGPGIQHAALHSSDIVASLRRLR----- |
| VIBRIO-SP.-MED222 | PINE-SSDDK--SQIEEFIREYNGEGIQHIALATDDIYKTVKTLRDR--- |
| MARINOMONAS-SP.-MED121 | PINE-SSDDK--SQIEEFLNQYNGEGIQHIAMSSSDIYETVRQLKAG--- |
| PSEUDOMONAS-FLUORESCENS | PLNEESSKGA--GQIEEFLMQFNGEGIQHVAFLTDDLVKTWDALKKI--- |
| SULFITOBACTER-SP.-NAS-14.1 | PINE-DRDEK--GQIVAYLKKYNGEGIQHIAVGARNIYDATDAIADN--- |
| OCEANICOLA-BATSENSIS-HTCC2597 | PINE-DRGET--GQIVAYLKKYNGEGIQHIAVGARDIYAATDAIAEN--- |
| BDELLOVIBRIO-BACTERIOVORUS | PINE-PTEEK--SQIQEYLDEYKGSGIQHIALLTHDINYSLESLKNS--- |
| RATTUS_NORVEGICUS | --GMEFLAVP-SSYYRLL--------RENLKTSK--IQVKEN-----MDV |
| HOMO-SAPIENS | --GLEFLSVP-STYYKQL--------REKLKTAK--IKVKEN-----IDA |
| XENOPUS-LAEVIS | --GLEFLTIP-RTYYKNL--------RARLSMSK--VQVEED-----LAE |
| BLEPHARISMA-JAPONICUS | --GVEFLEVP-PKYYDSL--------RKRLAHSA--VQIEED-----LKR |
| MYCOSPHAERELLA-GRAMINICOLA | --GVEFISVP-DTYYENM--------RLRLKAAG--MKLEES-----FDI |
| ASPERGILLUS-FUMIGATUS | --GVEFIKVP-DTYYEDI--------KVRLKKAG--LTLHED-----FET |
| MAGNAPORTHE-GRISEA | --GVEFIEVP-HTYYDTM--------RRRLKTEKRDWELQED-----FDR |
| CANDIDA-ALBICANS | --GVEFNHTS-ENYYNNL--------KQRLNNDG--IKLYED-----FDT |
| PICHIA-STIPITIS | --GIEFNTAS-DKYYTNL--------ERLLREDD--VALFED-----FDT |
| RHODOCOCCUS-SP. | --GVEFLATP-DSYYEDP--------ELRARIG----NVRAP-----IAE |
| RHODOCOCCUS-ERYTHROPOLIS | --GIEFLSTP-ASYYEDP--------ELRARIG----EVRVP-----IEE |
| JANIBACTER-SP.-HICC2649 | --GVEFLDTP-DAYYDDP--------EMRARIG----EVRVP-----IEE |
| STREPTOMYCES-AVERMITILIS | --GVQFLDTP-DSYYDT----------LGEWVG----DTRVP-----VDT |
| KORDIA-ALGICIDA | --GVEFLYVP-DTYYDD----------LLERVG----DIDED-----VEE |
| LEEUWENHOEKIELLA-BLANDENSIS-ME | --GVEFLPPPPQAYYDD----------IPRRLGAHMDTMKED-----LNK |
| GEMMATIMONAS-AURANTIACA | --GVEFLSVP-TSYYED----------LQERVG----KIDEK-----LEE |
| PICROPHILUS-TORRIDUS | --GIEFLKTP-GSYYES----------LSSRIG----SIDED-----LNE |
| ARABIDOPSIS-THALIANA | IGGFDFMPSPPPTYYQN----------LKKRVG---DVLSDD----QIKE |
| BRASSICA-RAPA-SUBSP.-PEKINENSI | VGGFDFMPSPPPTYYKN----------LKNRVG---DVLSEE----QIEE |
| COPTIS-JAPONICA-VAR.-DISSECTA | VGGFEFMPSPPPTYYKN----------LKNRAG---DVLSDE----QIKE |
| HEVEA-BRASILIENSIS | VGGFDFMPSPPPTYYRN----------LKNRVG---DVLTDE----QIKE |
| MEDICAGO-TRUNCATULA | VGGFEFMPSPPVTYYRN----------LKNRVG---DVLSDE----QIKE |
| GLYCINE-MAX | LGGFEFMPSPPPTYYAN----------LHNRAA---DVLTVD----QIKQ |
| SOLANUM-LYCOPERSICUM | VGGFEFMPAPPPTYYKN----------LRSRAG---DVLSDE----QIQA |
| SOLANUM-TUBEROSUM | VGGFEFMPSPPPTYYKN----------LKSRAG---DVLSDE----QIQA |
| NICOTIANA-BENTHAMIANA | VGGFEFMPSPPPTYYKN----------LKNRAG---DVLTDE----QIQA |
| DAUCUS-CARDIA | LGGFEFMPSPPPTYYKN----------LKNRVG---DVLSDE----QIKE |
| SOLENOSTEMON-SCUTELLARIOIDES | VGGFEFMPSPPPTYYRN----------LKSRAG---DVLSDE----QIEE |
| HORDEUM-VULGARE | MGGFDFLPPPLPKYYEG----------VRRLAG---DVLSEA----QIKE |
| TRITICUM-AESTIVUM | MGGFDFLPPRCRKYYEG----------VRRIAG---DVLSEA----QIKE |
| AVENA-SATIVA | MGGFEFMAPPQAKYYEG----------VRRIAG---DVLSEE----QIKE |
| ORYZA-SATIVA | MGGFEFLAPPPPNYYDG----------VRRRAG---DVLSEE----QINE |
| SORGHUM-BICOLOR | MGGFEFMAPPAPEYYDG----------VRRRAG---DVLTEA----QIKE |
| ZEA-MAYS | MGGFEFMAPPTSDYYDG----------VRRRAG---DVLTEA----QIKE |
| ABO95005_OLUCIMARINUS | RGGFDFQAPASDDYYKQ----------LKAKIG---DALNDE----QYAL |
| OTAURI | HGGFDFQAPASDDYYKH----------LKEKIG---DALTDE----QYAL |
| MICROMONAS-PUSILLA-CCMP1545 | RGGFEFQKPASADYYAN----------LKARVGE--DALTER----QFKE |
| SYNECHOCOCCUS-SP. | QGGVDFLQVAP-QYYTS----------LERELGL--ALRSALGQAISWQD |
| VIBRIO-SP.-MED222 | --GMDFMPTP-DTYYEKVDDRVKGHG--------------ED-----TDL |
| MARINOMONAS-SP.-MED121 | --GLKFMSTP-DTYYAKVNDRVVGHG--------------ED-----LEK |
| PSEUDOMONAS-FLUORESCENS | --GMRFMTAPPDTYYEMLEGRLPDHG--------------EP-----VDQ |
| SULFITOBACTER-SP.-NAS-14.1 | --GLKFMPGPPETYYKMSKDRVTGHQ--------------EP-----LDR |
| OCEANICOLA-BATSENSIS-HTCC2597 | --GVTYMPGPPDAYYDMSHDRVKDHG--------------EP-----IDR |
| BDELLOVIBRIO-BACTERIOVORUS | --EIQFLIPPPHSYYEMIPERVPGVT--------------ED-----ISR |
| RATTUS_NORVEGICUS | LEELKILVDYD---------------EKGYLLQIFTKPMQDRPTLFLEVI |
| HOMO-SAPIENS | LEELKILVDYD---------------EKGYLLQIFTKPVQDRPTLFLEVI |
| XENOPUS-LAEVIS | IEKLSILVDFD---------------EEGYLLQIFTKPLEDRPTLFIEII |
| BLEPHARISMA-JAPONICUS | IEDLHILVDFD---------------DRGYLLQIFTKPVEDRPTLFYEII |
| MYCOSPHAERELLA-GRAMINICOLA | IQKLNILIDFD---------------EGGYLLQLFTKPLMDRPTVFIEII |
| ASPERGILLUS-FUMIGATUS | IRSLDILIDFD---------------EGGYLLQLFTKHLMDRPTVFIEII |
| MAGNAPORTHE-GRISEA | LVRNNILIDYD---------------EGGYLLQLFTRPLMDRPTVFIEII |
| CANDIDA-ALBICANS | LRSLNILIDYDPSTKPKPKSKRKRNNKCNYLLQIFSKPLHDRPTLFIEII |
| PICHIA-STIPITIS | LRKLNILVDYDISTR------NKKTGICNYLLQIFTKPLHDRPTLFIEII |
| RHODOCOCCUS-SP. | LQKRGILVDRD---------------EDGYLLQIFTKPLVDRPTVFFELI |
| RHODOCOCCUS-ERYTHROPOLIS | LQKRGILVDRD---------------EDGYLLQIFTKPIGDRPTVFFEII |
| JANIBACTER-SP.-HICC2649 | LKSRKILVDRD---------------EDGYLLQIFTKPLGDRPTVFFEII |
| STREPTOMYCES-AVERMITILIS | LRELKILADRD---------------EDGYLLQIFTKPVQDRPTVFFEII |
| KORDIA-ALGICIDA | LKKHGILIDRD---------------EEGYLLQLFTKTIVDRPTMFFEVI |
| LEEUWENHOEKIELLA-BLANDENSIS-ME | LQELSILVDAD---------------EEGYLLQIFTKPLQDRPTLFFEII |
| GEMMATIMONAS-AURANTIACA | LAALGILVDRD---------------PDGYLLQIFTKPVEDRPTLFFEII |
| PICROPHILUS-TORRIDUS | IEKHNILVDRD---------------ENGYLLQIFTKPVTDRPTFFFEVI |
| ARABIDOPSIS-THALIANA | CEELGILVDR---------------DDQGTLLQIFTKPLGDRPTIFIEII |
| BRASSICA-RAPA-SUBSP.-PEKINENSI | CEELGILVDR---------------DDQGTLLQIFTKPLGDRPTIFIEII |
| COPTIS-JAPONICA-VAR.-DISSECTA | CEELGILVDR---------------DAQGTLLQIFTKPVGDRPTIFVEII |
| HEVEA-BRASILIENSIS | CEELGILVDR---------------DDQGTLLQIFTKPVGDRPTIFIEII |
| MEDICAGO-TRUNCATULA | CEELGILVDR---------------DDQGTLLQIFTKPIGDRPTIFIEII |
| GLYCINE-MAX | CEELGILVDR---------------DDQGTLLQIFTKPVGDRPTIFIXII |
| SOLANUM-LYCOPERSICUM | CEELGILVDR---------------DDQGTLLQIFTKPVGDRPTIFIEII |
| SOLANUM-TUBEROSUM | CEDLGILVDR---------------DDQGTLLQIFTKPVGDRPTIFIEII |
| NICOTIANA-BENTHAMIANA | CEDLGILVDR---------------DDQGTLLQIFTKPVGDRPTIFIEII |
| DAUCUS-CARDIA | CEDLGILVDR---------------DDQGTLLQIFTKPVGDRPTLFIEII |
| SOLENOSTEMON-SCUTELLARIOIDES | CEKLGILIDR---------------DDQGTLLQIFTKPVGDRPTLFIEII |
| HORDEUM-VULGARE | CQELGVLVDR---------------DDQGVLLQIFTKPVGDRPTLFLEMI |
| TRITICUM-AESTIVUM | CQELGVLVDR---------------DDQGVLLQIFTKPVGDRPTLFLEMI |
| AVENA-SATIVA | CQELGVLVDR---------------DDQGVLLQIFTKPVGDRPTFFLEMI |
| ORYZA-SATIVA | CQELGVLVDR---------------DDQGVLLQIFTKPVGDRPTFFLEMI |
| SORGHUM-BICOLOR | CQELGVLVDR---------------DDQGVLLQIFTKPVGDRPTLFLEII |
| ZEA-MAYS | CQELGVLVDR---------------DDQGVLLQIFTKPVGDRPTLFLEII |
| ABO95005_OLUCIMARINUS | VEELGLLVDK---------------DDQGVLIQVFTKPVGDRPTLFLEII |
| OTAURI | VEELGLLVDK---------------DDQGVLIQVFTKPVGDRPTLFLEII |
| MICROMONAS-PUSILLA-CCMP1545 | VEELGLLVDR---------------DDQGVLVQIFTKPLGDRPTVFIEII |
| SYNECHOCOCCUS-SP. | LVEQQILLDATLPASDG--------QDRPLLLQTFTQPLFGRPTFFFEVI |
| VIBRIO-SP.-MED222 | LRDLRVLIDGAPTKDG-------------ILLQIFTQTVIG--PVFFEII |
| MARINOMONAS-SP.-MED121 | LQDLNILIDGAPLKDG-------------TLLQIFTDTVIG--PVFFEII |
| PSEUDOMONAS-FLUORESCENS | LQARGILLDGSSVEGDK-----------RLLLQIFSETLMG--PVFFEFI |
| SULFITOBACTER-SP.-NAS-14.1 | MKKHGILIDGEGVVDGGE---------TRILLQIFSKTVIG--PIFFEFI |
| OCEANICOLA-BATSENSIS-HTCC2597 | MKKHGILIDGEGVVDGGE---------TRILLQIFSKTVIG--PIFFEFI |
| BDELLOVIBRIO-BACTERIOVORUS | LEKNAILVDGD--KTG------------KYLLQIFTKNTFG--PIFYELI |
| RATTUS_NORVEGICUS | QRHN---------------------HQGFGAGNFNSLFKAFEEEQALRGN |
| HOMO-SAPIENS | QRHN---------------------HQGFGAGNFNSLFKAFEEEQNLRGN |
| XENOPUS-LAEVIS | QRHN---------------------HQGFGAGNFKALFESIEMEQAIRGN |
| BLEPHARISMA-JAPONICUS | QRHN---------------------NNGFGIGNFKALFESLEQEQERRGN |
| MYCOSPHAERELLA-GRAMINICOLA | QRNN---------------------FDGFGAGNFKSLFEAIEREQDLRGN |
| ASPERGILLUS-FUMIGATUS | QRHN---------------------FSGFGAGNFKSLFEAIEREQALRGN |
| MAGNAPORTHE-GRISEA | QRNE---------------------FDGFGAGNFKSLFEAIEREQAERGN |
| CANDIDA-ALBICANS | QRHH---------------------HNGFGKGTFKGLFESIEEQQKLRGT |
| PICHIA-STIPITIS | QRHH---------------------HNGFGKGTFKGLFETIEEQQRIRGT |
| RHODOCOCCUS-SP. | ERHG---------------------SLGFGIGNFKALFEAIEREQAARGN |
| RHODOCOCCUS-ERYTHROPOLIS | ERHG---------------------SLGFGLGNFKALFEAIEREQAARGN |
| JANIBACTER-SP.-HICC2649 | ERHG---------------------SLGFGKGNFKALFESIEREQDARGN |
| STREPTOMYCES-AVERMITILIS | ERHG---------------------SMGFGKGNFKALFEAIEREQEKRGN |
| KORDIA-ALGICIDA | QRKG---------------------AQSFGVGNFKALFEAIEREQAARGT |
| LEEUWENHOEKIELLA-BLANDENSIS-ME | QRMG---------------------AKGFGAGNFKALFESIEREQAQRGT |
| GEMMATIMONAS-AURANTIACA | QRKG---------------------ATSFGKGNFRALFEAIEREQELRGN |
| PICROPHILUS-TORRIDUS | QRKG---------------------ARSFGNGNFKALFEAIEREQAKRGN |
| ARABIDOPSIS-THALIANA | QRVGCMMKDEEGKA------YQSGGCGGFGKGNFSELFKSIEEYEKTLEA |
| BRASSICA-RAPA-SUBSP.-PEKINENSI | QRIGCMKKDEEGRV------YQSGGCGGFGKGNFSELFKSIEEYEKTLEA |
| COPTIS-JAPONICA-VAR.-DISSECTA | QRLGCMLKDEEGKT------YQKAGCGGFGKGNFSELFKSIEEYEKTLEA |
| HEVEA-BRASILIENSIS | QRVGCMIKDETGKE------YQKGGCGGFGKGNFSELFKSIEEYEKTLEA |
| MEDICAGO-TRUNCATULA | QRVGCMLKDEEGKE------YQKGGCGGFGKGNFSELFKSIEEYEKTLET |
| GLYCINE-MAX | QRIGCMVEDEEGKV------YQKGACGGFGKGNFSELFKSIEEYEKTLEA |
| SOLANUM-LYCOPERSICUM | QRIGCMLKDEKGQV------YQKGGCGGFGKGNFSELFRSIEEYEKMLEA |
| SOLANUM-TUBEROSUM | QRIGCMLKDENGQV------YQKGGCGGFGKGNFSELFRSIEEYEKMLEA |
| NICOTIANA-BENTHAMIANA | QRIGCMLKDEKGQV------YQKGGCGGFGKGNFWELFRSIEEYEKTL-- |
| DAUCUS-CARDIA | QRVGCMLKDDAGQM------YQKGGCGGFGKGNFSELFKSIEEYEKTLEA |
| SOLENOSTEMON-SCUTELLARIOIDES | QRVGCMMKDEEGKM------YQKGGCGGFGKGNFSELFKSIEEYEKMLES |
| HORDEUM-VULGARE | QRIGCMEKDERGEE------YQKGGCGGFGKGNFSELFKSIEDYEKSLEA |
| TRITICUM-AESTIVUM | QRIGCMEKDERGEE------YQKGGCGGFGKGNFSELFKSIEDYEKSLEA |
| AVENA-SATIVA | QRIGCMEKDEVGQE------YQKGGCGGFGKGNFSELFKSIEDYEKSLEV |
| ORYZA-SATIVA | QRIGCMEKDESGQE------YQKGGCGGFGKGNFSELFKSIEEYEKSLEA |
| SORGHUM-BICOLOR | QRIGCMEKDEKGQE------YQKGGCGGFGKGNFSQLFKSIEDYEKSLEA |
| ZEA-MAYS | QRIGCMEKDEKGQE------YQKGGCGGFGKGNFSQLFKSIEDYEKSLEA |
| ABO95005_OLUCIMARINUS | QRIGCMRRKADSES-----FEQAAGCGGFGKGNFSELFKSIEAYEATLQI |
| OTAURI | QRVGCMRKKADTDE-----LEQVAGCGGFGKGNFSELFKSIEAYEKTLNI |
| MICROMONAS-PUSILLA-CCMP1545 | QRIGCLREVKSADANAPPRIEQAGGCGGFGKGNFSELFKSIENYERTLKM |
| SYNECHOCOCCUS-SP. | QRLG--------------------GATGFGEANFQALFEALERQQRQRHQ |
| VIBRIO-SP.-MED222 | QRKG---------------------NEGFGEGNFKALFESIEEDQIRRGV |
| MARINOMONAS-SP.-MED121 | QRKG---------------------NEGFGEGNFKALFESIEEDQIRRGV |
| PSEUDOMONAS-FLUORESCENS | QRKG---------------------DDGFGEGNFKALFESIERDQVRRGV |
| SULFITOBACTER-SP.-NAS-14.1 | ERKG---------------------DDGFGEGNFKALFESIEQEQIDSGE |
| OCEANICOLA-BATSENSIS-HTCC2597 | QRKG---------------------DDGFGEGNFKALFESIEQEQIDNGE |
| BDELLOVIBRIO-BACTERIOVORUS | QRKG---------------------HDGFGDGNFQALFDAIERDQRERGY |
| RATTUS_NORVEGICUS | LTDLETNGVRSGM------- |
| HOMO-SAPIENS | LTNMETNGVVPGMAENLYFQ |
| XENOPUS-LAEVIS | L------------------- |
| BLEPHARISMA-JAPONICUS | LI------------------ |
| MYCOSPHAERELLA-GRAMINICOLA | L------------------- |
| ASPERGILLUS-FUMIGATUS | LV------------------ |
| MAGNAPORTHE-GRISEA | L------------------- |
| CANDIDA-ALBICANS | FVKSQNN------------- |
| PICHIA-STIPITIS | LVQVDEDDDSQQST------ |
| RHODOCOCCUS-SP. | F------------------- |
| RHODOCOCCUS-ERYTHROPOLIS | F------------------- |
| JANIBACTER-SP.-HICC2649 | L------------------- |
| STREPTOMYCES-AVERMITILIS | L------------------- |
| KORDIA-ALGICIDA | L------------------- |
| LEEUWENHOEKIELLA-BLANDENSIS-ME | L------------------- |
| GEMMATIMONAS-AURANTIACA | L------------------- |
| PICROPHILUS-TORRIDUS | L------------------- |
| ARABIDOPSIS-THALIANA | KQLVG--------------- |
| BRASSICA-RAPA-SUBSP.-PEKINENSI | KQLVG--------------- |
| COPTIS-JAPONICA-VAR.-DISSECTA | KANVVAA------------- |
| HEVEA-BRASILIENSIS | KRNAEAR------------- |
| MEDICAGO-TRUNCATULA | RRTA---------------- |
| GLYCINE-MAX | KRTA---------------- |
| SOLANUM-LYCOPERSICUM | KHVNQVAAVE---------- |
| SOLANUM-TUBEROSUM | KHVNQVAAA----------- |
| NICOTIANA-BENTHAMIANA | -------------------- |
| DAUCUS-CARDIA | KQITGSAAA----------- |
| SOLENOSTEMON-SCUTELLARIOIDES | KLVTKTAMA----------- |
| HORDEUM-VULGARE | KQSAAVQGS----------- |
| TRITICUM-AESTIVUM | KQSAAVQGS----------- |
| AVENA-SATIVA | KQSVVAQKS----------- |
| ORYZA-SATIVA | KQAPTVQGS----------- |
| SORGHUM-BICOLOR | KQAAAAQGS----------- |
| ZEA-MAYS | KQAAAAAAAQGS-------- |
| ABO95005_OLUCIMARINUS | -------------------- |
| OTAURI | -------------------- |
| MICROMONAS-PUSILLA-CCMP1545 | -------------------- |
| SYNECHOCOCCUS-SP. | ALTP---------------- |
| VIBRIO-SP.-MED222 | LDDA---------------- |
| MARINOMONAS-SP.-MED121 | LNDA---------------- |
| PSEUDOMONAS-FLUORESCENS | LATD---------------- |
| SULFITOBACTER-SP.-NAS-14.1 | LSEA---------------- |
| OCEANICOLA-BATSENSIS-HTCC2597 | IAAE---------------- |
| BDELLOVIBRIO-BACTERIOVORUS | LT------------------ |

The overall sequence identity between individual full length HPPD sequences is in general quite low and is shown for the representative HHPD proteins in Table 3. Table 4a shows the sequence alignment of the binding pocket. In contrast, the sequence identity of the 36 amino acids forming the binding site is significantly higher which is shown for the representative HPPD proteins in Table 4b. In particular, the amino acids at 8 positions are strictly conserved in all species and illustrate that these amino acids have a key role (e.g. His226, His308, Glu394 binding the iron required for catalysis). These positions, with reference to the HPPD from Arabidopsis (SEQ ID No. 2) are His226, Ser267, Asn282, His308, Tyr342, Glu394, Gly420, Asn423 (Table 5a). A mutation of any of the amino acids at either of these positions will most likely lead to an inactive protein. The variability at other positions within the binding site is higher. Table 5b shows the 28 variable positions in the binding site and the amino acids which were identified at these positions using the sequence alignment. Some positions have only limited variability which reflects their role in the 3D environment. An example for this represents the position 269. All HPPD proteins have at this position either a Val, Ala or Thr. Looking at the 3D structures it seems that at this position a small apolar amino acid is required and a mutation to a polar amino acid such as Arg, His or Lys will disturb the protein structure locally. Another example represents position 379. Most sequences have a glutamine at position 379. However, there are also some bacterial sequences which have a histidine at this position. Looking at the 3D structure, it seems that only few amino acids are tolerated at this position. Gln379 in A. thaliana stabilizes via its H-bond donor the side chain conformation of the strictly conserved Glu394 which in turn interacts with the catalytic iron. In addition, with its H-bond acceptors Gln379 stabilizes the side chain conformation of the strictly conserved Asn423 which in turn interacts with the strictly conserved Tyr342. Only glutamine, asparagine and histidine have an H-bond donor and acceptor required for the stabilization of this particular 3D arrangement which very likely play a key role in the interaction of the C-terminal helix with the core of the HPPD protein. Limited variability is also seen at position 381 with either a phenylalanine or a tyrosine in all HPPD sequences. The aromatic ring stabilizes the binding of HPPD inhibitors and very likely also the binding of the substrate to the HPPD binding. However, the presence of the additional hydroxyl group in tyrosine compared to phenylalanine does not disturb the catalytic activity. The third category of positions includes those positions which display a very high natural variability. These positions may not be crucial for substrate binding and catalysis but influence inhibitor binding. These positions include adjacent positions in strand 248 to 255 and positions in the C-terminal helix 419-427. It can be assumed that the interaction of this particular strand and the C-terminal helix with the core of the protein plays a crucial role in inhibitor binding. Table 5b includes for each variable position those amino acids which have been identified in the sequence alignments using all known HPPD sequences.

TABLE 3
Pairwise sequence identity of HPPD sequences from the representative set of HPPD proteins

| | | | | | | | | | | | | | | | | |
|---|---|---|---|---|---|---|---|---|---|---|---|---|---|---|---|---|
| RATTUS_NORVEGICUS | 100 | 90 | 62 | 55 | 52 | 53 | 50 | 44 | 41 | 42 | 39 | 41 | 45 | 45 | 48 | 48 |
| HOMO-SAPIENS | 90 | 100 | 63 | 55 | 53 | 55 | 51 | 42 | 41 | 42 | 40 | 42 | 46 | 46 | 49 | 48 |
| XENOPUS-LAEVIS | 62 | 63 | 100 | 60 | 54 | 57 | 52 | 44 | 44 | 43 | 42 | 43 | 49 | 48 | 51 | 49 |
| BLEPHARISMA-JAPONICUS | 55 | 55 | 60 | 100 | 49 | 49 | 47 | 41 | 40 | 42 | 41 | 41 | 45 | 42 | 46 | 48 |
| MYCOSPHAERELLA- | 52 | 53 | 54 | 49 | 100 | 65 | 61 | 45 | 40 | 44 | 43 | 41 | 46 | 48 | 47 | 48 |
| GRAMINICOLA | | | | | | | | | | | | | | | | |
| ASPERGILLUS-FUMIGATUS | 53 | 55 | 57 | 49 | 65 | 100 | 63 | 45 | 43 | 43 | 42 | 44 | 46 | 49 | 51 | 49 |
| MAGNAPORTHE-GRISEA | 50 | 51 | 52 | 47 | 61 | 63 | 100 | 42 | 40 | 41 | 40 | 40 | 46 | 45 | 45 | 47 |
| CANDIDA-ALBICANS | 44 | 42 | 44 | 41 | 45 | 45 | 42 | 100 | 66 | 37 | 37 | 37 | 38 | 41 | 41 | 43 |
| PICHIA-STIPITIS | 41 | 41 | 44 | 40 | 40 | 43 | 40 | 66 | 100 | 36 | 36 | 36 | 37 | 37 | 38 | 39 |
| RHODOCOCCUS-SP. | 42 | 42 | 43 | 42 | 44 | 43 | 41 | 37 | 36 | 100 | 85 | 77 | 62 | 48 | 49 | 55 |
| RHODOCOCCUS- | 39 | 40 | 42 | 41 | 43 | 42 | 40 | 37 | 36 | 85 | 100 | 75 | 60 | 47 | 49 | 55 |
| ERYTHROPOLIS | | | | | | | | | | | | | | | | |
| JANIBACTER-SP.-HTCC2649 | 41 | 42 | 43 | 41 | 41 | 44 | 40 | 37 | 36 | 77 | 75 | 100 | 63 | 47 | 49 | 53 |
| STREPTOMYCES- | 45 | 46 | 49 | 45 | 46 | 46 | 46 | 38 | 37 | 62 | 60 | 63 | 100 | 50 | 53 | 58 |
| AVERMITILIS | | | | | | | | | | | | | | | | |
| KORDIA-ALGICIDA | 45 | 46 | 48 | 42 | 48 | 49 | 45 | 41 | 37 | 48 | 47 | 47 | 50 | 100 | 73 | 60 |
| LEEUWENHOEKIELLA- | 48 | 49 | 51 | 46 | 47 | 51 | 45 | 41 | 38 | 49 | 49 | 49 | 53 | 73 | 100 | 63 |
| BLANDENSIS-ME | | | | | | | | | | | | | | | | |
| GEMMATIMONAS- | 48 | 48 | 49 | 48 | 48 | 49 | 47 | 43 | 39 | 55 | 55 | 53 | 58 | 60 | 63 | 100 |
| AURANTIACA | | | | | | | | | | | | | | | | |
| PICROPHILUS-TORRIDUS | 45 | 45 | 45 | 45 | 44 | 46 | 41 | 38 | 38 | 46 | 46 | 44 | 46 | 53 | 51 | 53 |
| ARABIDOPSIS-THALIANA | 37 | 36 | 37 | 35 | 34 | 33 | 33 | 28 | 26 | 34 | 35 | 34 | 38 | 35 | 38 | 39 |
| BRASSICA-RAPA-SUBSP.- | 37 | 37 | 37 | 35 | 34 | 33 | 34 | 28 | 26 | 34 | 35 | 35 | 38 | 35 | 38 | 39 |
| PEKINENSI | | | | | | | | | | | | | | | | |
| COPTIS-JAPOMICA-VAR.- | 36 | 37 | 36 | 35 | 34 | 32 | 34 | 28 | 28 | 36 | 36 | 34 | 38 | 34 | 36 | 39 |
| DISSECTA | | | | | | | | | | | | | | | | |
| HEVEA-BRASILIENSIS | 36 | 37 | 34 | 35 | 32 | 31 | 32 | 27 | 25 | 35 | 34 | 34 | 37 | 32 | 36 | 39 |
| MEDICAGO-TRUNCATULA | 37 | 37 | 34 | 34 | 32 | 32 | 32 | 29 | 27 | 35 | 36 | 34 | 37 | 34 | 37 | 39 |
| GLYCINE-MAX | 37 | 38 | 34 | 36 | 33 | 32 | 32 | 28 | 26 | 36 | 36 | 36 | 38 | 32 | 37 | 39 |
| SOLANUM-LYCOPERSICUM | 36 | 37 | 38 | 35 | 34 | 33 | 33 | 28 | 26 | 36 | 36 | 36 | 37 | 34 | 39 | 39 |
| SOLANUM-TUBEROSUM | 36 | 37 | 38 | 35 | 33 | 33 | 33 | 28 | 25 | 35 | 36 | 35 | 37 | 35 | 38 | 39 |
| NICOTIANA-BENTHAMIANA | 36 | 38 | 38 | 35 | 33 | 33 | 34 | 29 | 27 | 35 | 35 | 35 | 37 | 34 | 38 | 38 |
| DAUCUS-CAROTA | 36 | 37 | 38 | 36 | 33 | 34 | 33 | 28 | 26 | 36 | 37 | 35 | 38 | 34 | 37 | 40 |
| SOLENOSTEMON- | 37 | 37 | 37 | 36 | 34 | 35 | 35 | 28 | 26 | 38 | 39 | 37 | 39 | 36 | 39 | 43 |
| SCUTELLARIOIDES | | | | | | | | | | | | | | | | |
| HORDEUM-VULGARE | 35 | 35 | 36 | 35 | 31 | 32 | 32 | 28 | 26 | 35 | 34 | 32 | 35 | 34 | 38 | 39 |
| TRITICUM-AESTIVUM | 35 | 35 | 36 | 34 | 30 | 31 | 31 | 28 | 26 | 34 | 33 | 32 | 34 | 34 | 37 | 37 |
| AVENA-SATIVA | 35 | 35 | 36 | 36 | 32 | 32 | 33 | 27 | 26 | 37 | 36 | 33 | 35 | 34 | 37 | 38 |
| ORYZA-SATIVA | 35 | 35 | 36 | 35 | 32 | 32 | 32 | 28 | 26 | 36 | 34 | 33 | 35 | 35 | 38 | 38 |
| SORGHUM-BICOLOR | 36 | 35 | 35 | 35 | 32 | 33 | 32 | 28 | 27 | 37 | 36 | 34 | 36 | 34 | 38 | 39 |
| ZEA-MAYS | 35 | 35 | 36 | 34 | 31 | 32 | 32 | 29 | 26 | 36 | 35 | 34 | 36 | 34 | 39 | 39 |
| ABO95005_OLUCIMARINUS | 34 | 34 | 36 | 34 | 33 | 32 | 34 | 26 | 25 | 36 | 35 | 36 | 38 | 34 | 38 | 40 |
| OTAURI | 34 | 35 | 36 | 34 | 33 | 33 | 33 | 26 | 24 | 36 | 35 | 36 | 38 | 35 | 38 | 40 |
| MICROMONAS- | 32 | 34 | 35 | 32 | 31 | 30 | 33 | 26 | 25 | 34 | 34 | 33 | 35 | 34 | 36 | 38 |
| PUSILLA-CCMP1545 | | | | | | | | | | | | | | | | |
| SYNECHOCOCCUS-SP. | 31 | 31 | 30 | 31 | 30 | 29 | 30 | 27 | 26 | 34 | 32 | 32 | 34 | 31 | 34 | 36 |
| VIBRIO-SP.-MED222 | 29 | 29 | 31 | 30 | 33 | 35 | 33 | 33 | 32 | 30 | 30 | 31 | 34 | 37 | 37 | 37 |
| MARINOMONAS-SP.-MED121 | 28 | 29 | 32 | 31 | 34 | 34 | 30 | 32 | 29 | 31 | 30 | 30 | 33 | 35 | 38 | 35 |
| PSEDUMONOASE- | 27 | 27 | 31 | 26 | 32 | 32 | 29 | 33 | 30 | 32 | 31 | 31 | 32 | 34 | 36 | 35 |
| FLUORESCENCE | | | | | | | | | | | | | | | | |
| SULFITOBACTER-SP.-NAS-14.1 | 24 | 25 | 28 | 26 | 29 | 27 | 26 | 28 | 25 | 29 | 30 | 29 | 32 | 33 | 32 | 34 |
| OCEANICOLA- | 24 | 25 | 25 | 26 | 28 | 28 | 26 | 30 | 26 | 29 | 29 | 29 | 30 | 34 | 33 | 33 |
| BATSENSIS-HTCC2597 | | | | | | | | | | | | | | | | |
| BDELLOVIBRIO- | 30 | 31 | 31 | 29 | 32 | 33 | 31 | 31 | 32 | 32 | 31 | 32 | 33 | 35 | 36 | 36 |
| BACTERIOVORUS | | | | | | | | | | | | | | | | |

| | | | | | | | | | | | | | | |
|---|---|---|---|---|---|---|---|---|---|---|---|---|---|---|
| RATTUS_NORVEGICUS | 45 | 37 | 37 | 36 | 36 | 37 | 37 | 36 | 36 | 36 | 36 | 37 | 35 | 35 |
| HOMO-SAPIENS | 45 | 36 | 37 | 37 | 37 | 37 | 38 | 37 | 37 | 38 | 37 | 37 | 35 | 35 |
| XENOPUS-LAEVIS | 45 | 37 | 37 | 36 | 34 | 34 | 34 | 38 | 38 | 38 | 38 | 37 | 36 | 36 |
| BLEPHARISMA-JAPONICUS | 45 | 35 | 35 | 35 | 35 | 34 | 36 | 35 | 35 | 35 | 36 | 36 | 35 | 34 |
| MYCOSPHAERELLA- | 44 | 34 | 34 | 34 | 32 | 32 | 33 | 34 | 33 | 33 | 33 | 34 | 31 | 30 |
| GRAMINICOLA | | | | | | | | | | | | | | |
| ASPERGILLUS-FUMIGATUS | 46 | 33 | 33 | 32 | 31 | 32 | 32 | 33 | 33 | 33 | 34 | 35 | 32 | 31 |
| MAGNAPORTHE-GRISEA | 41 | 33 | 34 | 34 | 32 | 32 | 32 | 33 | 33 | 34 | 33 | 35 | 32 | 31 |
| CANDIDA-ALBICANS | 38 | 28 | 28 | 28 | 27 | 29 | 28 | 28 | 28 | 29 | 28 | 28 | 28 | 28 |
| PICHIA-STIPITIS | 38 | 26 | 26 | 28 | 25 | 27 | 26 | 26 | 25 | 27 | 26 | 26 | 26 | 26 |
| RHODOCOCCUS-SP. | 46 | 34 | 34 | 36 | 35 | 35 | 36 | 36 | 35 | 35 | 36 | 38 | 35 | 34 |
| RHODOCOCCUS- | 46 | 35 | 35 | 36 | 34 | 36 | 36 | 36 | 36 | 35 | 37 | 39 | 34 | 33 |
| ERYTHROPOLIS | | | | | | | | | | | | | | |
| JANIBACTER-SP.-HTCC2649 | 44 | 34 | 35 | 34 | 34 | 34 | 36 | 36 | 35 | 35 | 35 | 37 | 32 | 32 |
| STREPTOMYCES- | 46 | 38 | 38 | 38 | 37 | 37 | 38 | 37 | 37 | 37 | 38 | 39 | 35 | 34 |
| AVERMITILIS | | | | | | | | | | | | | | |
| KORDIA-ALGICIDA | 53 | 35 | 35 | 34 | 32 | 34 | 32 | 34 | 35 | 34 | 34 | 36 | 34 | 34 |
| LEEUWENHOEKIELLA- | 51 | 38 | 38 | 36 | 36 | 37 | 37 | 39 | 38 | 38 | 37 | 39 | 38 | 37 |
| BLANDENSIS-ME | | | | | | | | | | | | | | |
| GEMMATIMONAS- | 53 | 39 | 39 | 39 | 39 | 39 | 39 | 39 | 39 | 38 | 40 | 43 | 39 | 37 |
| AURANTIACA | | | | | | | | | | | | | | |
| PICROPHILUS-TORRIDUS | 100 | 35 | 35 | 35 | 36 | 35 | 35 | 35 | 36 | 36 | 36 | 37 | 32 | 32 |
| ARABIDOPSIS-THALIANA | 35 | 100 | 90 | 75 | 76 | 75 | 73 | 72 | 72 | 70 | 73 | 71 | 60 | 60 |
| BRASSICA-RAPA-SUBSP.- | 35 | 90 | 100 | 75 | 76 | 76 | 75 | 74 | 74 | 73 | 73 | 72 | 60 | 60 |
| PEKINENSI | | | | | | | | | | | | | | |
| COPTIS-JAPOMICA-VAR.- | 35 | 75 | 75 | 100 | 78 | 74 | 71 | 73 | 73 | 72 | 73 | 72 | 61 | 61 |
| DISSECTA | | | | | | | | | | | | | | |
| HEVEA-BRASILIENSIS | 36 | 76 | 76 | 78 | 100 | 78 | 74 | 75 | 75 | 74 | 74 | 72 | 62 | 62 |
| MEDICAGO-TRUNCATULA | 35 | 75 | 76 | 74 | 78 | 100 | 76 | 74 | 75 | 74 | 75 | 74 | 62 | 62 |
| GLYCINE-MAX | 35 | 73 | 75 | 71 | 74 | 76 | 100 | 75 | 74 | 74 | 75 | 73 | 62 | 62 |
| SOLANUM-LYCOPERSICUM | 35 | 72 | 74 | 73 | 75 | 74 | 75 | 100 | 96 | 87 | 77 | 76 | 63 | 62 |
| SOLANUM-TUBEROSUM | 36 | 72 | 74 | 73 | 75 | 75 | 74 | 96 | 100 | 88 | 78 | 76 | 62 | 61 |
| NICOTIANA-BENTHAMIANA | 36 | 70 | 73 | 72 | 74 | 74 | 74 | 87 | 88 | 100 | 76 | 75 | 61 | 60 |
| DAUCUS-CAROTA | 36 | 73 | 73 | 73 | 74 | 75 | 75 | 77 | 78 | 76 | 100 | 76 | 63 | 62 |
| SOLENOSTEMON- | 37 | 71 | 72 | 72 | 72 | 74 | 73 | 76 | 76 | 75 | 76 | 100 | 62 | 60 |
| SCUTELLARIOIDES | | | | | | | | | | | | | | |
| HORDEUM-VULGARE | 32 | 60 | 60 | 61 | 62 | 62 | 62 | 63 | 62 | 61 | 63 | 62 | 100 | 95 |
| TRITICUM-AESTIVUM | 32 | 60 | 60 | 61 | 62 | 62 | 62 | 62 | 61 | 60 | 62 | 60 | 95 | 100 |
| AVENA-SATIVA | 32 | 61 | 61 | 62 | 62 | 63 | 62 | 62 | 62 | 61 | 64 | 62 | 83 | 83 |
| ORYZA-SATIVA | 33 | 61 | 61 | 62 | 63 | 63 | 63 | 65 | 64 | 63 | 65 | 64 | 83 | 82 |
| SORGHUM-BICOLOR | 33 | 63 | 62 | 64 | 64 | 65 | 66 | 64 | 62 | 64 | 65 | 64 | 83 | 82 |
| ZEA-MAYS | 34 | 63 | 63 | 64 | 64 | 65 | 67 | 64 | 63 | 64 | 65 | 65 | 83 | 82 |
| ABO95005_OLUCIMARINUS | 32 | 55 | 57 | 55 | 56 | 55 | 54 | 56 | 56 | 55 | 58 | 58 | 54 | 53 |
| OTAURI | 33 | 56 | 57 | 56 | 57 | 56 | 55 | 57 | 57 | 56 | 59 | 59 | 55 | 54 |
| MICROMONAS- | 33 | 57 | 58 | 58 | 57 | 59 | 56 | 57 | 57 | 56 | 59 | 57 | 54 | 54 |
| PUSILLA-CCMP1545 | | | | | | | | | | | | | | |
| SYNECHOCOCCUS-SP. | 33 | 31 | 31 | 29 | 29 | 29 | 29 | 30 | 30 | 31 | 29 | 30 | 33 | 32 |
| VIBRIO-SP.-MED222 | 33 | 28 | 28 | 29 | 28 | 29 | 30 | 28 | 29 | 31 | 29 | 30 | 30 | 30 |
| MARINOMONAS-SP.-MED121 | 33 | 29 | 28 | 31 | 29 | 30 | 30 | 29 | 29 | 30 | 30 | 31 | 30 | 30 |
| PSEDUMONOASE- | 33 | 25 | 26 | 29 | 27 | 27 | 26 | 26 | 26 | 27 | 27 | 27 | 27 | 26 |
| FLUORESCENCE | | | | | | | | | | | | | | |
| SULFITOBACTER-SP.-NAS-14.1 | 29 | 27 | 27 | 28 | 27 | 27 | 27 | 27 | 27 | 27 | 27 | 28 | 27 | 28 |
| OCEANICOLA- | 29 | 27 | 27 | 28 | 27 | 27 | 26 | 26 | 26 | 26 | 26 | 27 | 28 | 28 |
| BATSENSIS-HTCC2597 | | | | | | | | | | | | | | |
| BDELLOVIBRIO- | 34 | 30 | 29 | 31 | 30 | 29 | 31 | 30 | 30 | 30 | 29 | 30 | 30 | 30 |
| BACTERIOVORUS | | | | | | | | | | | | | | |

| | | | | | | | | | | | | | | |
|---|---|---|---|---|---|---|---|---|---|---|---|---|---|---|
| RATTUS_NORVEGICUS | 35 | 35 | 36 | 35 | 34 | 34 | 32 | 31 | 29 | 28 | 27 | 24 | 24 | 30 |
| HOMO-SAPIENS | 35 | 35 | 35 | 35 | 34 | 35 | 34 | 31 | 29 | 29 | 27 | 25 | 25 | 31 |
| XENOPUS-LAEVIS | 36 | 36 | 35 | 36 | 36 | 36 | 35 | 30 | 31 | 32 | 31 | 28 | 25 | 31 |
| BLEPHARISMA-JAPONICUS | 36 | 35 | 35 | 34 | 34 | 34 | 32 | 31 | 30 | 31 | 26 | 26 | 26 | 29 |
| MYCOSPHAERELLA- | 32 | 32 | 32 | 31 | 33 | 33 | 31 | 30 | 33 | 34 | 32 | 29 | 28 | 32 |
| GRAMINICOLA | | | | | | | | | | | | | | |
| ASPERGILLUS-FUMIGATUS | 32 | 32 | 33 | 32 | 32 | 33 | 30 | 29 | 35 | 34 | 32 | 27 | 28 | 33 |
| MAGNAPORTHE-GRISEA | 33 | 32 | 32 | 32 | 34 | 33 | 33 | 30 | 33 | 30 | 29 | 26 | 26 | 31 |
| CANDIDA-ALBICANS | 27 | 28 | 28 | 29 | 26 | 26 | 26 | 27 | 33 | 32 | 33 | 28 | 30 | 31 |
| PICHIA-STIPITIS | 26 | 26 | 27 | 26 | 25 | 24 | 25 | 26 | 32 | 29 | 30 | 25 | 26 | 32 |
| RHODOCOCCUS-SP. | 37 | 36 | 37 | 36 | 36 | 36 | 34 | 34 | 30 | 31 | 32 | 29 | 29 | 32 |
| RHODOCOCCUS- | 36 | 34 | 36 | 35 | 35 | 35 | 34 | 32 | 30 | 30 | 31 | 30 | 29 | 31 |
| ERYTHROPOLIS | | | | | | | | | | | | | | |
| JANIBACTER-SP.-HTCC2649 | 33 | 33 | 34 | 34 | 36 | 36 | 33 | 32 | 31 | 30 | 31 | 29 | 29 | 32 |
| STREPTOMYCES- | 35 | 35 | 36 | 36 | 38 | 38 | 35 | 34 | 34 | 33 | 32 | 32 | 30 | 33 |
| AVERMITILIS | | | | | | | | | | | | | | |
| KORDIA-ALGICIDA | 34 | 35 | 34 | 34 | 34 | 35 | 34 | 31 | 37 | 35 | 34 | 33 | 34 | 35 |
| LEEUWENHOEKIELLA- | 37 | 38 | 38 | 39 | 38 | 38 | 36 | 34 | 37 | 38 | 36 | 32 | 33 | 36 |
| BLANDENSIS-ME | | | | | | | | | | | | | | |
| GEMMATIMONAS- | 38 | 38 | 39 | 39 | 40 | 40 | 38 | 36 | 37 | 35 | 35 | 34 | 33 | 36 |
| AURANTIACA | | | | | | | | | | | | | | |
| PICROPHILUS-TORRIDUS | 32 | 33 | 33 | 34 | 32 | 33 | 33 | 33 | 33 | 33 | 33 | 29 | 29 | 34 |
| ARABIDOPSIS-THALIANA | 61 | 61 | 63 | 63 | 55 | 56 | 57 | 31 | 28 | 29 | 25 | 27 | 27 | 30 |
| BRASSICA-RAPA-SUBSP.- | 61 | 61 | 62 | 63 | 57 | 57 | 58 | 31 | 28 | 28 | 26 | 27 | 27 | 29 |
| PEKINENSI | | | | | | | | | | | | | | |
| COPTIS-JAPOMICA-VAR.- | 62 | 62 | 64 | 64 | 55 | 56 | 58 | 29 | 29 | 31 | 29 | 28 | 28 | 31 |
| DISSECTA | | | | | | | | | | | | | | |
| HEVEA-BRASILIENSIS | 62 | 63 | 64 | 64 | 56 | 57 | 57 | 29 | 28 | 29 | 27 | 27 | 27 | 30 |
| MEDICAGO-TRUNCATULA | 63 | 63 | 65 | 65 | 55 | 56 | 59 | 29 | 29 | 30 | 27 | 27 | 27 | 29 |
| GLYCINE-MAX | 62 | 63 | 66 | 67 | 54 | 55 | 56 | 29 | 30 | 30 | 26 | 27 | 26 | 31 |
| SOLANUM-LYCOPERSICUM | 62 | 65 | 64 | 64 | 56 | 57 | 57 | 30 | 28 | 29 | 26 | 27 | 26 | 30 |
| SOLANUM-TUBEROSUM | 62 | 64 | 62 | 63 | 56 | 57 | 57 | 30 | 29 | 29 | 26 | 27 | 26 | 30 |
| NICOTIANA-BENTHAMIANA | 61 | 63 | 64 | 64 | 55 | 56 | 56 | 31 | 31 | 30 | 27 | 27 | 26 | 30 |
| DAUCUS-CAROTA | 64 | 65 | 65 | 65 | 58 | 59 | 59 | 29 | 29 | 30 | 27 | 27 | 26 | 29 |
| SOLENOSTEMON- | 62 | 64 | 64 | 65 | 58 | 59 | 57 | 30 | 30 | 31 | 27 | 28 | 27 | 30 |
| SCUTELLARIOIDES | | | | | | | | | | | | | | |
| HORDEUM-VULGARE | 83 | 83 | 83 | 83 | 54 | 55 | 54 | 33 | 30 | 30 | 27 | 27 | 28 | 30 |
| TRITICUM-AESTIVUM | 83 | 82 | 82 | 82 | 53 | 54 | 54 | 32 | 30 | 30 | 26 | 28 | 28 | 30 |
| AVENA-SATIVA | 100 | 81 | 80 | 79 | 54 | 53 | 54 | 30 | 30 | 29 | 27 | 27 | 28 | 31 |
| ORYZA-SATIVA | 81 | 100 | 84 | 83 | 54 | 56 | 55 | 32 | 29 | 29 | 27 | 26 | 28 | 31 |
| SORGHUM-BICOLOR | 80 | 84 | 100 | 93 | 54 | 56 | 56 | 30 | 29 | 30 | 27 | 26 | 27 | 29 |
| ZEA-MAYS | 79 | 83 | 93 | 100 | 55 | 57 | 57 | 32 | 30 | 31 | 27 | 26 | 26 | 29 |
| ABO95005_OLUCIMARINUS | 54 | 54 | 54 | 55 | 100 | 84 | 63 | 28 | 30 | 27 | 25 | 25 | 27 | 27 |
| OTAURI | 53 | 56 | 56 | 57 | 84 | 100 | 62 | 28 | 31 | 29 | 25 | 26 | 27 | 27 |
| MICROMONAS- | 54 | 55 | 56 | 57 | 63 | 62 | 100 | 29 | 27 | 28 | 24 | 25 | 25 | 27 |
| PUSILLA-CCMP1545 | | | | | | | | | | | | | | |
| SYNECHOCOCCUS-SP. | 30 | 32 | 30 | 32 | 28 | 28 | 29 | 100 | 26 | 27 | 28 | 23 | 24 | 28 |
| VIBRIO-SP.-MED222 | 30 | 29 | 29 | 30 | 30 | 31 | 27 | 26 | 100 | 74 | 53 | 53 | 50 | 46 |
| MARINOMONAS-SP.-MED121 | 29 | 29 | 30 | 31 | 27 | 29 | 28 | 27 | 74 | 100 | 56 | 51 | 49 | 44 |
| PSEDUMONOASE- | 27 | 27 | 27 | 27 | 25 | 25 | 24 | 28 | 53 | 56 | 100 | 48 | 50 | 43 |
| FLUORESCENCE | | | | | | | | | | | | | | |
| SULFITOBACTER-SP.-NAS-14.1 | 27 | 26 | 26 | 26 | 25 | 26 | 25 | 23 | 53 | 51 | 48 | 100 | 81 | 45 |
| OCEANICOLA- | 28 | 28 | 27 | 26 | 27 | 27 | 25 | 24 | 50 | 49 | 50 | 81 | 100 | 45 |
| BATSENSIS-HTCC2597 | | | | | | | | | | | | | | |
| BDELLOVIBRIO- | 31 | 31 | 29 | 29 | 27 | 27 | 27 | 28 | 46 | 44 | 43 | 45 | 45 | 100 |
| BACTERIOVORUS | | | | | | | | | | | | | | |

TABLE 4a
Sequence alignment of amino acids forming the binding pocket
in the representative set of HPPD sequences

| | |
|---|---|
| HOMO-SAPIENS | HVHFWSVLSIVVAPNQIQHLYLQFFEFGAGNFNSLF |
| RATTUS_NORVEGICUS | HVHFWSVLSIVVAPNQIQHLYLQFFEFGAGNFNSLF |
| XENOPUS-LAEVIS | HVHFWSVLSIVVTPNQIQHLYLQFFEFGAGNFKALI |
| ASPERGILLUS-FUMIGATUS | HVHFWSVLSIVMAPNQIQHIYLQFFEFGAGNFKSLI |
| MAGNAPORTHE-GRISEA | HVHFWSVLSIVMAPNQIQHIYLQFFEFGAGNFKSLI |
| MYCOSPHAERELLA-GRAMINICOLA | HVHFWSVLSIVMSPNQIQHIYLQFFEFGAGNFKSLI |
| CANDIDA-ALBICANS | HVHYWSVLSIVMSPNQIQHNYLQFFEFGKGTFKGLI |
| PICHIA-STIPITIS | HVHYWSALSIVMAPNQIQHNYLQFFEFGKGTFKGLI |
| ARABIDOPSIS-THALIANA | HVHFAEFLSAVLAPNQIQHMYLQFFEFGKGNFSELI |
| BRASSICA-RAPA-SUBSP.-PEKINENSI | HVHFAEFLSAVLAPNQIQHMYLQFFEFGKGNFSELI |
| HEVEA-BRASILIENSIS | HVHFAEFLSLVLAPNQIQHMYLQFFEFGKGNFSELI |
| COPTIS-JAPONICA-VAR.-DISSECTA | HVHFAEFLSIVLAPNQIQHMYLQFFEFGKGNFSELI |
| SOLENOSTEMON-SCUTELLARIOIDES | HVHFAEFLSVVLAPNQIQHMYLQFFEFGKGNFSELI |
| AVENA-SATIVA | HVHFAEFLSVVLAPNQIQHMYLQFFEFGKGNFSELI |
| DAUCUS-CAROTA | HVHFAEFLSVVLAPNQIQHMYLQFFEFGKGNFSELI |
| SOLANUM-TUBEROSUM | HVHFAEFLSVVLAPNQIQHMYLQFFEFGKGNFSELI |
| SOLANUM-LYCOPERSICUM | HVHFAEFLSVVLAPNQIQHMYLQFFEFGKGNFSELI |
| MEDICAGO-TRUNCATULA | HVHFAEFLSVVLAPNQIQHMYLQFFEFGKGNFSELI |
| GLYCINE-MAX | HVHFAEFLSVVLAPNQIQHMYLQFFXFGKGNFSELI |
| HORDEUM-VULGARE | HVHFAEFLSVVLAPNQIQHLYLQFFEFGKGNFSELI |
| ORYZA-SATIVA | HVHFAEFLSVVLAPNQIQHLYLQFFEFGKGNFSELI |
| SORGHUM-BICOLOR | HVHFAEFLSMVLAPNQIQHMYLQFFEFGKGNFSQLI |
| ZEA-MAYS | HVHFAEFLSMVLAPNQIQHMYLQFFEFGKGNFSQLI |
| ABO95005_OLUCIMARINUS | HVHFAEFLSMVLAPNQIQHQYLQFFEFGKGNFSELI |
| OTAURI | HVHFAEFLSMVLAPNQIQHQYLQFFEFGKGNFSELI |
| MICROMONAS-PUSILLA-CCMP1545 | HVHFAEFLSMVLAPNQIQHQYLQFFEFGKGNFSELI |
| TRITICUM-AESTIVUM | HVHFAEFLSMVLAPNQIQHLYLQFFEFGKGNFSELI |
| NICOTIANA-BENTHAMIANA | HVHFAEFLSMVVAPNQIQHMYLQFFEFGKGNFWELI |
| RHODOCOCCUS-SP. | HVTMAEFLSKVVSPNQIQHLYLQFFEFGIGNFKALI |
| RHODOCOCCUS-ERYTHROPOLIS | HVTMAEFLSKVVSPNQIQHLYLQFFEFGLGNFKALI |
| STREPTOMYCES-AVERMITILIS | HVTMKEFLSKVVAPNQIQHLYLQFFEFGKGNFKALI |
| JANIBACTER-SP.-HTCC2649 | HVVMAEFLSKVVAPNQIQHLYLQFFEFGKGNFKALI |
| KORDIA-ALGICIDA | HVAIISFLSKVMSPNQIQHLYLQFFEFGVGNFKALI |
| LEEUWENHOEKIELLA-BLANDENSIS-ME | HVVFLTFLSKVMSPNQIQHLYLQFFEFGAGNFKALI |
| PICROPHILUS-TORRIDUS | HVELITFLSKVVKPNQIQHLYLQFFEFGNGNFKALI |
| GEMMATIMONAS-AURANTIACA | HVRLITFLSKVMAPNQIQHLYLQFFEFGKGNFRALI |
| VIBRIO-SP.-MED222 | HTRIRYFLSRAMTPNQIQHMYLQFFEFGEGNFKALI |
| MARINOMONAS-SP.-MED121 | HTRIRYFLSRAMTPNQIQHMYLQFFEFGEGNFKALI |
| SULFITOBACTER-SP.-NAS-14.1 | HTRIRFFLSRALTPNQIQHMYLQFFEFGEGNFKALI |
| OCEANICOLA-BATSENSIS-HTCC2597 | HTRIRFFLSRALTPNQIQHMYLQFFEFGEGNFKALI |
| PSEUDOMONAS-FLUORESCENS | HTRARYFLSKAMSPNQIQHMYLQFFEFGEGNFKALI |
| BDELLOVIBRIO-BACTERIOVORUS | HTYAKYFLSRAMRPNQIQHLYLQFFEFGDGNFQALI |
| BLEPHARISMA-JAPONICUS | HVHYWSALSVVVAPNQIQHLYLQFFEFGIGNFKALL |
| SYNECHOCOCCUS-SP. | HVRLYRYLSVVVGANQIQHLYLQFFEFGEANFQALL |

TABLE 4b
Pairwise sequence identity of amino acids forming the binding site HPPD in the representative set

| | | | | | | | | | | | | | | | | |
|---|---|---|---|---|---|---|---|---|---|---|---|---|---|---|---|---|
| HOMO-SAPIENS | 100 | 100 | 89 | 89 | 89 | 86 | 75 | 75 | 72 | 72 | 72 | 75 | 72 | 72 | 72 | 72 |
| RATTUS_NORVEGICUS | 100 | 100 | 89 | 89 | 89 | 86 | 75 | 75 | 72 | 72 | 72 | 75 | 72 | 72 | 72 | 72 |
| XENOPUS-LAEVIS | 89 | 89 | 100 | 89 | 89 | 89 | 81 | 78 | 72 | 72 | 72 | 75 | 72 | 72 | 72 | 72 |
| ASPERGILLUS-FUMIGATUS | 89 | 89 | 89 | 100 | 100 | 97 | 83 | 83 | 75 | 75 | 75 | 78 | 75 | 75 | 75 | 75 |
| MAGNAPORTHE-GRISEA | 89 | 89 | 89 | 100 | 100 | 97 | 83 | 83 | 75 | 75 | 75 | 78 | 75 | 75 | 75 | 75 |
| MYCOSPHAERELLA- | 86 | 86 | 89 | 97 | 97 | 100 | 86 | 81 | 72 | 72 | 72 | 75 | 72 | 72 | 72 | 72 |
| GRAMINICOLA | | | | | | | | | | | | | | | | |
| CANDIDA-ALBICANS | 75 | 75 | 81 | 83 | 83 | 86 | 100 | 94 | 69 | 69 | 69 | 72 | 69 | 69 | 69 | 69 |
| PICHIA-STIPITIS | 75 | 75 | 78 | 83 | 83 | 81 | 94 | 100 | 72 | 72 | 72 | 75 | 72 | 72 | 72 | 72 |
| ARABIDOPSIS-THALIANA | 72 | 72 | 72 | 75 | 75 | 72 | 69 | 72 | 100 | 100 | 97 | 97 | 97 | 97 | 97 | 97 |
| BRASSICA-RAPA-SUBSP.- | 72 | 72 | 72 | 75 | 75 | 72 | 69 | 72 | 100 | 100 | 97 | 97 | 97 | 97 | 97 | 97 |
| PEKINENSI | | | | | | | | | | | | | | | | |
| HEVEA-BRASILIENSIS | 72 | 72 | 72 | 75 | 75 | 72 | 69 | 72 | 97 | 97 | 100 | 97 | 97 | 97 | 97 | 97 |
| COPTIS-JAPONICA-VAR.- | 75 | 75 | 75 | 78 | 78 | 75 | 72 | 75 | 97 | 97 | 97 | 100 | 97 | 97 | 97 | 97 |
| DISSECTA | | | | | | | | | | | | | | | | |
| SOLENOSTEMON- | 72 | 72 | 72 | 75 | 75 | 72 | 69 | 72 | 97 | 97 | 97 | 97 | 100 | 100 | 100 | 100 |
| SCUTELLARIOIDES | | | | | | | | | | | | | | | | |
| AVENA-SATIVA | 72 | 72 | 72 | 75 | 75 | 72 | 69 | 72 | 97 | 97 | 97 | 97 | 100 | 100 | 100 | 100 |
| DAUCUS-CAROTA | 72 | 72 | 72 | 75 | 75 | 72 | 69 | 72 | 97 | 97 | 97 | 97 | 100 | 100 | 100 | 100 |
| SOLANUM-TUBEROSUM | 72 | 72 | 72 | 75 | 75 | 72 | 69 | 72 | 97 | 97 | 97 | 97 | 100 | 100 | 100 | 100 |
| SOLANUM-LYCOPERSICUM | 72 | 72 | 72 | 75 | 75 | 72 | 69 | 72 | 97 | 97 | 97 | 97 | 100 | 100 | 100 | 100 |
| MEDICAGO-TRUNCATULA | 72 | 72 | 72 | 75 | 75 | 72 | 69 | 72 | 97 | 97 | 97 | 97 | 100 | 100 | 100 | 100 |
| GLYCINE-MAX | 69 | 69 | 69 | 72 | 72 | 69 | 67 | 69 | 94 | 94 | 94 | 94 | 97 | 97 | 97 | 97 |
| HORDEUM-VULGARE | 75 | 75 | 75 | 75 | 75 | 72 | 69 | 72 | 94 | 94 | 94 | 94 | 97 | 97 | 97 | 97 |
| ORYZA-SATIVA | 75 | 75 | 75 | 75 | 75 | 72 | 69 | 72 | 94 | 94 | 94 | 94 | 97 | 97 | 97 | 97 |
| SORGHUM-BICOLOR | 72 | 72 | 72 | 75 | 75 | 72 | 69 | 72 | 94 | 94 | 94 | 94 | 94 | 94 | 94 | 94 |
| ZEA-MAYS | 72 | 72 | 72 | 75 | 75 | 72 | 69 | 72 | 94 | 94 | 94 | 94 | 94 | 94 | 94 | 94 |
| ABO95005_OLUCIMARINUS | 72 | 72 | 72 | 75 | 75 | 72 | 69 | 72 | 94 | 94 | 94 | 94 | 94 | 94 | 94 | 94 |
| OTAURI | 72 | 72 | 72 | 75 | 75 | 72 | 69 | 72 | 94 | 94 | 94 | 94 | 94 | 94 | 94 | 94 |
| MICROMONAS- | 72 | 72 | 72 | 75 | 75 | 72 | 69 | 72 | 94 | 94 | 94 | 94 | 94 | 94 | 94 | 94 |
| PUSILLA-CCMP1545 | | | | | | | | | | | | | | | | |
| TRITICUM-AESTIVUM | 75 | 75 | 75 | 75 | 75 | 72 | 69 | 72 | 94 | 94 | 94 | 94 | 94 | 94 | 94 | 94 |
| NICOTIANA-BENTHAMIANA | 75 | 75 | 75 | 75 | 75 | 72 | 69 | 72 | 92 | 92 | 92 | 92 | 92 | 92 | 92 | 92 |
| RHODOCOCCUS-SP. | 69 | 69 | 78 | 69 | 69 | 72 | 69 | 67 | 75 | 75 | 75 | 75 | 75 | 75 | 75 | 75 |
| RHODOCOCCUS- | 69 | 69 | 78 | 69 | 69 | 72 | 69 | 67 | 75 | 75 | 75 | 75 | 75 | 75 | 75 | 75 |
| ERYTHROPOLIS | | | | | | | | | | | | | | | | |
| STREPTOMYCES- | 72 | 72 | 78 | 72 | 72 | 69 | 69 | 72 | 78 | 78 | 78 | 78 | 78 | 78 | 78 | 78 |
| AVERMITILIS | | | | | | | | | | | | | | | | |
| JANIBACTER-SP.-HTCC2649 | 72 | 72 | 78 | 72 | 72 | 69 | 69 | 72 | 81 | 81 | 81 | 81 | 81 | 81 | 81 | 81 |
| KORDIA-ALGICIDA | 69 | 69 | 78 | 75 | 75 | 78 | 75 | 72 | 69 | 69 | 69 | 69 | 69 | 69 | 69 | 69 |
| LEEUWENHOEKIELLA- | 72 | 72 | 81 | 78 | 78 | 81 | 72 | 69 | 72 | 72 | 72 | 72 | 72 | 72 | 72 | 72 |
| BLANDENSIS-ME | | | | | | | | | | | | | | | | |
| PICROPHILUS-TORRIDUS | 69 | 69 | 78 | 69 | 69 | 69 | 67 | 67 | 69 | 69 | 69 | 69 | 69 | 69 | 69 | 69 |
| GEMMATIMONAS- | 69 | 69 | 72 | 72 | 72 | 69 | 69 | 72 | 75 | 75 | 75 | 75 | 75 | 75 | 75 | 75 |
| AURANTIACA | | | | | | | | | | | | | | | | |
| VIBRIO-SP.-MED222 | 58 | 58 | 69 | 67 | 67 | 67 | 64 | 64 | 67 | 67 | 67 | 67 | 67 | 67 | 67 | 67 |
| MARINOMONAS-SP.-MED121 | 58 | 58 | 69 | 67 | 67 | 67 | 64 | 64 | 67 | 67 | 67 | 67 | 67 | 67 | 67 | 67 |
| SULFITOBACTER-SP.-NAS-14.1 | 58 | 58 | 69 | 64 | 64 | 64 | 61 | 61 | 69 | 69 | 69 | 69 | 69 | 69 | 69 | 69 |
| OCEANICOLA- | 58 | 58 | 69 | 64 | 64 | 64 | 61 | 61 | 69 | 69 | 69 | 69 | 69 | 69 | 69 | 69 |
| BATSENSIS-HTCC2597 | | | | | | | | | | | | | | | | |
| PSEDUMONOASE- | 58 | 58 | 67 | 67 | 67 | 69 | 67 | 64 | 67 | 67 | 67 | 67 | 67 | 67 | 67 | 67 |
| FLUORESCENCE | | | | | | | | | | | | | | | | |
| BDELLOVIBRIO- | 61 | 61 | 67 | 64 | 64 | 64 | 61 | 61 | 64 | 64 | 64 | 64 | 64 | 64 | 64 | 64 |
| BACTERIOVORUS | | | | | | | | | | | | | | | | |
| BLEPHARISMA-JAPONICUS | 81 | 81 | 83 | 78 | 78 | 75 | 75 | 81 | 69 | 69 | 69 | 69 | 72 | 72 | 72 | 72 |
| SYNECHOCOCCUS-SP. | 64 | 64 | 67 | 58 | 58 | 58 | 56 | 56 | 58 | 58 | 58 | 58 | 61 | 61 | 61 | 61 |

| HOMO-SAPIENS | 72 | 72 | 69 | 75 | 75 | 72 | 72 | 72 | 72 | 72 | 75 | 75 | 69 | 69 |
|---|---|---|---|---|---|---|---|---|---|---|---|---|---|---|
| RATTUS_NORVEGICUS | 72 | 72 | 69 | 75 | 75 | 72 | 72 | 72 | 72 | 72 | 75 | 75 | 69 | 69 |
| XENOPUS-LAEVIS | 72 | 72 | 69 | 75 | 75 | 72 | 72 | 72 | 72 | 72 | 75 | 75 | 78 | 78 |
| ASPERGILLUS-FUMIGATUS | 75 | 75 | 72 | 75 | 75 | 75 | 75 | 75 | 75 | 75 | 75 | 75 | 69 | 69 |
| MAGNAPORTHE-GRISEA | 75 | 75 | 72 | 75 | 75 | 75 | 75 | 75 | 75 | 75 | 75 | 75 | 69 | 69 |
| MYCOSPHAERELLA- | 72 | 72 | 69 | 72 | 72 | 72 | 72 | 72 | 72 | 72 | 72 | 72 | 72 | 72 |
| GRAMINICOLA | | | | | | | | | | | | | | |
| CANDIDA-ALBICANS | 69 | 69 | 67 | 69 | 69 | 69 | 69 | 69 | 69 | 69 | 69 | 69 | 69 | 69 |
| PICHIA-STIPITIS | 72 | 72 | 69 | 72 | 72 | 72 | 72 | 72 | 72 | 72 | 72 | 72 | 67 | 67 |
| ARABIDOPSIS-THALIANA | 97 | 97 | 94 | 94 | 94 | 94 | 94 | 94 | 94 | 94 | 94 | 92 | 75 | 75 |
| BRASSICA-RAPA-SUBSP.- | 97 | 97 | 94 | 94 | 94 | 94 | 94 | 94 | 94 | 94 | 94 | 92 | 75 | 75 |
| PEKINENSI | | | | | | | | | | | | | | |
| HEVEA-BRASILIENSIS | 97 | 97 | 94 | 94 | 94 | 94 | 94 | 94 | 94 | 94 | 94 | 92 | 75 | 75 |
| COPTIS-JAPONICA-VAR.- | 97 | 97 | 94 | 94 | 94 | 94 | 94 | 94 | 94 | 94 | 94 | 92 | 75 | 75 |
| DISSECTA | | | | | | | | | | | | | | |
| SOLENOSTEMON- | 100 | 100 | 97 | 97 | 97 | 94 | 94 | 94 | 94 | 94 | 94 | 92 | 75 | 75 |
| SCUTELLARIOIDES | | | | | | | | | | | | | | |
| AVENA-SATIVA | 100 | 100 | 97 | 97 | 97 | 94 | 94 | 94 | 94 | 94 | 94 | 92 | 75 | 75 |
| DAUCUS-CAROTA | 100 | 100 | 97 | 97 | 97 | 94 | 94 | 94 | 94 | 94 | 94 | 92 | 75 | 75 |
| SOLANUM-TUBEROSUM | 100 | 100 | 97 | 97 | 97 | 94 | 94 | 94 | 94 | 94 | 94 | 92 | 75 | 75 |
| SOLANUM-LYCOPERSICUM | 100 | 100 | 97 | 97 | 97 | 94 | 94 | 94 | 94 | 94 | 94 | 92 | 75 | 75 |
| MEDICAGO-TRUNCATULA | 100 | 100 | 97 | 97 | 97 | 94 | 94 | 94 | 94 | 94 | 94 | 92 | 75 | 75 |
| GLYCINE-MAX | 97 | 97 | 100 | 94 | 94 | 92 | 92 | 92 | 92 | 92 | 92 | 89 | 72 | 72 |
| HORDEUM-VULGARE | 97 | 97 | 94 | 100 | 100 | 92 | 92 | 94 | 94 | 94 | 97 | 89 | 78 | 78 |
| ORYZA-SATIVA | 97 | 97 | 94 | 100 | 100 | 92 | 92 | 94 | 94 | 94 | 97 | 89 | 78 | 78 |
| SORGHUM-BICOLOR | 94 | 94 | 92 | 92 | 92 | 100 | 100 | 94 | 94 | 94 | 94 | 92 | 75 | 75 |
| ZEA-MAYS | 94 | 94 | 92 | 92 | 92 | 100 | 100 | 94 | 94 | 94 | 94 | 92 | 75 | 75 |
| ABO95005_OLUCIMARINUS | 94 | 94 | 92 | 94 | 94 | 94 | 94 | 100 | 100 | 100 | 97 | 92 | 75 | 75 |
| OTAURI | 94 | 94 | 92 | 94 | 94 | 94 | 94 | 100 | 100 | 100 | 97 | 92 | 75 | 75 |
| MICROMONAS- | 94 | 94 | 92 | 94 | 94 | 94 | 94 | 100 | 100 | 100 | 97 | 92 | 75 | 75 |
| PUSILLA-CCMP1545 | | | | | | | | | | | | | | |
| TRITICUM-AESTIVUM | 94 | 94 | 92 | 97 | 97 | 94 | 94 | 97 | 97 | 97 | 100 | 92 | 78 | 78 |
| NICOTIANA-BENTHAMIANA | 92 | 92 | 89 | 89 | 89 | 92 | 92 | 92 | 92 | 92 | 92 | 100 | 78 | 78 |
| RHODOCOCCUS-SP. | 75 | 75 | 72 | 78 | 78 | 75 | 75 | 75 | 75 | 75 | 78 | 78 | 100 | 97 |
| RHODOCOCCUS- | 75 | 75 | 72 | 78 | 78 | 75 | 75 | 75 | 75 | 75 | 78 | 78 | 97 | 100 |
| ERYTHROPOLIS | | | | | | | | | | | | | | |
| STREPTOMYCES- | 78 | 78 | 75 | 81 | 81 | 78 | 78 | 78 | 78 | 78 | 81 | 81 | 92 | 92 |
| AVERMITILIS | | | | | | | | | | | | | | |
| JANIBACTER-SP.-HTCC2649 | 81 | 81 | 78 | 83 | 83 | 81 | 81 | 81 | 81 | 81 | 83 | 83 | 92 | 92 |
| KORDIA-ALGICIDA | 69 | 69 | 67 | 72 | 72 | 69 | 69 | 69 | 69 | 69 | 72 | 69 | 83 | 83 |
| LEEUWENHOEKIELLA- | 72 | 72 | 69 | 75 | 75 | 72 | 72 | 72 | 72 | 72 | 75 | 72 | 83 | 83 |
| BLANDENSIS-ME | | | | | | | | | | | | | | |
| PICROPHILUS-TORRIDUS | 69 | 69 | 67 | 72 | 72 | 69 | 69 | 69 | 69 | 69 | 72 | 72 | 83 | 83 |
| GEMMATIMONAS- | 75 | 75 | 72 | 78 | 78 | 75 | 75 | 75 | 75 | 75 | 78 | 75 | 78 | 78 |
| AURANTIACA | | | | | | | | | | | | | | |
| VIBRIO-SP.-MED222 | 67 | 67 | 64 | 64 | 64 | 67 | 67 | 64 | 64 | 64 | 64 | 67 | 69 | 69 |
| MARINOMONAS-SP.-MED121 | 67 | 67 | 64 | 64 | 64 | 67 | 67 | 64 | 64 | 64 | 64 | 67 | 69 | 69 |
| SULFITOBACTER-SP.-NAS-14.1 | 69 | 69 | 67 | 67 | 67 | 69 | 69 | 67 | 67 | 67 | 67 | 67 | 69 | 69 |
| OCEANICOLA- | 69 | 69 | 67 | 67 | 67 | 69 | 69 | 67 | 67 | 67 | 67 | 67 | 69 | 69 |
| BATSENSIS-HTCC2597 | | | | | | | | | | | | | | |
| PSEDUMONOASE- | 67 | 67 | 64 | 64 | 64 | 67 | 67 | 64 | 64 | 64 | 64 | 67 | 75 | 75 |
| FLUORESCENCE | | | | | | | | | | | | | | |
| BDELLOVIBRIO- | 64 | 64 | 61 | 67 | 67 | 64 | 64 | 64 | 64 | 64 | 67 | 64 | 69 | 69 |
| BACTERIOVORUS | | | | | | | | | | | | | | |
| BLEPHARISMA-JAPONICUS | 72 | 72 | 69 | 75 | 75 | 69 | 69 | 69 | 69 | 69 | 72 | 72 | 78 | 75 |
| SYNECHOCOCCUS-SP. | 61 | 61 | 58 | 64 | 64 | 58 | 58 | 58 | 58 | 58 | 61 | 61 | 67 | 67 |
| HOMO-SAPIENS | 72 | 72 | 69 | 72 | 69 | 69 | 58 | 58 | 58 | 58 | 58 | 61 | 81 | 64 |
| RATTUS_NORVEGICUS | 72 | 72 | 69 | 72 | 69 | 69 | 58 | 58 | 58 | 58 | 58 | 61 | 81 | 64 |
| XENOPUS-LAEVIS | 78 | 78 | 78 | 81 | 78 | 72 | 69 | 69 | 69 | 69 | 67 | 67 | 83 | 67 |
| ASPERGILLUS-FUMIGATUS | 72 | 72 | 75 | 78 | 69 | 72 | 67 | 67 | 64 | 64 | 67 | 64 | 78 | 58 |
| MAGNAPORTHE-GRISEA | 72 | 72 | 75 | 78 | 69 | 72 | 67 | 67 | 64 | 64 | 67 | 64 | 78 | 58 |
| MYCOSPHAERELLA- | 69 | 69 | 78 | 81 | 69 | 69 | 67 | 67 | 64 | 64 | 69 | 64 | 75 | 58 |
| GRAMINICOLA | | | | | | | | | | | | | | |
| CANDIDA-ALBICANS | 69 | 69 | 75 | 72 | 67 | 69 | 64 | 64 | 61 | 61 | 67 | 61 | 75 | 56 |
| PICHIA-STIPITIS | 72 | 72 | 72 | 69 | 67 | 72 | 64 | 64 | 61 | 61 | 64 | 61 | 81 | 56 |
| ARABIDOPSIS-THALIANA | 78 | 81 | 69 | 72 | 69 | 75 | 67 | 67 | 69 | 69 | 67 | 64 | 69 | 58 |
| BRASSICA-RAPA-SUBSP.- | 78 | 81 | 69 | 72 | 69 | 75 | 67 | 67 | 69 | 69 | 67 | 64 | 69 | 58 |
| PEKINENSI | | | | | | | | | | | | | | |
| HEVEA-BRASILIENSIS | 78 | 81 | 69 | 72 | 69 | 75 | 67 | 67 | 69 | 69 | 67 | 64 | 69 | 58 |
| COPTIS-JAPONICA-VAR.- | 78 | 81 | 69 | 72 | 69 | 75 | 67 | 67 | 69 | 69 | 67 | 64 | 69 | 58 |
| DISSECTA | | | | | | | | | | | | | | |
| SOLENOSTEMON- | 78 | 81 | 69 | 72 | 69 | 75 | 67 | 67 | 69 | 69 | 67 | 64 | 72 | 61 |
| SCUTELLARIOIDES | | | | | | | | | | | | | | |
| AVENA-SATIVA | 78 | 81 | 69 | 72 | 69 | 75 | 67 | 67 | 69 | 69 | 67 | 64 | 72 | 61 |
| DAUCUS-CAROTA | 78 | 81 | 69 | 72 | 69 | 75 | 67 | 67 | 69 | 69 | 67 | 64 | 72 | 61 |
| SOLANUM-TUBEROSUM | 78 | 81 | 69 | 72 | 69 | 75 | 67 | 67 | 69 | 69 | 67 | 64 | 72 | 61 |
| SOLANUM-LYCOPERSICUM | 78 | 81 | 69 | 72 | 69 | 75 | 67 | 67 | 69 | 69 | 67 | 64 | 72 | 61 |
| MEDICAGO-TRUNCATULA | 78 | 81 | 69 | 72 | 69 | 75 | 67 | 67 | 69 | 69 | 67 | 64 | 72 | 61 |
| GLYCINE-MAX | 75 | 78 | 67 | 69 | 67 | 72 | 64 | 64 | 67 | 67 | 64 | 61 | 69 | 58 |
| HORDEUM-VULGARE | 81 | 83 | 72 | 75 | 72 | 78 | 64 | 64 | 67 | 67 | 64 | 67 | 75 | 64 |
| ORYZA-SATIVA | 81 | 83 | 72 | 75 | 72 | 78 | 64 | 64 | 67 | 67 | 64 | 67 | 75 | 64 |
| SORGHUM-BICOLOR | 78 | 81 | 69 | 72 | 69 | 75 | 67 | 67 | 69 | 69 | 67 | 64 | 69 | 58 |
| ZEA-MAYS | 78 | 81 | 69 | 72 | 69 | 75 | 67 | 67 | 69 | 69 | 67 | 64 | 69 | 58 |
| ABO95005_OLUCIMARINUS | 78 | 81 | 69 | 72 | 69 | 75 | 64 | 64 | 67 | 67 | 64 | 64 | 69 | 58 |
| OTAURI | 78 | 81 | 69 | 72 | 69 | 75 | 64 | 64 | 67 | 67 | 64 | 64 | 69 | 58 |
| MICROMONAS- | 78 | 81 | 69 | 72 | 69 | 75 | 64 | 64 | 67 | 67 | 64 | 64 | 69 | 58 |
| PUSILLA-CCMP1545 | | | | | | | | | | | | | | |
| TRITICUM-AESTIVUM | 81 | 83 | 72 | 75 | 72 | 78 | 64 | 64 | 67 | 67 | 64 | 67 | 72 | 61 |
| NICOTIANA-BENTHAMIANA | 81 | 83 | 69 | 72 | 72 | 75 | 67 | 67 | 67 | 67 | 67 | 64 | 72 | 61 |
| RHODOCOCCUS-SP. | 92 | 92 | 83 | 83 | 83 | 78 | 69 | 69 | 69 | 69 | 75 | 69 | 78 | 67 |
| RHODOCOCCUS- | 92 | 92 | 83 | 83 | 83 | 78 | 69 | 69 | 69 | 69 | 75 | 69 | 75 | 67 |
| ERYTHROPOLIS | | | | | | | | | | | | | | |
| STREPTOMYCES- | 100 | 94 | 81 | 81 | 83 | 83 | 69 | 69 | 69 | 69 | 72 | 72 | 78 | 67 |
| AVERMITILIS | | | | | | | | | | | | | | |
| JANIBACTER-SP.-HTCC2649 | 94 | 100 | 81 | 83 | 83 | 83 | 69 | 69 | 69 | 69 | 72 | 69 | 78 | 67 |
| KORDIA-ALGICIDA | 81 | 81 | 100 | 86 | 83 | 83 | 75 | 75 | 72 | 72 | 78 | 72 | 75 | 64 |
| LEEUWENHOEKIELLA- | 81 | 83 | 86 | 100 | 83 | 83 | 72 | 72 | 69 | 69 | 78 | 72 | 72 | 64 |
| BLANDENSIS-ME | | | | | | | | | | | | | | |
| PICROPHILUS-TORRIDUS | 83 | 83 | 83 | 83 | 100 | 86 | 69 | 69 | 69 | 69 | 72 | 69 | 75 | 69 |
| GEMMATIMONAS- | 83 | 83 | 83 | 83 | 86 | 100 | 72 | 72 | 69 | 69 | 75 | 72 | 72 | 69 |
| AURANTIACA | | | | | | | | | | | | | | |
| VIBRIO-SP.-MED222 | 69 | 69 | 75 | 72 | 69 | 72 | 100 | 100 | 94 | 94 | 92 | 81 | 64 | 61 |
| MARINOMONAS-SP.-MED121 | 69 | 69 | 75 | 72 | 69 | 72 | 100 | 100 | 94 | 94 | 92 | 81 | 64 | 61 |
| SULFITOBACTER-SP.-NAS-14.1 | 69 | 69 | 72 | 69 | 69 | 69 | 94 | 94 | 100 | 100 | 86 | 75 | 64 | 61 |
| OCEANICOLA- | 69 | 69 | 72 | 69 | 69 | 69 | 94 | 94 | 100 | 100 | 86 | 75 | 64 | 61 |
| BATSENSIS-HTCC2597 | | | | | | | | | | | | | | |
| PSEDUMONOASE- | 72 | 72 | 78 | 78 | 72 | 75 | 92 | 92 | 86 | 86 | 100 | 81 | 64 | 61 |
| FLUORESCENCE | | | | | | | | | | | | | | |
| BDELLOVIBRIO- | 72 | 69 | 72 | 72 | 69 | 72 | 81 | 81 | 75 | 75 | 81 | 100 | 64 | 61 |
| BACTERIOVORUS | | | | | | | | | | | | | | |
| BLEPHARISMA-JAPONICUS | 78 | 78 | 75 | 72 | 75 | 72 | 64 | 64 | 64 | 64 | 64 | 64 | 100 | 72 |
| SYNECHOCOCCUS-SP. | 67 | 67 | 64 | 64 | 69 | 69 | 61 | 61 | 61 | 61 | 61 | 61 | 72 | 100 |

TABLE 5a
Amino acid positions which are strictly conserved shown for the crystal structures

| Arabidopsis | | Pseudomonas | | Streptomyces | | Homo | | Rattus | |
|---|---|---|---|---|---|---|---|---|---|
| thaliana | | fluorescens | | avermitilis | | sapiens | | norvegicus | |
| | Amino | | Amino | | Amino | | Amino | | Amino |
| Position | Acid | Position | Acid | Position | Acid | Position | Acid | Position | Acid |
| 226 | H | 162 | H | 187 | H | 183 | H | 183 | H |
| 267 | S | 202 | S | 230 | S | 226 | S | 226 | S |
| 282 | N | 217 | N | 245 | N | 241 | N | 241 | N |
| 308 | H | 241 | H | 270 | H | 266 | H | 266 | H |
| 342 | Y | 271 | Y | 299 | Y | 295 | Y | 295 | Y |
| 394 | E | 323 | E | 349 | E | 349 | E | 349 | E |
| 420 | G | 334 | G | 360 | G | 360 | G | 360 | G |
| 423 | N | 337 | N | 363 | N | 363 | N | 363 | N |

TABLE 5b
Amino acid position with the amino acids at this positions in
the known crystal structures and the amino acids a these position within
all HPPD sequences

| | | | | | |
|---|---|---|---|---|---|
| Arabidopsis | Pseudomonas | Streptomyces | Homo | Rattus | Natural |
| thaliana | fluorescens | avermitilis | sapiens | norvegicus | Variabaility |

| | Amino | | Amino | | Amino | | Amino | | Amino | Amino |
|---|---|---|---|---|---|---|---|---|---|---|
| Pos. | Acid | Pos. | Acid | Pos. | Acid | Pos. | Acid | Pos. | Acid | Acid |
| 228 | V | 164 | T | 189 | V | 185 | V | 185 | V | VTCAG |
| 248 | H | 186 | R | 211 | T | 207 | H | 207 | H | HRQTKEYLAGSN |
| 250 | F | 188 | A | 213 | M | 209 | F | 209 | F | FYILVAQEDGTSMRK |
| 251 | A | 189 | R | 214 | K | 210 | W | 210 | W | AWILSRKHDEPGNY |
| 252 | E | 190 | Y | 215 | E | 211 | S | 211 | S | ESTYFHQNGLMVILIR |
| 253 | F | 191 | F | 216 | F | 212 | V | 212 | V | FVIALWMQHY |
| 265 | L | 200 | L | 228 | L | 224 | L | 224 | L | LMVIA |
| 268 | A | 203 | K | 231 | K | 227 | I | 227 | I | AVLMIKRQY |
| 269 | V | 204 | A | 232 | V | 228 | V | 228 | V | VAT |
| 270 | L | 205 | M | 233 | V | 229 | V | 229 | V | LVMIA |
| 271 | A | 206 | S | 234 | A | 230 | A | 230 | A | ASTVRKELIMHG |
| 280 | P | 215 | P | 243 | P | 239 | P | 239 | P | PAVTNI |
| 293 | Q | 226 | Q | 255 | Q | 251 | Q | 251 | Q | QLAVRSGVFM |
| 294 | I | 227 | I | 256 | I | 252 | I | 252 | I | IMVTASP |
| 307 | Q | 240 | Q | 269 | Q | 265 | Q | 265 | Q | QHN |
| 335 | M | 264 | M | 293 | L | 289 | L | 289 | L | MLIN |
| 368 | L | 295 | L | 323 | L | 323 | L | 323 | L | LM |
| 379 | Q | 310 | Q | 334 | Q | 334 | Q | 334 | Q | QH |
| 381 | F | 312 | F | 336 | F | 336 | F | 336 | F | FY |
| 392 | F | 321 | F | 347 | F | 347 | F | 347 | F | FS |
| 419 | F | 333 | F | 359 | F | 359 | F | 359 | F | FY |
| 421 | K | 335 | E | 361 | K | 361 | A | 361 | A | KQAILVNDEGS |
| 422 | G | 336 | G | 362 | G | 362 | G | 362 | G | GAPVTM |
| 424 | F | 338 | F | 364 | F | 364 | F | 364 | F | FAVIL |
| 425 | S | 339 | K | 365 | K | 365 | N | 365 | N | SNKGRAPSIKQR |
| 426 | E | 340 | A | 366 | A | 366 | S | 366 | S | EQSAIVFT |
| 427 | L | 341 | L | 367 | L | 367 | L | 367 | L | LR |
| 431 | I | 345 | I | 371 | I | 371 | F | 371 | F | IFVMLQR |

TABLE 5c
Amino acid position with the amino acids at this positions in
the known crystal structures and most common amino acids a these position
within all HPPD sequences

| Arabidopsis | | Pseudomonas | | Streptomyces | | Homo | | Rattus | | Most |
|---|---|---|---|---|---|---|---|---|---|---|
| thaliana | | fluorescens | | avermitilis | | sapiens | | norvegicus | | common |
| | Amino | | Amino | | Amino | | Amino | | Amino | Amino |
| Pos. | Acid | Pos. | Acid | Pos. | Acid | Pos. | Acid | Pos. | Acid | Acid |
| 228 | V | 164 | T | 189 | V | 185 | V | 185 | V | VTCA |
| 248 | H | 186 | R | 211 | T | 207 | H | 207 | H | HRQTKEYA |
| 250 | F | 188 | A | 213 | M | 209 | F | 209 | F | FYILVAEDTM |
| 251 | A | 189 | R | 214 | K | 210 | W | 210 | W | AWILRKHDEPY |
| 252 | E | 190 | Y | 215 | E | 211 | S | 211 | S | ESTRY |
| 253 | F | 191 | F | 216 | F | 212 | V | 212 | V | FVIALWY |
| 265 | L | 200 | L | 228 | L | 224 | L | 224 | L | LMVA |
| 268 | A | 203 | K | 231 | K | 227 | I | 227 | I | AVLMIKRQY |
| 269 | V | 204 | A | 232 | V | 228 | V | 228 | V | VA |
| 270 | L | 205 | M | 233 | V | 229 | V | 229 | V | LVMI |
| 271 | A | 206 | S | 234 | A | 230 | A | 230 | A | ASTVREGK |
| 280 | P | 215 | P | 243 | P | 239 | P | 239 | P | PAVT |
| 293 | Q | 226 | Q | 255 | Q | 251 | Q | 251 | Q | QLA |
| 294 | I | 227 | I | 256 | I | 252 | I | 252 | I | IMVA |
| 307 | Q | 240 | Q | 269 | Q | 265 | Q | 265 | Q | QHN |
| 335 | M | 264 | M | 293 | L | 289 | L | 289 | L | MLI |
| 368 | L | 295 | L | 323 | L | 323 | L | 323 | L | LM |
| 379 | Q | 310 | Q | 334 | Q | 334 | Q | 334 | Q | QH |
| 381 | F | 312 | F | 336 | F | 336 | F | 336 | F | FY |
| 392 | F | 321 | F | 347 | F | 347 | F | 347 | F | F |
| 419 | F | 333 | F | 359 | F | 359 | F | 359 | F | FY |
| 421 | K | 335 | E | 361 | K | 361 | A | 361 | A | KQAIVLDE |
| 422 | G | 336 | G | 362 | G | 362 | G | 362 | G | GA |
| 424 | F | 338 | F | 364 | F | 364 | F | 364 | F | FAVL |
| 425 | S | 339 | K | 365 | K | 365 | N | 365 | N | SNKRAIQ |
| 426 | E | 340 | A | 366 | A | 366 | S | 366 | S | ESAIVF |
| 427 | L | 341 | L | 367 | L | 367 | L | 367 | L | LR |
| 431 | I | 345 | I | 371 | I | 371 | F | 371 | F | IFVMQRL |

Not all amino acids occurring at the variable positions have the same likelihood to be present in an active protein. In some cases most sequences have the same amino acids at a particular position while other amino acids are present at that position in only few HPPD sequences. An example is position 392. Most sequences have at a corresponding position a phenylalanine while few i.e. the burkholderia sequences have a serine. Whereas in some rare cases, the rare amino acids may be the result of a sequencing error, in most other cases the resulting protein is active. Table 5c shows a list with the most common amino acids at the variable positions.

The influence of the amino acids at the variable positions is different. Some of these positions are crucial for catalysis and/or the interaction of HPPD with an inhibitor while others may have less impact. For instances changes at positions 269 and 280 which are in direct contact with the inhibitor and the substrate will very likely have a great impact upon catalysis and inhibitor binding. Also modifications at positions involved in the helix movement induced by inhibitor or substrate binding such as positions 252, 421 and 422 are likely to have high impact on inhibitor binding. In contrast modifications at positions such as 293 are less likely to influence inhibitor binding because this position is quite distant from the active site. Table 6a shows the position with very high impact and Table 6b with high impact on activity and inhibitor binding.

From these observations, the present inventors drew the conclusion that amino acids are preferred in the binding sites which naturally occur at the corresponding positions. This means, that those modifications which exchange a naturally occurring amino acid into another are likely providing a catalytically active HPPD protein which may exert a modified or even increased tolerance to HPPD herbicide inhibitors. Even more promising are those mutant proteins which have the amino acid most often found in nature at the variable position chosen from Table 5c.

TABLE 6a
Amino acid positions with high priority shown for the x-ray structures

| Arabidopsis | | Pseudomonas | | Streptomyces | | Homo | | Rattus | |
|---|---|---|---|---|---|---|---|---|---|
| thaliana | | fluorescens | | avermitilis | | sapiens | | norvegicus | |
| | Amino | | Amino | | Amino | | Amino | | Amino |
| Pos. | Acid | Pos. | Acid | Pos. | Acid | Pos. | Acid | Pos. | Acid |
| 228 | V | 164 | T | 189 | V | 185 | V | 185 | V |
| 250 | F | 188 | A | 213 | M | 209 | F | 209 | F |
| 251 | A | 189 | R | 214 | K | 210 | W | 210 | W |
| 252 | E | 190 | Y | 215 | E | 211 | S | 211 | S |
| 253 | F | 191 | F | 216 | F | 212 | V | 212 | V |
| 265 | L | 200 | L | 228 | L | 224 | L | 224 | L |
| 268 | A | 203 | K | 231 | K | 227 | I | 227 | I |
| 269 | V | 204 | A | 232 | V | 228 | V | 228 | V |
| 270 | L | 205 | M | 233 | V | 229 | V | 229 | V |
| 271 | A | 206 | S | 234 | A | 230 | A | 230 | A |
| 280 | P | 215 | P | 243 | P | 239 | P | 239 | P |
| 307 | Q | 240 | Q | 269 | Q | 265 | Q | 265 | Q |
| 335 | M | 264 | M | 293 | L | 289 | L | 289 | L |
| 368 | L | 295 | L | 323 | L | 323 | L | 323 | L |
| 379 | Q | 310 | Q | 334 | Q | 334 | Q | 334 | Q |
| 392 | F | 321 | F | 347 | F | 347 | F | 347 | F |
| 421 | K | 335 | E | 361 | K | 361 | A | 361 | A |
| 422 | G | 336 | G | 362 | G | 362 | G | 362 | G |
| 426 | E | 340 | A | 366 | A | 366 | S | 366 | S |
| 427 | L | 341 | L | 367 | L | 367 | L | 367 | L |

TABLE 6b
Amino acid positions with very high priority shown for the x-ray structures

| Arabidopsis | | Pseudomonas | | Streptomyces | | Homo | | Rattus | |
|---|---|---|---|---|---|---|---|---|---|
| thaliana | | fluorescens | | avermitilis | | sapiens | | norvegicus | |
| | Amino | | Amino | | Amino | | Amino | | Amino |
| Pos. | Acid | Pos. | Acid | Pos. | Acid | Pos. | Acid | Pos. | Acid |
| 252 | E | 190 | Y | 215 | E | 211 | S | 211 | S |
| 269 | V | 204 | A | 232 | V | 228 | V | 228 | V |
| 280 | P | 215 | P | 243 | P | 239 | P | 239 | P |
| 335 | M | 264 | M | 293 | L | 289 | L | 289 | L |
| 368 | L | 295 | L | 323 | L | 323 | L | 323 | L |
| 421 | K | 335 | E | 361 | K | 361 | A | 361 | A |
| 422 | G | 336 | G | 362 | G | 362 | G | 362 | G |

In another embodiment, in the isolated nucleic acid of the invention as defined above, said at least on amino acid is selected from
Ala, Asp, Glu, Phe, Thr, Val or Tyr at a position in an HPPD protein, said position corresponding to position 250 of the amino acid sequence of SEQ ID No. 2;
Ala, Asp, Glu, Ile, Lys, Leu, Pro or Arg at a position in an HPPD protein, said position corresponding to position 251 of the amino acid sequence of SEQ ID No. 2;
Glu, Thr, Arg, Ser or Tyr at a position in an HPPD protein, said position corresponding to position 252 of the amino acid sequence of SEQ ID No. 2;
Ala, Phe, Val or Trp at a position in an HPPD protein, said position corresponding to position 253 of the amino acid sequence of SEQ ID No. 2;
Leu or Val at a position in an HPPD protein, said position corresponding to position 265 of the amino acid sequence of SEQ ID No. 2;
Leu, Gln, Arg, Val or Tyr at a position in an HPPD protein, said position corresponding to position 268 of the amino acid sequence of SEQ ID NO: 2;
Ala or Val at a position in an HPPD protein, said position corresponding to position 269 of the amino acid sequence of SEQ ID No. 2;
Ala, Pro, Thr or Val at a position in an HPPD protein, said position corresponding to position 280 of the amino acid sequence of SEQ ID No. 2;
Gln at a position in an HPPD protein, said position corresponding to position 307 of the amino acid sequence of SEQ ID No. 2;
Ile or Met at a position in an HPPD protein, said position corresponding to position 335 of the amino acid sequence of SEQ ID No. 2;
Leu at a position in an HPPD protein, said position corresponding to position 368 of the amino acid sequence of SEQ ID No. 2;
Phe at a position in an HPPD protein, said position corresponding to position 392 of the amino acid sequence of SEQ ID No. 2;
Asp, Lys, or Asn at a position in an HPPD protein, said position corresponding to position 421 of the amino acid sequence of SEQ ID No. 2;
Ala or Gly at a position in an HPPD protein, said position corresponding to position 422 of the amino acid sequence of SEQ ID No. 2;
Ile, Arg or Ser at a position in an HPPD protein, said position corresponding to position 425 of the amino acid sequence of SEQ ID No. 2
Glu, Phe or Val at a position in an HPPD protein, said position corresponding to position 426 of the amino acid sequence of SEQ ID No. 2;
Ile, Met, Gln or Val or Phe at a position in an HPPD protein, said position corresponding to position 431 of the amino acid sequence of SEQ ID No. 2;
Ala, Cys, Thr or Val at a position in an HPPD protein, said position corresponding to position 228 of the amino acid sequence of SEQ ID No. 2;
Ala, Glu, His, Lys, Gln, Arg, Thr or Tyr at a position in an HPPD protein, said position corresponding to position 248 of the amino acid sequence of SEQ ID No. 2;
Ile, Leu, Met or Val at a position in an HPPD protein, said position corresponding to position 270 of the amino acid sequence of SEQ ID No. 2;
Ala, Glu, Lys, Arg, Ser, Thr or Val at a position in an HPPD protein, said position corresponding to position 271 of the amino acid sequence of SEQ ID No. 2;
His or Gln at a position in an HPPD protein, said position corresponding to position 379 of the amino acid sequence of SEQ ID No. 2;
Leu or Arg at a position in an HPPD protein, said position corresponding to position 427 of the amino acid sequence of SEQ ID No. 2.

In an alternative embodiment of the nucleic acid of the invention, said at least one amino acid is selected from
Glu, Thr, Arg, Ser or Tyr at a position in an HPPD protein, said position corresponding to position 252 of the amino acid sequence of SEQ ID No. 2;
Ala or Val at a position in an HPPD protein, said position corresponding to position 269 of the amino acid sequence of SEQ ID No. 2;
Ala, Pro, Thr or Val at a position in an HPPD protein, said position corresponding to position 280 of the amino acid sequence of SEQ ID No. 2;
Ile or Met at a position in an HPPD protein, said position corresponding to position 335 of the amino acid sequence of SEQ ID No. 2;
Leu at a position in an HPPD protein, said position corresponding to position 368 of the amino acid sequence of SEQ ID No. 2;
Asp, Asn or Lys at a position in an HPPD protein, said position corresponding to position 421 of the amino acid sequence of SEQ ID No. 2;
Ala or Gly at a position in an HPPD protein, said position corresponding to position 422 of the amino acid sequence of SEQ ID No. 2;
Ala, Cys, Thr or Val at a position in an HPPD protein, said position corresponding to position 228 of the amino acid sequence of SEQ ID No. 2;
Ala, Glu, His, Lys, Gln, Arg, Thr or Tyr at a position in an HPPD protein, said position corresponding to position 248 of the amino acid sequence of SEQ ID No. 2;
Ile, Leu, Met or Val at a position in an HPPD protein, said position corresponding to position 270 of the amino acid sequence of SEQ ID No. 2;
Ala, Glu, Lys, Arg, Ser, Thr or Val at a position in an HPPD protein, said position corresponding to position 271 of the amino acid sequence of SEQ ID No. 2;
His or Gln at a position in an HPPD protein, said position corresponding to position 379 of the amino acid sequence of SEQ ID No. 2;
Leu or Arg at a position in an HPPD protein, said position corresponding to position 427 of the amino acid sequence of SEQ ID No. 2.

Various sequences of HPPD proteins or predicted HPPD proteins are known in the art. These include the HPPD sequences of Streptomyces avermitilis (Genebank SAV11864), Daucus carota (Genebank DCU 87257), Arabidopsis thaliana (Genebank AF047834), Mycosphaerella graminicola (Genebank AF038152), Oryza sativa/rice [BAD26248], Zea mays/corn [ACN36372], Avena sativa [ABZ23427], Pseudomonas fluorescens [ABF50055], Synechococcus sp. [YP_473959], Blepharisma japonicum [BAF91881], Rhodococcus RHA1 sp. ro0240 [YP_702005], Rhodococcus RHA1 sp. ro0341 [YP_703002], Picrophilus torridus [YP_024147], Kordia algicida [ZP_02161490], Sorghum bicolor [XP_002453359], Triticum aestivum/wheat [AAZ67144], or Hordeum vulgare/barley [O48604].

The sequence of the HPPD protein taken as a starting point may be any amino acid sequence encoding a catalytically active HPPD protein. In one embodiment of the isolated nucleic acid of the invention, said HPPD protein comprises the amino acid sequence of SEQ ID No. 4 [Oryza sativa], wherein the resulting amino acid sequence comprises at least one amino acid selected from
Ala, Asp, Glu, Gly, Lys, Gln, Arg, Ser, Thr, Val, Tyr, Ile, Leu or Met at position 247 of the amino acid sequence of SEQ ID No. 4 (corresponding to position 250 of SEQ ID No. 2);
Asp, Glu, Ile, Lys, Leu, Asn, Pro, Arg, Ser, Gly, His or Tyr at position 248 of the amino acid sequence of SEQ ID No. 4 (corresponding to position 251 of SEQ ID No. 2);
Phe, Gly, His, Ile, Leu, Met, Asn, Gln, Arg, Ser, Val, Tyr or Thr at position 249 of the amino acid sequence of SEQ ID No. 4 (corresponding to position 252 of SEQ ID No. 2);
Ala, His, Gln, Val, Trp, Ile, Leu or Met at position 250 of the amino acid sequence of SEQ ID No. 4 (corresponding to position 253 of SEQ ID No. 2);
Val or Met at position 262 of the amino acid sequence of SEQ ID No. 4 (corresponding to position 265 of SEQ ID No. 2);
Leu, Gln, Arg, Tyr, Ala, Ile, Lys or Met at position 265 of the amino acid sequence of SEQ ID No. 4 (corresponding to position 268 of SEQ ID No. 2);
Ala or Thr at position 266 of the amino acid sequence of SEQ ID No. 4 (corresponding to position 269 of SEQ ID No. 2);
Ala, Ile, Asn, Thr or Val at position 277 of the amino acid sequence of SEQ ID No. 4 (corresponding to position 280 of SEQ ID No. 2);
Leu, Val, Ala, Phe, Gly, Met, Arg or Ser at position 290 of the amino acid sequence of SEQ ID No. 4 (corresponding to position 293 of SEQ ID No. 2);
Met, Ala, Pro, Ser, Thr or Val at position 291 of the amino acid sequence of SEQ ID No. 4 (corresponding to position 294 of SEQ ID No. 2);
Ile, Met or Asn at position 332 of the amino acid sequence of SEQ ID No. 4 (corresponding to position 335 of SEQ ID No. 2);
Met at position 365 of the amino acid sequence of SEQ ID No. 4 (corresponding to position 368 of SEQ ID No. 2);
Tyr at position 378 of the amino acid sequence of SEQ ID No. 4 (corresponding to position 381 of SEQ ID No. 2);
Ser at position 389 of the amino acid sequence of SEQ ID No. 4 (corresponding to position 392 of SEQ ID No. 2);
Tyr at position 416 of the amino acid sequence of SEQ ID No. 4 (corresponding to position 419 of SEQ ID No. 2);
Asp, Phe, Asn, Ala, Cys, Glu, Gly, His, Ile, Leu, Met, Gln, Ser, Thr or Val at position 418 of the amino acid sequence of SEQ ID No. 4 (corresponding to position 421 of SEQ ID No. 2);
Ala, Met, Pro or Thr at position 419 of the amino acid sequence of SEQ ID No. 4 (corresponding to position 422 of SEQ ID No. 2);
Ala, Ile or Val at position 421 of the amino acid sequence of SEQ ID No. 4 (corresponding to position 424 of SEQ ID No. 2);
Ile, Pro, Arg, Ala, Gly, Lys, Asn or Gln at position 422 of the amino acid sequence of SEQ ID No. 4 (corresponding to position 425 of SEQ ID No. 2);
Phe, Thr, Val, Ala, Gln or Ser at position 423 of the amino acid sequence of SEQ ID No. 4 (corresponding to position 426 of SEQ ID No. 2);
Met, Gln, Val, Phe or Leu at position 428 of the amino acid sequence of SEQ ID No. 4 (corresponding to position 431 of SEQ ID No. 2);
Phe at position 431 of the amino acid sequence of SEQ ID No. 4 (corresponding to position 434 of SEQ ID No. 2);
Ala, Cys, Gly or Thr at position 225 of the amino acid sequence of SEQ ID No. 4 (corresponding to position 228 of SEQ ID No. 2);
Ala, Glu, Gly, Lys, Leu, Asn, Gln, Arg, Ser, Thr or Tyr at position 245 of the amino acid sequence of SEQ ID No. 4 (corresponding to position 248 of SEQ ID No. 2);
Ala, Ile, Met or Val at position 267 of the amino acid sequence of SEQ ID No. 4 (corresponding to position 270 of SEQ ID No. 2);
Glu, His, Ile, Lys, Leu, Met, Arg, Ser, Thr or Val at position 268 of the amino acid sequence of SEQ ID No. 4 (corresponding to position 271 of SEQ ID No. 2);
His at position 376 of the amino acid sequence of SEQ ID No. 4 (corresponding to position 379 of SEQ ID No. 2);
Arg at a position in an HPPD protein, said position corresponding to position 424 of the amino acid sequence of SEQ ID No. 4 (corresponding to position 427 of SEQ ID No. 2).

In another embodiment of the isolated nucleic acid of the invention, said HPPD protein comprises the amino acid sequence of SEQ ID No. 6 [Zea mays], wherein the resulting amino acid sequence comprises at least one amino acid selected from
a) Ala, Asp, Glu, Gly, Lys, Gln, Arg, Ser, Thr, Val, Tyr, Ile, Leu or Met at position 224 of the amino acid sequence of SEQ ID No. 6 (corresponding to position 250 of SEQ ID No. 2);
b) Asp, Glu, Ile, Lys, Leu, Asn, Pro, Arg, Ser, Gly, His or Tyr at position 225 of the amino acid sequence of SEQ ID No. 6 (corresponding to position 251 of SEQ ID No. 2);
c) Phe, Gly, His, Ile, Leu, Met, Asn, Gln, Arg, Ser, Val, Tyr or Thr at position 226 of the amino acid sequence of SEQ ID No. 6 (corresponding to position 252 of SEQ ID No. 2);
d) Ala, His, Gln, Val, Trp, Ile, Leu or Met at position 227 of the amino acid sequence of SEQ ID No. 6 (corresponding to position 253 of SEQ ID No. 2);
e) Val or Met at position 239 of the amino acid sequence of SEQ ID No. 6 (corresponding to position 265 of SEQ ID No. 2);
f) Leu, Gln, Arg, Val, Tyr, Ala, Ile or Lys at position 242 of the amino acid sequence of SEQ ID No. 6 (corresponding to position 268 of SEQ ID No. 2);
g) Ala or Thr at position 243 of the amino acid sequence of SEQ ID No. 6 (corresponding to position 269 of SEQ ID No. 2);
h) Ala, Ile, Asn, Thr or Val at position 254 of the amino acid sequence of SEQ ID No. 6 (corresponding to position 280 of SEQ ID No. 2);
i) Leu, Val, Ala, Phe, Gly, Met, Arg or Ser at position 267 of the amino acid sequence of SEQ ID No. 6 (corresponding to position 293 of SEQ ID No. 2);
j) Met at position 268 of the amino acid sequence of SEQ ID No. 6 (corresponding to position 294 of SEQ ID No. 2);
k) His or Asn at position 281 of the amino acid sequence of SEQ ID No. 6 (corresponding to position 307 of SEQ ID No. 2);
l) Ile, Asn or Leu at position 309 of the amino acid sequence of SEQ ID No. 6 (corresponding to position 335 of SEQ ID No. 2);
m) Met at position 342 of the amino acid sequence of SEQ ID No. 6 (corresponding to position 368 of SEQ ID No. 2);
n) Tyr at position 355 of the amino acid sequence of SEQ ID No. 6 (corresponding to position 381 of SEQ ID No. 2);
o) Ser at position 366 of the amino acid sequence of SEQ ID No. 6 (corresponding to position 392 of SEQ ID No. 2);
p) Tyr at position 393 of the amino acid sequence of SEQ ID No. 6 (corresponding to position 419 of SEQ ID No. 2);
q) Asp, Phe, Asn, Ala, Cys, Glu, Gly, His, Ile, Leu, Met, Gln, Ser, Thr or Val at position 395 of the amino acid sequence of SEQ ID No. 6 (corresponding to position 421 of SEQ ID No. 2);
r) Ala, Met, Pro or Thr at position 396 of the amino acid sequence of SEQ ID No. 6 (corresponding to position 422 of SEQ ID No. 2);
s) Ala, Ile or Val at position 398 of the amino acid sequence of SEQ ID No. 6 (corresponding to position 424 of SEQ ID No. 2);
t) Ile, Pro, Arg, Ala, Gly, Lys, Asn or Gln at position 399 of the amino acid sequence of SEQ ID No. 6 (corresponding to position 425 of SEQ ID No. 2);
u) Glu, Phe, Thr, Val, Ala or Ser at position 400 of the amino acid sequence of SEQ ID No. 6 (corresponding to position 426 of SEQ ID No. 2);
v) Met, Gln, Val, Phe or Leu at position 405 of the amino acid sequence of SEQ ID No. 6 (corresponding to position 431 of SEQ ID No. 2);
w) Ala, Cys, Gly or Thr at a position in an HPPD protein, said position corresponding to position 202 of the amino acid sequence of SEQ ID No. 6 (corresponding to position 228 of SEQ ID No. 2);
x) Ala, Glu, Gly, Lys, Leu, Asn, Gln, Arg, Ser, Thr or Tyr at position 222 of the amino acid sequence of SEQ ID No. 6 (corresponding to position 248 of SEQ ID No. 2);
y) Ala, Ile, Met or Val at position 244 of the amino acid sequence of SEQ ID No. 6 (corresponding to position 270 of SEQ ID No. 2);
z) Glu, His, Ile, Lys, Leu, Met, Arg, Ser, Thr or Val at position 245 of the amino acid sequence of SEQ ID No. 6 (corresponding to position 271 of SEQ ID No. 2);
aa) Gln at position 353 of the amino acid sequence of SEQ ID No. 6 (corresponding to position 379 of SEQ ID No. 2);
ab) Arg at position 401 of the amino acid sequence of SEQ ID No. 6 (corresponding to position 427 of SEQ ID No. 2).

In another embodiment of the isolated nucleic acid of the invention, said HPPD protein comprises the amino acid sequence of SEQ ID No. 8 [Avena sativa], wherein the resulting amino acid sequence comprises at least one amino acid selected from
Ala, Asp, Glu, Gly, Lys, Gln, Arg, Ser, Thr, Val, Tyr, Ile, Leu or Met at position 241 of the amino acid sequence of SEQ ID No. 8 (corresponding to position 250 of SEQ ID No. 2), preferably Ala;
Asp, Glu, Ile, Lys, Leu, Asn, Pro, Arg, Ser, Gly, His or Tyr at position 242 of the amino acid sequence of SEQ ID No. 8 (corresponding to position 251 of SEQ ID No. 2), preferably Arg or Lys;
Phe, Gly, His, Ile, Leu, Met, Asn, Gln, Arg, Ser, Val, Tyr or Thr at position 243 of the amino acid sequence of SEQ ID No. 8 (corresponding to position 252 of SEQ ID No. 2), preferably Tyr;
Ala, His, Gln, Val, Trp, Ile, Leu or Met at position 244 of the amino acid sequence of SEQ ID No. 8 (corresponding to position 253 of SEQ ID No. 2), preferably Val;
Val or Met at position 256 of the amino acid sequence of SEQ ID No. 8 (corresponding to position 265 of SEQ ID No. 2);
Leu, Gln, Arg, Tyr, Ala, Ile, Lys or Met at position 259 of the amino acid sequence of SEQ ID No. 8 (corresponding to position 268 of SEQ ID No. 2);
Ala or Thr at position 260 of the amino acid sequence of SEQ ID No. 8 (corresponding to position 269 of SEQ ID No. 2), preferably Ala;
Ala, Ile, Asn, Thr or Val at position 271 of the amino acid sequence of SEQ ID No. 8 (corresponding to position 280 of SEQ ID No. 2);
Leu, Val, Ala, Phe, Gly, Met, Arg or Ser at position 284 of the amino acid sequence of SEQ ID No. 8 (corresponding to position 293 of SEQ ID No. 2);
Met, Ala, Pro, Ser, Thr or Val at position 285 of the amino acid sequence of SEQ ID No. 8 (corresponding to position 294 of SEQ ID No. 2);
His or Asn at position 265 of the amino acid sequence of SEQ ID No. 8 (corresponding to position 307 of SEQ ID No. 2);
Ile or Asn or Leu at position 326 of the amino acid sequence of SEQ ID No. 8 (corresponding to position 335 of SEQ ID No. 2);
Met at position 359 of the amino acid sequence of SEQ ID No. 8 (corresponding to position 368 of SEQ ID No. 2);
Tyr at position 372 of the amino acid sequence of SEQ ID No. 8 (corresponding to position 381 of SEQ ID No. 2);
Ser at position 383 of the amino acid sequence of SEQ ID No. 8 (corresponding to position 392 of SEQ ID No. 2);
Tyr at position 410 of the amino acid sequence of SEQ ID No. 8 (corresponding to position 419 of SEQ ID No. 2);
Asp, Phe, Asn, Ala, Cys, Glu, Gly, His, Ile, Leu, Met, Gln, Ser, Thr or Val at position 412 of the amino acid sequence of SEQ ID No. 8 (corresponding to position 421 of SEQ ID No. 2), preferably Lys;
Ala, Met, Pro or Thr at position 413 of the amino acid sequence of SEQ ID No. 8 (corresponding to position 422 of SEQ ID No. 2);
Ala, Ile or Val at position 415 of the amino acid sequence of SEQ ID No. 8 (corresponding to position 424 of SEQ ID No. 2);
Ile, Pro, Arg, Ala, Gly, Lys, Asn or Gln at position 416 of the amino acid sequence of SEQ ID No. 8 (corresponding to position 425 of SEQ ID No. 2);
Phe, Thr, Val, Ala, Gln or Ser at position 417 of the amino acid sequence of SEQ ID No. 8 (corresponding to position 426 of SEQ ID No. 2);
Met, Gln, Val, Phe or Leu at position 422 of the amino acid sequence of SEQ ID No. 8 (corresponding to position 431 of SEQ ID No. 2);
Ala, Cys, Gly or Thr at position 219 of the amino acid sequence of SEQ ID No. 8 (corresponding to position 228 of SEQ ID No. 2);
Ala, Glu, Gly, Lys, Leu, Asn, Gln, Arg, Ser, Thr or Tyr at position 239 of the amino acid sequence of SEQ ID No. 8 (corresponding to position 248 of SEQ ID No. 2);
Ala, Ile, Met or Val at position 261 of the amino acid sequence of SEQ ID No. 8 (corresponding to position 270 of SEQ ID No. 2);
Glu, His, Ile, Lys, Leu, Met, Arg, Ser, Thr or Val at position 262 of the amino acid sequence of SEQ ID No. 8 (corresponding to position 271 of SEQ ID No. 2);
His at position 370 of the amino acid sequence of SEQ ID No. 8 (corresponding to position 379 of SEQ ID No. 2);
Arg at position 418 of the amino acid sequence of SEQ ID No. 8 (corresponding to position 427 of SEQ ID No. 2).

In another embodiment, of the isolated nucleic acid of the invention, said HPPD protein comprises the amino acid sequence of SEQ ID No. 10 [Pseudomonas fluorescens], wherein the resulting amino acid sequence comprises at least one amino acid selected from
Phe, Asp, Glu, Gly, Lys, Gln, Arg, Ser, Thr, Val, Tyr, Ile, Leu or Met at position 188 of the amino acid sequence of SEQ ID No. 10 (corresponding to position 250 of SEQ ID No. 2), preferably Ala;
Ala, Asp, Glu, Ile, Lys, Leu, Asn, Pro, Ser, Gly, His or Tyr at position 189 of the amino acid sequence of SEQ ID No. 10 (corresponding to position 251 of SEQ ID No. 2), preferably Ala or Lys;
Phe, Gly, His, Ile, Leu, Met, Asn, Gln, Arg, Ser, Val, Glu or Thr at position 190 of the amino acid sequence of SEQ ID No. 10 (corresponding to position 252 of SEQ ID No. 2), preferably Ser or Tyr;
Ala, His, Gln, Val, Trp, Ile, Leu or Met at position 191 of the amino acid sequence of SEQ ID No. 10 (corresponding to position 253 of SEQ ID No. 2), preferably Phe or Val;
Val or Met at position 200 of the amino acid sequence of SEQ ID No. 10 (corresponding to position 265 of SEQ ID No. 2);
Leu, Gln, Arg, Val Tyr, Ala, Ile or Met at position 203 of the amino acid sequence of SEQ ID No. 10 (corresponding to position 268 of SEQ ID No. 2);
Thr or Val at position 204 of the amino acid sequence of SEQ ID No. 10 (corresponding to position 269 of SEQ ID No. 2), preferably Val;
Ala, Ile, Asn, Thr or Val at position 215 of the amino acid sequence of SEQ ID No. 10 (corresponding to position 280 of SEQ ID No. 2);
Leu, Val, Ala, Phe, Gly, Met, Arg or Ser at position 226 of the amino acid sequence of SEQ ID NO:SEQ ID No. 10 (corresponding to position 293 of SEQ ID No. 2);
Met, Thr, Ala, Pro, Ser or Val at position 227 of the amino acid sequence of SEQ ID No. 10 (corresponding to position 294 of SEQ ID No. 2);
His or Asn at position 240 of the amino acid sequence of SEQ ID No. 10 (corresponding to position 307 of SEQ ID No. 2);
Ile, Asn or Leu at position 264 of the amino acid sequence of SEQ ID No. 10 (corresponding to position 335 of SEQ ID No. 2);
Met at position 295 of the amino acid sequence of SEQ ID No. 10 (corresponding to position 368 of SEQ ID No. 2);
Tyr at position 312 of the amino acid sequence of SEQ ID No. 10 (corresponding to position 381 of SEQ ID No. 2);
Ser at position 321 of the amino acid sequence of SEQ ID No. 10 (corresponding to position 392 of SEQ ID No. 2);
Tyr at position 333 of the amino acid sequence of SEQ ID No. 10 (corresponding to position 419 of SEQ ID No. 2);
Asp, Lys, Asn, Ala, Cys, Gly, His, Ile, Leu, Met, Gln, Ser, Thr or Val at position 335 of the amino acid sequence of SEQ ID No. 10 (corresponding to position 421 of SEQ ID No. 2), preferably Lys;
Ala, Met, Pro or Thr at position 336 of the amino acid sequence of SEQ ID No. 10 (corresponding to position 422 of SEQ ID No. 2);
Ala, Ile or Val at position 338 of the amino acid sequence of SEQ ID No. 10 (corresponding to position 424 of SEQ ID No. 2);
Ile, Pro, Ala, Asn, Gly or Gln at position 339 of the amino acid sequence of SEQ ID No. 10 (corresponding to position 425 of SEQ ID No. 2);
Phe, Thr or Val at position 340 of the amino acid sequence of SEQ ID No. 10 (corresponding to position 426 of SEQ ID No. 2);
Met, Phe, Leu, Gln or Val at position 345 of the amino acid sequence of SEQ ID No. 10 (corresponding to position 431 of SEQ ID No. 2);
Ala, Cys, Gly or Val at position 164 of the amino acid sequence of SEQ ID No. 10 (corresponding to position 228 of SEQ ID No. 2);
Ala, Glu, Gly, His, Lys, Leu, Asn, Gln, Ser, Thr or Tyr at position 186 of the amino acid sequence of SEQ ID No. 10 (corresponding to position 248 of SEQ ID No. 2);
Ala, Ile, Leu or Val at position 205 of the amino acid sequence of SEQ ID No. 10 (corresponding to position 270 of SEQ ID No. 2);
Ala, Glu, His, Ile, Lys, Leu, Met, Arg, Thr or Val at position 206 of the amino acid sequence of SEQ ID No. 10 (corresponding to position 271 of SEQ ID No. 2);
His at position 310 of the amino acid sequence of SEQ ID No. 10 (corresponding to position 379 of SEQ ID No. 2);
Arg at position 341 of the amino acid sequence of SEQ ID No. 10 (corresponding to position 427 of SEQ ID No. 2).

In another embodiment of the isolated nucleic acid of the invention, said HPPD protein comprises the amino acid sequence of SEQ ID No. 14 [Synechococcus sp.], wherein the resulting amino acid sequence comprises at least one amino acid selected from
Ala, Asp, Glu, Phe, Gly, Lys, Gln, Arg, Ser, Thr, Val, Tyr, Ile or Met at position 169 of the amino acid sequence of SEQ ID No. 14 (corresponding to position 250 of SEQ ID No. 2), preferably Ala or Phe;
Ala, Asp, Glu, Ile, Lys, Leu, Asn, Pro, Arg or Ser, Gly or His at position 170 of the amino acid sequence of SEQ ID No. 14 (corresponding to position 251 of SEQ ID No. 2), preferably Ala, Arg or Lys;
Glu, Phe, Gly, His, Ile, Leu, Met, Asn, Gln, Ser, Val, Tyr or Thr at position 171 of the amino acid sequence of SEQ ID No. 14 (corresponding to position 252 of SEQ ID No. 2), preferably Glu, Ser or Tyr;
Ala, Phe, His, Gln, Val or Ile, Leu or Met at position 172 of the amino acid sequence of SEQ ID No. 14 (corresponding to position 253 of SEQ ID No. 2), preferably Phe or Val;
Val or Met at position 181 of the amino acid sequence of SEQ ID No. 14 (corresponding to position 265 of SEQ ID No. 2);
Leu, Gln, Arg, Tyr, Ala, Ile, Lys or Met at position 184 of the amino acid sequence of SEQ ID No. 14 (corresponding to position 268 of SEQ ID No. 2);
Ala or Thr at position 185 of the amino acid sequence of SEQ ID No. 14 (corresponding to position 269 of SEQ ID No. 2), preferably Ala;
Ile, Asn, Pro, Thr or Val at position 196 of the amino acid sequence of SEQ ID No. 14 (corresponding to position 280 of SEQ ID No. 2), preferably Pro;
Leu, Val, Ala, Phe, Gly, Met, Arg or Ser at position 206 of the amino acid sequence of SEQ ID No. 14 (corresponding to position 293 of SEQ ID No. 2);
Met, Ala, Pro, Ser, Thr or Val at position 207 of the amino acid sequence of SEQ ID No. 14 (corresponding to position 294 of SEQ ID No. 2);
His or Asn at position 220 of the amino acid sequence of SEQ ID No. 14 (corresponding to position 307 of SEQ ID No. 2);
Ile, Met or Asn at position 244 of the amino acid sequence of SEQ ID No. 14 (corresponding to position 335 of SEQ ID No. 2), preferably Met;
Met at position 286 of the amino acid sequence of SEQ ID No. 14 (corresponding to position 368 of SEQ ID No. 2);
Phe or Tyr at position 301 of the amino acid sequence of SEQ ID No. 14 (corresponding to position 381 of SEQ ID No. 2);
Ser at position 312 of the amino acid sequence of SEQ ID No. 14 (corresponding to position 392 of SEQ ID No. 2);
Tyr at position 325 of the amino acid sequence of SEQ ID No. 14 (corresponding to position 419 of SEQ ID No. 2);
Asp, Phe, Lys, Asn, Ala, Cys, Gly, His, Ile, Leu, Met, Gln, Ser, Thr or Val at position 327 of the amino acid sequence of SEQ ID No. 14 (corresponding to position 421 of SEQ ID No. 2), preferably Lys;
Gly, Met, Pro or Thr at position 328 of the amino acid sequence of SEQ ID No. 14 (corresponding to position 422 of SEQ ID No. 2);
Ala, Ile or Val at position 330 of the amino acid sequence of SEQ ID No. 14 (corresponding to position 424 of SEQ ID No. 2);
Ile, Pro, Arg, Ser, Ala, Gly, Lys or Asn at position 331 of the amino acid sequence of SEQ ID No. 14 (corresponding to position 425 of SEQ ID No. 2);
Glu, Phe, Thr, Val, Gln or Ser at position 332 of the amino acid sequence of SEQ ID No. 14 (corresponding to position 426 of SEQ ID No. 2), preferably Glu;
Ile, Met, Gln, Val or Phe at position 337 of the amino acid sequence of SEQ ID No. 14 (corresponding to position 431 of SEQ ID No. 2);
Ala, Cys, Gly or Thr at position 145 of the amino acid sequence of SEQ ID No. 14 (corresponding to position 228 of SEQ ID No. 2);
Ala, Glu, Gly, His, Lys, Leu, Asn, Gln, Ser, Thr or Tyr at position 167 of the amino acid sequence of SEQ ID No. 14 (corresponding to position 248 of SEQ ID No. 2);
Ala, Ile, Leu or Met at position 186 of the amino acid sequence of SEQ ID No. 14 (corresponding to position 270 of SEQ ID No. 2);
Ala, Glu, His, Ile, Lys, Leu, Met, Arg, Ser, Thr or Val at position 187 of the amino acid sequence of SEQ ID No. 14 (corresponding to position 271 of SEQ ID No. 2);
His at a position at position 299 of the amino acid sequence of SEQ ID No. 14 (corresponding to position 379 of SEQ ID No. 2);
Arg at a position at position 333 of the amino acid sequence of SEQ ID No. 14 (corresponding to position 427 of SEQ ID No. 2).

In another embodiment of the isolated nucleic acid of the invention, said HPPD protein comprises the amino acid sequence of SEQ ID No. 16 [Blepharisma japonicum], wherein the resulting amino acid sequence comprises at least one amino acid selected from
Ala, Asp, Glu, Phe, Gly, Lys, Gln, Arg, Ser, Thr, Val, Ile, Leu or Met at position 209 of the amino acid sequence of SEQ ID No. 16 (corresponding to position 250 of SEQ ID No. 2), preferably Ala or Phe;
Ala, Asp, Glu, Ile, Lys, Leu, Asn, Pro, Arg, Ser, Gly, His or Tyr at position 210 of the amino acid sequence of SEQ ID No. 16 (corresponding to position 251 of SEQ ID No. 2), preferably Ala, Arg, Lys;
Glu, Phe, Gly, His, Ile, Leu, Met, Asn, Gln, Arg, Val, Tyr or Thr at position 211 of the amino acid sequence of SEQ ID No. 16 (corresponding to position 252 of SEQ ID No. 2), preferably Glu or Tyr;
Phe, His, Gln, Val, Trp, Ile, Leu or Met at position 212 of the amino acid sequence of SEQ ID No. 16 (corresponding to position 253 of SEQ ID No. 2), preferably Phe or Val;
Val or Met at position 224 of the amino acid sequence of SEQ ID No. 16 (corresponding to position 265 of SEQ ID No. 2);
Leu, Gln, Arg, Tyr, Ala, Ile, Lys or Met at position 227 of the amino acid sequence of SEQ ID No. 16 (corresponding to position 2268 of SEQ ID No. 2);
Ala or Thr at position 228 of the amino acid sequence of SEQ ID No. 16 (corresponding to position 269 of SEQ ID No. 2), preferably Ala;
Ala, Ile, Asn, Thr or Val at position 239 of the amino acid sequence of SEQ ID No. 16 (corresponding to position 280 of SEQ ID No. 2);
Leu, Val, Ala, Phe, Gly, Met, Arg or Ser at position 251 of the amino acid sequence of SEQ ID No. 16 (corresponding to position 293 of SEQ ID No. 2);
Met, Ala, Pro, Ser, Thr or Val at position 252 of the amino acid sequence of SEQ ID No. 16 (corresponding to position 294 of SEQ ID No. 2);
His or Asn at position 265 of the amino acid sequence of SEQ ID No. 16 (corresponding to position 307 of SEQ ID No. 2);
Ile, Met or Asn at position 289 of the amino acid sequence of SEQ ID No. 16 (corresponding to position 335 of SEQ ID No. 2), preferably Met;
Met at position 323 of the amino acid sequence of SEQ ID No. 16 (corresponding to position 368 of SEQ ID No. 2);
Tyr at position 336 of the amino acid sequence of SEQ ID No. 16 (corresponding to position 381 of SEQ ID No. 2);
Ser at position 347 of the amino acid sequence of SEQ ID No. 16 (corresponding to position 292 of SEQ ID No. 2);
Tyr at position 359 of the amino acid sequence of SEQ ID No. 16 (corresponding to position 419 of SEQ ID No. 2);
Asp, Phe, Lys, Asn, Ala, Cys, Glu, Gly, His, Leu, Met, Gln, Ser, Thr or Val at position 361 of the amino acid sequence of SEQ ID No. 16 (corresponding to position 421 of SEQ ID No. 2), preferably Lys;
Ala, Met, Pro or Thr at position 362 of the amino acid sequence of SEQ ID No. 16 (corresponding to position 422 of SEQ ID No. 2);
Ala, Ile or Val at position 364 of the amino acid sequence of SEQ ID No. 16 (corresponding to position 424 of SEQ ID No. 2);
Ile, Pro, Arg, Ser, Ala, Gly, Asn or Gln at position 365 of the amino acid sequence of SEQ ID No. 16 (corresponding to position 425 of SEQ ID No. 2);
Glu, Phe, Thr, Val, Gln or Ser at position 366 of the amino acid sequence of SEQ ID No. 16 (corresponding to position 426 of SEQ ID No. 2), preferably Glu;
Ile, Met, Gln, Val or Phe at position 371 of the amino acid sequence of SEQ ID No. 16 (corresponding to position 431 of SEQ ID No. 2);
Ala, Cys, Gly or Thr at a position in an HPPD protein, said position corresponding to position 185 of the amino acid sequence of SEQ ID No. 16 (corresponding to position 228 of SEQ ID No. 2);
Ala, Glu, Gly, Lys, Leu, Asn, Gln, Arg, Ser, Thr or Tyr at a position in an HPPD protein, said position corresponding to position 207 of the amino acid sequence of SEQ ID No. 16 (corresponding to position 248 of SEQ ID No. 2);
Ala, Ile, Leu or Met at a position in an HPPD protein, said position corresponding to position 229 of the amino acid sequence of SEQ ID No. 16 (corresponding to position 270 of SEQ ID No. 2);
Glu, His, Ile, Lys, Leu, Met, Arg, Ser, Thr or Val at a position in an HPPD protein, said position corresponding to position 230 of the amino acid sequence of SEQ ID No. 16 (corresponding to position 271 of SEQ ID No. 2);
His at a position in an HPPD protein, said position corresponding to position 334 of the amino acid sequence of SEQ ID No. 16 (corresponding to position 379 of SEQ ID No. 2);
Arg at a position in an HPPD protein, said position corresponding to position 367 of the amino acid sequence of SEQ ID No. 16 (corresponding to position 427 of SEQ ID No. 2).

In another embodiment, of the isolated nucleic acid of the invention, said HPPD protein comprises the amino acid sequence of SEQ ID No. 18 [Rhodococcus RHA1 sp. ro0240], wherein the resulting amino acid sequence comprises at least one amino acid selected from
Ala, Asp, Glu, Phe, Gly, Lys, Gln, Arg, Ser, Thr, Val, Tyr, Ile or Leu at position 231 of the amino acid sequence of SEQ ID No. 18 (corresponding to position 250 of SEQ ID No. 2), preferably Phe or Ala;
Asp, Glu, Ile, Lys, Leu, Asn, Pro, Arg, Ser, Gly, His or Tyr at position 232 of the amino acid sequence of SEQ ID No. 18 (corresponding to position 251 of SEQ ID No. 2), preferably Arg or Lys;
Phe, Gly, His, Ile, Leu, Met, Asn, Gln, Arg, Ser, Val, Tyr or Thr at position 233 of the amino acid sequence of SEQ ID No. 18 (corresponding to position 252 of SEQ ID No. 2), preferably Ser or Tyr;
Ala, His, Gln, Val, Trp, Ile, Leu or Met at position 234 of the amino acid sequence of SEQ ID No. 18 (corresponding to position 253 of SEQ ID No. 2), preferably Val;
Val or Met at position 246 of the amino acid sequence of SEQ ID No. 18 (corresponding to position 265 of SEQ ID No. 2);
Leu, Gln, Arg, Val, Tyr, Ala, Ile or Met at position 249 of the amino acid sequence of SEQ ID No. 18 (corresponding to position 268 of SEQ ID No. 2);
Ala or Thr at position 250 of the amino acid sequence of SEQ ID No. 18 (corresponding to position 269 of SEQ ID No. 2), preferably Ala;
Ala, Ile, Asn, Thr or Val at position 261 of the amino acid sequence of SEQ ID No. 18 (corresponding to position 280 of SEQ ID No. 2);
Leu, Val, Ala, Phe, Gly, Met, Arg or Ser at position 273 of the amino acid sequence of SEQ ID No. 18 (corresponding to position 293 of SEQ ID No. 2);
Met, Ala, Pro, Ser, Thr or Val at position 274 of the amino acid sequence of SEQ ID No. 18 (corresponding to position 294 of SEQ ID No. 2);
His or Asn at position 287 of the amino acid sequence of SEQ ID No. 18 (corresponding to position 307 of SEQ ID No. 2);
Ile, Met or Asn at position 311 of the amino acid sequence of SEQ ID No. 18 (corresponding to position 335 of SEQ ID No. 2), preferably Met;
Met at position 343 of the amino acid sequence of SEQ ID No. 18 (corresponding to position 368 of SEQ ID No. 2);
Tyr at position 356 of the amino acid sequence of SEQ ID No. 18 (corresponding to position 381 of SEQ ID No. 2);
Ser at position 367 of the amino acid sequence of SEQ ID No. 18 (corresponding to position 392 of SEQ ID No. 2);
Tyr at position 379 of the amino acid sequence of SEQ ID No. 18 (corresponding to position 419 of SEQ ID No. 2);
Asp, Lys, Asn, Ala, Cys, Glu, Gly, His, Leu, Met, Gln, Ser, Thr or Val at position 381 of the amino acid sequence of SEQ ID No. 18 (corresponding to position 421 of SEQ ID No. 2), preferably Lys;
Ala, Met, Pro or Thr at position 382 of the amino acid sequence of SEQ ID No. 18 (corresponding to position 422 of SEQ ID No. 2);
Ala, Ile or Val at position 384 of the amino acid sequence of SEQ ID No. 18 (corresponding to position 424 of SEQ ID No. 2);
Ile, Pro, Arg, Ser, Ala, Gly, Asn or Gln at position 385 of the amino acid sequence of SEQ ID No. 18 (corresponding to position 425 of SEQ ID No. 2);
Glu, Phe, Thr, Val, Gln or Ser at position 386 of the amino acid sequence of SEQ ID No. 18 (corresponding to position 426 of SEQ ID No. 2), preferably Glu;
Met, Gln, Phe, Val, Phe or Leu at position 391 of the amino acid sequence of SEQ ID No. 18 (corresponding to position 431 of SEQ ID No. 2);
Ala, Cys, Gly or Thr at a position in an HPPD protein, said position corresponding to position 207 of the amino acid sequence of SEQ ID No. 1 (corresponding to position 228 of SEQ ID No. 2)8;
Ala, Glu, Gly, His, Lys, Leu, Asn, Gln, Arg, Ser or Tyr at a position in an HPPD protein, said position corresponding to position 229 of the amino acid sequence of SEQ ID No. 18 (corresponding to position 248 of SEQ ID No. 2);
Ala, Ile, Leu or Met at a position in an HPPD protein, said position corresponding to position 251 of the amino acid sequence of SEQ ID No. 18 (corresponding to position 270 of SEQ ID No. 2);
Ala, Glu, His, Ile, Lys, Leu, Met, Arg, Thr or Val at a position in an HPPD protein, said position corresponding to position 252 of the amino acid sequence of SEQ ID No. 18 (corresponding to position 271 of SEQ ID No. 2);
His at a position in an HPPD protein, said position corresponding to position 354 of the amino acid sequence of SEQ ID No. 18 (corresponding to position 379 of SEQ ID No. 2);
Arg at a position in an HPPD protein, said position corresponding to position 387 of the amino acid sequence of SEQ ID No. 18 (corresponding to position 427 of SEQ ID No. 2).

In another embodiment, of the isolated nucleic acid of the invention, said HPPD protein comprises the amino acid sequence of SEQ ID No. 30 [Rhodococcus RHA1 sp. 0341], wherein the resulting amino acid sequence comprises at least one amino acid selected from
Ala, Asp, Glu, Phe, Gly, Lys, Gln, Arg, Ser, Thr, Val, Tyr, Ile or Leu at position 232 of the amino acid sequence of SEQ ID No. 30 (corresponding to position 250 of SEQ ID No. 2), preferably Phe or Ala;
Asp, Glu, Ile, Lys, Leu, Asn, Pro, Arg, Ser, Gly, His or Tyr at position 233 of the amino acid sequence of SEQ ID No. 30 (corresponding to position 251 of SEQ ID No. 2), preferably Arg or Lys;
Phe, Gly, His, Ile, Leu, Met, Asn, Gln, Arg, Ser, Val, Tyr or Thr at position 234 of the amino acid sequence of SEQ ID No. 30 (corresponding to position 252 of SEQ ID No. 2), preferably Ser or Tyr;
Ala, His, Gln, Val, Trp, Ile, Leu or Met at position 235 of the amino acid sequence of SEQ ID No. 30 (corresponding to position 253 of SEQ ID No. 2), preferably Val;
Val or Met at position 247 of the amino acid sequence of SEQ ID No. 30 (corresponding to position 265 of SEQ ID No. 2);
Leu, Gln, Arg, Val, Tyr, Ala, Ile or Met at position 250 of the amino acid sequence of SEQ ID No. 30 (corresponding to position 268 of SEQ ID No. 2);
Ala or Thr at position 251 of the amino acid sequence of SEQ ID No. 30 (corresponding to position 269 of SEQ ID No. 2), preferably Ala;
Ala, Ile, Asn, Thr or Val at position 262 of the amino acid sequence of SEQ ID No. 30 (corresponding to position 280 of SEQ ID No. 2);
Leu, Val, Ala, Phe, Gly, Met, Arg or Ser at position 274 of the amino acid sequence of SEQ ID No. 30 (corresponding to position 293 of SEQ ID No. 2);
Met, Ala, Pro, Ser, Thr or Val at position 275 of the amino acid sequence of SEQ ID No. 30 (corresponding to position 294 of SEQ ID No. 2);
His or Asn at position 288 of the amino acid sequence of SEQ ID No. 30 (corresponding to position 307 of SEQ ID No. 2);
Ile, Met or Asn at position 312 of the amino acid sequence of SEQ ID No. 30 (corresponding to position 335 of SEQ ID No. 2), preferably Met;
Met at position 344 of the amino acid sequence of SEQ ID No. 30 (corresponding to position 368 of SEQ ID No. 2);
Tyr at position 357 of the amino acid sequence of SEQ ID No. 30 (corresponding to position 381 of SEQ ID No. 2);
Ser at position 368 of the amino acid sequence of SEQ ID No. 30 (corresponding to position 392 of SEQ ID No. 2);
Tyr at position 380 of the amino acid sequence of SEQ ID No. 30 (corresponding to position 419 of SEQ ID No. 2);
Asp, Phe, Lys, Asn, Ala, Cys, Glu, Gly, His, Leu, Met, Gln, Ser, Thr or Val at position 382 of the amino acid sequence of SEQ ID No. 30 (corresponding to position 421 of SEQ ID No. 2), preferably Lys;
Ala, Met, Pro or Thr at position 383 of the amino acid sequence of SEQ ID No. 30 (corresponding to position 422 of SEQ ID No. 2);
Ala, Ile or Val at position 385 of the amino acid sequence of SEQ ID No. 30 (corresponding to position 424 of SEQ ID No. 2);
Ile, Pro, Arg, Ser, Ala, Gly, Asn or Gln at position 386 of the amino acid sequence of SEQ ID No. 30 (corresponding to position 425 of SEQ ID No. 2);
Glu, Phe, Thr, Val, Gln or Ser at position 387 of the amino acid sequence of SEQ ID No. 30 (corresponding to position 426 of SEQ ID No. 2), preferably Glu;
Met, Gln, Phe, Val, Phe or Leu at position 392 of the amino acid sequence of SEQ ID No. 30 (corresponding to position 431 of SEQ ID No. 2);
Ala, Cys, Gly or Thr at a position in an HPPD protein, said position corresponding to position 208 of the amino acid sequence of SEQ ID No. 30 (corresponding to position 228 of SEQ ID No. 2);
Ala, Glu, Gly, His, Lys, Leu, Asn, Gln, Arg, Ser or Tyr at a position in an HPPD protein, said position corresponding to position 230 of the amino acid sequence of SEQ ID No. 30 (corresponding to position 248 of SEQ ID No. 2);
Ala, Ile, Leu or Met at a position in an HPPD protein, said position corresponding to position 252 of the amino acid sequence of SEQ ID No. 30 (corresponding to position 270 of SEQ ID No. 2);
Glu, His, Ile, Lys, Leu, Met, Arg, Thr or Val at a position in an HPPD protein, said position corresponding to position 253 of the amino acid sequence of SEQ ID No. 30 (corresponding to position 271 of SEQ ID No. 2);
His at a position in an HPPD protein, said position corresponding to position 355 of the amino acid sequence of SEQ ID No. 30 (corresponding to position 379 of SEQ ID No. 2);
Arg at a position in an HPPD protein, said position corresponding to position 388 of the amino acid sequence of SEQ ID No. 30 (corresponding to position 427 of SEQ ID No. 2).

In another embodiment, of the isolated nucleic acid of the invention, said HPPD protein comprises the amino acid sequence of SEQ ID No. 20 [Picrophilus torridus], wherein the resulting amino acid sequence comprises at least one amino acid selected from
Ala, Asp, Glu, Phe, Gly, Lys, Gln, Arg, Ser, Thr, Val, Tyr, Ile or Met at position 201 of the amino acid sequence of SEQ ID No. 20 (corresponding to position 250 of SEQ ID No. 2), preferably Phe or Ala;
Ala, Asp, Glu, Lys, Leu, Asn, Pro, Arg or Ser, Gly, His or Trp at position 202 of the amino acid sequence of SEQ ID No. 20 (corresponding to position 251 of SEQ ID No. 2), preferably Ala, Arg or Lys;
Glu, Phe, Gly, His, Ile, Leu, Met, Asn, Gln, Arg, Ser, Val or Tyr at position 203 of the amino acid sequence of SEQ ID No. 20 (corresponding to position 252 of SEQ ID No. 2), preferably Glu, Ser or Tyr;
Ala, His, Gln, Val, Trp, Ile, Leu or Met at position 204 of the amino acid sequence of SEQ ID No. 20 (corresponding to position 253 of SEQ ID No. 2), preferably Val;
Val or Met at position 216 of the amino acid sequence of SEQ ID No. 20 (corresponding to position 265 of SEQ ID No. 2);
Leu, Gln, Arg, Val, Tyr, Ala, Ile or Met at position 219 of the amino acid sequence of SEQ ID No. 20 (corresponding to position 268 of SEQ ID No. 2);
Ala or Thr at position 220 of the amino acid sequence of SEQ ID No. 20 (corresponding to position 269 of SEQ ID No. 2), preferably Ala;
Ala, Ile, Asn, Thr or Val at position 230 of the amino acid sequence of SEQ ID No. 20 (corresponding to position 280 of SEQ ID No. 2);
Leu, Val, Ala, Phe, Gly, Met, Arg or Ser at position 242 of the amino acid sequence of SEQ ID No. 20 (corresponding to position 293 of SEQ ID No. 2);
Met, Ala, Pro, Ser, Thr or Val at position 243 of the amino acid sequence of SEQ ID No. 20 (corresponding to position 294 of SEQ ID No. 2);
His or Asn at position 256 of the amino acid sequence of SEQ ID No. 20 (corresponding to position 307 of SEQ ID No. 2);
Ile, Met or Asn at position 280 of the amino acid sequence of SEQ ID No. 20 (corresponding to position 335 of SEQ ID No. 2), preferably Met;
Met at position 310 of the amino acid sequence of SEQ ID No. 20 (corresponding to position 368 of SEQ ID No. 2);
Phe or Tyr at position 323 of the amino acid sequence of SEQ ID No. 20 (corresponding to position 381 of SEQ ID No. 2);
Ser at position 334 of the amino acid sequence of SEQ ID No. 20 (corresponding to position 392 of SEQ ID No. 2);
Tyr at position 346 of the amino acid sequence of SEQ ID No. 20 (corresponding to position 419 of SEQ ID No. 2);
Asp, Phe, Lys, Ala, Cys, Glu, Gly, His, Ile, Leu, Met, Gln, Ser, Thr or Val at position 348 of the amino acid sequence of SEQ ID No. 20 (corresponding to position 421 of SEQ ID No. 2), preferably Lys;
Ala, Met, Pro or Thr at position 349 of the amino acid sequence of SEQ ID No. 20 (corresponding to position 422 of SEQ ID No. 2);
Ala, Ile or Val at position 351 of the amino acid sequence of SEQ ID No. 20 (corresponding to position 424 of SEQ ID No. 2);
Ile, Pro, Arg, Ser, Ala, Gly, Lys or Gln at position 352 of the amino acid sequence of SEQ ID No. 20 (corresponding to position 425 of SEQ ID No. 2);
Glu, Phe, Thr, Val, Ala or Gln at position 353 of the amino acid sequence of SEQ ID No. 20 (corresponding to position 426 of SEQ ID No. 2), preferably Glu;
Met, Gln, Phe, Val, Phe or Leu at position 358 of the amino acid sequence of SEQ ID No. 20 (corresponding to position 431 of SEQ ID No. 2);
Ala, Cys, Gly or Thr at position 177 of the amino acid sequence of SEQ ID No. 20 (corresponding to position 228 of SEQ ID No. 2);
Ala, Glu, Gly, His, Lys, Leu, Asn, Gln, Arg, Ser, Thr or Tyr at position 199 of the amino acid sequence of SEQ ID No. 20 (corresponding to position 248 of SEQ ID No. 2);
Ala, Ile, Leu or Met at a position at position 221 of the amino acid sequence of SEQ ID No. 20 (corresponding to position 270 of SEQ ID No. 2);
Ala, Glu, His, Ile, Leu, Met, Arg, Ser, Thr or Val at position 222 of the amino acid sequence of SEQ ID No. 20 (corresponding to position 271 of SEQ ID No. 2);
His at position 321 of the amino acid sequence of SEQ ID No. 20 (corresponding to position 379 of SEQ ID No. 2);
Arg at position 354 of the amino acid sequence of SEQ ID No. 20 (corresponding to position 427 of SEQ ID No. 2).

In another embodiment, of the isolated nucleic acid of the invention, said HPPD protein comprises the amino acid sequence of SEQ ID No. 22 [Kordia algicida], wherein the resulting amino acid sequence comprises at least one amino acid selected from
Ala, Asp, Glu, Phe, Gly, Lys, Gln, Arg, Ser, Thr, Val, Tyr, Leu or Met at position 219 of the amino acid sequence of SEQ ID No. 22 (corresponding to position 250 of SEQ ID No. 2), preferably Phe or Ala;
Ala, Asp, Glu, Lys, Leu, Asn, Pro, Arg or Ser, Gly, His or Trp at position 220 of the amino acid sequence of SEQ ID No. 22 (corresponding to position 251 of SEQ ID No. 2), preferably Ala, Arg or Lys;
Glu, Phe, Gly, His, Ile, Leu, Met, Asn, Gln, Arg, Val, Tyr or Thr at position 221 of the amino acid sequence of SEQ ID No. 22 (corresponding to position 252 of SEQ ID No. 2), preferably Glu or Tyr;
Ala, His, Gln, Val Trp, Ile, Leu or Met at position 222 of the amino acid sequence of SEQ ID No. 22 (corresponding to position 253 of SEQ ID No. 2), preferably Val;
Val or Met at position 234 of the amino acid sequence of SEQ ID No. 22 (corresponding to position 265 of SEQ ID No. 2);
Leu, Gln, Arg, Val, Tyr, Ala, Ile or Met at position 237 of the amino acid sequence of SEQ ID No. 22 (corresponding to position 268 of SEQ ID No. 2);
Ala or Thr at position 238 of the amino acid sequence of SEQ ID No. 22 (corresponding to position 269 of SEQ ID No. 2), preferably Ala;
Ala, Ile, Asn, Thr or Val at position 249 of the amino acid sequence of SEQ ID No. 22 (corresponding to position 280 of SEQ ID No. 2);
Leu, Val, Ala, Phe, Gly, Met, Arg or Ser at position 261 of the amino acid sequence of SEQ ID No. 22 (corresponding to position 293 of SEQ ID No. 2);
Met, Ala, Pro, Ser, Thr or Val at position 262 of the amino acid sequence of SEQ ID No. 22 (corresponding to position 294 of SEQ ID No. 2);
His or Asn at position 275 of the amino acid sequence of SEQ ID No. 22 (corresponding to position 307 of SEQ ID No. 2);
Ile, Met or Asn at position 299 of the amino acid sequence of SEQ ID No. 22 (corresponding to position 335 of SEQ ID No. 2), preferably Met;
Met at position 329 of the amino acid sequence of SEQ ID No. 22 (corresponding to position 368 of SEQ ID No. 2);
Tyr at position 342 of the amino acid sequence of SEQ ID No. 22 (corresponding to position 381 of SEQ ID No. 2);
Ser at position 353 of the amino acid sequence of SEQ ID No. 22 (corresponding to position 392 of SEQ ID No. 2);
Tyr at position 365 of the amino acid sequence of SEQ ID No. 22 (corresponding to position 419 of SEQ ID No. 2);
Asp, Phe, Lys, Asn, Ala, Cys, Glu, Gly, His, Ile, Leu, Met, Gln, Ser or Thr at position 367 of the amino acid sequence of SEQ ID No. 22 (corresponding to position 421 of SEQ ID No. 2), preferably Lys;
Ala, Met, Pro or Thr at position 368 of the amino acid sequence of SEQ ID No. 22 (corresponding to position 422 of SEQ ID No. 2);
Ala, Ile or Val at position 370 of the amino acid sequence of SEQ ID No. 22 (corresponding to position 424 of SEQ ID No. 2);
Ile, Pro, Arg, Ser, Ala, Gly, Asn or Gln at position 371 of the amino acid sequence of SEQ ID No. 22 (corresponding to position 425 of SEQ ID No. 2);
Glu, Phe, Thr, Val, Gln or Ser at position 372 of the amino acid sequence of SEQ ID No. 22 (corresponding to position 426 of SEQ ID No. 2), preferably Glu;
Met, Gln, Phe, Val, Phe or Leu at position 377 of the amino acid sequence of SEQ ID No. 22 (corresponding to position 431 of SEQ ID No. 2);
Ala, Cys, Gly or Thr at position 195 of the amino acid sequence of SEQ ID No. 22 (corresponding to position 228 of SEQ ID No. 2);
Glu, Gly, His, Lys, Leu, Asn, Gln, Arg, Ser, Thr or Tyr at position 217 of the amino acid sequence of SEQ ID No. 22 (corresponding to position 248 of SEQ ID No. 2);
Ala, Ile, Leu or Val at position 239 of the amino acid sequence of SEQ ID No. 22 (corresponding to position 270 of SEQ ID No. 2);
Ala, Glu, His, Ile, Lys, Leu, Met, Arg, Thr or Val at position 240 of the amino acid sequence of SEQ ID No. 22 (corresponding to position 271 of SEQ ID No. 2);
His at a position at position 340 of the amino acid sequence of SEQ ID No. 22 (corresponding to position 379 of SEQ ID No. 2);
Arg at a position at position 373 of the amino acid sequence of SEQ ID No. 22 (corresponding to position 427 of SEQ ID No. 2).

In another embodiment, of the isolated nucleic acid of the invention, said HPPD protein comprises the amino acid sequence of SEQ ID No. 24 [Sorghum bicolor], wherein the resulting amino acid sequence comprises at least one amino acid selected from
Ala, Asp, Glu, Gly, Lys, Gln, Arg, Ser, Thr, Val Tyr, Ile, Leu or Met at position 241 of the amino acid sequence of SEQ ID No. 24 (corresponding to position 250 of SEQ ID No. 2), preferably Ala;
Asp, Glu, Lys, Leu, Asn, Pro, Arg, Ser, Gly, His or Trp at position 242 of the amino acid sequence of SEQ ID No. 24 (corresponding to position 251 of SEQ ID No. 2), preferably Arg or Lys;
Phe, Gly, His, Ile, Leu, Met, Asn, Gln, Arg, Val, Tyr or Thr at position 243 of the amino acid sequence of SEQ ID No. 24 (corresponding to position 252 of SEQ ID No. 2), preferably Tyr;
Ala, His, Gln, Val, Trp, Ile, Leu or Met at position 244 of the amino acid sequence of SEQ ID No. 24 (corresponding to position 253 of SEQ ID No. 2), preferably Val;
Val or Met at position 256 of the amino acid sequence of SEQ ID No. 24 (corresponding to position 265 of SEQ ID No. 2);
Leu, Gln, Arg, Val, Tyr, Ala, Ile or Lys at position 259 of the amino acid sequence of SEQ ID No. 24 (corresponding to position 268 of SEQ ID No. 2);
Ala or Thr at position 260 of the amino acid sequence of SEQ ID No. 24 (corresponding to position 269 of SEQ ID No. 2), preferably Ala;
Ala, Ile, Asn, Thr or Val at position 271 of the amino acid sequence of SEQ ID No. 24 (corresponding to position 280 of SEQ ID No. 2);
Leu or Val, Ala, Phe, Gly, Met, Arg or Ser at position 284 of the amino acid sequence of SEQ ID No. 24 (corresponding to position 293 of SEQ ID No. 2);
Met, Ala, Pro, Ser, Thr or Val at position 285 of the amino acid sequence of SEQ ID No. 24 (corresponding to position 294 of SEQ ID No. 2);
His or Asn at position 298 of the amino acid sequence of SEQ ID No. 24 (corresponding to position 307 of SEQ ID No. 2);
Ile, Asn or Leu at position 326 of the amino acid sequence of SEQ ID No. 24 (corresponding to position 335 of SEQ ID No. 2);
Met at position 359 of the amino acid sequence of SEQ ID No. 24 (corresponding to position 368 of SEQ ID No. 2);
Tyr at position 372 of the amino acid sequence of SEQ ID No. 24 (corresponding to position 381 of SEQ ID No. 2);
Ser at position 383 of the amino acid sequence of SEQ ID No. 24 (corresponding to position 392 of SEQ ID No. 2);
Tyr at position 410 of the amino acid sequence of SEQ ID No. 24 (corresponding to position 419 of SEQ ID No. 2);
Asp, Phe, Asn, Ala, Cys, Glu, Gly, His, Ile, Leu, Met, Gln, Ser, Thr or Val at position 412 of the amino acid sequence of SEQ ID No. 24 (corresponding to position 421 of SEQ ID No. 2);
Ala, Met, Pro or Thr at position 413 of the amino acid sequence of SEQ ID No. 24 (corresponding to position 422 of SEQ ID No. 2);
Ala, Ile or Val at position 415 of the amino acid sequence of SEQ ID No. 24 (corresponding to position 424 of SEQ ID No. 2);
Ile, Pro, Arg, Als, Gly, Lys, Asn or Gln at position 416 of the amino acid sequence of SEQ ID No. 24 (corresponding to position 425 of SEQ ID No. 2);
Phe, Thr, Val, Ala, Gln or Ser at position 417 of the amino acid sequence of SEQ ID No. 24 (corresponding to position 426 of SEQ ID No. 2);
Met, Gln, Phe, Val, Phe or Leu at position 422 of the amino acid sequence of SEQ ID No. 24 (corresponding to position 431 of SEQ ID No. 2);
Ala, Cys, Gly or Thr at position 219 of the amino acid sequence of SEQ ID No. 24 (corresponding to position 228 of SEQ ID No. 2);
Ala, Glu, Gly, Lys, Leu, Asn, Gln, Arg, Ser, Thr or Tyr at position 239 of the amino acid sequence of SEQ ID No. 24 (corresponding to position 248 of SEQ ID No. 2);
Ala, Ile, Met or Val at position 261 of the amino acid sequence of SEQ ID No. 24 (corresponding to position 270 of SEQ ID No. 2);
Glu, His, Ile, Lys, Leu, Met, Arg, Ser, Thr or Val at position 262 of the amino acid sequence of SEQ ID No. 24 (corresponding to position 271 of SEQ ID No. 2);
His at position 370 of the amino acid sequence of SEQ ID No. 24 (corresponding to position 379 of SEQ ID No. 2);
Arg at position 418 of the amino acid sequence of SEQ ID No. 24 (corresponding to position 427 of SEQ ID No. 2).

In another embodiment, of the isolated nucleic acid of the invention, said HPPD protein comprises the amino acid sequence of SEQ ID No. 26 [Triticum aestivum/wheat], wherein the resulting amino acid sequence comprises at least one amino acid selected from
Ala, Asp, Glu, Gly, Lys, Gln, Arg, Ser, Thr, Val, Tyr, Ile, Leu or Met at position 237 of the amino acid sequence of SEQ ID No. 26 (corresponding to position 250 of SEQ ID No. 2), preferably Ala;
Asp, Glu, Lys, Leu, Asn, Pro, Arg or Ser, Gly, His or Trp at position 238 of the amino acid sequence of SEQ ID No. 26 (corresponding to position 251 of SEQ ID No. 2), preferably Arg or Lys;
Phe, Gly, His, Ile, Leu, Met, Asn, Gln, Arg, Val or Tyr or Thr at position 239 of the amino acid sequence of SEQ ID No. 26 (corresponding to position 252 of SEQ ID No. 2), preferably Tyr;
Ala, His, Gln, Val Trp, Ile, Leu or Met at position 240 of the amino acid sequence of SEQ ID No. 26 (corresponding to position 253 of SEQ ID No. 2), preferably Val;
Val or Met at position 252 of the amino acid sequence of SEQ ID No. 26 (corresponding to position 265 of SEQ ID No. 2);
Leu, Gln, Arg, Val, Tyr, Ala, Ile or Lys at position 255 of the amino acid sequence of SEQ ID No. 26 (corresponding to position 268 of SEQ ID No. 2);
Ala or Thr at position 256 of the amino acid sequence of SEQ ID No. 26 (corresponding to position 269 of SEQ ID No. 2), preferably Ala;
Ala, Ile, Asn, Thr or Val at position 267 of the amino acid sequence of SEQ ID No. 26 (corresponding to position 280 of SEQ ID No. 2);
Leu, Val, Ala, Phe, Gly, Met, Arg or Ser at position 280 of the amino acid sequence of SEQ ID No. 26 (corresponding to position 293 of SEQ ID No. 2);
Met, Ala, Pro, Ser, Thr or Val at position 281 of the amino acid sequence of SEQ ID No. 26 (corresponding to position 294 of SEQ ID No. 2);
His or Asn at position 294 of the amino acid sequence of SEQ ID No. 26 (corresponding to position 307 of SEQ ID No. 2);
Ile, Met or Asn at position 322 of the amino acid sequence of SEQ ID No. 26 (corresponding to position 335 of SEQ ID No. 2), preferably Met;
Met at position 355 of the amino acid sequence of SEQ ID No. 26 (corresponding to position 368 of SEQ ID No. 2);
Tyr at position 368 of the amino acid sequence of SEQ ID No. 26 (corresponding to position 381 of SEQ ID No. 2);
Ser at position 379 of the amino acid sequence of SEQ ID No. 26 (corresponding to position 392 of SEQ ID No. 2);
Tyr at position 406 of the amino acid sequence of SEQ ID No. 26 (corresponding to position 419 of SEQ ID No. 2);
Asp, Phe, Asn, Ala, Cys, Glu, Gly, His, Ile, Leu, Met, Gln, Ser, Thr or Val at position 408 of the amino acid sequence of SEQ ID No. 26 (corresponding to position 421 of SEQ ID No. 2);
Ala, Met, Pro or Thr at position 409 of the amino acid sequence of SEQ ID No. 26 (corresponding to position 422 of SEQ ID No. 2);
Ala, Ile or Val at position 411 of the amino acid sequence of SEQ ID No. 26 (corresponding to position 424 of SEQ ID No. 2);
Ile, Pro, Arg, Ala, Gly, Lys, Asn or Gln at position 412 of the amino acid sequence of SEQ ID No. 26 (corresponding to position 425 of SEQ ID No. 2);
Phe, Thr or Val, Ala, Gln or Ser at position 413 of the amino acid sequence of SEQ ID No. 26 (corresponding to position 426 of SEQ ID No. 2);
Met, Gln, Phe, Leu or Val at position 418 of the amino acid sequence of SEQ ID No. 26 (corresponding to position 431 of SEQ ID No. 2);
Ala, Cys, Gly or Thr at a position in an HPPD protein, said position corresponding to position 215 of the amino acid sequence of SEQ ID No. 26 (corresponding to position 228 of SEQ ID No. 2);
Ala, Glu, Gly, Lys, Leu, Asn, Gln, Arg, Ser, Thr or Tyr at position 235 of the amino acid sequence of SEQ ID No. 26 (corresponding to position 248 of SEQ ID No. 2);
Ala, Ile, Met or Val at position 257 of the amino acid sequence of SEQ ID No. 26 (corresponding to position 270 of SEQ ID No. 2);
Glu, His, Ile, Lys, Leu, Met, Arg, Ser, Thr or Val at position 258 of the amino acid sequence of SEQ ID No. 26 (corresponding to position 271 of SEQ ID No. 2);
His at a position at position 366 of the amino acid sequence of SEQ ID No. 26 (corresponding to position 379 of SEQ ID No. 2);
Arg at position 414 of the amino acid sequence of SEQ ID No. 26 (corresponding to position 427 of SEQ ID No. 2).

In another embodiment, of the isolated nucleic acid of the invention, said HPPD protein comprises the amino acid sequence of SEQ ID No. 2 [Arabidopsis thaliana], wherein the resulting amino acid sequence comprises at least one amino acid selected from
Ala, Asp, Glu, Gly, Lys, Gln, Arg, Ser, Thr, Val, Tyr, Ile, Leu or Met at position 250 of the amino acid sequence of SEQ ID No. 2;
Asp, Glu, Ile, Lys, Leu, Asn, Pro, Arg, Ser, Gly, His or Trp at position 251 of the amino acid sequence of SEQ ID No. 2;
Phe, Gly, His, Ile, Leu, Met, Asn, Gln, Arg, Ser, Val, Tyr or Thr at position 252 of the amino acid sequence of SEQ ID No. 2;
Ala, His, Gln, Val, Trp, Ile, Leu or Met at position 253 of the amino acid sequence of SEQ ID No. 2;
Val or Met at a position at position 265 of the amino acid sequence of SEQ ID No. 2;
Leu, Gln, Arg, Val, Tyr, Ile, Lys or Met at position 268 of the amino acid sequence of SEQ ID No. 2;
Ala or Thr at position 269 of the amino acid sequence of SEQ ID No. 2;
Ala, Ile, Asn, Thr or Val at position 280 of the amino acid sequence of SEQ ID No. 2;
Gln, Val, Ala, Phe, Gly, Met, Arg or Ser at a in an HPPD protein, said position corresponding to position 293 of the amino acid sequence of SEQ ID No. 2;
Met, Ala, Pro, Ser, Thr or Val at position 294 of the amino acid sequence of SEQ ID No. 2;
His or Asn at position 307 of the amino acid sequence of SEQ ID No. 2;
Ile, Asn or Leu at position 335 of the amino acid sequence of SEQ ID No. 2;
Met at position 368 of the amino acid sequence of SEQ ID No. 2;
Tyr at position 381 of the amino acid sequence of SEQ ID No. 2;
Ser at position 392 of the amino acid sequence of SEQ ID No. 2;
Tyr at position 419 of the amino acid sequence of SEQ ID No. 2;
Asp, Phe, Asn, Ala, Cys, Glu, Gly, His, Ile, Leu, Met, Gln, Ser, Thr or Val at position 421 of the amino acid sequence of SEQ ID No. 2;
Ala, Met, Pro or Thr at position 422 of the amino acid sequence of SEQ ID No. 2;
Ala, Ile or Val at position 424 of the amino acid sequence of SEQ ID No. 2;
Ile, Pro, Arg, Ala, Gly, Lys, Asn or Gln at position 425 of the amino acid sequence of SEQ ID No. 2;
Phe, Thr, Val, Ala, Gln or Ser at position 426 of the amino acid sequence of SEQ ID No. 2;
Met, Gln, Val, Phe or Leu position 431 of the amino acid sequence of SEQ ID No. 2;
Ala, Cys, Gly or Thr at position 228 of the amino acid sequence of SEQ ID No. 2;
Ala, Glu, Gly, Lys, Leu, Asn, Gln, Arg, Ser, Thr or Tyr at position 248 of the amino acid sequence of SEQ ID No. 2;
Ala, Ile, Met or Val at position 270 of the amino acid sequence of SEQ ID No. 2;
Glu, His, Ile, Lys, Leu, Met, Arg, Ser, Thr or Val at position 271 of the amino acid sequence of SEQ ID No. 2;
His at position 379 of the amino acid sequence of SEQ ID No. 2; or
Arg at position 427 of the amino acid sequence of SEQ ID No. 2.

In a further embodiment of the nucleic acid of the invention, in said mutated HPPD protein at least one amino acid has been replaced so that the resulting amino acid sequence comprises (a) Glu, Ser or Tyr at position 252; (b) Val or Ala at position 269; (c) Pro, Val or Ala at position 280; (d) Asp, Lys or Asn at position 421; and (e) Gly or Ala at postion 422. These positions correspond to amino acid sequence of SEQ ID No 2.

In a further embodiment of the nucleic acid of the invention, in said mutated HPPD protein at least two amino acids have been replaced.

An isolated nucleic acid comprising a nucleotide sequence encoding a mutated HPPD protein,
wherein said mutated HPPD protein has HPPD activity,
wherein in said mutated HPPD protein at least one amino acid at position 228, 248, 270, 271, 379 and/or 427 has been replaced by another amino acid.

In an alternative embodiment of the nucleic acid of the invention having at least one amino acid at position 228, 248, 270, 271, 379 and/or 427 deleted or replaced by another amino acid as defined above, said mutated HPPD protein comprises
a His at a position in an HPPD protein, said position corresponding to position 226 of the amino acid sequence of SEQ ID No. 2;
a Ser at a position in an HPPD protein, said position corresponding to position 267 comprising the amino acid sequence of SEQ ID No. 2 or at a position corresponding thereto in a different HPPD enzyme;
an Asn at a position in an HPPD protein, said position corresponding to position 282 of the amino acid sequence of SEQ ID No. 2;
a His at a position in an HPPD protein, said position corresponding to position 308 of the amino acid sequence of SEQ ID No. 2;
a Tyr at a position in an HPPD protein, said position corresponding to position 342 of the amino acid sequence of SEQ ID No. 2;
a Glu at a position in an HPPD protein, said position corresponding to position 394 of the amino acid sequence of SEQ ID No. 2;
a Gly at a position in an HPPD protein, said position corresponding to position 420 of the amino acid sequence of SEQ ID No. 2; and
an Asn at a position in an HPPD protein, said position corresponding to position 423 of the amino acid sequence of SEQ ID No. 2.

In another embodiment of the isolated nucleic acid as defined above, in said mutated HPPD protein at least one amino acid has been replaced so that the resulting amino acid sequence has at least one selected from
Ala, Cys, Gly, Thr or Val at a position in an HPPD protein, said position corresponding to position 228 of the amino acid sequence of SEQ ID No. 2;
Ala, Glu, Gly, His, Lys, Leu, Asn, Gln, Arg, Ser, Thr or Tyr at a position in an HPPD protein, said position corresponding to position 248 of the amino acid sequence of SEQ ID No. 2;
Ala, Ile, Leu, Met or Val at a position in an HPPD protein, said position corresponding to position 270 of the amino acid sequence of SEQ ID No. 2;
Ala, Glu, Hils, Ile, Lys, Leu, Met, Arg, Ser, Thr or Val at a position in an HPPD protein, said position corresponding to position 271 of the amino acid sequence of SEQ ID No. 2;
His or Gln at a position in an HPPD protein, said position corresponding to position 379 of the amino acid sequence of SEQ ID No. 2; and
Leu or Arg at a position in an HPPD protein, said position corresponding to position 427 of the amino acid sequence of SEQ ID No. 2.

In another embodiment of the isolated nucleic acid as defined above, in said mutated HPPD protein at least one amino acid has been replaced so that the resulting amino acid sequence has at least one selected from
Val or Thr at a position in an HPPD protein, said position corresponding to position 228 of the amino acid sequence of SEQ ID No. 2;
Leu, Met or Val at a position in an HPPD protein, said position corresponding to position 270 of the amino acid sequence of SEQ ID No. 2;
Ala or Ser at a position in an HPPD protein, said position corresponding to position 271 of the amino acid sequence of SEQ ID No. 2,
Gln at a position in an HPPD protein, said position corresponding to position 379 of the amino acid sequence of SEQ ID No. 2; and
Leu at a position in an HPPD protein, said position corresponding to position 427 of the amino acid sequence of SEQ ID No. 2.

In another embodiment of the nucleic acid as defined above, said mutated HPPD protein is capable of increasing the tolerance of a plant to at least one herbicide acting on HPPD (also called HPPD inhibitor herbicide).

In another embodiment, the present invention relates to a protein encoded by the isolated nucleic acid of the invention.

In a further embodiment, the present invention relates to a chimeric gene comprising a coding sequence comprising the nucleic acid of the invention operably linked to a plant-expressible promoter and optionally a transcription termination and polyadenylation region.

As a regulatory sequence which functions as a promoter in plant cells and plants, use may be made of any promoter sequence of a gene which is naturally expressed in plants, in particular a promoter which is expressed especially in the leaves of plants, such as for example “constitutive” promoters of bacterial, viral or plant origin, or “light-dependent” promoters, such as that of a plant ribulose-biscarboxylase/oxygenase (RuBisCO) small subunit gene, or any suitable known promoter-expressible which may be used. Among the promoters of plant origin, mention will be made of the histone promoters as described in EP 0 507 698 A1, the rice actin promoter (U.S. Pat. No. 5,641,876), or a plant ubiquitin promoter (U.S. Pat. No. 5,510,474). Among the promoters of a plant virus gene, mention will be made of that of the cauliflower mosaic virus (CaMV 19S or 35S, Sanders et al. (1987), Nucleic Acids Res. 15(4):1543-58.), the circovirus (AU 689 311) or the Cassava vein mosaic virus (CsVMV, U.S. Pat. No. 7,053,205).

In one embodiment of this invention, a promoter sequence specific for particular regions or tissues of plants can be used to express the HPPD proteins of the invention, such as promoters specific for seeds (Datla, R. et al., 1997, Biotechnology Ann. Rev. 3, 269-296), especially the napin promoter (EP 255 378 A1), the phaseolin promoter, the glutenin promoter, the helianthinin promoter (WO 92/17580), the albumin promoter (WO 98/45460), the oleosin promoter (WO 98/45461), the SAT1 promoter or the SAT3 promoter (PCT/US98/06978).

Use may also be made of an inducible promoter advantageously chosen from the phenylalanine ammonia lyase (PAL), HMG-CoA reductase (HMG), chitinase, glucanase, proteinase inhibitor (PI), PR1 family gene, nopaline synthase (nos) and vspB promoters (U.S. Pat. No. 5,670,349, Table 3), the HMG2 promoter (U.S. Pat. No. 5,670,349), the apple beta-galactosidase (ABG1) promoter and the apple aminocyclopropane carboxylate synthase (ACC synthase) promoter (WO 98/45445).

According to the invention, use may also be made, in combination with the promoter, of other regulatory sequences, which are located between the promoter and the coding sequence, such as transcription activators (“enhancers”), for instance the translation activator of the tobacco mosaic virus (TMV) described in Application WO 87/07644, or of the tobacco etch virus (TEV) described by Carrington & Freed 1990, J. Virol. 64: 1590-1597, for example, or introns such as the adhI intron of maize or intron 1 of rice actin.

As a regulatory terminator or polyadenylation sequence, use may be made of any corresponding sequence of bacterial origin, such as for example the nos terminator of Agrobacterium tumefaciens, of viral origin, such as for example the CaMV 35S terminator, or of plant origin, such as for example a histone terminator as described in published Patent Application EP 0 633 317 A1.

A method of obtaining a mutated HPPD protein capable of modulating the tolerance of a plant to at least one herbicide acting on HPPD, wherein said mutated HPPD protein has HPPD activity, the method comprising
providing an HPPD protein, said HPPD optionally comprising an amino acid sequence, wherein
a His is present at a position in an HPPD protein, said position corresponding to position 226 of the amino acid sequence of SEQ ID No. 2;
a Ser is present at a position in an HPPD protein, said position corresponding to position 267 of the amino acid sequence of SEQ ID No. 2;
an Asn is present at a position in an HPPD protein, said position corresponding to position 282 of the amino acid sequence of SEQ ID No. 2;
a His is present at a position in an HPPD protein, said position corresponding to position 308 of the amino acid sequence of SEQ ID No. 2;
a Tyr is present at a position in an HPPD protein, said position corresponding to position 342 of the amino acid sequence of SEQ ID No. 2;
a Glu is present at a position in an HPPD protein, said position corresponding to position 394 of the amino acid sequence of SEQ ID No. 2;
a Gly is present at a position in an HPPD protein, said position corresponding to position 420 of the amino acid sequence of SEQ ID No. 2; and
an Asn is present at a position in an HPPD protein, said position corresponding to position 423 of the amino acid sequence of SEQ ID No. 2
ii) replacing at least one amino acid in said HPPD enzyme so that the resulting amino acid sequence has at least one selected from
Ala, Asp, Glu, Phe, Gly, Lys, Gln, Arg, Ser, Thr, Val, Ile, Leu, Met or Tyr at a position in an HPPD protein, said position corresponding to position 250 of the amino acid sequence of SEQ ID No. 2;
Ala, Asp, Glu, Ile, Lys, Leu, Asn, Pro, Arg, Gly, His, Tyr or Ser at a position in an HPPD protein, said position corresponding to position 251 of the amino acid sequence of SEQ ID No. 2;
Glu, Phe, Gly, His, Ile, Leu, Met, Asn, Gln, Arg, Ser, Val, Thr or Tyr at a position in an HPPD protein, said position corresponding to position 252 of the amino acid sequence of SEQ ID No. 2;
Ala, Phe, His, Gln, Val, Ile, Leu, Met or Trp at a position in an HPPD protein, said position corresponding to position 253 of the amino acid sequence of SEQ ID No. 2;
Leu, Met or Val at a position in an HPPD protein, said position corresponding to position 265 of the amino acid sequence of SEQ ID No. 2;
Leu, Gln, Arg, Val, Ala, Ile, Lys, Met or Tyr at a position in an HPPD protein, said position corresponding to position 268 of the amino acid sequence of SEQ ID No. 2;
Ala, Thr or Val at a position in an HPPD protein, said position corresponding to position 269 of the amino acid sequence of SEQ ID No. 2;
Ala, Ile, Asn, Pro, Thr or Val at a position in an HPPD protein, said position corresponding to position 280 of the amino acid sequence of SEQ ID No. 2;
Leu, Gln, Ala, Phe, Gly, Met, Arg, Ser or Val at a position in an HPPD protein, said position corresponding to position 293 of the amino acid sequence of SEQ ID No. 2;
Ile, ala, Pro, Ser, Thr, Val or Met at a position in an HPPD protein, said position corresponding to position 294 of the amino acid sequence of SEQ ID No. 2;
Gln, His or Asn at a position in an HPPD protein, said position corresponding to position 307 of the amino acid sequence of SEQ ID No. 2;
Ile, Met, Leu or Asn at a position in an HPPD protein, said position corresponding to position 335 of the amino acid sequence of SEQ ID No. 2;
Leu or Met at a position in an HPPD protein, said position corresponding to position 368 of the amino acid sequence of SEQ ID No. 2;
Phe or Tyr at a position in an HPPD protein, said position corresponding to position 381 of the amino acid sequence of SEQ ID No. 2;
Phe or Ser at a position in an HPPD protein, said position corresponding to position 392 of the amino acid sequence of SEQ ID No. 2;
Phe or Tyr at a position in an HPPD protein, said position corresponding to position 419 of the amino acid sequence of SEQ ID No. 2;
Asp, Phe, Lys, Asn, Ala, Cys, Glu, Gly, His, Ile, Leu, Met, Gln, Ser, Thr or Val at a position in an HPPD protein, said position corresponding to position 421 of the amino acid sequence of SEQ ID No. 2;
Ala, Gly, Met, Pro or Thr at a position in an HPPD protein, said position corresponding to position 422 of the amino acid sequence of SEQ ID No. 2;
Ala, Phe, Ile or Val at a position in an HPPD protein, said position corresponding to position 424 of the amino acid sequence of SEQ ID No. 2 or;
Ile, Pro, Arg, Ala, Gly, Lys, Asn, Gln or Ser at a position in an HPPD protein, said position corresponding to position 425 of the amino acid sequence of SEQ ID No. 2;
Glu, Phe, Thr, Ala, Gln, Ser or Val at a position in an HPPD protein, said position corresponding to position 426 of the amino acid sequence of SEQ ID No. 2;
Ile, Met, Gln, Phe, Leu or Val at a position in an HPPD protein, said position corresponding to position 431 of the amino acid sequence of SEQ ID No. 2;
an amino acid deletion or replacement at least one position in an HPPD protein, said position corresponding to at least one of positions 228, 248, 270, 271, 379 and 427 of the amino acid sequence of SEQ ID No. 2;
iii) determining the inhibition of the resulting HPPD protein by at least one herbicide acting on HPPD;
wherein an inhibition of the resulting protein of less or more than that observed with a reference HPPD protein is indicative that the resulting protein is capable of modulating the tolerance of a plant to said herbicide.

It is to be understood that also the (more specific) amino acids and positions listed above for other embodiments, such as the nucleic acid of the invention, may be applied to the method of obtaining a mutated HPPD protein as described above.

In an alternative embodiment of the method of obtaining a mutated HPPD protein as described above, said mutated HPPD protein is capable of increasing the tolerance of a plant to at least one herbicide acting on HPPD.

Within the above method of obtaining in mutated HPPD protein, different herbicides acting on HPPD may be chosen. Accordingly, in another embodiment of the method of obtaining a mutated HPPD protein as described above, wherein said mutated HPPD protein is capable of increasing the tolerance of a plant to at least one herbicide acting on HPPD, the herbicide acting on HPPD is selected from triketones, or pyrazolinates, preferably tembotrione, mesotrione, topramezone or sulcotrione, bicyclopyrone, pyrasulfotole, pyrazolate, benzofenap and tefuryltrione, particularly tembotrione and such plants containing the HPPD of the invention have an agronomically acceptable tolerance to an HPPD inhibitor herbicide particularly to triketones, or pyrazolinates, preferably tembotrione, mesotrione, topramezone or sulcotrione, bicyclopyrone, pyrasulfotole, pyrazolate, benzofenap and tefuryltrione, particularly tembotrione.

In another embodiment, the present invention relates to a method of producing a transgenic plant comprising introducing into a said plant genome the nucleic acid of the present invention operably linked to a plant expressible promoter, the chimeric gene of the invention or a nucleic acid encoding the HPPD enzyme identified by the method of claim 27 or 28.

In an alternative embodiment of the method of producing a transgenic plant as described above, the nucleic acid of the invention, wherein said mutated HPPD protein is capable of increasing the tolerance of a plant to at least one herbicide acting on HPPD, or a nucleic acid identified by the method of obtaining a mutated HPPD protein, wherein said mutated HPPD protein is capable of increasing the tolerance of a plant to at least one herbicie acting on HPPD, both operably linked to a plant expressible promoter, or the chimeric gene of the invention comprising a nucleic acid, wherein said mutated HPPD protein is capable of increasing the tolerance of a plant to at least one herbicie acting on HPPD, is introduced into said plant.

In another embodiment, the present invention relates to a plant cell comprising the isolated nucleic acid of the invention or the chimeric gene of the invention in its genetic information.

The present invention also relates to a plant, a part of a plant or plant tissue consisting essentially of the plant cells of the invention.

Furthermore, the present invention relates to a plant obtainable from the method of obtaining a mutated HPPD protein capable of modulating or increasing the tolerance of a plant to at least one herbicide acting on HPPD in all alternative aspects described above.

The plant of the present invention can be any plant. Non-limiting examples of plants of the invention include wheat, cotton, canola, rice, corn, soy bean, sorghum, canola, sunflower, tobacco, sugarbeet, cotton, maize, wheat, barley, rice, sorghum, tomato, mango, peach, apple, pear, strawberry, banana, melon, potato, carrot, lettuce, cabbage, onion, soya spp, sugar cane, pea, field beans, poplar, grape, citrus, alfalfa, rye, oats, turf and forage grasses, flax and oilseed rape, and nut producing plants.

The present invention also relates to a seed of the plant of the invention.

Furthermore, the invention relates to progeny of the plant of the invention or the seed of the invention.

In a further embodiment, the present invention relates to a method of modulating a plant's tolerance to at least one herbicide acting on HPPD comprising introducing the isolated nucleic acid of the invention operably linked to a plant expressible promoter or the chimeric gene of the invention into a plant's genome

In an alternative embodiment, the present invention relates to a method of increasing a plant's tolerance to at least one herbicide acting on HPPD or of obtaining a plant tolerant to an HPPD inhibitor herbicide comprising introducing the isolated nucleic acid of the invention, wherein said nucleic acid encodes a mutated HPPD protein which is capable of increasing the tolerance of a plant to at least one herbicide acting on HPPD, operably linked to a plant expressible promoter or the chimeric gene of the invention comprising a nucleic acid of the invention, wherein said nucleic acid encodes a mutated HPPD protein which is capable of increasing the tolerance of a plant to at least one herbicide acting on HPPD, into a plant's genome.

Furthermore, the present invention relates to a method for controlling weeds comprising spraying at least one herbicide acting on HPPD on or around a crop plant, wherein said crop plant comprises the nucleic acid of the present invention, wherein said mutated HPPD protein is capable of increasing the tolerance of a plant to at least one herbicide acting on HPPD, operably linked to a plant expressible promoter or the chimeric gene of the invention comprising the nucleic acid of the invention, wherein said mutated HPPD protein is capable of increasing the tolerance of a plant to at least one herbicide acting on HPPD. In alternative embodiment of the method of controlling weeds, the tolerance of said plant to at least one herbicie acting on HPPD is increased.

In addition, the present invention relates to the use of a chimeric gene of the invention or the nucleic acid of the invention operably linked to a plant expressible promoter for modulating the tolerance of a plant to at least one herbicide acting on HPPD.

In an alternative embodiment, the present invention relates to the use of a chimeric gene of the invention or the nucleic acid of the invention operably linked to a plant expressible promoter for increasing the tolerance of a plant to at least one herbicide acting on HPPD. In this embodiment of the invention, the chimeric gene used comprises the nucleic acid of the invention, wherein the mutated HPPD protein encoded thereby is capable of increasing the tolerance of a plant to at least one herbicide acting on HPPD. Alternatively, if a nucleic acid operably linked to a plant expressible promoter is used, said nucleic is chose that the mutated HPPD protein encoded thereby is capable of increasing the tolerance of a plant to at least one herbicide acting on HPPD.

The present invention also relates to the plant cell of the invention and the plant of the invention which may comprise a further useful trait as described further below.

While a number of herbicide-tolerant crop plants are presently commercially available, one issue that has arisen for many commercial herbicides and herbicide/crop combinations is that individual herbicides typically have incomplete spectrum of activity against common weed species. For most individual herbicides which have been in use for some time, populations of herbicide resistant weed species and biotypes have become more prevalent (see, e.g., Tranel and Wright (2002) Weed Science 50: 700-712; Owen and Zelaya (2005) Pest Manag. Sci. 61: 301-311). Transgenic plants which are resistant to more than one herbicide have been described (see, e.g., WO2005/012515). However, improvements in every aspect of crop production, weed control options, extension of residual weed control, and improvement in crop yield are continuously in demand.

The HPPD protein or gene of the invention is advantageously combined in plants with other genes which encode proteins or RNAs that confer useful agronomic properties to such plants. Among the genes which encode proteins or RNAs that confer useful agronomic properties on the transformed plants, mention can be made of the DNA sequences encoding proteins which confer tolerance to one or more herbicides that, according to their chemical structure, differ from HPPD inhibitor herbicides, and others which confer tolerance to certain insects, those which confer tolerance to certain diseases, DNAs that encodes RNAs that provide nematode or insect control, etc. . . .

Such genes are in particular described in published PCT Patent Applications WO 91/02071 and WO95/06128.

Among the DNA sequences encoding proteins which confer tolerance to certain herbicides on the transformed plant cells and plants, mention can be made of a bar or PAT gene or the Streptomyces coelicolor gene described in WO2009/152359 which confers tolerance to glufosinate herbicides, a gene encoding a suitable EPSPS which confers tolerance to herbicides having EPSPS as a target, such as glyphosate and its salts (U.S. Pat. Nos. 4,535,060, 4,769,061, 5,094,945, 4,940,835, 5,188,642, 4,971,908, 5,145,783, 5,310,667, 5,312,910, 5,627,061, 5,633,435), or a gene encoding glyphosate oxydoreductase (U.S. Pat. No. 5,463,175).

Among the DNA sequences encoding a suitable EPSPS which confer tolerance to the herbicides which have EPSPS as a target, mention will more particularly be made of the gene which encodes a plant EPSPS, in particular maize EPSPS, particularly a maize EPSPS which comprises two mutations, particularly a mutation at amino acid position 102 and a mutation at amino acid position 106 (WO 2004/074443), and which is described in U.S. Pat. No. 6,566,587, hereinafter named double mutant maize EPSPS or 2mEPSPS, or the gene which encodes an EPSPS isolated from Agrobacterium and which is described by sequence ID No. 2 and sequence ID No. 3 of U.S. Pat. No. 5,633,435, also named CP4.

Among the DNA sequences encoding a suitable EPSPS which confer tolerance to the herbicides which have EPSPS as a target, mention will more particularly be made of the gene which encodes an EPSPS GRG23 from Arthrobacter globiformis, but also the mutants GRG23 ACE1, GRG23 ACE2, or GRG23 ACE3, particularly the mutants or variants of GRG23 as described in WO2008/100353, such as GRG23(ace3)R173K of SEQ ID No. 29 in WO2008/100353.

In the case of the DNA sequences encoding EPSPS, and more particularly encoding the above genes, the sequence encoding these enzymes is advantageously preceded by a sequence encoding a transit peptide, in particular the “optimized transit peptide” described in U.S. Pat. No. 5,510,471 or 5,633,448.

In WO 2007/024782, plants being tolerant to glyphosate and at least one ALS (acetolactate synthase) inhibitor are disclosed. More specifically plants containing genes encoding a GAT (Glyphosate-N-Acetyltransferase) polypeptide and a polypeptide conferring resistance to ALS inhibitors are disclosed.

In U.S. Pat. No. 6,855,533, transgenic tobacco plants containing mutated Arabidopsis ALS/AHAS genes were disclosed.

In U.S. Pat. No. 6,153,401, plants containing genes encoding 2,4-D-monooxygenases conferring tolerance to 2,4-D (2,4-dichlorophenoxyacetic acid) by metabolisation are disclosed.

In US 2008/0119361 and US 2008/0120739, plants containing genes encoding Dicamba monooxygenases conferring tolerance to dicamba (3,6-dichloro-2-methoxybenzoic acid) by metabolisation are disclosed.

All the above mentioned herbicide tolerance traits can be combined with those performing HPPD tolerance which are subject matter of this invention.

Among the DNA sequences encoding proteins concerning properties of tolerance to insects, mention will more particularly be made of the Bt proteins widely described in the literature and well known to those skilled in the art. Mention will also be made of proteins extracted from bacteria such as Photorhabdus (WO 97/17432 & WO 98/08932).

Among such DNA sequences encoding proteins of interest which confer novel properties of tolerance to insects, mention will more particularly be made of the Bt Cry or VIP proteins widely described in the literature and well known to those skilled in the art. These include the Cry1F protein or hybrids derived from a Cry1F protein (e.g., the hybrid Cry1A-Cry1F proteins described in U.S. Pat. Nos. 6,326,169; 6,281,016; 6,218,188, or toxic fragments thereof), the Cry1A-type proteins or toxic fragments thereof, preferably the Cry1Ac protein or hybrids derived from the Cry1Ac protein (e.g., the hybrid Cry1Ab-Cry1Ac protein described in U.S. Pat. No. 5,880,275) or the Cry1Ab or Bt2 protein or insecticidal fragments thereof as described in EP451878, the Cry2Ae, Cry2Af or Cry2Ag proteins as described in WO02/057664 or toxic fragments thereof, the Cry1A.105 protein described in WO 2007/140256 (SEQ ID No. 7) or a toxic fragment thereof, the VIP3Aa19 protein of NCBI accession ABG20428, the VIP3Aa20 protein of NCBI accession ABG20429 (SEQ ID No. 2 in WO 2007/142840), the VIP3A proteins produced in the COT202 or COT203 cotton events (WO 2005/054479 and WO 2005/054480, respectively), the Cry proteins as described in WO01/47952, the VIP3Aa protein or a toxic fragment thereof as described in Estruch et al. (1996), Proc Natl Acad Sci USA. 28; 93(11):5389-94 and U.S. Pat. No. 6,291,156, the insecticidal proteins from Xenorhabdus (as described in WO98/50427), Serratia (particularly from S. entomophila) or Photorhabdus species strains, such as Tc-proteins from Photorhabdus as described in WO98/08932 (e.g., Waterfield et al., 2001, Appl Environ Microbiol. 67(11):5017-24; Ffrench-Constant and Bowen, 2000, Cell Mol Life Sci.; 57(5):828-33). Also any variants or mutants of any one of these proteins differing in some (1-10, preferably 1-5) amino acids from any of the above sequences, particularly the sequence of their toxic fragment, or which are fused to a transit peptide, such as a plastid transit peptide, or another protein or peptide, is included herein.

### THE FIGURES SHOW

FIGS. 1a-1d: Superposition of the x-ray structure of HPPD from Arabidopsis thaliana (dark grey) and (a) Pseudomonas fluorescens (light grey), (b) Streptomyces avermitilis, (c) Homo sapiens, (d) Rattus norvegicus. The structures are shown as ribbon plot.

FIGS. 2a-2e: Amino acids forming the binding site of (a) Arabidopsis thaliana, (b) Pseudomonas fluorescens (c) Streptomyces avermitilis (d) Homo sapiens, and (e) Rattus norvegicus (represented by bold lines). The core of the HPPD protein is shown as Cₐₗₚₕₐ trace and the iron is marked.

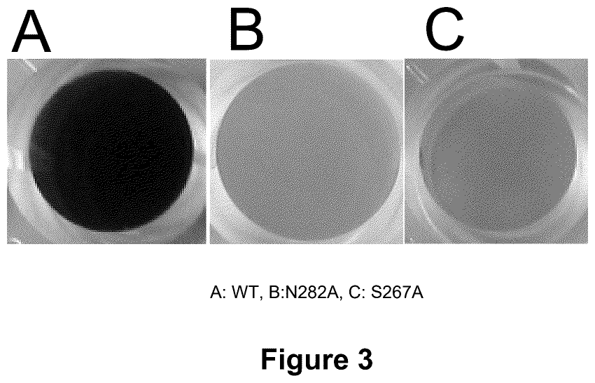

FIG. 3: Brown test color in Escherichia coli

### SEQUENCES LISTING

SEQ ID No. 1: Nucleic acid sequence encoding Arabidopsis thaliana HPPD
SEQ ID No. 2: Protein encoded by SEQ ID No. 1
SEQ ID No. 3: Nucleic acid sequence encoding Oryza sativa HPPD
SEQ ID No. 4: Protein encoded by SEQ ID No. 3
SEQ ID No. 5: Nucleic acid sequence encoding Zea mays HPPD
SEQ ID No. 6: Protein encoded by SEQ ID No. 5
SEQ ID No. 7: Nucleic acid sequence encoding Avena sativa HPPD
SEQ ID No. 8: Protein encoded by SEQ ID No. 7
SEQ ID No. 9: Nucleic acid sequence encoding Pseudomonas fluorescens HPPD
SEQ ID No. 10: Protein encoded by SEQ ID No. 9
SEQ ID No. 13: Nucleic acid sequence encoding Synechococcus sp. HPPD
SEQ ID No. 14: Protein encoded by SEQ ID No. 13
SEQ ID No. 15: Nucleic acid sequence encoding Blepharsima japonicum HPPD
SEQ ID No. 16: Protein encoded by SEQ ID No. 15
SEQ ID No. 17: Nucleic acid sequence encoding Rhodococcus RHA1 isolate ro0341 HPPD
SEQ ID No. 18: Protein encoded by SEQ ID No. 17
SEQ ID No. 19: Nucleic acid sequence encoding Picrophilus torridus HPPD
SEQ ID No. 20: Protein encoded by SEQ ID No. 19
SEQ ID No. 21: Nucleic acid sequence encoding Kordia algicida HPPD
SEQ ID No. 22: Protein encoded by SEQ ID No. 21
SEQ ID No. 23: Nucleic acid sequence encoding Sorghum bicolor HPPD
SEQ ID No. 24: Protein encoded by SEQ ID No. 23
SEQ ID No. 25: Nucleic acid sequence encoding Triticum aestivum HPPD
SEQ ID No. 26: Protein encoded by SEQ ID No. 25
SEQ ID No. 27: Nucleic acid sequence encoding Hordeum vulgare HPPD
SEQ ID No. 28: Protein encoded by SEQ ID No. 27
SEQ ID No. 29: Nucleic acid sequence encoding Rhodococcus RHA1 isolate ro0240 HPPD
SEQ ID No. 30: Protein encoded by SEQ ID No. 29
SEQ ID No. 31: Nucleic acid sequence encoding Arabidopsis thaliana wild type HPPD polypeptide, plus containing at the 5′ end a nucleic acid encoding an alanine and 6 histidine amino acids
SEQ ID No. 32: Protein encoded by SEQ ID No. 31
SEQ ID No. 33: Nucleic acid sequence encoding mutant HPPD polypeptide, plus containing at the 5′ end a nucleic acid encoding an alanine and 6 histidine amino acids
SEQ ID No. 34: Protein encoded by SEQ ID No. 33,
SEQ ID No. 35: Nucleic acid sequence encoding mutant HPPD polypeptide, plus containing at the 5′ end a nucleic acid encoding an alanine and 6 histidine amino acids
SEQ ID No. 36: Protein encoded by SEQ ID No. 35,
SEQ ID No. 37: Nucleic acid sequence encoding mutant HPPD polypeptide, plus containing at the 5′ end a nucleic acid encoding an alanine and 6 histidine amino acids
SEQ ID No. 38: Protein encoded by SEQ ID No. 37,
SEQ ID No. 39: Nucleic acid sequence encoding mutant HPPD polypeptide, plus containing at the 5′ end a nucleic acid encoding an alanine and 6 histidine amino acids
SEQ ID No. 40: Protein encoded by SEQ ID No. 39,
SEQ ID No. 41: Nucleic acid sequence encoding mutant HPPD polypeptide, plus containing at the 5′ end a nucleic acid encoding an alanine and 6 histidine amino acids
SEQ ID No. 42: Protein encoded by SEQ ID No. 41,
SEQ ID No. 43: Nucleic acid sequence encoding mutant HPPD polypeptide, plus containing at the 5′ end a nucleic acid encoding an alanine and 6 histidine amino acids
SEQ ID No. 44: Protein encoded by SEQ ID No. 43,
SEQ ID No. 45: Nucleic acid sequence encoding mutant HPPD polypeptide, plus containing at the 5′ end a nucleic acid encoding an alanine and 6 histidine amino acids
SEQ ID No. 46: Protein encoded by SEQ ID No. 45
SEQ ID No. 47: Nucleic acid sequence encoding mutant HPPD polypeptide, plus containing at the 5′ end a nucleic acid encoding an alanine and 6 histidine amino acids
SEQ ID No. 48: Protein encoded by SEQ ID No. 47
SEQ ID No. 49: Nucleic acid sequence encoding mutant HPPD polypeptide, plus containing at the 5′ end a nucleic acid encoding an alanine and 6 histidine amino acids
SEQ ID No. 50: Protein encoded by SEQ ID No. 49,

### Example 1

A mutation of strictly conserved amino acids leads to an inactive protein.

The point mutants S267A (SEQ ID No. 34) in A. thaliana and N282A (SEQ ID No. 36) in A. thaliana are inactive. The amino acid position given are referring to the position of SEQ ID No. 2.

Mutant HPPD polypeptides of the presenting invention have amino acids changes at one or more positions relative to the starting wild type sequence from which they are derived.

The DNA sequence SEQ ID No. 2 encoding the wild HPPD protein from Arabidopsis thaliana (1335 bp; Genebank AF047834; WO 96/38567) was cloned into the vector pSE420(RI)NX (modified from the cloning and expression vector pSE420(RI)NX (5261 bp) is based on the plasmid pSE420 by Invitrogen (Karlsruhe, Germany)). At the 5′ end, directly downstream to the ATG was inserted a nucleic acid sequence coding for an alanine amino acid and a nucleic acid sequence encoding a N-terminal HIS6-Tag (6×HIS, encoded by: cat cat cat cac cat cat). The resulting sequence is presented as SEQ ID No. 31. Upstream to the ATG, two additional cysteine base pairs were added in order to obtain a sequence corresponding to the recognition site of the restriction enzyme NcoI and downstream to the stop codon the sequences corresponding to the recognition site of the restriction enzyme XbaI were added. The resulting plasmid was then used to transform E. Coli cells BL21 (DE3) with 50 μg/ml kanamycin or 100 μg/ml carbenicillin selection as described in European patent applications “EPO9015984.9”; EP09015985.6”; EP 09015986.4” EP 09015987.2”; and EP09015988.0” all filed on 23 Dec. 2009.

### Quick Test of the Activity of HPPD Protein: Production of Brown Color

### Colorimetric Screening Test for Active HPPD Enzymes:

A YT-broth-type culture medium with 1% agarose, 5 mM L-Tyrosine and 42 mM Succinate, which contains the selection agent for the vector pSE420 is poured into deep well plates. E. coli culture in the exponentional growth phase which contains the vector pSE420-HPPDx (any gene coding for a putative HPPD enzyme/protein) is applied to each well. After 16 hours at 37° C., the wells which do not contain the culture medium, those which have been seeded with an E. coli culture containing the empty vector pSE420 are transparent, or those which have been seeded with an E. coli culture containing a vector pSE420 containing a gene coding for an inactive HPPD are transparent, while the wells seeded with an E. coli culture containing the vector pSE420-HPPD coding for active HPPD are brown. It has been previously demonstrated that this test reflects the HPPD activity, whatever the origin of this activity, and allows the identification of HPPD activities (U.S. Pat. No. 6,768,044).

As it can be seen on FIG. 3, the culture from bacteria containing the gene coding for the mutant HPPD did not develop a brown color while the one containing the gene coding of the wild type HPPD developed a strong brown color reflecting the activity of the HPPD enzymes. It can be concluded that the two HPPD mutants are not able to convert HPP into homogentisate. The two HPPD mutants are inactive. One can conclude that the amino acid positions 267 and 282 (referring to position in SEQ ID No. 2) are essential for HPPD activity.

The expression of the HPPD protein was then done as following.

Overnight cultures grown at 37° C. were used to inoculate LB media at a ratio 1:100. Cells were allowed to grow until OD reached 0.5, then expression was initiated from the trp-lac (trc) promoter by induction with 1 mM IPTG which binds to the lac repressor and causes its dissociation from the lac operon. Expression was carried out over 15 h at 28° C.

To prepare the pre-starter culture, 2 mL of TB medium (100 μg*mL-1 carbenicillin) were inoculated with 50 μL of an E. coli K-12 BL21 glycerol stock. The pre-starter culture was incubated at 37° C. with shaking at 140 rpm for 15 h. 200 μl of the pre-starter culture was used to initiate the starter culture (5 mL TB supplement with 100 μg*L-1), which was incubated 3 h at 37° C.

To prepare the main culture, 400 mL of TB medium (100 μg*mL-1 carbenicillin) were inoculated with 4 mL of the starter culture. This starter culture was incubated at 37° C. with shaking at 140 rpm until OD600 0.5 was reached. Then recombinant protein expression was induced with 400 μl of 1M IPTG solution. The cells were allowed to grow for an additional hour under these conditions, then the temperature was lowered to 28° C. and the culture was shaken at 140 rpm for 15 h. Cells were harvested by centrifugation at 6000×g for 15 min at 4° C. Then cell pellets were stored at −80° C.

### Isolation and Purification of His6-AtHPPD in Native Form

### Lysis of Cells

Cells were lysed using Lysozyme, an enzyme that cleaves the 1,4-β-linkages between N-acetylmuramic acid and N-acetyl-D-glucosamine residues in peptidoglycan which forms the bacterial cell wall. Cell membranes were then disrupted by the internal pressure of the bacterial cell. In addition, the lysis buffer contained Benzonase® Nuclease, an endonuclease that hydrolyzes all forms of DNA and RNA without damaging proteins and thereby largely reduces viscosity of the cell lysate. Lysis under native conditions was carried out on ice.

For purification of His6-tagged proteins the QIAexpress® Ni-NTA Fast Start Kit was used following the user manual instruction.

### Purification of His6-Tagged Proteins by Immobilized Metal Ion Affinity Chromatography (IMAC)

The cleared cell lysate (10 mL) obtained after centrifugation of the lysis reaction was loaded onto a Ni-NTA Fast Start Column from the QIAexpress® Ni-NTA Fast Start Kit (Qiagen, Hilden, Germany) and purification was carried out according to the instruction manual. The His₆-tagged protein was eluted with 2.5 mL of elution buffer.

### Desalting of HPPD Solutions by Gel Filtration

HPPD solutions eluted from a Ni-NTA Fast Start Column with 2.5 mL of elution buffer were applied to a Sephadex G-25 PD-10 column (GE Healthcare, Freiburg, Germany) following the user manual instruction. After the whole sample had entered the gel bed, elution was performed with 3.5 mL of storage buffer.

The HPPD solutions eluted from the desalting column were frozen at −80° C. in 1 mL aliquots.

### Determination of HPPD Protein Concentration Using the Bradford Protein Assay

Protein concentration was determined using the standard Bradford assay (Bradford, (1976), Anal Biochem 72: 248-254). Determination of purity of HPPD solutions using SDS-PAGE

The integrity of the eluted protein was checked by SDS-PAGE protein gel electrophoresis using the gel NuPAGE® Novex 4-12% Bis-Tris Gels (Invitrogen, Karlsruhe, Germany), approximately 10 μg of protein were loaded. 10 μL of Laemmli Sample Buffer was added to 1-10 μL of protein solution and the mixture was incubated at 90° C. for 10 min. After short centrifugation step, the whole mixture was loaded into a slot of an SDS gel previously fixed in a XCell SureLock™ Novex Mini-Cell gel chamber filled with NuPAGE® MOPS SDS Running Buffer (diluted from the 20 x-solution with ddH2O). A voltage of 150 was then applied to the gel chamber for 1 h. For staining of protein bands, the gel was immersed in Coomassie Brilliant Blue R-250 Staining Solution. For destaining of the polyacrylamide gel, it was immersed in Coomassie Brilliant Blue R-250 Destaining Solution until protein bands appear blue on a white gel.

The HPPD activity was checked by the standard spectrophotmetric assay (method extensively described in WO 2009/144079)

In this content, pI50-value means the log value of the concentration of inhibitor necessary to inhibit 50% of the enzyme activity in molar concentration.

pI50-values for HPPD inhibitors were determined from dose-response plots of HPPD activity versus inhibitor concentration using the assay extensively described in WO 2009/144079 at 2 mM fixed HPP concentration and 3 minutes fixed incubation time using the ID Business Solutions Ltd. XLfit software suite.

TABLE 7
Determination of pI50 HPPD enzymes (Arabidopsis thaliana wild type
HPPD “SEQ ID No. 32”, the mutants S267A (SEQ ID No. 34) and N282A
(SEQ ID No. 36) and the tolerance to the several listed below HPPD
inhibitors tembotrione, diketonitrile, mesotrione, bicyclopyrone, pyrasulfotole,
sulcotrione, pyrazolate, tefuryltrione, and benzofenap.

| | Bicyclopyrone | Benzofenap | Diketonitrile | Mesotrione |
|---|---|---|---|---|
| WT | 5.2 | >5.6 | >5.6 | >5.6 |
| (SEQ ID No. 32) | | | | |
| S267A | nd - la (P) | nd - la (P) | nd - la (P) | nd - la (P) |
| (SEQ ID No. 34) | | | | |
| N282A | nd - la (P) | nd - la (P) | nd - la (P) | nd - la (P) |
| (SEQ ID No. 36) | | | | |

| | Pyrasulfotole | Pyrazolate | Sulcotrione | Tefuryltrione | Tembotrione |
|---|---|---|---|---|---|
| WT | 5.4 | 5.4 | >5.6 | >5.6 | >5.6 |
| (SEQ ID No. 32) | | | | | |
| S267A | nd - la (P) | nd - la (P) | nd - la (P) | nd - la (P) | nd - la (P) |
| (SEQ ID No. 34) | | | | | |
| N282A | nd - la (P) | nd - la (P) | nd - la (P) | nd - la (P) | nd - la (P) |
| (SEQ ID No. 36) | | | | | |

The symbol “>” means that the value was far higher than the one indicated but could not be precisely calculated within in the range of concentration of inhibitor tested (2.5 × 10⁻⁶, 5.0 × 10⁻⁶, 1.0 × 10⁻⁵, 2.5 × 10⁻⁵, 6.3 × 10⁻⁵, and 2.5 × 10⁻⁴ M).

As previously demonstrated, the 2 protein mutants are not able to convert HPP into Homogentisate, confirming that the 2 mutant proteins are inactive. It confirms the hypothesis that the position 267 and 282 (referring to position in SEQ ID No. 2) are absolutely essential for obtaining an active HPPD

### Example 2: Single Point Mutants Displayed Increased Tolerance to HPPD Inhibitor Herbicides

Kinetic characterization and evaluation of tolerance to HPPD inhibitors of HPPD enzyme “SEQ ID No. 32”.

The HPPD activity was checked by the standard spectrophotmetric assay (method extensively described in WO 2009/144079)

### Determination of HPPD In Vitro Kinetic Properties

Km, Vmax, and kcat values for different HPPD enzyme preparations and Ki, K1=Kon, and K−1=Koff for different HPPD inhibitors were determined using a HPLC assay for measurements of HPPD activity. The assay mixtures contained in a volume of 1 ml 150 mM Tris-HCl buffer at pH 7.8, 10 mM sodium ascorbate, 650 units of bovine catalase (Sigma C30 (Sigma-Aldrich, Munich, Germany), 34 mg protein/ml, 23,000 units/mg), and appropriate amounts of HPP, purified HPPD enzyme and HPPD inhibitors. For Km, Vmax, and kcat value determination HPP concentrations in the assay mixture were varied between 10 and 400 μM. For Ki, K1=Kon, and K−1=Koff value determination 2 mM HPP was used. All assays were started by the addition of HPPD enzyme to the assay mixture and stopped at a series of times between 0 and 240 s by addition of 200 μl of the reaction mixture to reaction assay tubes containing 20 μl 10% perchloric acid. Precipitated protein was pelleted by a 5 minute centrifugation at 10,000 g. 100 μl of the supernatant were loaded onto a 250×4 mm Knauer (Berlin, Germany) Eurospher 100-5 C18-column equilibrated with 10% methanol, 0.1% trifluoroacetic acid (buffer A). The column was eluted, also at 1.5 ml/min, using a 4 minute wash with buffer A, followed by a 3 min wash with 95% methanol and by a further 2 minute wash with buffer A. The elution of HGA (homogentisic acid) and HPP (hydroxyphenylpyruvate) was monitored at 292 nm. HGA elutes at around 5 minutes and HPP elutes later. A standard set of concentrations of HGA were used to provide a standard curve in order to calibrate the 292 nm absorbance of the HGA peak versus HGA concentration.

For Km and Vmax value determinations the initial rates of the HPPD reaction at different substrate concentrations were determined from plots of HGA formed versus time and fitted to the Michaelis-Menten equation for unireactant enzymes using the ID Business Solutions Ltd. (www.idbs.com) XLfit software suite. For the determination of Ki, K1=Kon, and K-1=Koff values the time-courses of the HPPD reaction at different inhibitor concentrations were fitted to the equations for Mechanism A, competitive inhibition, for tight-binding inhibitors (Cha, S. (1975) Tight-binding inhibitors—I. Kinetic behaviour. Biochemical Pharmacology 24, 2177-2185) using the ID Business Solutions Ltd. XLfit software suite

TABLE 8
In below given table1, “Km” (Michaelis-Menten constant) means
the kinetic parameter that is used to characterise an enzyme, and it is
defined as the concentration of substrate that permits half maximal rate
of the reaction. Km is further defined as the substrate concentration
at which the reaction rate reaches half of its maximum value (Vmax/2)
where Vmax has the meaning of being the maximum velocity of the reaction.

| | HPP | | Tembotrione | | | Diketonitrile | | |
|---|---|---|---|---|---|---|---|---|
| | Kₘ | Vₘₐₓ | k₁ | k₋₁ | Kᵢ | k₁ | k₋₁ | Kᵢ |
| | (μM) | (μM) | (M⁻¹ s⁻¹) | (s⁻¹) | (μM) | (M⁻¹ s⁻¹) | (s⁻¹) | (μM) |
| SEQ ID | 6.3 | 1.2 | 2.3E+05 | 3.5E−03 | 0.015 | 6.1E+05 | 1.1E−02 | 0.018 |
| No. 32 | | | | | | | | |

Protein SEQ ID No. 32 consists of protein SEQ ID No. 2 with an insertion of one amino acid Ala and six amino acids His directly behind the first methionine.
Kinetic characterization of HPPD enzymes (Arabidopsis thaliana “SEQ ID No. 32” and the respective tolerance to the HPPD inhibitors tembotrione and diketonitrile.
Kon = K1 equals the association rate constant of the enzyme-substrate binding and Koff = K−1 equals the rate constant of the enzyme-inhibitor complex dissociation. Ki defines the inhibition constant.

The kinetic parameters Km and Vmax of the plant HPPD “SEQ ID No. 32” summarized in Table 8 are well in the range of the specific activities measured from purified plant HPPD as for example for the carrot HPPD (Garcia et al. 2000, Biochemistry, 39, 7501-7507). The Arabidopsis thaliana HPPD is also sensitive in the similar range as the carrot HPPD to diketonitrile. It is the first time, that the kinetic parameter of Arabidopsis thaliana HPPD in presence of tembotrione are measured and being in a comparable range as the one measured in presence of the HPPD inhibitor diketonitrile.

### Determination of HPPD Activity in Presence of Several HPPD Inhibitors

In this content, pI50-value means the log value of the concentration of inhibitor necessary to inhibit 50% of the enzyme activity in molar concentration.

pI50-values for HPPD inhibitors were determined from dose-response plots of HPPD activity versus inhibitor concentration using the assay extensively described in WO 2009/144079 at 2 mM fixed HPP concentration and 3 minutes fixed incubation time using the ID Business Solutions Ltd. XLfit software suite.

TABLE 9
Determination of pI50 HPPD enzymes (Arabidopsis thaliana “SEQ ID No.
32” and some Arabidopsis HPPD single point mutants “SEQ ID No.
35, SEQ ID No. 40, SEQ ID No. 42, SEQ ID No. 44, SEQ ID No. 46, SEQ ID No.
48, and SEQ ID No. 50”) and their respective tolerance to the several
listed below HPPD inhibitors tembotrione, diketonitrile, mesotrione, bicyclopyrone,
pyrasulfotole, sulcotrione, pyrazolate, tefuryltrione, and benzofenap.

| | Bicyclopyrone | Benzofenap | Diketonitrile | Mesotrione | Pyrasulfotole |
|---|---|---|---|---|---|
| SEQ ID No. 32 | 5.2 | >5.6 | >5.6 | >5.6 | 5.4 |
| E252S | 4.3 | 5.2 | 4.9 | 5.3 | 4.3 |
| (SEQ ID No. 36) | | | | | |
| V269A | 4.3 | 5.2 | 5.2 | 5.2 | 4.4 |
| (SEQ ID No. 40) | | | | | |
| P280A | 4.3 | 5.2 | 5.2 | 5.3 | 4.4 |
| (SEQ ID No. 42) | | | | | |
| L368M | 4.5 | >5.6 | 5.6 | 5.4 | 5.0 |
| (SEQ ID No. 44) | | | | | |
| K421I | 4.2 | 5.1 | 5.0 | 5.2 | 4.3 |
| (SEQ ID No. 46) | | | | | |
| K421Q | 4.7 | >5.6 | >5.6 | >5.6 | 4.8 |
| (SEQ ID No. 48) | | | | | |
| K421V | 4.2 | 5.2 | 5.1 | 5.3 | 4.4 |
| (SEQ ID No. 50) | | | | | |

| | Pyrazolat | Sulcotrione | Tefuryltrione | Tembotrione |
|---|---|---|---|---|
| SEQ ID No. 32 | 5.4 | >5.6 | >5.6 | >5.6 |
| E252S | 4.7 | 5.1 | 5.2 | 5.5 |
| (SEQ ID No. 35) | | | | |
| V269A | 4.4 | 5.0 | 5.4 | >5.6 |
| (SEQ ID No. 40) | | | | |
| P280A | 4.7 | 5.0 | 5.5 | >5.6 |
| (SEQ ID No. 42) | | | | |
| L368M | 4.9 | 5.2 | >5.6 | >5.6 |
| (SEQ ID No. 44) | | | | |
| K421I | 4.5 | 5.0 | 5.2 | 5.6 |
| (SEQ ID No. 46) | | | | |
| K421Q | 5.0 | >5.6 | >5.6 | >5.6 |
| (SEQ ID No. 48) | | | | |
| K421V | 4.5 | 5.1 | 5.4 | >5.6 |
| (SEQ ID No. 50) | | | | |

The symbol “>” means that the value was far higher than the one indicated but could not be precisely calculated within in the range of concentration of inhibitor tested (2.5 × 10⁻⁶, 5.0 × 10⁻⁶, 1.0 × 10⁻⁵, 2.5 × 10⁻⁵, 6.3 × 10⁻⁵, and 2.5 × 10⁻⁴ M).

Measurements show that the pI50 in case of the point mutants is reduced compared to the wild-type protein.

In Table 9, it can be clearly seen that the HPPD mutants are all affected in their tolerance to at least one HPPD inhibitors and some to several HPPD inhibitors. All the tested mutants are more active (do show an increased tolerance concerning the various applied HPPD inhibitors) in the presence of at least one of the selected HPPD inhibitors, therefore the mutant enzyme-inhibitor complex should be less stable the wild-type enzyme-inhibitor complex. It can be concluded that the positions 252, 269, 280, 368 and 421 (reference taken in SEQ ID No. 2) are all important in the modulation of tolerance of HPPD enzyme to HPPD inhibitors.

1. An isolated nucleic acid comprising a nucleotide sequence encoding a mutated HPPD protein,
wherein said mutated HPPD protein has HPPD activity, and
wherein in said mutated HPPD protein at least one amino acid has been replaced so that the resulting amino acid sequence comprises at least one amino acid selected from:
a) Ala, Asp, Glu, Phe, Gly, Lys, Gln, Arg, Ser, Thr, Val or Tyr at a position in an HPPD protein, said position corresponding to position 250 of the amino acid sequence of SEQ ID No. 2;
b) Ala, Asp, Glu, Ile, Lys, Leu, Asn, Pro, Arg or Ser at a position in an HPPD protein, said position corresponding to position 251 of the amino acid sequence of SEQ ID No. 2;
c) Glu, Phe, Gly, His, Ile, Leu, Met, Asn, Gln, Arg, Ser, Val or Tyr at a position in an HPPD protein, said position corresponding to position 252 of the amino acid sequence of SEQ ID No. 2;
d) Ala, Phe, His, Gln, Val or Trp at a position in an HPPD protein, said position corresponding to position 253 of the amino acid sequence of SEQ ID No. 2;
e) Leu or Val at a position in an HPPD protein, said position corresponding to position 265 of the amino acid sequence of SEQ ID No. 2;
f) Leu, Gln, Arg, Val or Tyr at a position in an HPPD protein, said position corresponding to position 268 of the amino acid sequence of SEQ ID No. 2;
g) Ala, Thr or Val at a position in an HPPD protein, said position corresponding to position 269 of the amino acid sequence of SEQ ID No. 2;
h) Ala, Ile, Asn, Pro, Thr or Val at a position in an HPPD protein, said position corresponding to position 280 of the amino acid sequence of SEQ ID No. 2;
i) Leu, Gln or Val at a position in an HPPD protein, said position corresponding to position 293 of the amino acid sequence of SEQ ID No. 2;
j) Ile or Met at a position in an HPPD protein, said position corresponding to position 294 of the amino acid sequence of SEQ ID No. 2;
k) Gln at a position in an HPPD protein, said position corresponding to position 307 of the amino acid sequence of SEQ ID No. 2;
l) Ile, Met or Asn at a position in an HPPD protein, said position corresponding to position 335 of the amino acid sequence of SEQ ID No. 2;
m) Leu at a position in an HPPD protein, said position corresponding to position 368 of the amino acid sequence of SEQ ID No. 2;
n) Phe or Tyr at a position in an HPPD protein, said position corresponding to position 381 of the amino acid sequence of SEQ ID No. 2;
o) Phe or Ser at a position in an HPPD protein, said position corresponding to position 392 of the amino acid sequence of SEQ ID No. 2;
p) Phe or Tyr at a position in an HPPD protein, said position corresponding to position 419 of the amino acid sequence of SEQ ID No. 2;
q) Asp, Lys or Asn at a position in an HPPD protein, said position corresponding to position 421 of the amino acid sequence of SEQ ID No. 2;
r) Ala, Gly, Met, Pro or Thr at a position in an HPPD protein, said position corresponding to position 422 of the amino acid sequence of SEQ ID No. 2;
s) Ala, Phe, Ile or Val at a position in an HPPD protein, said position corresponding to position 424 of the amino acid sequence of SEQ ID No. 2;
t) Ile, Pro, Arg or Ser at a position in an HPPD protein, said position corresponding to position 425 of the amino acid sequence of SEQ ID No. 2;
u) Glu, Phe, Thr or Val at a position in an HPPD protein, said position corresponding to position 426 of the amino acid sequence of SEQ ID No. 2;
v) Ile, Met, Gln or Val at a position in an HPPD protein, said position corresponding to position 431 of the amino acid sequence of SEQ ID No. 2; and;
w) at least one amino acid deletion or replacement at any one of positions 228, 248, 270, 271, 379 and/or 427.

2. The isolated nucleic acid of claim 1, wherein said at least one amino acid is selected from:
a) Ala, Asp, Glu, Phe, Thr, Val or Tyr at a position in an HPPD protein, said position corresponding to position 250 of the amino acid sequence of SEQ ID No. 2;
b) Ala, Asp, Glu, Ile, Lys, Leu, Pro or Arg at a position in an HPPD protein, said position corresponding to position 251 of the amino acid sequence of SEQ ID No. 2;
C) Glu, Thr, Arg, Ser or Tyr at a position in an HPPD protein, said position corresponding to position 252 of the amino acid sequence of SEQ ID No. 2;
d) Ala, Phe, Val or Trp at a position in an HPPD protein, said position corresponding to position 253 of the amino acid sequence of SEQ ID No. 2;
e) Leu or Val at a position in an HPPD protein, said position corresponding to position 265 of the amino acid sequence of SEQ ID No. 2;
f) Leu, Gln, Arg, Val or Tyr at a position in an HPPD protein, said position corresponding to position 268 of the amino acid sequence of SEQ ID NO. 2;
g) Ala or Val at a position in an HPPD protein, said position corresponding to position 269 of the amino acid sequence of SEQ ID No. 2;
h) Ala, Pro, Thr or Val at a position in an HPPD protein, said position corresponding to position 280 of the amino acid sequence of SEQ ID No. 2;
i) Gln at a position in an HPPD protein, said position corresponding to position 307 of the amino acid sequence of SEQ ID No. 2;
j) Ile or Met at a position in an HPPD protein, said position corresponding to position 335 of the amino acid sequence of SEQ ID No. 2;
k) Leu at a position in an HPPD protein, said position corresponding to position 368 of the amino acid sequence of SEQ ID No. 2;
l) Phe at a position in an HPPD protein, said position corresponding to position 392 of the amino acid sequence of SEQ ID No. 2;
m) Asp, Lys, or Asn at a position in an HPPD protein, said position corresponding to position 421 of the amino acid sequence of SEQ ID No. 2;
n) Ala or Gly at a position in an HPPD protein, said position corresponding to position 422 of the amino acid sequence of SEQ ID No. 2;
o) Ile, Arg or Ser at a position in an HPPD protein, said position corresponding to position 425 of the amino acid sequence of SEQ ID No. 2
p) Glu, Phe or Val at a position in an HPPD protein, said position corresponding to position 426 of the amino acid sequence of SEQ ID No. 2;
q) Ile, Met, Gln or Val at a position in an HPPD protein, said position corresponding to position 431 of the amino acid sequence of SEQ ID No. 2;
r) Ala, Cys, Thr, Val or Gly at a position in an HPPD protein, said position corresponding to position 228 of the amino acid sequence of SEQ ID No. 2;
s) Ala, Glu, His, Lys, Gln, Arg, Thr, Gly, Leu, Asn, Ser or Tyr at a position in an HPPD protein, said position corresponding to position 248 of the amino acid sequence of SEQ ID No. 2;
t) Ala, Ile, Leu, Met or Val at a position in an HPPD protein, said position corresponding to position 270 of the amino acid sequence of SEQ ID No. 2;
u) Ala, Glu, Lys, Arg, Ser, Thr, His, Ile, Leu, Met or Val at a position in an HPPD protein, said position corresponding to position 271 of the amino acid sequence of SEQ ID No. 2;
v) His or Gln at a position in an HPPD protein, said position corresponding to position 379 of the amino acid sequence of SEQ ID No. 2; and
w) Leu or Arg at a position in an HPPD protein, said position corresponding to position 427 of the amino acid sequence of SEQ ID No. 2.

3. The nucleic acid of claim 1, wherein said at least one amino acid is selected from:
a) Glu, Thr, Arg, Ser or Tyr at a position in an HPPD protein, said position corresponding to position 252 of the amino acid sequence of SEQ ID No. 2;
b) Ala or Val at a position in an HPPD protein, said position corresponding to position 269 of the amino acid sequence of SEQ ID No. 2;
c) Ala, Pro, Thr or Val at a position in an HPPD protein, said position corresponding to position 280 of the amino acid sequence of SEQ ID No. 2;
d) Ile or Met at a position in an HPPD protein, said position corresponding to position 335 of the amino acid sequence of SEQ ID No. 2;
e) Leu at a position in an HPPD protein, said position corresponding to position 368 of the amino acid sequence of SEQ ID No. 2;
f) Asp, Asn or Lys at a position in an HPPD protein, said position corresponding to position 421 of the amino acid sequence of SEQ ID No. 2;
g) Ala or Gly at a position in an HPPD protein, said position corresponding to position 422 of the amino acid sequence of SEQ ID No. 2;
h) Ala, Cys, Thr or Val at a position in an HPPD protein, said position corresponding to position 228 of the amino acid sequence of SEQ ID No. 2;
i) Ala, Glu, His, Lys, Gln, Arg, Thr or Tyr at a position in an HPPD protein, said position corresponding to position 248 of the amino acid sequence of SEQ ID No. 2;
j) Ile, Leu, Met or Val at a position in an HPPD protein, said position corresponding to position 270 of the amino acid sequence of SEQ ID No. 2;
k) Ala, Glu, Lys, Arg, Ser, Thr or Val at a position in an HPPD protein, said position corresponding to position 271 of the amino acid sequence of SEQ ID No. 2;
l) His or Gln at a position in an HPPD protein, said position corresponding to position 379 of the amino acid sequence of SEQ ID No. 2; and
m) Leu or Arg at a position in an HPPD protein, said position corresponding to position 427 of the amino acid sequence of SEQ ID No. 2.

4. The isolated nucleic acid of claim 1, wherein said HPPD protein is derived from Streptomyces avermitilis (Genebank SAV11864), Daucus carota (Genebank DCU 87257), Arabidopsis thaliana (Genebank AF047834), Mycosphaerella graminicola (Genebank AF038152), Oryza sativa/rice [BAD26248], Zea mays/corn [ACN36372], Avena sativa [ABZ23427], Pseudomonas fluorescens [ABF50055], Synechococcus sp. [YP_473959], Blepharisma japonicum [BAF91881], Rhodococcus RHA1 sp. ro0240 [YP_702005], Rhodococcus RHA1 sp. ro0341 [YP_703002], Picrophilus torridus [YP_024147], Kordia algicida [ZP_02161490], Sorghum bicolor [XP_002453359], Triticum aestivum/wheat [AAZ67144], or Hordeum vulgare/barley [O48604].

5. The isolated nucleic acid of claim 1, wherein said HPPD protein comprises the amino acid sequence of SEQ ID No. 4 and wherein the resulting amino acid sequence comprises at least one amino acid selected from:
a) Ala, Asp, Glu, Gly, Lys, Gln, Arg, Ser, Thr, Val or Tyr, at position 247 of the amino acid sequence of SEQ ID No. 4 (corresponding to position 250 of SEQ ID No. 2);
b) Asp, Glu, Ile, Lys, Leu, Asn, Pro, Arg or Ser, at position 248 of the amino acid sequence of SEQ ID No. 4 (corresponding to position 251 of SEQ ID No. 2);
c) Phe, Gly, His, Ile, Leu, Met, Asn, Gln, Arg, Ser, Val or Tyr at position 249 of the amino acid sequence of SEQ ID No. 4 (corresponding to position 252 of SEQ ID No. 2);
d) Ala, His, Gln, Trp or Val, at position 250 of the amino acid sequence of SEQ ID No. 4 (corresponding to position 253 of SEQ ID No. 2);
e) Val at position 262 of the amino acid sequence of SEQ ID No. 4 (corresponding to position 265 of SEQ ID No. 2);
f) Leu, Gln, Arg or Tyr, at position 265 of the amino acid sequence of SEQ ID No. 4 (corresponding to position 268 of SEQ ID No. 2);
g) Ala or Thr at position 266 of the amino acid sequence of SEQ ID No. 4 (corresponding to position 269 of SEQ ID No. 2);
h) Ala, Ile, Asn, Thr or Val at position 277 of the amino acid sequence of SEQ ID No. 4 (corresponding to position 280 of SEQ ID No. 2);
i) Leu or Val, at position 290 of the amino acid sequence of SEQ ID No. 4 (corresponding to position 293 of SEQ ID No. 2);
j) Met at position 291 of the amino acid sequence of SEQ ID No. 4 (corresponding to position 294 of SEQ ID No. 2);
k) Ile, Met or Asn at position 332 of the amino acid sequence of SEQ ID No. 4 (corresponding to position 335 of SEQ ID No. 2);
l) Tyr at position 378 of the amino acid sequence of SEQ ID No. 4 (corresponding to position 381 of SEQ ID No. 2);
m) Ser at position 389 of the amino acid sequence of SEQ ID No. 4 (corresponding to position 392 of SEQ ID No. 2);
n) Tyr at position 416 of the amino acid sequence of SEQ ID No. 4 (corresponding to position 419 of SEQ ID No. 2);
o) Asp or Asn, at position 418 of the amino acid sequence of SEQ ID No. 4 (corresponding to position 421 of SEQ ID No. 2);
p) Ala, Met, Pro or Thr at position 419 of the amino acid sequence of SEQ ID No. 4 (corresponding to position 422 of SEQ ID No. 2);
q) Ala, Ile or Val at position 421 of the amino acid sequence of SEQ ID No. 4 (corresponding to position 424 of SEQ ID No. 2);
r) Ile, Pro or Arg, at position 422 of the amino acid sequence of SEQ ID No. 4 (corresponding to position 425 of SEQ ID No. 2);
s) Phe, Thr or Val, at position 423 of the amino acid sequence of SEQ ID No. 4 (corresponding to position 426 of SEQ ID No. 2);
t) Met, Gln or Val at position 428 of the amino acid sequence of SEQ ID No. 4 (corresponding to position 431 of SEQ ID No. 2);
u) Ala, Cys, Gly or Thr at position 225 of the amino acid sequence of SEQ ID No. 4 (corresponding to position 228 of SEQ ID No. 2);
v) Ala, Glu, Gly, Lys, Leu, Asn, Gln, Arg, Ser, Thr or Tyr at position 245 of the amino acid sequence of SEQ ID No. 4 (corresponding to position 248 of SEQ ID No. 2);
w) Ala, Ile, Met or Val at position 267 of the amino acid sequence of SEQ ID No. 4 (corresponding to position 270 of SEQ ID No. 2);
x) Glu, His, Ile, Lys, Leu, Met, Arg, Ser, Thr or Val at position 268 of the amino acid sequence of SEQ ID No. 4 (corresponding to position 271 of SEQ ID No. 2);
y) His at position 376 of the amino acid sequence of SEQ ID No. 4 (corresponding to position 379 of SEQ ID No. 2); and
z) Arg at a position in an HPPD protein, said position corresponding to position 424 of the amino acid sequence of SEQ ID No. 4 (corresponding to position 427 of SEQ ID No. 2).

6. The isolated nucleic acid of claim 1, wherein said HPPD protein comprises the amino acid sequence of SEQ ID No. 6 and wherein the resulting amino acid sequence comprises at least one amino acid selected from:
a) Ala, Asp, Glu, Gly, Lys, Gln, Arg, Ser, Thr, Val or Tyr at position 224 of the amino acid sequence of SEQ ID No. 6 (corresponding to position 250 of SEQ ID No. 2);
b) Asp, Glu, Ile, Lys, Leu, Asn, Pro, Arg or Ser at position 225 of the amino acid sequence of SEQ ID No. 6 (corresponding to position 251 of SEQ ID No. 2);
c) Phe, Gly, His, Ile, Leu, Met, Asn, Gln, Arg, Ser, Val or Tyr at position 226 of the amino acid sequence of SEQ ID No. 6 (corresponding to position 252 of SEQ ID No. 2);
d) Ala, His, Gln, Val or Trp at position 227 of the amino acid sequence of SEQ ID No. 6 (corresponding to position 253 of SEQ ID No. 2);
e) Val at position 239 of the amino acid sequence of SEQ ID No. 6 (corresponding to position 265 of SEQ ID No. 2);
f) Leu, Gln, Arg, Val or Tyr at position 242 of the amino acid sequence of SEQ ID No. 6 (corresponding to position 268 of SEQ ID No. 2);
g) Ala or Thr at position 243 of the amino acid sequence of SEQ ID No. 6 (corresponding to position 269 of SEQ ID No. 2);
h) Ala, Ile, Asn, Thr or Val at position 254 of the amino acid sequence of SEQ ID No. 6 (corresponding to position 280 of SEQ ID No. 2);
i) Leu or Val at position 267 of the amino acid sequence of SEQ ID No. 6 (corresponding to position 293 of SEQ ID No. 2);
j) Met at position 268 of the amino acid sequence of SEQ ID No. 6 (corresponding to position 294 of SEQ ID No. 2);
k) Ile or Asn at position 309 of the amino acid sequence of SEQ ID No. 6 (corresponding to position 335 of SEQ ID No. 2);
l) Tyr at position 355 of the amino acid sequence of SEQ ID No. 6 (corresponding to position 381 of SEQ ID No. 2);
m) Ser at position 366 of the amino acid sequence of SEQ ID No. 6 (corresponding to position 392 of SEQ ID No. 2);
n) Tyr at position 393 of the amino acid sequence of SEQ ID No. 6 (corresponding to position 419 of SEQ ID No. 2);
o) Asp or Asn at position 395 of the amino acid sequence of SEQ ID No. 6 (corresponding to position 421 of SEQ ID No. 2);
p) Ala, Met, Pro or Thr at position 396 of the amino acid sequence of SEQ ID No. 6 (corresponding to position 422 of SEQ ID No. 2);
q) Ala, Ile or Val at position 398 of the amino acid sequence of SEQ ID No. 6 (corresponding to position 424 of SEQ ID No. 2);
r) Ile, Pro or Arg at position 399 of the amino acid sequence of SEQ ID No. 6 (corresponding to position 425 of SEQ ID No. 2);
s) Glu, Phe, Thr or Val at position 400 of the amino acid sequence of SEQ ID No. 6 (corresponding to position 426 of SEQ ID No. 2);
t) Met, Gln or Val at position 405 of the amino acid sequence of SEQ ID No. 6 (corresponding to position 431 of SEQ ID No. 2);
u) Ala, Cys, Gly or Thr at a position in an HPPD protein, said position corresponding to position 202 of the amino acid sequence of SEQ ID No. 6 (corresponding to position 228 of SEQ ID No. 2);
v) Ala, Glu, Gly, Lys, Leu, Asn, Gln, Arg, Ser, Thr or Tyr at position 222 of the amino acid sequence of SEQ ID No. 6 (corresponding to position 248 of SEQ ID No. 2);
w) Ala, Ile, Met or Val at position 244 of the amino acid sequence of SEQ ID No. 6 (corresponding to position 270 of SEQ ID No. 2);
x) Glu, His, Ile, Lys, Leu, Met, Arg, Ser, Thr or Val at position 245 of the amino acid sequence of SEQ ID No. 6 (corresponding to position 271 of SEQ ID No. 2);
y) Gln at position 353 of the amino acid sequence of SEQ ID No. 6 (corresponding to position 379 of SEQ ID No. 2); and
z) Arg at position 401 of the amino acid sequence of SEQ ID No. 6 (corresponding to position 427 of SEQ ID No. 2).

7. The isolated nucleic acid of claim 1, wherein said HPPD protein comprises the amino acid sequence of SEQ ID No. 8 and wherein the resulting amino acid sequence comprises at least one amino acid selected from:
a) Ala, Asp, Glu, Gly, Lys, Gln, Arg, Ser, Thr, Val or Tyr at position 241 of the amino acid sequence of SEQ ID No. 8 (corresponding to position 250 of SEQ ID No. 2), optionally Ala;
b) Asp, Glu, Ile, Lys, Leu, Asn, Pro, Arg or Ser at position 242 of the amino acid sequence of SEQ ID No. 8 (corresponding to position 251 of SEQ ID No. 2), optionally Arg or Lys;
C) Phe, Gly, His, Ile, Leu, Met, Asn, Gln, Arg, Ser, Val or Tyr at position 243 of the amino acid sequence of SEQ ID No. 8 (corresponding to position 252 of SEQ ID No. 2), optionally Tyr;
d) Ala, His, Gln, Val or Trp at position 244 of the amino acid sequence of SEQ ID No. 8 (corresponding to position 253 of SEQ ID No. 2), optionally Val;
e) Val at position 256 of the amino acid sequence of SEQ ID No. 8 (corresponding to position 265 of SEQ ID No. 2);
f) Leu, Gln, Arg or Tyr at position 259 of the amino acid sequence of SEQ ID No. 8 (corresponding to position 268 of SEQ ID No. 2);
g) Ala or Thr at position 260 of the amino acid sequence of SEQ ID No. 8 (corresponding to position 269 of SEQ ID No. 2), optionally Ala;
h) Ala, Ile, Asn, Thr or Val at position 271 of the amino acid sequence of SEQ ID No. 8 (corresponding to position 280 of SEQ ID No. 2);
i) Leu or Val at position 284 of the amino acid sequence of SEQ ID No. 8 (corresponding to position 293 of SEQ ID No. 2);
j) Met at position 285 of the amino acid sequence of SEQ ID No. 8 (corresponding to position 294 of SEQ ID No. 2);
k) Ile at position 326 of the amino acid sequence of SEQ ID No. 8 (corresponding to position 335 of SEQ ID No. 2);
l) Tyr at position 372 of the amino acid sequence of SEQ ID No. 8 (corresponding to position 381 of SEQ ID No. 2);
m) Ser at position 383 of the amino acid sequence of SEQ ID No. 8 (corresponding to position 392 of SEQ ID No. 2);
n) Tyr at position 410 of the amino acid sequence of SEQ ID No. 8 (corresponding to position 419 of SEQ ID No. 2);
o) Asp or Asn at position 412 of the amino acid sequence of SEQ ID No. 8 (corresponding to position 421 of SEQ ID No. 2), optionally Lys;
p) Ala, Met, Pro or Thr at position 413 of the amino acid sequence of SEQ ID No. 8 (corresponding to position 422 of SEQ ID No. 2);
q) Ala, Ile or Val at position 415 of the amino acid sequence of SEQ ID No. 8 (corresponding to position 424 of SEQ ID No. 2);
r) Ile, Pro or Arg at position 416 of the amino acid sequence of SEQ ID No. 8 (corresponding to position 425 of SEQ ID No. 2);
s) Phe, Thr or Val at position 417 of the amino acid sequence of SEQ ID No. 8 (corresponding to position 426 of SEQ ID No. 2);
v) Met, Gln, or Val at position 422 of the amino acid sequence of SEQ ID No. 8 (corresponding to position 431 of SEQ ID No. 2).
w) Ala, Cys, Gly or Thr at position 219 of the amino acid sequence of SEQ ID No. 8 (corresponding to position 228 of SEQ ID No. 2);
x) Ala, Glu, Gly, Lys, Leu, Asn, Gln, Arg, Ser, Thr or Tyr at position 239 of the amino acid sequence of SEQ ID No. 8 (corresponding to position 248 of SEQ ID No. 2);
y) Ala, Ile, Met or Val at position 261 of the amino acid sequence of SEQ ID No. 8 (corresponding to position 270 of SEQ ID No. 2);
z) Glu, His, Ile, Lys, Leu, Met, Arg, Ser, Thr or Val at position 262 of the amino acid sequence of SEQ ID No. 8 (corresponding to position 271 of SEQ ID No. 2);
aa) His at position 370 of the amino acid sequence of SEQ ID No. 8 (corresponding to position 379 of SEQ ID No. 2); and
bb) Arg at position 418 of the amino acid sequence of SEQ ID No. 8 (corresponding to position 427 of SEQ ID No. 2).

8. The isolated nucleic acid of claim 1, wherein said HPPD protein comprises the amino acid sequence of SEQ ID No. 10 and wherein the resulting amino acid sequence comprises at least one amino acid selected from:
a) Phe, Asp, Glu, Gly, Lys, Gln, Arg, Ser, Thr, Val or Tyr at position 188 of the amino acid sequence of SEQ ID No. 10 (corresponding to position 250 of SEQ ID No. 2);
b) Ala, Asp, Glu, Ile, Lys, Leu, Asn, Pro or Ser at position 189 of the amino acid sequence of SEQ ID No. 10 (corresponding to position 251 of SEQ ID No. 2), optionally Ala or Lys;
C) Phe, Gly, His, Ile, Leu, Met, Asn, Gln, Arg, Ser, Val or Glu at position 190 of the amino acid sequence of SEQ ID No. 10 (corresponding to position 252 of SEQ ID No. 2), optionally Ser;
d) Ala, His, Gln, Val or Trp at position 191 of the amino acid sequence of SEQ ID No. 10 (corresponding to position 253 of SEQ ID No. 2), optionally Phe or Val;
e) Val at position 200 of the amino acid sequence of SEQ ID No. 10 (corresponding to position 265 of SEQ ID No. 2);
f) Leu, Gln, Arg, Val or Tyr at position 203 of the amino acid sequence of SEQ ID No. 10 (corresponding to position 268 of SEQ ID No. 2);
g) Thr or Val at position 204 of the amino acid sequence of SEQ ID No. 10 (corresponding to position 269 of SEQ ID No. 2), optionally Val;
h) Ala, Ile, Asn, Thr or Val at position 215 of the amino acid sequence of SEQ ID No. 10 (corresponding to position 280 of SEQ ID No. 2);
i) Leu or Val at position 226 of the amino acid sequence of SEQ ID NO:SEQ ID No. 10 (corresponding to position 293 of SEQ ID No. 2);
j) Met at position 227 of the amino acid sequence of SEQ ID No. 10 (corresponding to position 294 of SEQ ID No. 2);
k) Ile or Asn at position 264 of the amino acid sequence of SEQ ID No. 10 (corresponding to position 335 of SEQ ID No. 2);
l) Tyr at position 312 of the amino acid sequence of SEQ ID No. 10 (corresponding to position 381 of SEQ ID No. 2);
m) Ser at position 321 of the amino acid sequence of SEQ ID No. 10 (corresponding to position 392 of SEQ ID No. 2);
n) Tyr at position 333 of the amino acid sequence of SEQ ID No. 10 (corresponding to position 419 of SEQ ID No. 2);
o) Asp, Lys or Asn at position 335 of the amino acid sequence of SEQ ID No. 10 (corresponding to position 421 of SEQ ID No. 2), optionally Lys;
p) Ala, Met, Pro or Thr at position 336 of the amino acid sequence of SEQ ID No. 10 (corresponding to position 422 of SEQ ID No. 2);
q) Ala, Ile or Val at position 338 of the amino acid sequence of SEQ ID No. 10 (corresponding to position 424 of SEQ ID No. 2);
r) Ile or Pro at position 339 of the amino acid sequence of SEQ ID No. 10 (corresponding to position 425 of SEQ ID No. 2);
s) Phe, Thr or Val at position 340 of the amino acid sequence of SEQ ID No. 10 (corresponding to position 426 of SEQ ID No. 2);
t) Met, Gln or Val at position 345 of the amino acid sequence of SEQ ID No. 10 (corresponding to position 431 of SEQ ID No. 2)
u) Ala, Cys, Gly or Val at position 164 of the amino acid sequence of SEQ ID No. 10 (corresponding to position 228 of SEQ ID No. 2);
v) Ala, Glu, Gly, His, Lys, Leu, Asn, Gln, Ser, Thr or Tyr at position 186 of the amino acid sequence of SEQ ID No. 10 (corresponding to position 248 of SEQ ID No. 2);
w) Ala, Ile, Leu or Val at position 205 of the amino acid sequence of SEQ ID No. 10 (corresponding to position 270 of SEQ ID No. 2);
x) Ala, Glu, His, Ile, Lys, Leu, Met, Arg, Thr or Val at position 206 of the amino acid sequence of SEQ ID No. 10 (corresponding to position 271 of SEQ ID No. 2);
y) His at position 310 of the amino acid sequence of SEQ ID No. 10 (corresponding to position 379 of SEQ ID No. 2); and
z) Arg at position 341 of the amino acid sequence of SEQ ID No. 10 (corresponding to position 427 of SEQ ID No. 2).

9. The isolated nucleic acid of claim 1, wherein said HPPD protein comprises the amino acid sequence of SEQ ID No. 14 and wherein the resulting amino acid sequence comprises at least one amino acid selected from
a) Ala, Asp, Glu, Phe, Gly, Lys, Gln, Arg, Ser, Thr, Val or Tyr at position 169 of the amino acid sequence of SEQ ID No. 14 (corresponding to position 250 of SEQ ID No. 2), preferably Ala or Phe;
b) Ala, Asp, Glu, Ile, Lys, Leu, Asn, Pro, Arg or Ser at position 170 of the amino acid sequence of SEQ ID No. 14 (corresponding to position 251 of SEQ ID No. 2), optionally Ala, Arg or Lys;
C) Glu, Phe, Gly, His, Ile, Leu, Met, Asn, Gln, Ser, Val or Tyr at position 171 of the amino acid sequence of SEQ ID No. 14 (corresponding to position 252 of SEQ ID No. 2), optionally Glu, Ser or Tyr;
d) Ala, Phe, His, Gln or Val at position 172 of the amino acid sequence of SEQ ID No. 14 (corresponding to position 253 of SEQ ID No. 2), optionally Phe or Val;
e) Val at position 181 of the amino acid sequence of SEQ ID No. 14 (corresponding to position 265 of SEQ ID No. 2);
f) Leu, Gln, Arg or Tyr at position 184 of the amino acid sequence of SEQ ID No. 14 (corresponding to position 268 of SEQ ID No. 2);
g) Ala or Thr at position 185 of the amino acid sequence of SEQ ID No. 14 (corresponding to position 269 of SEQ ID No. 2), optionally Ala;
h) Ile, Asn, Pro, Thr or Val at position 196 of the amino acid sequence of SEQ ID No. 14 (corresponding to position 280 of SEQ ID No. 2), optionally Pro;
i) Leu or Val at position 206 of the amino acid sequence of SEQ ID No. 14 (corresponding to position 293 of SEQ ID No. 2);
j) Met at position 207 of the amino acid sequence of SEQ ID No. 14 (corresponding to position 294 of SEQ ID No. 2);
k) Ile, Met or Asn at position 244 of the amino acid sequence of SEQ ID No. 14 (corresponding to position 335 of SEQ ID No. 2), optionally Met;
l) Phe or Tyr at position 301 of the amino acid sequence of SEQ ID No. 14 (corresponding to position 381 of SEQ ID No. 2);
m) Ser at position 312 of the amino acid sequence of SEQ ID No. 14 (corresponding to position 392 of SEQ ID No. 2);
n) Tyr at position 325 of the amino acid sequence of SEQ ID No. 14 (corresponding to position 419 of SEQ ID No. 2);
o) Asp, Lys or Asn at position 327 of the amino acid sequence of SEQ ID No. 14 (corresponding to position 421 of SEQ ID No. 2), optionally Lys;
p) Gly, Met, Pro or Thr at position 328 of the amino acid sequence of SEQ ID No. 14 (corresponding to position 422 of SEQ ID No. 2);
q) Ala, Ile or Val at position 330 of the amino acid sequence of SEQ ID No. 14 (corresponding to position 424 of SEQ ID No. 2);
r) Ile, Pro, Arg or Ser at position 331 of the amino acid sequence of SEQ ID No. 14 (corresponding to position 425 of SEQ ID No. 2);
s) Glu, Phe, Thr or Val at position 332 of the amino acid sequence of SEQ ID No. 14 (corresponding to position 426 of SEQ ID No. 2), optionally Glu;
t) Ile, Met, Gln or Val at position 337 of the amino acid sequence of SEQ ID No. 14 (corresponding to position 431 of SEQ ID No. 2);
u) Ala, Cys, Gly or Thr at position 145 of the amino acid sequence of SEQ ID No. 14 (corresponding to position 228 of SEQ ID No. 2);
v) Ala, Glu, Gly, His, Lys, Leu, Asn, Gln, Ser, Thr or Tyr at position 167 of the amino acid sequence of SEQ ID No. 14 (corresponding to position 248 of SEQ ID No. 2);
w) Ala, Ile, Leu or Met at position 186 of the amino acid sequence of SEQ ID No. 14 (corresponding to position 270 of SEQ ID No. 2);
x) Ala, Glu, His, Ile, Lys, Leu, Met, Arg, Ser, Thr or Val at position 187 of the amino acid sequence of SEQ ID No. 14 (corresponding to position 271 of SEQ ID No. 2);
y) His at a position at position 299 of the amino acid sequence of SEQ ID No. 14 (corresponding to position 379 of SEQ ID No. 2); and
z) Arg at a position at position 333 of the amino acid sequence of SEQ ID No. 14 (corresponding to position 427 of SEQ ID No. 2).

10. The isolated nucleic acid of claim 1, wherein said HPPD protein comprises the amino acid sequence of SEQ ID No. 16 and wherein the resulting amino acid sequence comprises at least one amino acid selected from:
a) Ala, Asp, Glu, Phe, Gly, Lys, Gln, Arg, Ser, Thr or Val at position 209 of the amino acid sequence of SEQ ID No. 16 (corresponding to position 250 of SEQ ID No. 2), optionally Ala or Phe;
b) Ala, Asp, Glu, Ile, Lys, Leu, Asn, Pro, Arg or Ser at position 210 of the amino acid sequence of SEQ ID No. 16 (corresponding to position 251 of SEQ ID No. 2), optionally Ala, Arg, Lys;
C) Glu, Phe, Gly, His, Ile, Leu, Met, Asn, Gln, Arg, Val or Tyr at position 211 of the amino acid sequence of SEQ ID No. 16 (corresponding to position 252 of SEQ ID No. 2), optionally Tyr;
d) Phe, His, Gln, Val or Trp at position 212 of the amino acid sequence of SEQ ID No. 16 (corresponding to position 253 of SEQ ID No. 2), optionally Phe or Val;
e) Val at position 224 of the amino acid sequence of SEQ ID No. 16 (corresponding to position 265 of SEQ ID No. 2);
f) Leu, Gln, Arg or Tyr at position 227 of the amino acid sequence of SEQ ID No. 16 (corresponding to position 268 of SEQ ID No. 2);
g) Ala or Thr at position 228 of the amino acid sequence of SEQ ID No. 16 (corresponding to position 269 of SEQ ID No. 2), optionally Ala;
h) Ala, Ile, Asn, Thr or Val at position 239 of the amino acid sequence of SEQ ID No. 16 (corresponding to position 280 of SEQ ID No. 2);
i) Leu or Val at position 251 of the amino acid sequence of SEQ ID No. 16 (corresponding to position 293 of SEQ ID No. 2);
j) Met at position 252 of the amino acid sequence of SEQ ID No. 16 (corresponding to position 294 of SEQ ID No. 2);
k) Ile, Met or Asn at position 289 of the amino acid sequence of SEQ ID No. 16 (corresponding to position 335 of SEQ ID No. 2), optionally Met;
l) Tyr at position 336 of the amino acid sequence of SEQ ID No. 16 (corresponding to position 381 of SEQ ID No. 2);
m) Ser at position 347 of the amino acid sequence of SEQ ID No. 16 (corresponding to position 392 of SEQ ID No. 2);
n) Tyr at position 359 of the amino acid sequence of SEQ ID No. 16 (corresponding to position 419 of SEQ ID No. 2);
o) Asp, Lys or Asn at position 361 of the amino acid sequence of SEQ ID No. 16 (corresponding to position 421 of SEQ ID No. 2), optionally Lys;
p) Ala, Met, Pro or Thr at position 362 of the amino acid sequence of SEQ ID No. 16 (corresponding to position 422 of SEQ ID No. 2);
q) Ala, Ile or Val at position 364 of the amino acid sequence of SEQ ID No. 16 (corresponding to position 424 of SEQ ID No. 2);
r) Ile, Pro, Arg or Ser at position 365 of the amino acid sequence of SEQ ID No. 16 (corresponding to position 425 of SEQ ID No. 2);
s) Glu, Phe, Thr or Val at position 366 of the amino acid sequence of SEQ ID No. 16 (corresponding to position 426 of SEQ ID No. 2), preferably Glu;
t) Ile, Met, Gln or Val at position 371 of the amino acid sequence of SEQ ID No. 16 (corresponding to position 431 of SEQ ID No. 2);
u) Ala, Cys, Gly or Thr at a position in an HPPD protein, said position corresponding to position 185 of the amino acid sequence of SEQ ID No. 2 (corresponding to position 228 of SEQ ID No. 2);
v) Ala, Glu, Gly, Lys, Leu, Asn, Gln, Arg, Ser, Thr or Tyr at a position in an HPPD protein, said position corresponding to position 207 of the amino acid sequence of SEQ ID No. 2 (corresponding to position 248 of SEQ ID No. 2);
w) Ala, Ile, Leu or Met at a position in an HPPD protein, said position corresponding to position 229 of the amino acid sequence of SEQ ID No. 2 (corresponding to position 270 of SEQ ID No. 2);
x) Glu, His, Ile, Lys, Leu, Met, Arg, Ser, Thr or Val at a position in an HPPD protein, said position corresponding to position 230 of the amino acid sequence of SEQ ID No. 2 (corresponding to position 271 of SEQ ID No. 2);
y) His at a position in an HPPD protein, said position corresponding to position 334 of the amino acid sequence of SEQ ID No. 2 (corresponding to position 379 of SEQ ID No. 2); and
z) Arg at a position in an HPPD protein, said position corresponding to position 367 of the amino acid sequence of SEQ ID No. 2 (corresponding to position 427 of SEQ ID No. 2).

11. The isolated nucleic acid of claim 1, wherein said HPPD protein comprises the amino acid sequence of SEQ ID No. 18 and wherein the resulting amino acid sequence comprises at least one amino acid selected from:
a) Ala, Asp, Glu, Phe, Gly, Lys, Gln, Arg, Ser, Thr, Val or Tyr at position 231 of the amino acid sequence of SEQ ID No. 18 (corresponding to position 250 of SEQ ID No. 2), optionally Phe or Ala;
b) Asp, Glu, Ile, Lys, Leu, Asn, Pro, Arg or Ser at position 232 of the amino acid sequence of SEQ ID No. 18 (corresponding to position 251 of SEQ ID No. 2), optionally Arg or Lys;
c) Phe, Gly, His, Ile, Leu, Met, Asn, Gln, Arg, Ser, Val or Tyr at position 233 of the amino acid sequence of SEQ ID No. 18 (corresponding to position 252 of SEQ ID No. 2), optionally Ser or Tyr;
d) Ala, His, Gln, Val or Trp at position 234 of the amino acid sequence of SEQ ID No. 18 (corresponding to position 253 of SEQ ID No. 2), optionally Val;
e) Val at position 246 of the amino acid sequence of SEQ ID No. 18 (corresponding to position 265 of SEQ ID No. 2);
f) Leu, Gln, Arg, Val or Tyr at position 249 of the amino acid sequence of SEQ ID No. 18 (corresponding to position 268 of SEQ ID No. 2);
g) Ala or Thr at position 250 of the amino acid sequence of SEQ ID No. 18 (corresponding to position 269 of SEQ ID No. 2), optionally Ala;
h) Ala, Ile, Asn, Thr or Val at position 261 of the amino acid sequence of SEQ ID No. 18 (corresponding to position 280 of SEQ ID No. 2);
i) Leu or Val at position 273 of the amino acid sequence of SEQ ID No. 18 (corresponding to position 293 of SEQ ID No. 2);
j) Met at position 274 of the amino acid sequence of SEQ ID No. 18 (corresponding to position 294 of SEQ ID No. 2);
k) Ile, Met or Asn at position 311 of the amino acid sequence of SEQ ID No. 18 (corresponding to position 335 of SEQ ID No. 2), optionally Met;
l) Tyr at position 356 of the amino acid sequence of SEQ ID No. 18 (corresponding to position 381 of SEQ ID No. 2);
m) Ser at position 367 of the amino acid sequence of SEQ ID No. 18 (corresponding to position 392 of SEQ ID No. 2);
n) Tyr at position 379 of the amino acid sequence of SEQ ID No. 18 (corresponding to position 419 of SEQ ID No. 2);
o) Asp, Lys or Asn at position 381 of the amino acid sequence of SEQ ID No. 18 (corresponding to position 421 of SEQ ID No. 2), optionally Lys;
p) Ala, Met, Pro or Thr at position 382 of the amino acid sequence of SEQ ID No. 18 (corresponding to position 422 of SEQ ID No. 2);
q) Ala, Ile or Val at position 384 of the amino acid sequence of SEQ ID No. 18 (corresponding to position 424 of SEQ ID No. 2);
r) Ile, Pro, Arg or Ser at position 385 of the amino acid sequence of SEQ ID No. 18 (corresponding to position 425 of SEQ ID No. 2);
s) Glu, Phe, Thr or Val at position 386 of the amino acid sequence of SEQ ID No. 18 (corresponding to position 426 of SEQ ID No. 2), optionally Glu;
t) Met, Gln or Val at position 391 of the amino acid sequence of SEQ ID No. 18 (corresponding to position 431 of SEQ ID No. 2);
u) Ala, Cys, Gly or Thr at a position in an HPPD protein, said position corresponding to position 207 of the amino acid sequence of SEQ ID No. 1 (corresponding to position 228 of SEQ ID No. 2)8;
v) Ala, Glu, Gly, His, Lys, Leu, Asn, Gln, Arg, Ser or Tyr at a position in an HPPD protein, said position corresponding to position 229 of the amino acid sequence of SEQ ID No. 18 (corresponding to position 248 of SEQ ID No. 2);
w) Ala, Ile, Leu or Met at a position in an HPPD protein, said position corresponding to position 251 of the amino acid sequence of SEQ ID No. 18 (corresponding to position 270 of SEQ ID No. 2);
x) Ala, Glu, His, Ile, Lys, Leu, Met, Arg, Thr or Val at a position in an HPPD protein, said position corresponding to position 252 of the amino acid sequence of SEQ ID No. 18 (corresponding to position 271 of SEQ ID No. 2);
y) His at a position in an HPPD protein, said position corresponding to position 354 of the amino acid sequence of SEQ ID No. 18 (corresponding to position 379 of SEQ ID No. 2); and
z) Arg at a position in an HPPD protein, said position corresponding to position 387 of the amino acid sequence of SEQ ID No. 18 (corresponding to position 427 of SEQ ID No. 2).

12. The isolated nucleic acid of claim 1, wherein said HPPD protein comprises the amino acid sequence of SEQ ID No. 30 and wherein the resulting amino acid sequence comprises at least one amino acid selected from:
a) Ala, Asp, Glu, Phe, Gly, Lys, Gln, Arg, Ser, Thr, Val or Tyr at position 232 of the amino acid sequence of SEQ ID No. 30 (corresponding to position 250 of SEQ ID No. 2), optionally Phe or Ala;
b) Asp, Glu, Ile, Lys, Leu, Asn, Pro, Arg or Ser at position 233 of the amino acid sequence of SEQ ID No. 30 (corresponding to position 251 of SEQ ID No. 2), optionally Arg or Lys;
C) Phe, Gly, His, Ile, Leu, Met, Asn, Gln, Arg, Ser, Val or Tyr at position 234 of the amino acid sequence of SEQ ID No. 30 (corresponding to position 252 of SEQ ID No. 2), optionally Ser or Tyr;
d) Ala, His, Gln, Val or Trp at position 235 of the amino acid sequence of SEQ ID No. 30 (corresponding to position 253 of SEQ ID No. 2), optionally Val;
e) Val at position 247 of the amino acid sequence of SEQ ID No. 30 (corresponding to position 265 of SEQ ID No. 2);
f) Leu, Gln, Arg, Val or Tyr at position 250 of the amino acid sequence of SEQ ID No. 30 (corresponding to position 268 of SEQ ID No. 2);
g) Ala or Thr at position 251 of the amino acid sequence of SEQ ID No. 30 (corresponding to position 269 of SEQ ID No. 2), optionally Ala;
h) Ala, Ile, Asn, Thr or Val at position 262 of the amino acid sequence of SEQ ID No. 30 (corresponding to position 280 of SEQ ID No. 2);
i) Leu or Val at position 274 of the amino acid sequence of SEQ ID No. 30 (corresponding to position 293 of SEQ ID No. 2);
j) Met at position 275 of the amino acid sequence of SEQ ID No. 30 (corresponding to position 294 of SEQ ID No. 2);
k) Ile, Met or Asn at position 312 of the amino acid sequence of SEQ ID No. 30 (corresponding to position 335 of SEQ ID No. 2), optionally Met;
l) Tyr at position 357 of the amino acid sequence of SEQ ID No. 30 (corresponding to position 381 of SEQ ID No. 2);
m) Ser at position 368 of the amino acid sequence of SEQ ID No. 30 (corresponding to position 392 of SEQ ID No. 2);
n) Tyr at position 380 of the amino acid sequence of SEQ ID No. 30 (corresponding to position 419 of SEQ ID No. 2);
o) Asp, Lys or Asn at position 382 of the amino acid sequence of SEQ ID No. 30 (corresponding to position 421 of SEQ ID No. 2), optionally Lys;
p) Ala, Met, Pro or Thr at position 383 of the amino acid sequence of SEQ ID No. 30 (corresponding to position 422 of SEQ ID No. 2);
q) Ala, Ile or Val at position 385 of the amino acid sequence of SEQ ID No. 30 (corresponding to position 424 of SEQ ID No. 2);
r) Ile, Pro, Arg or Ser at position 386 of the amino acid sequence of SEQ ID No. 30 (corresponding to position 425 of SEQ ID No. 2);
s) Glu, Phe, Thr or Val at position 387 of the amino acid sequence of SEQ ID No. 30 (corresponding to position 426 of SEQ ID No. 2), optionally Glu;
t) Met, Gln or Val at position 392 of the amino acid sequence of SEQ ID No. 30 (corresponding to position 431 of SEQ ID No. 2);
u) Ala, Cys, Gly or Thr at a position in an HPPD protein, said position corresponding to position 208 of the amino acid sequence of SEQ ID No. 30 (corresponding to position 228 of SEQ ID No. 2);
v) Ala, Glu, Gly, His, Lys, Leu, Asn, Gln, Arg, Ser or Tyr at a position in an HPPD protein, said position corresponding to position 230 of the amino acid sequence of SEQ ID No. 30 (corresponding to position 248 of SEQ ID No. 2);
w) Ala, Ile, Leu or Met at a position in an HPPD protein, said position corresponding to position 252 of the amino acid sequence of SEQ ID No. 30 (corresponding to position 270 of SEQ ID No. 2);
x) Glu, His, Ile, Lys, Leu, Met, Arg, Thr or Val at a position in an HPPD protein, said position corresponding to position 253 of the amino acid sequence of SEQ ID No. 30 (corresponding to position 271 of SEQ ID No. 2);
y) His at a position in an HPPD protein, said position corresponding to position 355 of the amino acid sequence of SEQ ID No. 30 (corresponding to position 379 of SEQ ID No. 2); and
z) Arg at a position in an HPPD protein, said position corresponding to position 388 of the amino acid sequence of SEQ ID No. 30 (corresponding to position 427 of SEQ ID No. 2).

13. The isolated nucleic acid of claim 1, wherein said HPPD protein comprises the amino acid sequence of SEQ ID No. 20 and wherein the resulting amino acid sequence comprises at least one amino acid selected from:
a) Ala, Asp, Glu, Phe, Gly, Lys, Gln, Arg, Ser, Thr, Val or Tyr at position 201 of the amino acid sequence of SEQ ID No. 20 (corresponding to position 250 of SEQ ID No. 2), optionally Phe or Ala;
b) Ala, Asp, Glu, Lys, Leu, Asn, Pro, Arg or Ser at position 202 of the amino acid sequence of SEQ ID No. 20 (corresponding to position 251 of SEQ ID No. 2), optionally Ala, Arg or Lys;
C) Glu, Phe, Gly, His, Ile, Leu, Met, Asn, Gln, Arg, Ser, Val or Tyr at position 203 of the amino acid sequence of SEQ ID No. 20 (corresponding to position 252 of SEQ ID No. 2), optionally Glu, Ser or Tyr;
d) Ala, His, Gln, Val or Trp at position 204 of the amino acid sequence of SEQ ID No. 20 (corresponding to position 253 of SEQ ID No. 2), optionally Val;
e) Val at position 216 of the amino acid sequence of SEQ ID No. 20 (corresponding to position 265 of SEQ ID No. 2);
f) Leu, Gln, Arg, Val or Tyr at position 219 of the amino acid sequence of SEQ ID No. 20 (corresponding to position 268 of SEQ ID No. 2);
g) Ala or Thr at position 220 of the amino acid sequence of SEQ ID No. 20 (corresponding to position 269 of SEQ ID No. 2), optionally Ala;
h) Ala, Ile, Asn, Thr or Val at position 230 of the amino acid sequence of SEQ ID No. 20 (corresponding to position 280 of SEQ ID No. 2);
i) Leu or Val at position 242 of the amino acid sequence of SEQ ID No. 20 (corresponding to position 293 of SEQ ID No. 2);
j) Met at position 243 of the amino acid sequence of SEQ ID No. 20 (corresponding to position 294 of SEQ ID No. 2);
k) Ile, Met or Asn at position 280 of the amino acid sequence of SEQ ID No. 20 (corresponding to position 335 of SEQ ID No. 2), optionally Met;
l) Phe or Tyr at position 323 of the amino acid sequence of SEQ ID No. 20 (corresponding to position 381 of SEQ ID No. 2);
m) Ser at position 334 of the amino acid sequence of SEQ ID No. 20 (corresponding to position 392 of SEQ ID No. 2);
n) Tyr at position 346 of the amino acid sequence of SEQ ID No. 20 (corresponding to position 419 of SEQ ID No. 2);
o) Asp or Lys at position 348 of the amino acid sequence of SEQ ID No. 20 (corresponding to position 421 of SEQ ID No. 2), optionally Lys;
p) Ala, Met, Pro or Thr at position 349 of the amino acid sequence of SEQ ID No. 20 (corresponding to position 422 of SEQ ID No. 2);
q) Ala, Ile or Val at position 351 of the amino acid sequence of SEQ ID No. 20 (corresponding to position 424 of SEQ ID No. 2);
r) Ile, Pro, Arg or Ser at position 352 of the amino acid sequence of SEQ ID No. 20 (corresponding to position 425 of SEQ ID No. 2);
s) Glu, Phe, Thr or Val at position 353 of the amino acid sequence of SEQ ID No. 20 (corresponding to position 426 of SEQ ID No. 2), preferably Glu;
t) Met, Gln, Phe or Val at position 358 of the amino acid sequence of SEQ ID No. 20 (corresponding to position 431 of SEQ ID No. 2);
u) Ala, Cys, Gly or Thr at position 177 of the amino acid sequence of SEQ ID No. 20 (corresponding to position 228 of SEQ ID No. 2);
v) Ala, Glu, Gly, His, Lys, Leu, Asn, Gln, Arg, Ser, Thr or Tyr at position 199 of the amino acid sequence of SEQ ID No. 20 (corresponding to position 248 of SEQ ID No. 2);
w) Ala, Ile, Leu or Met at a position at position 221 of the amino acid sequence of SEQ ID No. 20 (corresponding to position 270 of SEQ ID No. 2);
x) Ala, Glu, His, Ile, Leu, Met, Arg, Ser, Thr or Val at position 222 of the amino acid sequence of SEQ ID No. 20 (corresponding to position 271 of SEQ ID No. 2);
y) His at position 321 of the amino acid sequence of SEQ ID No. 20 (corresponding to position 379 of SEQ ID No. 2); and
z) Arg at position 354 of the amino acid sequence of SEQ ID No. 20 (corresponding to position 427 of SEQ ID No. 2).

14. The isolated nucleic acid of claim 1, wherein said HPPD protein comprises the amino acid sequence of SEQ ID No. 22 and wherein the resulting amino acid sequence comprises at least one amino acid selected from:
a) Ala, Asp, Glu, Phe, Gly, Lys, Gln, Arg, Ser, Thr, Val or Tyr at position 219 of the amino acid sequence of SEQ ID No. 22 (corresponding to position 250 of SEQ ID No. 2), optionally Phe or Ala;
b) Ala, Asp, Glu, Lys, Leu, Asn, Pro, Arg or Ser at position 220 of the amino acid sequence of SEQ ID No. 22 (corresponding to position 251 of SEQ ID No. 2), optionally Ala, Arg or Lys;
C) Glu, Phe, Gly, His, Ile, Leu, Met, Asn, Gln, Arg, Val or Tyr at position 221 of the amino acid sequence of SEQ ID No. 22 (corresponding to position 252 of SEQ ID No. 2), optionally Glu or Tyr;
d) Ala, His, Gln, Val or Trp at position 222 of the amino acid sequence of SEQ ID No. 22 (corresponding to position 253 of SEQ ID No. 2), optionally Val;
e) Val at position 234 of the amino acid sequence of SEQ ID No. 22 (corresponding to position 265 of SEQ ID No. 2);
f) Leu, Gln, Arg, Val or Tyr at position 237 of the amino acid sequence of SEQ ID No. 22 (corresponding to position 268 of SEQ ID No. 2);
g) Ala or Thr at position 238 of the amino acid sequence of SEQ ID No. 22 (corresponding to position 269 of SEQ ID No. 2), optionally Ala;
h) Ala, Ile, Asn, Thr or Val at position 249 of the amino acid sequence of SEQ ID No. 22 (corresponding to position 280 of SEQ ID No. 2);
i) Leu or Val at position 261 of the amino acid sequence of SEQ ID No. 22 (corresponding to position 293 of SEQ ID No. 2);
j) Met at position 262 of the amino acid sequence of SEQ ID No. 22 (corresponding to position 294 of SEQ ID No. 2);
k) Ile, Met or Asn at position 299 of the amino acid sequence of SEQ ID No. 22 (corresponding to position 335 of SEQ ID No. 2), optionally Met;
l) Tyr at position 342 of the amino acid sequence of SEQ ID No. 22 (corresponding to position 381 of SEQ ID No. 2);
m) Ser at position 353 of the amino acid sequence of SEQ ID No. 22 (corresponding to position 392 of SEQ ID No. 2);
n) Tyr at position 365 of the amino acid sequence of SEQ ID No. 22 (corresponding to position 419 of SEQ ID No. 2);
o) Asp, Lys or Asn at position 367 of the amino acid sequence of SEQ ID No. 22 (corresponding to position 421 of SEQ ID No. 2), optionally Lys;
p) Ala, Met, Pro or Thr at position 368 of the amino acid sequence of SEQ ID No. 22 (corresponding to position 422 of SEQ ID No. 2);
q) Ala, Ile or Val at position 370 of the amino acid sequence of SEQ ID No. 22 (corresponding to position 424 of SEQ ID No. 2);
r) Ile, Pro, Arg or Ser at position 371 of the amino acid sequence of SEQ ID No. 22 (corresponding to position 425 of SEQ ID No. 2);
s) Glu, Phe, Thr or Val at position 372 of the amino acid sequence of SEQ ID No. 22 (corresponding to position 426 of SEQ ID No. 2), optionally Glu;
t) Met, Gln or Val at position 377 of the amino acid sequence of SEQ ID No. 22 (corresponding to position 431 of SEQ ID No. 2);
u) Ala, Cys, Gly or Thr at position 195 of the amino acid sequence of SEQ ID No. 22 (corresponding to position 228 of SEQ ID No. 2);
v) Glu, Gly, His, Lys, Leu, Asn, Gln, Arg, Ser, Thr or Tyr at position 217 of the amino acid sequence of SEQ ID No. 22 (corresponding to position 248 of SEQ ID No. 2);
w) Ala, Ile, Leu or Val at position 239 of the amino acid sequence of SEQ ID No. 22 (corresponding to position 270 of SEQ ID No. 2);
x) Ala, Glu, His, Ile, Lys, Leu, Met, Arg, Thr or Val at position 240 of the amino acid sequence of SEQ ID No. 22 (corresponding to position 271 of SEQ ID No. 2);
y) His at a position at position 340 of the amino acid sequence of SEQ ID No. 22 (corresponding to position 379 of SEQ ID No. 2); and
z) Arg at a position at position 373 of the amino acid sequence of SEQ ID No. 22 (corresponding to position 427 of SEQ ID No. 2).

15. The isolated nucleic acid of claim 1, wherein said HPPD protein comprises the amino acid sequence of SEQ ID No. 24 and wherein the resulting amino acid sequence comprises at least one amino acid selected from
a) Ala, Asp, Glu, Gly, Lys, Gln, Arg, Ser, Thr, Val or Tyr at position 241 of the amino acid sequence of SEQ ID No. 24 (corresponding to position 250 of SEQ ID No. 2), optionally Ala;
b) Asp, Glu, Lys, Leu, Asn, Pro, Arg or Ser at position 242 of the amino acid sequence of SEQ ID No. 24 (corresponding to position 251 of SEQ ID No. 2), optionally Arg or Lys;
C) Phe, Gly, His, Ile, Leu, Met, Asn, Gln, Arg, Val, or Tyr at position 243 of the amino acid sequence of SEQ ID No. 24 (corresponding to position 252 of SEQ ID No. 2), optionally Tyr;
d) Ala, His, Gln, Val or Trp at position 244 of the amino acid sequence of SEQ ID No. 24 (corresponding to position 253 of SEQ ID No. 2), optionally Val;
e) Val at position 256 of the amino acid sequence of SEQ ID No. 24 (corresponding to position 265 of SEQ ID No. 2);
f) Leu, Gln, Arg, Val or Tyr at position 259 of the amino acid sequence of SEQ ID No. 24 (corresponding to position 268 of SEQ ID No. 2);
g) Ala or Thr at position 260 of the amino acid sequence of SEQ ID No. 24 (corresponding to position 269 of SEQ ID No. 2), optionally Ala;
h) Ala, Ile, Asn, Thr or Val at position 271 of the amino acid sequence of SEQ ID No. 24 (corresponding to position 280 of SEQ ID No. 2);
i) Leu or Val at position 284 of the amino acid sequence of SEQ ID No. 24 (corresponding to position 293 of SEQ ID No. 2);
j) Met at position 285 of the amino acid sequence of SEQ ID No. 24 (corresponding to position 294 of SEQ ID No. 2);
k) Ile or Asn at position 326 of the amino acid sequence of SEQ ID No. 24 (corresponding to position 335 of SEQ ID No. 2);
l) Tyr at position 372 of the amino acid sequence of SEQ ID No. 24 (corresponding to position 381 of SEQ ID No. 2);
m) Ser at position 383 of the amino acid sequence of SEQ ID No. 24 (corresponding to position 392 of SEQ ID No. 2);
n) Tyr at position 410 of the amino acid sequence of SEQ ID No. 24 (corresponding to position 419 of SEQ ID No. 2);
o) Asp or Asn at position 412 of the amino acid sequence of SEQ ID No. 24 (corresponding to position 421 of SEQ ID No. 2);
p) Ala, Met, Pro or Thr at position 413 of the amino acid sequence of SEQ ID No. 24 (corresponding to position 422 of SEQ ID No. 2);
q) Ala, Ile or Val at position 415 of the amino acid sequence of SEQ ID No. 24 (corresponding to position 424 of SEQ ID No. 2);
r) Ile, Pro or Arg at position 416 of the amino acid sequence of SEQ ID No. 24 (corresponding to position 425 of SEQ ID No. 2);
s) Phe, Thr or Val at position 417 of the amino acid sequence of SEQ ID No. 24 (corresponding to position 426 of SEQ ID No. 2);
t) Met, Gln or Val at position 422 of the amino acid sequence of SEQ ID No. 24 (corresponding to position 431 of SEQ ID No. 2);
u) Ala, Cys, Gly or Thr at position 219 of the amino acid sequence of SEQ ID No. 24 (corresponding to position 228 of SEQ ID No. 2);
v) Ala, Glu, Gly, Lys, Leu, Asn, Gln, Arg, Ser, Thr or Tyr at position 239 of the amino acid sequence of SEQ ID No. 24 (corresponding to position 248 of SEQ ID No. 2);
w) Ala, Ile, Met or Val at position 261 of the amino acid sequence of SEQ ID No. 24 (corresponding to position 270 of SEQ ID No. 2);
x) Glu, His, Ile, Lys, Leu, Met, Arg, Ser, Thr or Val at position 262 of the amino acid sequence of SEQ ID No. 24 (corresponding to position 271 of SEQ ID No. 2);
y) His at position 370 of the amino acid sequence of SEQ ID No. 24 (corresponding to position 379 of SEQ ID No. 2); and
z) Arg at position 418 of the amino acid sequence of SEQ ID No. 24 (corresponding to position 427 of SEQ ID No. 2).

16. The isolated nucleic acid of claim 1, wherein said HPPD protein comprises the amino acid sequence of SEQ ID No. 26 and wherein the resulting amino acid sequence comprises at least one amino acid selected from
a) Ala, Asp, Glu, Gly, Lys, Gln, Arg, Ser, Thr, Val or Tyr at position 237 of the amino acid sequence of SEQ ID No. 26 (corresponding to position 250 of SEQ ID No. 2), optionally Ala;
b) Asp, Glu, Lys, Leu, Asn, Pro, Arg or Ser at position 238 of the amino acid sequence of SEQ ID No. 26 (corresponding to position 251 of SEQ ID No. 2), optionally Arg or Lys;
C) Phe, Gly, His, Ile, Leu, Met, Asn, Gln, Arg, Val or Tyr at position 239 of the amino acid sequence of SEQ ID No. 26 (corresponding to position 252 of SEQ ID No. 2), optionally Tyr;
d) Ala, His, Gln, Val or Trp at position 240 of the amino acid sequence of SEQ ID No. 26 (corresponding to position 253 of SEQ ID No. 2), optionally Val;
e) Val at position 252 of the amino acid sequence of SEQ ID No. 26 (corresponding to position 265 of SEQ ID No. 2);
f) Leu, Gln, Arg, Val or Tyr at position 255 of the amino acid sequence of SEQ ID No. 26 (corresponding to position 268 of SEQ ID No. 2);
g) Ala or Thr at position 256 of the amino acid sequence of SEQ ID No. 26 (corresponding to position 269 of SEQ ID No. 2), optionally Ala;
h) Ala, Ile, Asn, Thr or Val at position 267 of the amino acid sequence of SEQ ID No. 26 (corresponding to position 280 of SEQ ID No. 2);
i) Leu or Val at position 280 of the amino acid sequence of SEQ ID No. 26 (corresponding to position 293 of SEQ ID No. 2);
j) Met at position 281 of the amino acid sequence of SEQ ID No. 26 (corresponding to position 294 of SEQ ID No. 2);
k) Ile, Met or Asn at position 322 of the amino acid sequence of SEQ ID No. 26 (corresponding to position 335 of SEQ ID No. 2), optionally Met;
l) Tyr at position 368 of the amino acid sequence of SEQ ID No. 26 (corresponding to position 381 of SEQ ID No. 2);
m) Ser at position 379 of the amino acid sequence of SEQ ID No. 26 (corresponding to position 392 of SEQ ID No. 2);
n) Tyr at position 406 of the amino acid sequence of SEQ ID No. 26 (corresponding to position 419 of SEQ ID No. 2);
o) Asp or Asn at position 408 of the amino acid sequence of SEQ ID No. 26 (corresponding to position 421 of SEQ ID No. 2);
p) Ala, Met, Pro or Thr at position 409 of the amino acid sequence of SEQ ID No. 26 (corresponding to position 422 of SEQ ID No. 2);
q) Ala, Ile or Val at position 411 of the amino acid sequence of SEQ ID No. 26 (corresponding to position 424 of SEQ ID No. 2);
r) Ile, Pro, Arg at position 412 of the amino acid sequence of SEQ ID No. 26 (corresponding to position 425 of SEQ ID No. 2);
s) Phe, Thr or Val at position 413 of the amino acid sequence of SEQ ID No. 26 (corresponding to position 426 of SEQ ID No. 2);
t) Met or Gln or Val at position 418 of the amino acid sequence of SEQ ID No. 26 (corresponding to position 431 of SEQ ID No. 2);
u) Ala, Cys, Gly or Thr at a position in an HPPD protein, said position corresponding to position 215 of the amino acid sequence of SEQ ID No. 26 (corresponding to position 228 of SEQ ID No. 2);
v) Ala, Glu, Gly, Lys, Leu, Asn, Gln, Arg, Ser, Thr or Tyr at position 235 of the amino acid sequence of SEQ ID No. 26 (corresponding to position 248 of SEQ ID No. 2);
w) Ala, Ile, Met or Val at position 257 of the amino acid sequence of SEQ ID No. 26 (corresponding to position 270 of SEQ ID No. 2);
x) Glu, His, Ile, Lys, Leu, Met, Arg, Ser, Thr or Val at position 258 of the amino acid sequence of SEQ ID No. 26 (corresponding to position 271 of SEQ ID No. 2);
y) His at a position at position 366 of the amino acid sequence of SEQ ID No. 26 (corresponding to position 379 of SEQ ID No. 2); and
z) Arg at position 414 of the amino acid sequence of SEQ ID No. 26 (corresponding to position 427 of SEQ ID No. 2).

17. The isolated nucleic acid of claim 1, wherein said HPPD protein comprises the amino acid sequence of SEQ ID No. 2 and wherein the resulting amino acid sequence comprises at least one amino acid selected from
a) Ala, Asp, Glu, Gly, Lys, Gln, Arg, Ser, Thr, Val or Tyr at position 250 of the amino acid sequence of SEQ ID No. 2;
b) Asp, Glu, Ile, Lys, Leu, Asn, Pro, Arg or Ser at position 251 of the amino acid sequence of SEQ ID No. 2;
C) Phe, Gly, His, Ile, Leu, Met, Asn, Gln, Arg, Ser, Val or Tyr at position 252 of the amino acid sequence of SEQ ID No. 2;
d) Ala, His, Gln, Val or Trp at position 253 of the amino acid sequence of SEQ ID No. 2;
e) Val at a position at position 265 of the amino acid sequence of SEQ ID No. 2;
f) Leu, Gln, Arg, Val or Tyr at position 268 of the amino acid sequence of SEQ ID No. 2;
g) Ala or Thr at position 269 of the amino acid sequence of SEQ ID No. 2;
h) Ala, Ile, Asn, Thr or Val at position 280 of the amino acid sequence of SEQ ID No. 2;
i) Leu or Val at position 293 of the amino acid sequence of SEQ ID No. 2;
j) Met at position 294 of the amino acid sequence of SEQ ID No. 2;
k) Ile or Asn at position 335 of the amino acid sequence of SEQ ID No. 2;
l) Tyr at position 381 of the amino acid sequence of SEQ ID No. 2;
m) Ser at position 392 of the amino acid sequence of SEQ ID No. 2;
n) Tyr at position 419 of the amino acid sequence of SEQ ID No. 2;
o) Asp or Asn at position 421 of the amino acid sequence of SEQ ID No. 2;
p) Ala, Met, Pro or Thr at position 422 of the amino acid sequence of SEQ ID No. 2;
q) Ala, Ile or Val at position 424 of the amino acid sequence of SEQ ID No. 2;
r) Ile, Pro or Arg at position 425 of the amino acid sequence of SEQ ID No. 2;
s) Phe, Thr or Val at position 426 of the amino acid sequence of SEQ ID No. 2;
t) Met, Gln or Val position 431 of the amino acid sequence of SEQ ID No. 2;
u) Ala, Cys, Gly or Thr at position 228 of the amino acid sequence of SEQ ID No. 2;
v) Ala, Glu, Gly, Lys, Leu, Asn, Gln, Arg, Ser, Thr or Tyr at position 248 of the amino acid sequence of SEQ ID No. 2;
w) Ala, Ile, Met or Val at position 270 of the amino acid sequence of SEQ ID No. 2;
x) Glu, His, Ile, Lys, Leu, Met, Arg, Ser, Thr or Val at position 271 of the amino acid sequence of SEQ ID No. 2;
y) His at position 379 of the amino acid sequence of SEQ ID No. 2; and
z) Arg at position 427 of the amino acid sequence of SEQ ID No. 2.

18. The nucleic acid of claim 1, wherein in said mutated HPPD protein at least one amino acid has been replaced so that the resulting amino acid sequence comprises (positions corresponding to SEQ ID No. 2) (a) Glu, Ser or Tyr at position 252; (b) Val or Ala at position 269; (c) Pro, Val or Ala at position 280; (d) Asp, Lys or Asn at position 421; and (e) Gly or Ala at position 422.

19. The nucleic acid of claim 1, wherein in said mutated HPPD protein at least two amino acids have been replaced so that the resulting amino acid sequence comprises at least two amino acids selected from:
a) Ala, Asp, Glu, Phe, Gly, Lys, Gln, Arg, Ser, Thr, Val or Tyr at a position in an HPPD protein, said position corresponding to position 250 of the amino acid sequence of SEQ ID No. 2;
b) Ala, Asp, Glu, Ile, Lys, Leu, Asn, Pro, Arg or Ser at a position in an HPPD protein, said position corresponding to position 251 of the amino acid sequence of SEQ ID No. 2;
c) Glu, Phe, Gly, His, Ile, Leu, Met, Asn, Gln, Arg, Ser, Val or Tyr at a position in an HPPD protein, said position corresponding to position 252 of the amino acid sequence of SEQ ID No. 2;
d) Ala, Phe, His, Gln, Val or Trp at a position in an HPPD protein, said position corresponding to position 253 of the amino acid sequence of SEQ ID No. 2;
e) Leu or Val at a position in an HPPD protein, said position corresponding to position 265 of the amino acid sequence of SEQ ID No. 2;
f) Leu, Gln, Arg, Val or Tyr at a position in an HPPD protein, said position corresponding to position 268 of the amino acid sequence of SEQ ID No. 2;
g) Ala, Thr or Val at a position in an HPPD protein, said position corresponding to position 269 of the amino acid sequence of SEQ ID No. 2;
h) Ala, Ile, Asn, Pro, Thr or Val at a position in an HPPD protein, said position corresponding to position 280 of the amino acid sequence of SEQ ID No. 2;
i) Leu, Gln or Val at a position in an HPPD protein, said position corresponding to position 293 of the amino acid sequence of SEQ ID No. 2;
j) Ile or Met at a position in an HPPD protein, said position corresponding to position 294 of the amino acid sequence of SEQ ID No. 2;
k) Gln at a position in an HPPD protein, said position corresponding to position 307 of the amino acid sequence of SEQ ID No. 2;
l) Ile, Met or Asn at a position in an HPPD protein, said position corresponding to position 335 of the amino acid sequence of SEQ ID No. 2;
m) Leu at a position in an HPPD protein, said position corresponding to position 368 of the amino acid sequence of SEQ ID No. 2;
n) Phe or Tyr at a position in an HPPD protein, said position corresponding to position 381 of the amino acid sequence of SEQ ID No. 2;
o) Phe or Ser at a position in an HPPD protein, said position corresponding to position 392 of the amino acid sequence of SEQ ID No. 2;
p) Phe or Tyr at a position in an HPPD protein, said position corresponding to position 419 of the amino acid sequence of SEQ ID No. 2;
q) Asp, Lys or Asn at a position in an HPPD protein, said position corresponding to position 421 of the amino acid sequence of SEQ ID No. 2;
r) Ala, Gly, Met, Pro or Thr at a position in an HPPD protein, said position corresponding to position 422 of the amino acid sequence of SEQ ID No. 2;
s) Ala, Phe, Ile or Val at a position in an HPPD protein, said position corresponding to position 424 of the amino acid sequence of SEQ ID No. 2;
t) Ile, Pro, Arg or Ser at a position in an HPPD protein, said position corresponding to position 425 of the amino acid sequence of SEQ ID No. 2;
u) Glu, Phe, Thr or Val at a position in an HPPD protein, said position corresponding to position 426 of the amino acid sequence of SEQ ID No. 2;
v) Ile, Met, Gln or Val at a position in an HPPD protein, said position corresponding to position 431 of the amino acid sequence of SEQ ID No. 2; and;
w) at least one amino acid deletion or replacement at any one of positions 228, 248, 270, 271, 379 and/or 427.

20. An isolated nucleic acid comprising a nucleotide sequence encoding a mutated HPPD protein, wherein said mutated HPPD protein has HPPD activity, and wherein in said mutated HPPD protein at least one amino acid at position 228, 248, 270, 271, 379 and/or 427, according to the numbering of SEQ ID No. 2, has been replaced by another amino acid.

21. The isolated nucleic acid of claim 20, wherein in said mutated HPPD protein at least one amino acid has been replaced so that the resulting amino acid sequence comprises at least one amino acid selected from:
a. Ala, Cys, Gly, Thr or Val at a position in an HPPD protein, said position corresponding to position 228 of the amino acid sequence of SEQ ID No. 2;
b. Ala, Glu, Gly, His, Lys, Leu, Asn, Gln, Arg, Ser, Thr or Tyr at a position in an HPPD protein, said position corresponding to position 248 of the amino acid sequence of SEQ ID No. 2;
c. Ala, Ile, Leu, Met or Val at a position in an HPPD protein, said position corresponding to position 270 of the amino acid sequence of SEQ ID No. 2;
d. Ala, Glu, His, Ile, Lys, Leu, Met, Arg, Ser, Thr or Val at a position in an HPPD protein, said position corresponding to position 271 of the amino acid sequence of SEQ ID No. 2;
e. His or Gln at a position in an HPPD protein, said position corresponding to position 379 of the amino acid sequence of SEQ ID No. 2; and
f. Leu or Arg at a position in an HPPD protein, said position corresponding to position 427 of the amino acid sequence of SEQ ID No. 2.

22. The isolated nucleic acid of claim 21, wherein in said mutated HPPD protein at least one amino acid has been replaced so that the resulting amino acid sequence comprises at least one amino acid selected from
a) Val or Thr at a position in an HPPD protein, said position corresponding to position 228 of the amino acid sequence of SEQ ID No. 2;
b) Leu, Met or Val at a position in an HPPD protein, said position corresponding to position 270 of the amino acid sequence of SEQ ID No. 2;
c) Ala or Ser at a position in an HPPD protein, said position corresponding to position 271 of the amino acid sequence of SEQ ID No. 2,
d) Gln at a position in an HPPD protein, said position corresponding to position 379 of the amino acid sequence of SEQ ID No. 2; and
e) Leu at a position in an HPPD protein, said position corresponding to position 427 of the amino acid sequence of SEQ ID No. 2.

23. The nucleic acid of claim 1, wherein said mutated HPPD protein is capable of increasing the tolerance of a plant to at least one herbicide acting on HPPD.

24. A protein encoded by the isolated nucleic acid of claim 1.

25. A chimeric gene comprising a coding sequence comprising the nucleic acid of claim 1, operably linked to a plant-expressible promoter and optionally a transcription termination and polyadenylation region.

26. A method of obtaining a mutated HPPD protein capable of modulating the tolerance of a plant to at least one herbicide acting on HPPD, wherein said mutated HPPD protein has HPPD activity, the method comprising:
i) providing an HPPD protein;
ii) replacing at least one amino acid in said HPPD protein so that the resulting amino acid sequence comprises at least one selected from
a) Ala, Asp, Glu, Phe, Gly, Lys, Gln, Arg, Ser, Thr, Val or Tyr at a position in an HPPD protein, said position corresponding to position 250 of the amino acid sequence of SEQ ID No. 2;
b) Ala, Asp, Glu, Ile, Lys, Leu, Asn, Pro, Arg or Ser at a position in an HPPD protein, said position corresponding to position 251 of the amino acid sequence of SEQ ID No. 2;
c) Glu, Phe, Gly, His, Ile, Leu, Met, Asn, Gln, Arg, Ser, Val or Tyr at a position in an HPPD protein, said position corresponding to position 252 of the amino acid sequence of SEQ ID No. 2;
d) Ala, Phe, His, Gln, Val or Trp at a position in an HPPD protein, said position corresponding to position 253 of the amino acid sequence of SEQ ID No. 2;
e) Leu or Val at a position in an HPPD protein, said position corresponding to position 265 of the amino acid sequence of SEQ ID No. 2;
f) Leu, Gln, Arg, Val or Tyr at a position in an HPPD protein, said position corresponding to position 268 of the amino acid sequence of SEQ ID No. 2;
g) Ala, Thr or Val at a position in an HPPD protein, said position corresponding to position 269 of the amino acid sequence of SEQ ID No. 2;
h) Ala, Ile, Asn, Pro, Thr or Val at a position in an HPPD protein, said position corresponding to position 280 of the amino acid sequence of SEQ ID No. 2;
i) Leu, Gln or Val at a position in an HPPD protein, said position corresponding to position 293 of the amino acid sequence of SEQ ID No. 2;
j) Ile or Met at a position in an HPPD protein, said position corresponding to position 294 of the amino acid sequence of SEQ ID No. 2;
k) Gln at a position in an HPPD protein, said position corresponding to position 307 of the amino acid sequence of SEQ ID No. 2;
l) Ile, Met or Asn at a position in an HPPD protein, said position corresponding to position 335 of the amino acid sequence of SEQ ID No. 2;
m) Leu at a position in an HPPD protein, said position corresponding to position 368 of the amino acid sequence of SEQ ID No. 2;
n) Phe or Tyr at a position in an HPPD protein, said position corresponding to position 381 of the amino acid sequence of SEQ ID No. 2;
o) Phe or Ser at a position in an HPPD protein, said position corresponding to position 392 of the amino acid sequence of SEQ ID No. 2;
p) Phe or Tyr at a position in an HPPD protein, said position corresponding to position 419 of the amino acid sequence of SEQ ID No. 2;
q) Asp, Phe, Lys, Asn at a position in an HPPD protein, said position corresponding to position 421 of the amino acid sequence of SEQ ID No. 2;
r) Ala, Gly, Met, Pro or Thr at a position in an HPPD protein, said position corresponding to position 422 of the amino acid sequence of SEQ ID No. 2;
s) Ala, Phe, Ile or Val at a position in an HPPD protein, said position corresponding to position 424 of the amino acid sequence of SEQ ID No. 2;
t) Ile, Pro, Arg or Ser at a position in an HPPD protein, said position corresponding to position 425 of the amino acid sequence of SEQ ID No. 2;
u) Glu, Phe, Thr or Val at a position in an HPPD protein, said position corresponding to position 426 of the amino acid sequence of SEQ ID No. 2;
v) Ile, Met, Gln or Val at a position in an HPPD protein, said position corresponding to position 431 of the amino acid sequence of SEQ ID No. 2; and
w) an amino acid deletion or replacement at at least one position in an HPPD protein, said position corresponding to at least one of positions 228, 248, 270, 271, 379 and 427 of the amino acid sequence of SEQ ID No. 2;
iii) determining the inhibition of the resulting HPPD protein by at least one herbicide acting on HPPD,
wherein an inhibition of the resulting protein of less or more than that observed with a reference HPPD protein is indicative that the resulting protein is capable of modulating the tolerance of a plant to said herbicide.

27. The method of claim 26, wherein said mutated HPPD protein is capable of increasing the tolerance of a plant to at least one HPPD inhibitor herbicide.

28. The method of claim 27, wherein the herbicide acting on HPPD is tembotrione.

29. A method of producing a transgenic plant comprising introducing into a said plant the nucleic acid of claim 1, and obtaining progeny thereof comprising said operably linked nucleic acid.

30. The method of claim 29, wherein the nucleic acid is introduced into said plant.

31. The method of claim 26, wherein said HPPD protein comprises
a) a His at a position in an HPPD protein, said position corresponding to position 226 of the amino acid sequence of SEQ ID No. 2;
b) a Ser at a position in an HPPD protein, said position corresponding to position 267 of the amino acid sequence of SEQ ID No. 2;
c) an Asn at a position in an HPPD protein, said position corresponding to position 282 of the amino acid sequence of SEQ ID No. 2;
d) a His at a position in an HPPD protein, said position corresponding to position 308 of the amino acid sequence of SEQ ID No. 2;
e) a Tyr at a position in an HPPD protein, said position corresponding to position 342 of the amino acid sequence of SEQ ID No. 2;
f) a Glu at a position in an HPPD protein, said position corresponding to position 394 of the amino acid sequence of SEQ ID No. 2;
g) a Gly at a position in an HPPD protein, said position corresponding to position 420 of the amino acid sequence of SEQ ID No. 2; and
h) an Asn at a position in an HPPD protein, said position corresponding to position 423 of the amino acid sequence of SEQ ID No. 2

32. A plant cell comprising the isolated nucleic acid of claim 1 in genetic information thereof.

33. A plant, part thereof or plant tissue consisting essentially of plant cells of claim 32.

34. A plant obtainable from the method of claim 29.

35. The plant of claim 33, which is selected from the group consisting of wheat, cotton, canola, rice, corn, soy bean, and sorghum.

36. A seed of the plant of claim 33.

37. The progeny of the plant of claim 33.

38. A method of modulating a plant's tolerance to at least one herbicide acting on HPPD or of obtaining a plant tolerant to an HPPD inhibitor herbicide comprising introducing the isolated nucleic acid of claim 1, operably linked to a plant expressible promoter into a plant's genome.

39. A method for controlling weeds comprising spraying at least one HPPD inhibitor herbicide on or around a crop plant, wherein said crop plant contains the nucleic acid according to claim 23 operably linked to a plant expressible promoter.

40. The chimeric gene of claim 25, operably linked to a plant expressible promoter capable of being used for modulating the tolerance of a plant to at least one HPPD inhibitor herbicide applied for controlling weeds.

41. The method of claim 30 wherein the tolerance of a plant to at least one HPPD inhibitor herbicide is increased.
